SEQUENCE LISTING (12 sequences)

SEQ ID NO: 1 - protein, 210 aa, Homo sapiens
  REGION 1..210: note = misc_feature - full length human frataxin
MWTLGRRAVAGLLASPSPAQAQTLTRVPRPAELAPLCGRRGLRTDIDATCTPRRASSNQRGLNQIWNVKKQSVYLMNLRKSGTLGHPGSLDETTYERLAEETLDSLAEFFEDLADKPYTFEDYDVSFGSGVLTVKLGGDLGTYVINKQTPNKQIWLSSPSSGPKRYDWTGKNWVYSHDGVSLHELLAAELTKALKTKLDLSSLAYSGKDA

SEQ ID NO: 2 - protein, 130 aa, Homo sapiens
  REGION 1..130: note = misc_feature - mature human frataxin
SGTLGHPGSLDETTYERLAEETLDSLAEFFEDLADKPYTFEDYDVSFGSGVLTVKLGGDLGTYVINKQTPNKQIWLSSPSSGPKRYDWTGKNWVYSHDGVSLHELLAAELTKALKTKLDLSSLAYSGKDA

SEQ ID NO: 3 - protein, 80 aa, Homo sapiens
  REGION 1..80: note = misc_feature - Mitochondrial targeting sequence from full length human frataxin
MWTLGRRAVAGLLASPSPAQAQTLTRVPRPAELAPLCGRRGLRTDIDATCTPRRASSNQRGLNQIWNVKKQSVYLMNLRK

SEQ ID NO: 4 - protein, 11 aa, synthetic construct
  REGION 1..11: note = Synthetic: HIV-TAT
YGRKKRRQRRR

SEQ ID NO: 5 - protein, 12 aa, synthetic construct
  REGION 1..12: note = Synthetic: HIV-TAT+M
MYGRKKRRQRRR

SEQ ID NO: 6 - protein, 30 aa, synthetic construct
  REGION 1..30: note = Synthetic: Galanin
GWTLNSAGYLLGPHAVGNHRSFSDKNGLTS

SEQ ID NO: 7 - protein, 14 aa, synthetic construct
  REGION 1..14: note = Synthetic: Mastoparan
INLKALAALAKKIL

SEQ ID NO: 8 - protein, 27 aa, synthetic construct
  REGION 1..27: note = Synthetic: Transportan
GWTLNSAGYLLGKINLKALAALAKKIL

SEQ ID NO: 9 - protein, 16 aa, synthetic construct
  REGION 1..16: note = Synthetic: Penetratin
RQIKIWFQNRRMKWKK

SEQ ID NO: 10 - protein, 9 aa, synthetic construct
  REGION 1..9: note = Synthetic: Polyarginine
RRRRRRRRR

SEQ ID NO: 11 - protein, 34 aa, synthetic construct
  REGION 1..34: note = Synthetic: VP22
DAATATRGRSAASRPTERPRAPARSASRPRRPVE

SEQ ID NO: 12 - protein, 224 aa, synthetic construct
  REGION 1..224: note = Synthetic: exemplary FXN fusion protein
MYGRKKRRQRRRGGMWTLGRRAVAGLLASPSPAQAQTLTRVPRPAELAPLCGRRGLRTDIDATCTPRRASSNQRGLNQIWNVKKQSVYLMNLRKSGTLGHPGSLDETTYERLAEETLDSLAEFFEDLADKPYTFEDYDVSFGSGVLTVKLGGDLGTYVINKQTPNKQIWLSSPSSGPKRYDWTGKNWVYSHDGVSLHELLAAELTKALKTKLDLSSLAYSGKDA

(19) United States
(12) Patent Application Publication
(10) Pub. No.: US 2024/0142476 A1
(43) Pub. Date: May 2, 2024

(54) FRATAXIN-SENSITIVE LIPID MARKERS FOR MONITORING FRATAXIN REPLACEMENT THERAPY

(71) Applicant: Larimar Therapeutics, Inc., Bala Cynwyd, PA (US)

(72) Inventors: Joan David Bettoun, Elkins Park, PA (US); Christine Des Rosiers, Rigaud (CA); Anik Forest, Sainte-Lucie-Des-Laurentides (CA)

(21) Appl. No.: 18/315,445

(22) Filed: May 10, 2023

Related U.S. Application Data
(60) Provisional application No. 63/351,380, filed on Jun. 11, 2022.
(60) Provisional application No. 63/340,412, filed on May 10, 2022.

(51) Int. Cl. G01N 33/92

(52) U.S. Cl. CPC G01N 33/92; G01N 2405/02; G01N 2405/04; G01N 2800/28; G01N 2800/52

(57) ABSTRACT

The present disclosure is based, at least in part, on providing a set of markers, also referred to herein as FXN-sensitive lipid markers (or FSLMs), the respective levels of which are positively or negatively correlated to frataxin (FXN) levels in a cell. Therefore, these FSLMs can be used to determine, evaluate, and/or monitor the effectiveness of FXN replacement therapy in a subject.

### RELATED APPLICATIONS

This application claims priority to U.S. Provisional Application No. 63/340,412, filed on May 10, 2022 and U.S. Provisional Application No. 63/351,380, filed on Jun. 11, 2022. The entire contents of each of the foregoing applications are hereby incorporated herein by reference.

### SEQUENCE LISTING

The instant application contains a Sequence Listing which has been submitted electronically in ASCII format and is hereby incorporated by reference in its entirety. Said ASCII copy, created on May 8, 2023, is named 130197-01303_SL.xml and is 16,411 bytes in size.

### BACKGROUND

Mitochondrial diseases are a group of disorders caused by dysfunctional mitochondria, the cellular organelles that store potential energy in the form of adenosine triphosphate (ATP) molecules and are found in every cell of the human body except mature red blood cells.

Friedreich's Ataxia (FRDA) is the most common inherited ataxia in humans and results from a deficiency of the mitochondrial protein frataxin (FXN), and specifically human frataxin, hFXN). FRDA is a rare disease with an estimated incidence of 1:29,000, a carrier frequency of ˜1:85, and about 4,000-5,000 reported cases in the United States. FRDA is a progressive multisystem disease, typically beginning in mid-childhood. Patients suffer from multiple symptoms, including progressive neurologic and cardiac dysfunction. Other clinical findings can include scoliosis, fatigue, diabetes, visual impairment, and hearing loss. Inheritance is autosomal recessive and is predominantly caused by an inherited GAA triplet expansion in the first intron of both alleles of the hFXN gene. This triplet expansion causes transcriptional repression of the FRDA gene, which results in the production of very small amounts of hFXN in patients. hFXN heterozygotes typically have hFXN levels at ˜50% of normal but are phenotypically normal. hFXN levels of ˜45-70 pg/μl and ˜5-25 pg/μl in whole blood of heterozygotes and patients afflicted with FRDA respectively have been shown to be stable over time (Plasterer et al., 2013).

Currently, there is no FDA-approved treatment for FRDA. Antioxidants and iron chelation have not been overly effective, and, despite treatment, patients typically experience progressive loss of motor control and die, cardiomyopathy being the primary cause of death.

Protein replacement therapy is a well-established approach to metabolic diseases, such as diabetes, lysosomal storage disorders and hemophilia. Work in patient-derived cellular and animal models has demonstrated that replacement of functional FXN can correct or improve the FRDA disease phenotype. However, there is a need in the art for a reliable and efficient assay to measure clinical response and effectiveness of FXN replacement.

### SUMMARY

In one aspect, the present disclosure is based, at least in part, on providing a set of markers, also referred herein as FXN-sensitive lipid markers (FSLMs), whose respective levels are positively or negatively correlated to frataxin (FXN) levels in a cell or a subject (e.g., a sample from a subject).

In some embodiments, the FSLMs of the present disclosure are contrary regulated by FXN gene ablation or FXN deficiency in a subject followed by FXN protein replacement. Thus, said FSLMs of the present disclosure are both associated with FXN deficiency in a subject and conversely associated with FXN replacement. The FSLMs disclosed herein were found to be sensitive to FXN levels and are considered markers of FXN deficiency, and of FXN replacement therapy.

Therefore, any one or more of the FSLMs provided herein can serve as surrogate biomarkers for FXN levels in a subject. For example, the FSLMs provided herein can be used to evaluate or to monitor progression of an FXN deficiency in a subject, as described herein. Further, the FSLMs provided herein can be used to evaluate and/or monitor an FXN replacement therapy, e.g., determine, evaluate and/or monitor the efficacy of FXN replacement therapy in a subject, as described herein.

In some embodiments, an FXN replacement therapy, e.g., efficacy of an FXN replacement therapy, in a subject can be determined, evaluated, and/or monitored based on the analysis of one or more FXN lipid profiles in samples obtained from a subject before, during and/or after administration or initiation of FXN replacement therapy to the subject. Based on the results of the FXN replacement lipid profile analysis, adjustments can be made to the FXN replacement therapy in a subject, such as to, e.g., initiate an FXN replacement therapy, increase a dose and/or administration frequency of an FXN replacement therapy, decrease a dose and/or administration frequency of an FXN replacement therapy, or cease FXN replacement therapy in a subject.

Accordingly, in some aspects, the present disclosure provides a method for evaluating efficacy of a frataxin (FXN) replacement therapy, the method comprising: (a) determining a baseline FSLM(−) lipid profile for one or more FXN-sensitive lipid markers (FSLMs) in a sample obtained from an FXN deficient subject prior to administration of the FXN replacement therapy; (b) determining an FXN replacement lipid profile for the one or more FXN-sensitive lipid markers (FSLMs) in a sample obtained from the FXN deficient subject following administration of the FXN replacement therapy; (c) comparing the FXN replacement lipid profile determined in step (b) with the baseline FXN(−) lipid profile determined in step (a); and (d) determining efficacy of the FXN replacement therapy based on the comparison in step (c); wherein the one or more FSLMs are selected from a group consisting of: triglycerides (TGs), wherein the three acyl groups in each triglyceride molecule contain less than 56 carbons and/or wherein the three acyl groups in each triglyceride molecule contain 7 or less unsaturations; ether phospholipids; phosphatidylcholines (PCs), cholesteryl esters (CEs); and diglycerides (DGs).

In some aspects, the present disclosure also provides a method for evaluating efficacy of a frataxin (FXN) replacement therapy, the method comprising: (a) determining an FXN replacement lipid profile for one or more FXN-sensitive lipid markers (FSLMs) in a sample obtained from an FXN deficient subject following administration of an FXN replacement therapy; (b) comparing the subject FXN replacement lipid profile determined in step (a) with a reference FXN lipid profile for the one or more FSLMs; and (c) determining efficacy of the FXN replacement therapy based on the comparison in step (b); wherein the one or more FSLMs are selected from a group consisting of one or more of: triglycerides (TGs), wherein the three acyl groups in each triglyceride molecule contain less than 56 carbons and/or wherein the three acyl groups in each triglyceride molecule contain 7 or less unsaturations; ether phospholipids; phosphatidylcholines (PCs); cholesteryl esters (CEs); and diglycerides (DGs).

In some embodiments, the reference FXN lipid profile is a baseline FXN(−) lipid profile for the one or more FSLMs. In some embodiments, the baseline FXN(−) lipid profile for the one or more FSLMs is determined in a sample obtained from an FXN deficient subject prior to administration of an FXN replacement therapy. In some embodiments, the method further comprises determining a baseline FXN(−) lipid profile for the one or more FXN-sensitive lipid markers (FSLMs) in a sample obtained from the FXN deficient subject prior to administration of the FXN replacement therapy.

In some embodiments, the one or more FSLMs are selected from a group consisting of one or more triglycerides (TGs), wherein the three acyl groups in each triglyceride molecule contain less than 56 carbons and/or wherein the three acyl groups in each triglyceride molecule contain 7 or less unsaturations. In some embodiments, the one or more FSLMs are selected from the group consisting of TG45:1, TG46:1, TG46:3, TG47:1, TG47:2, TG48:0, TG48:1, TG48:2, TG48:3, TG49:1, TG49:2, TG49:3, TG49:4, TG50:1, TG50:2, TG50:3, TG50:4, TG50:5, TG51:1, TG51:2, TG51:3, TG51:4, TG52:2, TG52:3, TG52:4, TG52:5, TG52:6, TG53:2, TG53:3, TG53:4, TG53:5, TG54:4, TG54:5, TG54:6 and TG54:7. In some embodiments, the one or more FSLMs are selected from the group consisting of TG48:1, TG48:2, TG49:1, TG49:2, TG49:4, TG50:1, TG50:3, TG51:1, TG51:2, TG51:3, TG52:3, TG53:2 and TG56:8.

In some embodiments, the one or more FSLMs are selected from one or more ether phospholipids. In some embodiments, the one or more ether phospholipids comprise one or more ether diacylglycerophosphocholines (PCO-). In some embodiments, the one or more PCO- are selected from the group consisting of PC(O-16:0/14:0), PC(O-16:0/18:2), PC(O-16:0/20:3), PC(O-16:0/20:4), PC(O-16:0/22:6), PC(O-17:0/20:4), PC(O-18:0/18:1), PC(O-18:0/22:6), PC(O-18:0/18:2), PC(O-18:1/18:2), PC(O-18:1/20:4), PC(O-18:1/20:5), PC(O-18:1/22:6), PC(O-(20:0/22:6), PC(O-20:1/22.6), PC(O-20:2/20:4), PC(O-22:2/20:4), PC(O-22:1/22:6), PC(O-22:2/20:4), PC(O-(24:1/22:6), PC(O-24:2/20:4), PCO-34:2, PCO-36:3, PCO-34:2, PCO-38:3, PCO-40:2, PCO-40:6, and PCO-44:7 and PCO-46.8. In some embodiments, the one or more PCO- are selected from the group consisting of PC(O-16:0/18:2), PC(O-16:0/20:3), PC(O-18:1/18:2). PC(O-22:2/20:4) and PC(O-24:2/20:4). In some embodiments, the one or more ether phospholipids comprise one or more phosphatidylethanolamine ethers (PEO-).

In some embodiments, the one or more FSLMs are selected from one or more phosphatidylcholines (PCs). In some embodiments, the one or more PCs are selected from the group consisting of PC(15:0/20:3), PC(15:0/22:6), PC(16:0/14:0), PC(16:0/22:4), PC(16:1/16:0), PC(16:1/20:4), PC(16:1/22.5), PC(17:0/20:5), PC(18:0/20:3), PC(18:0/22:4), PC(18:1/20:3), PC(18:2/18:2), PC(20:4/15:0), PC40:6 and PC42:7. In some embodiments, the one or more PCs are selected from the group consisting of PC(15:0/22:6) and PC(16:0/14:0). In some embodiments, the one or more PCs are selected from the group consisting of PC(17:1/20:4), PC(18:2/18:3), PC(18:1/24:1), PC(18:2/20:5) and PC(20:4/20:0). In some embodiments, the one or more PCs is PC(18:2/18:3).

In some embodiments, the one or more FSLMs are selected from one or more cholesteryl esters (CEs). In some embodiments, the one or more CEs are selected from the group consisting of CE16:0 and CE20:5. In some embodiments, the one or more CEs is CE14:1.

In some embodiments, the one or more FSLMs are selected from one or more diglycerides (DGs). In some embodiments, the one or more DGs is DG18:1/18:2.

In some embodiments, the amount of at least one or more FSLMs is increased in the subject following treatment with FXN replacement therapy. In some embodiments, the one or more FSLMs are selected from the group consisting of PC(O-16:0/14:0), PC(O-16:0/18:2), PC(O-16:0/20:3), PC(O-16:0/20:4), PC(O-16:0/22:6), PC(O-17:0/20:4), PC(O-18:0/18:1), PC(O-18:0/22:6), PC(O-18:0/18:2), PC(O-18:1/18:2), PC(O-18:1/20:4), PC(O-18:1/20:5), PC(O-18:1/22:6), PC(O-(20:0/22:6), PC(O-20:1/22.6), PC(O-20:2/20:4), PC(O-22:2/20:4), PC(O-22:1/22:6), PC(O-22:2/20:4), PC(O-(24:1/22:6), PC(O-24:2/20:4), PCO-34:2, PCO-36:3, PCO-34:2, PCO-38:3, PCO-40:2, PCO-40:6, and PCO-44:7, PCO-46.8, PC(17:1/20:4), PC(18:2/18:3), PC(18:1/24:1), PC(18:2/20:5), PC(20:4/20:0), CE16:0 and CE20:5.

In some embodiments, the amount of at least one or more FSLMs is decreased in the subject following treatment with FXN replacement therapy. In some embodiments, the one or more FSLMs are selected from the group consisting of TG45:1, TG46:1, TG46:3, TG47:1, TG47:2, TG48:0, TG48:1, TG48:2, TG48:3, TG49:1, TG49:2, TG49:3, TG49:4, TG50:1, TG50:2, TG50:3, TG50:4, TG50:5, TG51:1, TG51:2, TG51:3, TG51:4, TG52:2, TG52:3, TG52:4, TG52:5, TG52:6, TG53:2, TG53:3, TG53:4, TG53:5, TG54:4, TG54:5, TG54:6, TG54:7, PC(15:0/20:3), PC(15:0/22:6), PC(16:0/14:0), PC(16:0/22:4), PC(16:1/16:0), PC(16:1/20:4), PC(16:1/22.5), PC(17:0/20:5), PC(18:0/20:3), PC(18:0/22:4), PC(18:1/20:3), PC(18:2/18:2), PC(20:4/15:0), PC40:6, PC42:7, DG18:1/18:2 and CE14:1.

In some embodiments, determining an FXN lipid profile for one or more FSLMs comprises determining the amount of the one or more FSLMs. In some embodiments, comparing the subject FXN replacement lipid profile with the baseline FXN(−) lipid profile comprises comparing the amount of one or more FSLMs in the FXN replacement lipid profile with the amount of the corresponding one or more FSLMs in the baseline FXN(−) lipid profile.

In some embodiments, the FXN replacement therapy is determined to be effective when the amount of one or more FSLMs is increased in the FXN replacement lipid profile as compared to the baseline FXN(−) lipid profile, wherein the one or more FSLMs are selected from the group consisting of PC(O-16:0/14:0), PC(O-16:0/18:2), PC(O-16:0/20:3), PC(O-16:0/20:4), PC(O-16:0/22:6), PC(O-17:0/20:4), PC(O-18:0/18:1), PC(O-18:0/22:6), PC(O-18:0/18:2), PC(O-18:1/18:2), PC(O-18:1/20:4), PC(O-18:1/20:5), PC(O-18:1/22:6), PC(O-(20:0/22:6), PC(O-20:1/22.6), PC(O-20:2/20:4), PC(O-22:2/20:4), PC(O-22:1/22:6), PC(O-22:2/20:4), PC(O-(24:1/22:6), PC(O-24:2/20:4), PCO-34:2, PCO-36:3, PCO-34:2, PCO-38:3, PCO-40:2, PCO-40:6, and PCO-44:7, PCO-46.8, PC(17:1/20:4), PC(18:2/18:3), PC(18:1/24:1), PC(18:2/20:5), PC(20:4/20:0), CE16:0 and CE20:5.

In some embodiments, the FXN replacement therapy is determined to be effective when the amount of one or more FSLMs is decreased in the FXN replacement lipid profile as compared to the baseline FXN(−) lipid profile, wherein the one or more FSLMs are selected from the group consisting of TG45:1, TG46:1, TG46:3, TG47:1, TG47:2, TG48:0, TG48:1, TG48:2, TG48:3, TG49:1, TG49:2, TG49:3, TG49:4, TG50:1, TG50:2, TG50:3, TG50:4, TG50:5, TG51:1, TG51:2, TG51:3, TG51:4, TG52:2, TG52:3, TG52:4, TG52:5, TG52:6, TG53:2, TG53:3, TG53:4, TG53:5, TG54:4, TG54:5, TG54:6, TG54:7, PC(15:0/20:3), PC(15:0/22:6), PC(16:0/14:0), PC(16:0/22:4), PC(16:1/16:0), PC(16:1/20:4), PC(16:1/22.5), PC(17:0/20:5), PC(18:0/20:3), PC(18:0/22:4), PC(18:1/20:3), PC(18:2/18:2), PC(20:4/15:0), PC40:6, PC42:7, DG18:1/18:2 and CE14:1.

In some embodiments, determining an FXN lipid profile for one or more FSLMs comprises determining an FXN lipid feature vector of values indicative of lipid of the one or more FSLMs. In some embodiments, determining efficacy of the FXN replacement therapy comprises determining a first FXN lipid feature vector for the subject FXN replacement lipid profile and a second FXN lipid feature vector for the baseline FXN (−) lipid profile and determining a distance between the first and second lipid feature vectors. In some embodiments, determining the distance between the lipid feature vectors comprises determining a scalar product of the first and second lipid feature vectors.

In some embodiments, determining an FXN lipid profile for one or more FSLMs further comprises determining a third lipid feature vector for a normal FXN lipid profile for the FSLMs for a healthy subject. In some embodiments, determining an FXN lipid profile for one or more FSLMs further comprises determining a distance between the second and third lipid feature vectors. In some embodiments, determining an FXN lipid profile for one or more FSLMs further comprises determining a distance between the first and third lipid feature vectors, and normalizing the distance between the first and third lipid feature vectors to the distance between the second and third lipid feature vectors. In some embodiments, determining an FXN lipid profile for one or more FSLMs further comprises using the normalized distance to determine effectiveness of the FXN replacement therapy.

In some embodiments, the FXN lipid profile is determined by mass spectrometry.

In some embodiments, methods of the disclosure further comprise recommending to a healthcare provider to modify the treatment with the FXN replacement therapy based on the determination of efficacy for the FXN replacement therapy.

In some embodiments, the subject has Friedreich's Ataxia (FRDA).

In some embodiments, methods of the present disclosure further comprise obtaining a sample from the FXN deficient subject. In some embodiments, the sample is selected from the group consisting of a buccal sample, a skin sample, a hair follicle or a blood-derived sample. In some embodiments, the sample is a blood-derived sample. In some embodiments, the blood-derived sample is a plasma sample.

In some aspects, the present disclosure also provides a method of monitoring treatment of a subject with a frataxin (FXN) replacement therapy, the method comprising: (a) determining a first FXN replacement lipid profile for one or more FXN-sensitive lipid markers (FSLMs) in a first sample obtained from an FXN deficient subject at a first time point following administration of an FXN replacement therapy to the subject, (b) determining a second FXN replacement lipid profile for the one or more FXN-sensitive lipid markers (FSLMs) in a second sample obtained from the subject at a second time point that is later than the first time point; (c) comparing the second FXN replacement lipid profile with the first FXN replacement profile; thereby monitoring treatment of the subject with the FXN replacement therapy; wherein the one or more FSLMs are selected from a group consisting of one or more of: triglycerides (TGs), wherein the three acyl groups in each triglyceride molecule contain less than 56 carbons and/or wherein the three acyl groups in each triglyceride molecule contain 7 or less unsaturations; ether phospholipids; phosphatidylcholines (PCs); cholesteryl esters (CEs); and diglycerides (DGs).

In some embodiments, the method further comprises making a determination to maintain, increase or decrease the dose or administration frequency of the FXN replacement therapy based on the comparison in step (c).

In some embodiments, at least one dose of the FXN replacement therapy is administered to the subject between obtaining the first time point and second time point. In some embodiments, the FXN replacement therapy is not administered to the subject between obtaining the first time point and second time point.

In some embodiments, the one or more FSLMs are selected from a group consisting of one or more triglycerides (TGs), wherein the three acyl groups in each triglyceride molecule contain less than 56 carbons and/or wherein the three acyl groups in each triglyceride molecule contain 7 or less unsaturations. In some embodiments, the one or more FSLMs are selected from the group consisting of TG45:1, TG46:1, TG46:3, TG47:1, TG47:2, TG48:0, TG48:1, TG48:2, TG48:3, TG49:1, TG49:2, TG49:3, TG49:4, TG50:1, TG50:2, TG50:3, TG50:4, TG50:5, TG51:1, TG51:2, TG51:3, TG51:4, TG52:2, TG52:3, TG52:4, TG52:5, TG52:6, TG53:2, TG53:3, TG53:4, TG53:5, TG54:4, TG54:5, TG54:6 and TG54:7.

In some embodiments, the one or more FSLMs are selected from one or more ether phospholipids. In some embodiments, the one or more ether phospholipids comprise one or more ether diacylglycerophosphocholines (PCO-). In some embodiments, the one or more PCO- are selected from the group consisting of PC(O-16:0/14:0), PC(O-16:0/18:2), PC(O-16:0/20:3), PC(O-16:0/20:4), PC(O-16:0/22:6), PC(O-17:0/20:4), PC(O-18:0/18:1), PC(O-18:0/22:6), PC(O-18:0/18:2), PC(O-18:1/18:2), PC(O-18:1/20:4), PC(O-18:1/20:5), PC(O-18:1/22:6), PC(O-(20:0/22:6), PC(O-20:1/22.6), PC(O-20:2/20:4), PC(O-22:2/20:4), PC(O-22:1/22:6), PC(O-22:2/20:4), PC(O-(24:1/22:6), PC(O-24:2/20:4), PCO-34:2, PCO-36:3, PCO-34:2, PCO-38:3, PCO-40:2, PCO-40:6, and PCO-44:7 and PCO-46.8. In some embodiments, the one or more ether phospholipids comprise one or more phosphatidylethanolamine ethers (PEO-).

In some embodiments, the one or more FSLMs are selected from one or more phosphatidylcholines (PCs). In some embodiments, the one or more PCs are selected from the group consisting of PC(15:0/20:3), PC(15:0/22:6), PC(16:0/14:0), PC(16:0/22:4), PC(16:1/16:0), PC(16:1/20:4), PC(16:1/22.5), PC(17:0/20:5), PC(18:0/20:3), PC(18:0/22:4), PC(18:1/20:3), PC(18:2/18:2), PC(20:4/15:0), PC40:6, PC42:7, PC(17:1/20:4), PC(18:2/18:3), PC(18:1/24:1), PC(18:2/20:5) and PC(20:4/20:0).

In some embodiments, the one or more FSLMs are selected from one or more cholesteryl esters (CEs). In some embodiments, the one or more CEs are selected from the group consisting of CE14:1, CE16:0 and CE20:5.

In some embodiments, the one or more FSLMs are selected from one or more diglycerides (DGs). In some embodiments, the one or more DGs is DG18:1/18:2.

In some aspects, the present disclosure also provides a method for treating an FXN deficiency, the method comprising: (a) determining an FXN lipid profile in a sample obtained from an FXN deficient subject for one or more FXN-sensitive lipid markers (FSLMs), (b) comparing the FXN lipid profile of the sample with at least one other lipid profile selected from the group consisting of normal FXN lipid profile for the one or more FSLMs, baseline FXN(−) lipid profile for the one or more FSLMs, and FXN replacement lipid profile for the one or more FSLMs, (c) classifying the FXN lipid profile determined in step (a) as corresponding to a normal FXN lipid profile, baseline FXN(−) lipid profile or an FXN replacement lipid profile, and (d) initiating or modulating an FXN replacement therapy based on the classification of the FXN lipid profile of the sample.

In some embodiments, modulating an FXN replacement therapy comprises increasing the dosage, decreasing the dosage, increasing the administration frequency, or decreasing the administration frequency, of the FXN replacement therapy. In some embodiments, the FXN deficient subject has Friedreich's Ataxia (FRDA).

In some aspects, the present disclosure also provides a method of treating an FXN deficiency in a subject, comprising: (a) determining an FXN lipid profile for one or more FSLMs in a sample from an FXN deficient subject; and (b) recommending to a healthcare provider to administer an FXN replacement therapy to the subject based on the subject FXN lipid profile determined in step (a).

In some aspects, the present disclosure also provides a method of treating an FXN deficiency in a subject, comprising: (a) obtaining an FXN lipid profile for one or more FSLMs in a sample obtained from an FXN deficient subject; and (b) administering an FXN replacement therapy to the subject based on the subject FXN lipid profile.

In some embodiments, methods of the present disclosure also comprise obtaining the sample from the FXN deficient subject for use in determining the FXN lipid profile for the one or more FSLMs.

In some aspects, the present disclosure also provides a method of detecting one or more frataxin-sensitive lipid markers (FSLMs) in a sample from a frataxin (FXN) deficient subject, comprising contacting the sample, or a portion thereof, with one or more reagents specific for detecting the level of each of the one or more FSLMs.

In some aspects, the present disclosure also provides a method of detecting one or more frataxin-sensitive lipid markers (FSLMs) in a sample from a frataxin (FXN) deficient subject, comprising subjecting the sample, or a portion thereof, to analysis by mass spectrometry.

In some embodiments, the subject is being treated or is scheduled to be treated with an FXN replacement therapy. In some embodiments, the methods of the disclosure further comprise obtaining the sample from the FXN deficient subject.

In some embodiments, the one or more FSLMs are selected from a group consisting of: triglycerides (TGs), wherein the three acyl groups in each triglyceride molecule contain less than 56 carbons and/or wherein the three acyl groups in each triglyceride molecule contain 7 or less unsaturations; ether phospholipids; phosphatidylcholines (PCs); cholesteryl esters (CEs); and diglycerides (DGs).

In some embodiments, the one or more FSLMs are selected from a group consisting of one or more triglycerides (TGs), wherein the three acyl groups in each triglyceride molecule contain less than 56 carbons and/or wherein the three acyl groups in each triglyceride molecule contain 7 or less unsaturations. In some embodiments, the one or more FSLMs are selected from the group consisting of TG45:1, TG46:1, TG46:3, TG47:1, TG47:2, TG48:0, TG48:1, TG48:2, TG48:3, TG49:1, TG49:2, TG49:3, TG49:4, TG50:1, TG50:2, TG50:3, TG50:4, TG50:5, TG51:1, TG51:2, TG51:3, TG51:4, TG52:2, TG52:3, TG52:4, TG52:5, TG52:6, TG53:2, TG53:3, TG53:4, TG53:5, TG54:4, TG54:5, TG54:6 and TG54:7.

In some embodiments, the one or more FSLMs are selected from one or more ether phospholipids. In some embodiments, the one or more ether phospholipids comprise one or more ether diacylglycerophosphocholines (PCO-). In some embodiments, the one or more PCO- are selected from the group consisting of PC(O-16:0/14:0), PC(O-16:0/18:2), PC(O-16:0/20:3), PC(O-16:0/20:4), PC(O-16:0/22:6), PC(O-17:0/20:4), PC(O-18:0/18:1), PC(O-18:0/22:6), PC(O-18:0/18:2), PC(O-18:1/18:2), PC(O-18:1/20:4), PC(O-18:1/20:5), PC(O-18:1/22:6), PC(O-(20:0/22:6), PC(O-20:1/22.6), PC(O-20:2/20:4), PC(O-22:2/20:4), PC(O-22:1/22:6), PC(O-22:2/20:4), PC(O-(24:1/22:6), PC(O-24:2/20:4), PCO-34:2, PCO-36:3, PCO-34:2, PCO-38:3, PCO-40:2, PCO-40:6, and PCO-44:7 and PCO-46.8. In some embodiments, the one or more ether phospholipids comprise one or more phosphatidylethanolamine ethers (PEO-).

In some embodiments, the one or more FSLMs are selected from one or more phosphatidylcholines (PCs). In some embodiments, the one or more PCs are selected from the group consisting of PC(15:0/20:3), PC(15:0/22:6), PC(16:0/14:0), PC(16:0/22:4), PC(16:1/16:0), PC(16:1/20:4), PC(16:1/22.5), PC(17:0/20:5), PC(18:0/20:3), PC(18:0/22:4), PC(18:1/20:3), PC(18:2/18:2), PC(20:4/15:0), PC40:6, PC42:7, PC(17:1/20:4), PC(18:2/18:3), PC(18:1/24:1), PC(18:2/20:5) and PC(20:4/20:0).

In some embodiments, the one or more FSLMs are selected from one or more cholesteryl esters (CEs). In some embodiments, the one or more CEs are selected from the group consisting of CE14:1, CE16:0 and CE20:5.

In some embodiments, the one or more FSLMs are selected from one or more diglycerides (DGs). In some embodiments, the one or more DGs is DG18:1/18:2.

In some embodiments, the FXN lipid profile is determined by mass spectrometry.

In some embodiments, the subject has Friedreich's Ataxia (FRDA).

In some embodiments, the methods of the disclosure further comprise obtaining a sample from the FXN deficient subject. In some embodiments, the sample is selected from the group consisting of a buccal sample, a skin sample, a hair follicle or a blood-derived sample. In some embodiments, the sample is a blood-derived sample. In some embodiments, the blood-derived sample is a plasma sample.

In some embodiments, the FXN replacement therapy comprises administration of an FXN fusion protein. In some embodiments, the FXN fusion protein comprises or consists of the amino acid sequence set forth in SEQ ID NO: 12.

In some aspects, the present disclosure also provides a kit for detecting one or more frataxin-sensitive lipid markers (FSLMs) in a sample obtained from a frataxin (FXN) deficient subject, comprising one or more isotopically labeled lipids for use as internal standards in a mass spectrometry-based analysis for detecting one or more FSLMs in the sample, and a set of instructions for detecting the level of the one or more FSLMs in the sample from the subject by mass spectrometry.

In some embodiments of the methods provided by the present disclosure, the subject is a human.

In some aspects, the present disclosure provides a method for evaluating efficacy of a frataxin (FXN) replacement therapy, the method comprising: (a) determining a baseline FSLM(−) lipid profile for one or more FXN-sensitive lipid markers (FSLMs) in a sample obtained from an FXN deficient subject prior to administration of the FXN replacement therapy; (b) determining an FXN replacement lipid profile for the one or more FXN-sensitive lipid markers (FSLMs) in a sample obtained from the FXN deficient subject following administration of the FXN replacement therapy; (c) comparing the FXN replacement lipid profile determined in step (b) with the baseline FXN(−) lipid profile determined in step (a); and (d) determining efficacy of the FXN replacement therapy based on the comparison in step (c); wherein the one or more FSLMs are selected from a group consisting of one or more triglycerides.

In some embodiments, the three acyl groups in each triglyceride molecule contain less than 56 carbon atoms.

In some embodiments, the three acyl groups in each triglyceride molecule contain a total of 56 or more carbon atoms.

In some embodiments, the three acyl groups in each triglyceride molecule contain 7 or less unsaturations.

In some embodiments, the three acyl groups in each triglyceride molecule contain more than 7 unsaturations, or 6 or more unsaturations.

In some aspects, the present disclosure provides a method for evaluating efficacy of a frataxin (FXN) replacement therapy, the method comprising: (a) determining a baseline FSLM(−) lipid profile for one or more FXN-sensitive lipid markers (FSLMs) in a sample obtained from an FXN deficient subject prior to administration of the FXN replacement therapy; (b) determining an FXN replacement lipid profile for the one or more FXN-sensitive lipid markers (FSLMs) in a sample obtained from the FXN deficient subject following administration of the FXN replacement therapy; (c) comparing the FXN replacement lipid profile determined in step (b) with the baseline FXN(−) lipid profile determined in step (a); and (d) determining efficacy of the FXN replacement therapy based on the comparison in step (c); wherein the one or more FSLM(s) are selected from the group consisting of one or more cholesteryl esters (CEs).

In some aspects, the present disclosure provides a method for evaluating efficacy of a frataxin (FXN) replacement therapy, the method comprising: (a) determining a baseline FSLM(−) lipid profile for one or more FXN-sensitive lipid markers (FSLMs) in a sample obtained from an FXN deficient subject prior to administration of the FXN replacement therapy; (b) determining an FXN replacement lipid profile for the one or more FXN-sensitive lipid markers (FSLMs) in a sample obtained from the FXN deficient subject following administration of the FXN replacement therapy; (c) comparing the FXN replacement lipid profile determined in step (b) with the baseline FXN(−) lipid profile determined in step (a); and (d) determining efficacy of the FXN replacement therapy based on the comparison in step (c); wherein the one or more FSLM(s) are selected from the group consisting of one or more ether phospholipids.

In some embodiments, the one or more ether phospholipids comprise one or more ether diacylglycerophosphocholines (PCO-).

In some embodiments, the one or more ether phospholipids comprise one or more phosphatidylethanolamine ethers (PEO-).

In some aspects, the present disclosure provides a method for evaluating efficacy of a frataxin (FXN) replacement therapy, the method comprising: (a) determining an FXN replacement lipid profile for one or more FXN-sensitive lipid markers (FSLMs) in a sample obtained from an FXN deficient subject following administration of an FXN replacement therapy; (b) comparing the subject FXN replacement lipid profile determined in step (a) with a reference FXN lipid profile for the one or more FSLMs; and (c) determining efficacy of the FXN replacement therapy based on the comparison in step (b); wherein the one or more FSLMs are selected from a group consisting of one or more triglycerides.

In some embodiments, the three acyl groups in each triglyceride molecule contain less than 56 carbon atoms.

In some embodiments, the three acyl groups in each triglyceride molecule contain a total of 56 or more carbon atoms.

In some embodiments, the three acyl groups in each triglyceride molecule contain 7 or less unsaturations.

In some embodiments, the three acyl groups in each triglyceride molecule contain more than 7 unsaturations, or 6 or more unsaturations.

In some aspects, the present disclosure provides a method for evaluating efficacy of a frataxin (FXN) replacement therapy, the method comprising: (a) determining an FXN replacement lipid profile for one or more FXN-sensitive lipid markers (FSLMs) in a sample obtained from an FXN deficient subject following administration of an FXN replacement therapy; (b) comparing the subject FXN replacement lipid profile determined in step (a) with a reference FXN lipid profile for the one or more FSLMs; and (c) determining efficacy of the FXN replacement therapy based on the comparison in step (b); wherein the one or more FSLM(s) are selected from the group consisting of one or more cholesteryl esters (CEs).

In some aspects, the present disclosure provides a method for evaluating efficacy of a frataxin (FXN) replacement therapy, the method comprising: (a) determining an FXN replacement lipid profile for one or more FXN-sensitive lipid markers (FSLMs) in a sample obtained from an FXN deficient subject following administration of an FXN replacement therapy; (b) comparing the subject FXN replacement lipid profile determined in step (a) with a reference FXN lipid profile for the one or more FSLMs; and (c) determining efficacy of the FXN replacement therapy based on the comparison in step (b); wherein the one or more FSLM(s) are selected from the group consisting of ether phospholipids.

In some embodiments, the one or more ether phospholipids comprise one or more ether diacylglycerophosphocholines (PCO-).

In some embodiments, the one or more ether phospholipids comprise one or more phosphatidylethanolamine ethers (PEO-).

In some embodiments, the reference FXN lipid profile is a baseline FXN(−) lipid profile for the one or more FSLMs. In some embodiments, the baseline FXN(−) lipid profile for the one or more FSLMs is determined in a sample obtained from an FXN deficient subject prior to administration of an FXN replacement therapy. In some embodiments, the method further comprises determining a baseline FXN(−) lipid profile for the one or more FXN-sensitive lipid markers (FSLMs) in a sample obtained from the FXN deficient subject prior to administration of the FXN replacement therapy.

In some embodiments, the one or more FSLMs comprise one or more of TG45:1, TG46:1, TG46:3, TG47:1, TG47:2, TG48:0, TG48:1, TG48:2, TG49:1, TG49:2, TG49:3, TG49:4, TG50:1, TG50:2, TG50:3, TG50:4, TG51:1, TG51:2, TG51:3, TG51:4, TG52:3, TG52:5, TG53:2, TG53:3, TG53:4, TG54:5, TG54:6 or TG54:7. In some embodiments, the one or more FSLMs comprise TG45:1, TG46:1, TG46:3, TG47:1, TG47:2, TG48:0, TG48:1, TG48:2, TG49:1, TG49:2, TG49:3, TG49:4, TG50:1, TG50:2, TG50:3, TG50:4, TG51:1, TG51:2, TG51:3, TG51:4, TG52:3, TG52:5, TG53:2, TG53:3, TG53:4, TG54:5, TG54:6 or TG54:7.

In some embodiments, the one or more FSLMs comprise one or more of TG56:6, TG56:7, TG56:8, TG56:9, TG56:10, TG58:8 and TG58:11. In some embodiments, the one or more FSLMs comprise TG56:6, TG56:7, TG56:8, TG56:9, TG56:10, TG58:8 and TG58:11.

In some embodiments, the one or more FSLMs comprise cholesteryl ester 16:0 (CE 16:0) or cholesteryl ester 20:5 (CE 20:5).

In some embodiments, the one or more FSLMs comprise PC(O-16:0/18:2), PC(O-16:0/22:6), PC(O-18:0/18:2), PC(O-18:1/20:5), PCO-36:3, PCO-34:2, PCO-40:2, PCO-44:7 and PC(O-17:0/20:4).

In some embodiments, at least one or more FSLMs are upregulated following treatment with FXN replacement therapy. In some embodiments, the one or more FSLMs upregulated following treatment with FXN replacement therapy comprise one or more of TG56:6, TG56:7, TG56:8, TG56:9, TG56:10, TG58:8, TG58:11, CE16:0, CE20:5, PC(O-16:0/18:2), PC(O-16:0/22:6), PC(O-18:0/18:2), PC(O-18:1/20:5), PCO-36:3, PCO-34:2, PCO-40:2, PCO-44:7 and PC(O-17:0/20:4).

In some embodiments, at least one or more FSLMs are downregulated following treatment with FXN replacement therapy. In some embodiments, the one or more FSLMs downregulated following treatment with FXN replacement therapy comprise one or more of TG45:1, TG46:1, TG46:3, TG47:1, TG47:2, TG48:0, TG48:1, TG48:2, TG49:1, TG49:2, TG49:3, TG49:4, TG50:1, TG50:2, TG50:3, TG50:4, TG51:1, TG51:2, TG51:3, TG51:4, TG52:3, TG52:5, TG53:2, TG53:3, TG53:4, TG54:5, TG54:6 or TG54:7.

In some embodiments, determining an FXN lipid profile for one or more FSLMs comprises detecting the level of lipid of the one or more FSLMs. In some embodiments, comparing the subject FXN replacement lipid profile with the baseline FXN(−) lipid profile comprises comparing the level of lipid of the one or more FSLMs in the FXN replacement lipid profile with the level of lipid of the corresponding one or more FSLMs in the baseline FXN(−) lipid profile.

In some embodiments, when the lipid level of one or more of TG56:6, TG56:7, TG56:8, TG56:9, TG56:10, TG58:8, TG58:11, CE16:0, CE20:5 PC(O-16:0/18:2), PC(O-16:0/22:6), PC(O-18:0/18:2), PC(O-18:1/20:5), PCO-36:3, PCO-34:2, PCO-40:2, PCO-44:7 and PC(O-17:0/20:4) is increased in the FXN replacement lipid profile as compared to the baseline FXN(−) lipid profile, the FXN replacement therapy is determined to be effective.

In some embodiments, when the lipid level of one or more of TG45:1, TG46:1, TG46:3, TG47:1, TG47:2, TG48:0, TG48:1, TG48:2, TG49:1, TG49:2, TG49:3, TG49:4, TG50:1, TG50:2, TG50:3, TG50:4, TG51:1, TG51:2, TG51:3, TG51:4, TG52:3, TG52:5, TG53:2, TG53:3, TG53:4, TG54:5, TG54:6 or TG54:7 is decreased in the FXN replacement lipid profile as compared to the baseline FXN(−) lipid profile, the FXN replacement therapy is determined to be effective.

In some embodiments, determining an FXN lipid profile for one or more FSLMs comprises determining an FXN lipid feature vector of values indicative of lipid of the one or more FSLMs. In some embodiments, determining efficacy of the FXN replacement therapy comprises determining a first FXN lipid feature vector for the subject FXN replacement lipid profile and a second FXN lipid feature vector for the baseline FXN (−) lipid profile and determining a distance between the first and second lipid feature vectors. In some embodiments, determining the distance between the lipid feature vectors comprises determining a scalar product of the first and second lipid feature vectors.

In some embodiments, methods of the disclosure further comprise determining a third lipid feature vector for a normal FXN lipid profile for the FSLMs for a healthy subject. In some embodiments, methods of the disclosure further comprise determining a distance between the second and third lipid feature vectors. In some embodiments, methods of the disclosure further comprise determining a distance between the first and third lipid feature vectors, and normalizing the distance between the first and third lipid feature vectors to the distance between the second and third lipid feature vectors. In some embodiments, methods of the disclosure further comprise using the normalized distance to determine effectiveness of the FXN replacement therapy.

In some embodiments, the FXN lipid profile is determined by mass spectrometry.

In some embodiments, methods of the disclosure further comprise recommending to a healthcare provider to modify the treatment with the FXN replacement therapy based on the determination of efficacy for the FXN replacement therapy.

In some embodiments, the subject has Friedreich's Ataxia (FRDA).

In some embodiments, methods of the present disclosure further comprise obtaining a sample from the FXN deficient subject. In some embodiments, the sample is selected from the group consisting of a buccal sample, a skin sample, a hair follicle or a blood-derived sample. In some embodiments, the sample is a blood-derived sample. In some embodiments, the blood-derived sample is a plasma sample.

In some aspects, the present disclosure provides a method of monitoring treatment of a subject with a frataxin (FXN) replacement therapy, the method comprising: (a) determining a first FXN replacement lipid profile for one or more FXN-sensitive lipid markers (FSLMs) in a first sample obtained from an FXN deficient subject at a first time point following administration of an FXN replacement therapy to the subject, wherein the one or more FSLMs are selected from a group consisting of one or more triglycerides; (b) determining a second FXN replacement lipid profile for the one or more FXN-sensitive lipid markers (FSLMs) in a second sample obtained from the subject at a second time point that is later than the first time point; (c) comparing the second FXN replacement lipid profile with the first FXN replacement profile; thereby monitoring treatment of the subject with the FXN replacement therapy.

In some embodiments, the three acyl groups in each triglyceride molecule contain less than 56 carbon atoms.

In some embodiments, the three acyl groups in each triglyceride molecule contain a total of 56 or more carbon atoms.

In some embodiments, the three acyl groups in each triglyceride molecule contain 7 or less unsaturations.

In some embodiments, the three acyl groups in each triglyceride molecule contain more than 7 unsaturations, or 6 or more unsaturations.

In some aspects, the present disclosure provides a method of monitoring treatment of a subject with a frataxin (FXN) replacement therapy, the method comprising: (a) determining a first FXN replacement lipid profile for one or more FXN-sensitive lipid markers (FSLMs) in a first sample obtained from an FXN deficient subject at a first time point following administration of an FXN replacement therapy to the subject, wherein the one or more FSLM(s) are selected from the group consisting of one or more cholesteryl esters (CEs); (b) determining a second FXN replacement lipid profile for the one or more FXN-sensitive lipid markers (FSLMs) in a second sample obtained from the subject at a second time point that is later than the first time point; and (c) comparing the second FXN replacement lipid profile with the first FXN replacement profile; thereby monitoring treatment of the subject with the FXN replacement therapy.

In some aspects, the present disclosure provides a method of monitoring treatment of a subject with a frataxin (FXN) replacement therapy, the method comprising: (a) determining a first FXN replacement lipid profile for one or more FXN-sensitive lipid markers (FSLMs) in a first sample obtained from an FXN deficient subject at a first time point following administration of an FXN replacement therapy to the subject, wherein the one or more FSLM(s) are selected from the group consisting of one or more ether phospholipids; (b) determining a second FXN replacement lipid profile for the one or more FXN-sensitive lipid markers (FSLMs) in a second sample obtained from the subject at a second time point that is later than the first time point; and (c) comparing the second FXN replacement lipid profile with the first FXN replacement profile; thereby monitoring treatment of the subject with the FXN replacement therapy.

In some embodiments, the one or more ether phospholipids comprise one or more ether diacylglycerophosphocholines (PCO-).

In some embodiments, the one or more ether phospholipids comprise one or more phosphatidylethanolamine ethers (PEO-).

In some embodiments, the methods of the disclosure further comprise making a determination to maintain, increase or decrease the dose or administration frequency of the FXN replacement therapy based on the comparison in step (c).

In some embodiments, at least one dose of the FXN replacement therapy is administered to the subject between obtaining the first time point and second time point.

In some embodiments, the FXN replacement therapy is not administered to the subject between obtaining the first time point and second time point.

In some aspects, the present disclosure provides a method for treating an FXN deficiency, the method comprising: (a) determining an FXN lipid profile in a sample obtained from an FXN deficient subject for one or more FXN-sensitive lipid markers (FSLMs), (b) comparing the FXN lipid profile of the sample with at least one other lipid profile selected from the group consisting of normal FXN lipid profile for the one or more FSLMs, baseline FXN(−) lipid profile for the one or more FSLMs, and FXN replacement lipid profile for the one or more FSLMs, (c) classifying the FXN lipid profile determined in step (a) as corresponding to a normal FXN lipid profile, baseline FXN(−) lipid profile or an FXN replacement lipid profile, and (d) initiating or modulating an FXN replacement therapy based on the classification of the FXN lipid profile of the sample, wherein the one or more FSLMs are selected from a group consisting of one or more triglycerides.

In some embodiments, the three acyl groups in each triglyceride molecule contain less than 56 carbon atoms.

In some embodiments, the three acyl groups in each triglyceride molecule contain a total of 56 or more carbon atoms.

In some embodiments, the three acyl groups in each triglyceride molecule contain 7 or less unsaturations.

In some embodiments, the three acyl groups in each triglyceride molecule contain more than 7 unsaturations, or 6 or more unsaturations.

In some aspects, the present disclosure provides a method for treating an FXN deficiency, the method comprising: (a) determining an FXN lipid profile in a sample obtained from an FXN deficient subject for one or more FXN-sensitive lipid markers (FSLMs), (b) comparing the FXN lipid profile of the sample with at least one other lipid profile selected from the group consisting of normal FXN lipid profile for the one or more FSLMs, baseline FXN(−) lipid profile for the one or more FSLMs, and FXN replacement lipid profile for the one or more FSLMs, (c) classifying the FXN lipid profile determined in step (a) as corresponding to a normal FXN lipid profile, baseline FXN(−) lipid profile or an FXN replacement lipid profile, and (d) initiating or modulating an FXN replacement therapy based on the classification of the FXN lipid profile of the sample, wherein the one or more FSLM(s) are selected from the group consisting of one or more cholesteryl esters (CEs).

In some aspects, the present disclosure provides a method for treating an FXN deficiency, the method comprising: (a) determining an FXN lipid profile in a sample obtained from an FXN deficient subject for one or more FXN-sensitive lipid markers (FSLMs), (b) comparing the FXN lipid profile of the sample with at least one other lipid profile selected from the group consisting of normal FXN lipid profile for the one or more FSLMs, baseline FXN(−) lipid profile for the one or more FSLMs, and FXN replacement lipid profile for the one or more FSLMs, (c) classifying the FXN lipid profile determined in step (a) as corresponding to a normal FXN lipid profile, baseline FXN(−) lipid profile or an FXN replacement lipid profile, and (d) initiating or modulating an FXN replacement therapy based on the classification of the FXN lipid profile of the sample, wherein the one or more FSLM(s) are selected from the group consisting of one or more ether phospholipids.

In some embodiments, the one or more ether phospholipids comprise one or more ether diacylglycerophosphocholines (PCO-).

In some embodiments, the one or more ether phospholipids comprise one or more phosphatidylethanolamine ethers (PEO-).

In some embodiments, modulating an FXN replacement therapy comprises increasing the dosage, decreasing the dosage, increasing the administration frequency, or decreasing the administration frequency, of the FXN replacement therapy.

In some embodiments, the FXN deficient subject is Friedreich's Ataxia (FRDA).

In some aspects, the present disclosure also provides a method of treating an FXN deficiency in a subject, comprising: (a) determining an FXN lipid profile for one or more FSLMs in a sample from an FXN deficient subject; and (b) recommending to a healthcare provider to administer an FXN replacement therapy to the subject based on the subject FXN lipid profile determined in step (a).

In some aspects, the present disclosure also provides a method of treating an FXN deficiency in a subject, comprising: (a) obtaining an FXN lipid profile for one or more FSLMs in a sample obtained from an FXN deficient subject; and (b) administering an FXN replacement therapy to the subject based on the subject FXN lipid profile.

In some embodiments, methods of the disclosure further comprise obtaining the sample from the FXN deficient subject for use in determining the FXN lipid profile for the one or more FSLMs.

In some aspects, the present disclosure also provides a method of detecting one or more frataxin-sensitive lipid markers (FSLMs) in a sample from a frataxin (FXN) deficient subject, comprising contacting the sample, or a portion thereof, with one or more reagents specific for detecting the level of each of one or more FSLMs, wherein the one or more FSLMs are selected from a group consisting of one or more triglycerides, thereby detecting the FSLMs in the sample.

In some embodiments, the three acyl groups in each triglyceride molecule contain less than 56 carbon atoms.

In some embodiments, the three acyl groups in each triglyceride molecule contain 7 or less unsaturations.

In some aspects, the present disclosure provides a method of detecting one or more frataxin-sensitive lipid markers (FSLMs) in a sample from a frataxin (FXN) deficient subject, comprising contacting the sample, or a portion thereof, with one or more reagents specific for detecting the level of each of one or more FSLMs, wherein the one or more FSLM(s) are selected from the group consisting of one or more cholesteryl esters (CEs), thereby detecting the FSLMs in the sample.

In some aspects, the present disclosure provides a method of detecting one or more frataxin-sensitive lipid markers (FSLMs) in a sample from a frataxin (FXN) deficient subject, comprising contacting the sample, or a portion thereof, with one or more reagents specific for detecting the level of each of one or more FSLMs, wherein the one or more FSLM(s) are selected from the group consisting of one or more ether phospholipids.

In some embodiments, the one or more ether phospholipids comprise one or more ether diacylglycerophosphocholines (PCO-).

In some embodiments, the one or more ether phospholipids comprise one or more phosphatidylethanolamine ethers (PEO-).

In some embodiments, the subject is being treated or is scheduled to be treated with an FXN replacement therapy.

In some embodiments, methods of the disclosure further comprise obtaining the sample from the FXN deficient subject.

In some embodiments, the one or more FSLMs comprise one or more of TG56:6, TG56:7, TG56:8, TG56:9, TG56:10, TG58:8 and TG58:11.

In some embodiments, the one or more FSLMs comprise one or more of TG45:1, TG46:1, TG46:3, TG47:1, TG47:2, TG48:0, TG48:1, TG48:2, TG49:1, TG49:2, TG49:3, TG49:4, TG50:1, TG50:2, TG50:3, TG50:4, TG51:1, TG51:2, TG51:3, TG51:4, TG52:3, TG52:5, TG53:2, TG53:3, TG53:4, TG54:5, TG54:6 or TG54:7.

In some embodiments, the one or more FSLMs comprise cholesteryl ester 16:0 (CE 16:0) or cholesteryl ester 20:5 (CE 20:5).

In some embodiments, the one or more FSLMs comprise PC(O-16:0/18:2), PC(O-16:0/22:6), PC(O-18:0/18:2), PC(O-18:1/20:5), PCO-36:3, PCO-34:2, PCO-40:2, PCO-44:7 and PC(O-17:0/20:4).

In some embodiments, the FXN lipid profile is determined by mass spectrometry.

In some embodiments, the subject has Friedreich's Ataxia (FRDA).

In some embodiments, methods of the disclosure further comprise obtaining a sample from the FXN deficient subject. In some embodiments, the sample is selected from the group consisting of a buccal sample, a skin sample, a hair follicle or a blood-derived sample. In some embodiments, the sample is a blood-derived sample. In some embodiments, the blood-derived sample is a plasma sample.

In some embodiments, the FXN replacement therapy comprises administration of an FXN fusion protein. In some embodiments, the FXN fusion protein comprises or consists of the amino acid sequence set forth in SEQ ID NO: 12.

In some aspects, the present disclosure also provides a kit for detecting one or more frataxin-sensitive lipid markers (FSLMs) in a sample obtained from a frataxin (FXN) deficient subject, comprising one or more isotopically labeled lipids for use as internal standards in a mass spectrometry-based analysis for detecting one or more FSLMs in the sample, wherein the one or more isotopically labeled lipids are selected from a group consisting of one or more isotopically labeled triglycerides, and a set of instructions for detecting the level of the one or more FSLMs in the sample from the subject by mass spectrometry.

In some embodiments, the three acyl groups in each isotopically labeled triglyceride molecule contain less than 56 carbon atoms. In some embodiments, the three acyl groups in each isotopically labeled triglyceride molecule contain 7 or less unsaturations. In some embodiments, the one or more isotopically labeled triglycerides are selected from the group consisting of isotopically labeled TG45:1, TG46:1, TG46:3, TG47:1, TG47:2, TG48:0, TG48:1, TG48:2, TG49:1, TG49:2, TG49:3, TG49:4, TG50:1, TG50:2, TG50:3, TG50:4, TG51:1, TG51:2, TG51:3, TG51:4, TG52:3, TG52:5, TG53:2, TG53:3, TG53:4, TG54:5, TG54:6 or TG54:7.

In some embodiments, the three acyl groups in each isotopically labeled triglyceride molecule contain a total of 56 or more carbon atoms. In some embodiments, the three acyl groups in each isotopically labeled triglyceride molecule contain more than 7 unsaturations, or 6 or more unsaturations. In some embodiments, the one or more isotopically labeled triglycerides are selected from the group consisting of isotopically labeled TG56:6, TG56:7, TG56:8, TG56:9, TG56:10, TG58:8 and TG58:11

In some aspects, the present disclosure also provides a kit for detecting one or more frataxin-sensitive lipid markers (FSLMs) in a sample obtained from a frataxin (FXN) deficient subject, comprising one or more isotopically labeled lipids for use as internal standards in a mass spectrometry-based analysis for detecting one or more FSLMs in the sample, wherein the one or more isotopically labeled lipids are selected from a group consisting of one or more isotopically labeled cholesteryl esters (CEs), and a set of instructions for detecting the level of the one or more FSLMs in the sample from the subject by mass spectrometry.

In some embodiments, the one or more isotopically labeled cholesteryl esters are selected from the group consisting of isotopically labeled cholesteryl ester 16:0 (CE 16:0) and isotopically labeled cholesteryl ester 20:5 (CE 20:5).

In some aspects, the present disclosure provides a kit for detecting one or more frataxin-sensitive lipid markers (FSLMs) in a sample obtained from a frataxin (FXN) deficient subject, comprising one or more isotopically labeled lipids for use as internal standards in a mass spectrometry-based analysis for detecting one or more FSLMs in the sample, wherein the one or more isotopically labeled lipids are selected from a group consisting of one or more isotopically labeled ether phospholipids, and a set of instructions for detecting the level of the one or more FSLMs in the sample from the subject by mass spectrometry.

In some embodiments, the one or more isotopically labeled ether phospholipids comprise one or more isotopically labeled ether diacylglycerophosphocholines (PCO-).

In some embodiments, the one or more isotopically labeled ether diacylglycerophosphocholines (PCO-) comprise PC(O-16:0/18:2), PC(O-16:0/22:6), PC(O-18:0/18:2), PC(O-18:1/20:5), PCO-36:3, PCO-34:2, PCO-40:2, PCO-44:7 and PC(O-17:0/20:4).

In some embodiments, the one or more isotopically labeled ether phospholipids comprise one or more isotopically labeled phosphatidylethanolamine ethers (PEO-).

### BRIEF DESCRIPTION OF THE DRAWINGS

FIG. 1 is a scatter plot showing the results of principal component analysis (PCA) of all MS signals from all analyzed human plasma samples, with different colors indicating FRDA subjects and healthy subjects.

FIG. 2 is a scatter plot showing the results of PCA of all MS signals from all human plasma samples, with different colors indicating subjects of different genders.

FIG. 3 is a volcano plot that presents the comparison of human plasma samples from FRDA subjects vs. healthy controls. The different lipid subclasses are indicated by different colors. Gray dots represent lipids that are were not identified. The horizontal red line indicates the threshold p-value of 0.04, and the vertical red lines represent fold change of 1.23.

FIG. 4 is a boxplot showing log2 fold change in the levels of the most significantly modulated lipids in human plasma samples obtained from FRDA subjects as compared to healthy subjects.

FIG. 5, panel A is a PCA loading plot of the 168 features discriminating FRDA subjects from healthy subjects with lipid subclasses for the 111 annotated features (66 unique lipids) using the color code for PC1 vs. PC2.

FIG. 5, panel B is a PCA loading plot of the 168 features discriminating FRDA subjects from healthy subjects with lipid subclasses for the 111 annotated features (66 unique lipids) using the color code for PC1 vs. PC2 (panel A) and PC1 vs. PC3 (panel B). Lipids that are increased in human plasma samples from FRDA subjects as compared to healthy subjects are on the left side, while those that are decreased are on the right.

FIG. 6 is a scatter plot showing the results of PCA of all MS signals from all mouse plasma samples, with different colors indicating different conditions: WT mice treated with vehicle (WTV), WT mice treated with FXN replacement therapy (WTTA), FXN-KO mice treated with vehicle (MV) and FXN-KO mice treated with FXN replacement therapy (MTA). The shape of the dots indicate different genders.

FIG. 7 is a scatter plot showing the results of PCA of all plasma samples from female mice with all MS signals, with different colors indicating different conditions: WTV, WTTA, MV and MTA.

FIG. 8 is a volcano plot presenting the comparison of plasma samples obtained from female MTA mice vs. female WTV mice. The different lipid subclasses are indicated by different colors, and gray dots represent lipids that were not identified. The horizontal red line indicates the threshold p-value of 0.05, and the vertical red line represents fold change of 1.35.

FIG. 9 is a panel of boxplots showing the most significantly modulated lipids (p<0.01) in plasma samples obtained from female MV mice vs. female WTV mice.

FIG. 10 is a volcano plot showing the comparison of plasma samples from female MTA mice vs. female WV. The different lipid subclasses are indicated by different colors, and gray dots represent lipids that were not identified. The horizontal red line indicates the threshold p-value of 0.05, and the vertical red line represents fold change of 1.35.

FIG. 11 shows boxplots of the 63 annotated lipids identified in plasma samples from female MTA mice vs. female MV mice.

FIG. 12 is a Venn Diagram in which the red (left) circle represents 167 entities which discriminate between plasma samples obtained from MTA mice and MV mice, and the blue circle represents 356 entities which discriminate between plasma samples obtained from MV mice and WTV mice. The overlapping portion of the red and blue circles represents 36 entities which represent lipids that are modulated as a result of FXN-KO mutation and which are further modulated by FXN replacement therapy administered to the FXN-KO mice.

FIG. 13 is a series of boxplots of representative lipids or features that are increased in FXN-KO mice (MV) as compared to the WT mice (WTV) and that are decreased following treatment with FXN replacement therapy (MTA).

FIG. 14, panel A is a series of boxplots of representative lipids that are decreased in FXN-KO mice (MV) as compared to the WT mice (WTV) and that are further decreased following treatment with FXN replacement therapy (MTA).

FIG. 14, panel B is a series of boxplots of representative lipids and features that are increased in FXN-KO mice treated with vehicle (MV) vs. WT mice treated with vehicle (WTV) and that are further increased following treatment with FXN replacement therapy (MTA).

FIG. 15, panel A is a dot plot showing log 2 fold change in the levels of various triglycerides (TGs) vs. number of carbons in their acyl chain in FXN-KO mice treated with vehicle vs. wild-type mice treated with vehicle (MV vs. WTV). The circled dots represent TGs that are increased in FXN-KO mice versus wild-type mice, representing the effect of mutation. Of these TGs, many have an acyl chain length of >55. Some TGs that are increased in FXN-KO mice as compared to the wild-type mice that do not decrease following FXN replacement therapy have an acyl chain of ≤50.

FIG. 15, panel B is a dot plot showing log 2 fold change in the levels of various TGs vs. number of carbons in their acyl chain in FXN-KO mice treated with FXN replacement therapy or vehicle (MTA vs. MV). The circled dots represent TGs which were decreased in the FXN-KO mice as compared to the wild-type mice and were increased following treatment with the FXN replacement therapeutic compound. These TGs have long chain length (<55).

FIG. 16, panel A is a dot plot showing log 2 fold change in the levels of various phosphatidylcholines (PCs) vs. number of carbons in their acyl chain in FXN-KO mice treated with vehicle vs. wild-type mice treated with vehicle (MV vs. WTV).

FIG. 16, panel B is a dot plot showing log 2 fold change in the levels of various PCs versus their acyl chain length in FXN-KO mice treated with FXN replacement therapy or vehicle (MTA vs. MV). The circled dots represent two PCs that are decreased in FXN-KO mice and are increased following FXN replacement therapy.

FIG. 17 is a scatter plot showing the results of Principal Component Analysis (PCA) of ratio of day 15/day −2 for all samples with MS signals, colored by group: Group 1, Group 2 and Group 3 (corresponding to subjects treated with 25 mg, 50 mg and 100 mg of the exemplary FXN replacement therapeutic compound, respectively). The PCA analysis reveals a well-defined separation between Group 1 and Group 3.

FIG. 18 is a volcano plot presenting the comparison for healthy human subjects vs. subjects with FRDA at day −2 (i.e., prior to treatment). The different lipid subclasses are indicated by different colors. Gray dots represent lipids that are were not identified. The horizontal lines indicates the threshold p-values of 0.03 and 0.05, and the vertical red lines represent fold change of 0.8 and 1.25.

FIG. 19 is a volcano plot presenting the comparison for day 15 vs. day −2 for subjects treated with placebo. Of all 3328 features, 200 discriminate day 15 vs. day −2 for subjects who received placebo, with a p-value <0.03 and a fold change of 1.25. From these 200 features, 93 were annotated to a lipid ID: 44 features (22 annotated) were increased, and 156 features (71 annotated) were decreased with placebo between day 15 and day −2.

FIG. 20 is a volcano plot presenting the comparison for day 15 vs. day −2 for subjects of Group 1 (dosed with 25 mg of the exemplary FXN replacement therapeutic compound). Of all 3328 features, 237 discriminate day 15 vs. day-2, with a p-value <0.03 and a fold change of 1.25. From these 237 features, 73 were annotated to a lipid ID: 125 features (49 annotated) were increased, and 112 features (24 annotated) were decreased at day 15 vs. day −2 after dosing with 25 mg of the exemplary FXN replacement therapeutic compound.

FIG. 21 is a volcano plot presenting the comparison for day 15 vs. day −2 for subjects of Group 2 (dosed with 50 mg of the exemplary FXN replacement therapeutic compound). Of all 3328 features, 169 discriminate day 15 vs. day-2, with a p-value <0.03 and a fold change of 1.25. From these 169 features, 59 were annotated to a lipid ID: 49 features (8 annotated) were increased, and 120 features (51 annotated) were decreased at day 15 vs. day −2 after dosing with 50 mg of the exemplary FXN replacement therapeutic compound.

FIG. 22 is a volcano plot presenting the comparison for day 15 vs. day −2 for subjects of Group 3 (dosed with 100 mg of the exemplary FXN replacement therapeutic compound). Of all 3328 features, 506 discriminate day 15 vs. day-2, with a p-value <0.03 (Q-value<0.05) and a fold change of 1.25. From these 506 features, 166 were annotated to a lipid ID: 218 features (51 annotated) were increased, and 288 features (115 annotated) were decreased at day 15 vs. day −2 after dosing with 100 mg of the exemplary FXN replacement therapeutic compound. The effect of dosing with the FXN replacement therapeutic compound observed for Group 3 (506 significant features) is more pronounced than the effect observed for Groups 1, 2 and placebo (<250 significant features).

FIG. 23, Panel A is a dot plot showing log 2 fold change in the levels of TGs vs. the number of acyl chain carbons for samples from FRDA subjects at day −2 (prior to treatment) vs. healthy control subjects.

FIG. 23, Panel B is a series of dot plots showing log 2 fold change in the levels of TGs on day 15 vs. day −2 vs. the number of carbons in their acyl chain for samples from subjects dosed with placebo, 100 mg (Group 3), 50 mg (Group 2) and 25 mg (Group 1) of the exemplary FXN replacement therapeutic compound. The TGs graphed in the dot plots are 49 unique TGs selected based on results for Group 3, with p-value<0.03, FC>1.25 or FC<0.8 for day 15 vs. day-2 for this group.

FIG. 24, Panel A is a dot plot showing log 2 fold change in the levels of TGs vs. the number of acyl chain unsaturations for samples from FRDA subjects at day −2 (prior to treatment) vs. healthy control subjects.

FIG. 24, Panel B is a series of dot plots showing log 2 fold change in the levels of TGs vs. the number of acyl chain unsaturations. The dot plot in the upper left quadrant is shown for samples obtained from FRDA subjects vs. healthy controls who were not dosed (see Example 1). The remaining dot plots are shown for samples obtained from subjects dosed with 100 mg (Group 3), 50 mg (Group 2) and 25 mg (Group 1) of the exemplary FXN replacement therapeutic compound on day 15 after dosing vs. day −2 before dosing. The TGs graphed in the dot plots are 49 unique TGs selected based on results for Group 3, with p-value<0.03, FC>1.25 or FC<0.8 for day 15 vs. day-2 for this group.

FIG. 25, panel A is a dot plot showing log 2 fold change in the levels of 49 unique TGs vs. the number of acyl chain carbons in sample from a representative patient treated with placebo. The number of unsaturations in each TG is presented using grayscale, with white dots representing 0 unsaturations and darker dots representing an increasing number of unsaturations.

FIG. 25, panel B is a dot plot showing log 2 fold change in the levels of 49 unique TGs vs. the number of acyl chain carbons in sample from a representative patient treated with 100 mg of the exemplary FXN replacement therapeutic compound. The number of unsaturations in each TG is presented using grayscale, with white dots representing 0 unsaturations and darker dots representing an increasing number of unsaturations.

FIG. 26 is a graphic summary of the comparisons of day 15 vs. day −2 for subjects treated with placebo, 25 mg (Group 1), 50 mg (Group 2) and 100 mg (Group 3) of the exemplary FXN replacement therapeutic compound for 49 TGs. The 49 TGs have been selected as being most significantly modulated TGs for Group 3. Dot intensity represents fold changes in log 2 of day 15 vs. day −2 for each group with p-value <0.05. A red dot represents an increase with the treatment, while a blue dot represents a decrease with the treatment. The dot color intensity is proportional to the value of fold-change: the stronger the color intensity, the larger the fold change.

FIG. 27 is a graphic summary of the correlation between frataxin levels and the levels of selected 49 TGs that were most significantly modulated in Group 3. The intensity of each dot reflects the strength of correlations between levels of lipids and frataxin levels with p-value <0.2, with blue dot representing negative correlation and red dot representing positive correlation.

FIG. 28 is a graphic summary of the correlation between frataxin levels and the levels of cholesteryls esters (CE). The results presented in FIG. 28 indicate that only CE16:0 and CE20:5 are correlated in a positive manner with frataxin levels from skin, with p-value <0.2, and to a lesser extent with frataxin levels in platelets.

FIG. 29, panel A is a dot plot of ratio of lipid levels at day 15 vs. day −2 vs. ratio of frataxin levels in skin samples at day 15 vs. day −2 for representative TGs with acyl chain length <56 carbons.

FIG. 29, panel B is a dot plot of ratio of lipid levels at day 15 vs. day −2 vs. ratio of frataxin levels in skin samples at day 15 vs. day −2 for representative TGs with acyl chain length ≥56 carbons.

FIG. 29, panel C is a dot plot of ratio of lipid levels at day 15 vs. day −2 vs. ratio of frataxin levels in skin samples at day 15 vs. day −2 for CD16:0.

FIG. 30, panel A is a volcano plot showing an effect (positive or negative) and the associated p-value for each feature. The threshold chosen was p-value=0.05, which is more stringent than for the Pearson correlation analysis, since the goal was to identify the most significant lipids that correlate with frataxin levels in skin other than TGs and CEs.

FIG. 30, panel B is a summary table of the data presented in the volcano plot in FIG. 30, panel A.

FIG. 31, panel A is a dot plot of ratio of lipid levels at day 15 vs. day −2 vs. ratio of frataxin levels in skin samples at day 15 vs. day −2 for a representative PC ether PC(O-17:0/20:4).

FIG. 31, panel B is a dot plot of ratio of lipid levels at day 15 vs. day −2 vs. ratio of frataxin levels in skin samples at day 15 vs. day −2 for a representative LPC18:0.

FIG. 31, panel C is a dot plot of ratio of lipid levels at day 15 vs. day −2 vs. ratio of frataxin levels in skin samples at day 15 vs. day −2 for a representative acylcarnitine 18:2.

FIG. 32 is an illustration of a 2-dimensional display obtained using the Fruchterman-Reingold layout algorithm of positive correlations between annotated lipids differentially expressed at day 15 vs. day −2 in subjects treated with 100 mg of the exemplary FXN replacement therapeutic compound (Group 3). Edges represent connections or correlations (R>0.6) between the nodes (lipids). The width of the link is proportional to the correlation.

FIG. 33 is an illustration of a 2-dimensional display obtained using the Fruchterman-Reingold layout algorithm of positive correlations between annotated lipids associated with frataxin.

FIG. 34 shows a Volcano plot showing log 2 fold change for each lipid feature MS signal intensity for FRDA subjects (Day −2) vs. healthy volunteers (To) on the x-axis and the p-value (−log 10) on the y-axis. The horizontal dotted lines correspond to p-values of 0.05 and 0.03. The vertical dotted lines correspond to fold-changes of 0.8 (1/1.25; left) and 1.25 (right). The number of annotated lipids per subclass are shown in the table to the right of the volcano plot. The table displays the number of lipid features and unique lipids that were changed (up or down) for each lipid subclass. Lipid features showing higher values of fold-change in MS signal intensity for FRDA subjects vs. healthy volunteers are on the right of the graph, and those showing lower values are on the left of the graph.

FIG. 35 is a graph showing log 2 fold-change in various TG species that are significantly changed in untreated FRDA subjects vs. healthy volunteers.

FIG. 36 is a dot plot showing log 2 fold change in the levels of TGs vs. the number of acyl chain carbons for samples from untreated FRDA subjects vs. healthy volunteers.

FIG. 37 is a dot plot showing log 2 fold change in the levels of TGs vs. the number of acyl chain unsaturations for samples from untreated FRDA subjects vs. healthy volunteers.

FIG. 38 is a graph showing log 2 fold-change in various PCO- species that are significantly changed in untreated FRDA subjects vs. healthy volunteers.

FIG. 39 is a graph showing log 2 fold-change in various PC species that are significantly changed in untreated FRDA subjects vs. healthy volunteers.

FIG. 40 is an illustration of a 2-dimensional display obtained using the Fruchterman-Reingold layout algorithm of positive correlations between annotated lipids differentially expressed in untreated FRDA subjects vs. healthy volunteers.

FIG. 41 is a forest plot representing log 2 fold change for the 29 unique lipids for FRDA subjects who were administered 100 mg of an exemplary FXN replacement therapeutic compound at Day 15 vs. Day −2 and for FRDA subjects vs. healthy volunteers.

FIG. 42, panel A shows representative box plots for TG48:1, which shows changes in the opposite directions for the effect of disease (FRDA vs. healthy volunteers) and treatment (Day 15 vs Day −2) in FRDA subjects treated with 25 mg (Cohort 1), 50 mg (Cohort 2) and 100 mg (Cohort 3) of an exemplary FXN replacement therapeutic compound.

FIG. 42, panel B shows representative box plots for TG49:4, which shows changes in the opposite directions for the effect of disease (FRDA vs. healthy volunteers) and treatment (Day 15 vs Day −2) in FRDA subjects treated with 25 mg (Cohort 1), 50 mg (Cohort 2) and 100 mg (Cohort 3) of an exemplary FXN replacement therapeutic compound.

FIG. 42, panel C shows representative box plots for PC(O-18:1/18:2), which shows changes in the opposite directions for the effect of disease (FRDA vs. healthy volunteers) and treatment (Day 15 vs Day −2) in FRDA subjects treated with 25 mg (Cohort 1), 50 mg (Cohort 2) and 100 mg (Cohort 3) of an exemplary FXN replacement therapeutic compound.

### DETAILED DESCRIPTION

### Overview

In one aspect, the present disclosure is based, at least in part, on providing a set of markers, also referred to herein as FXN-sensitive lipid markers (or FSLMs), whose respective levels are positively or negatively correlated to frataxin (FXN) levels in a cell. In some embodiments, the FSLMs of the present disclosure are contrary regulated by FXN gene ablation or deficiency, followed by FXN protein replacement. Thus, said FSLMs of the present disclosure are both associated with FXN deficiency in a subject and conversely associated with FXN replacement. The FSLMs disclosed herein were found to be sensitive to FXN and are considered markers of FXN replacement. Therefore, these FSLMs can be used to determine and/or monitor efficacy of FXN replacement therapy in a subject, as described herein.

In some embodiments, the FSLMs comprise one or more triglycerides (triacylglycerols, TGs). In some embodiments, the FSLMs comprise one or more triglycerides, wherein the three acyl groups in each triglyceride molecule contain 45 carbons to 55 carbons, e.g., 45, 46, 47, 48, 49, 50, 51, 52, 53, 54 or 55 carbons.

In some embodiments, the FSLMs comprise one or more triglycerides, wherein the three acyl groups in each triglyceride molecule contain less than 56 carbons. In some embodiments, the FSLMs comprise one or more triglycerides, wherein the three acyl groups in each triglyceride molecule contain 7 or less unsaturations. In some embodiments, the FSLMs comprise one or more of triacylglycerol 45:1 (TG45:1), triacylglycerol 46:1 (TG46:1), triacylglycerol 46:3 (TG46:3), triacylglycerol 47:1 (TG47:1), triacylglycerol 47:2 (TG47:2), triacylglycerol 48:0 (TG48:0), triacylglycerol 48:1 (TG48:1), triacylglycerol 48:2 (TG48:2), triacylglycerol 49:1 (TG49:1), triacylglycerol 49:2 (TG49:2), triacylglycerol 49:3 (TG49:3), triacylglycerol 49:4 (TG49:4), triacylglycerol 50:1 (TG50:1), triacylglycerol 50:2 (TG50:2), triacylglycerol 50:3 (TG50:3), triacylglycerol 50:4 (TG50:4), triacylglycerol 51:1 (TG51:1), triacylglycerol 51:2 (TG51:2), triacylglycerol 51:3 (TG51:3), triacylglycerol 51:4 (TG51:4), triacylglycerol 52:3 (TG52:3), triacylglycerol 52:5 (TG52:5), triacylglycerol 53:2 (TG53:2), triacylglycerol 53:3 (TG53:3), triacylglycerol 53:4 (TG53:4), triacylglycerol 54:5 (TG54:5), triacylglycerol 54:6 (TG54:6) and triacylglycerol 54:7 (TG54:7). In some embodiments, the FSLMs comprise two or more of TG45:1, TG46:1, TG46:3, TG47:1, TG47:2, TG48:0, TG48:1, TG48:2, TG49:1, TG49:2, TG49:3, TG49:4, TG50:1, TG50:2, TG50:3, TG50:4, TG51:1, TG51:2, TG51:3, TG51:4, TG52:3, TG52:5, TG53:2, TG53:3, TG53:4, TG54:5, TG54:6 and TG54:7. In some embodiments, the FSLMs comprise three or more of TG45:1, TG46:1, TG46:3, TG47:1, TG47:2, TG48:0, TG48:1, TG48:2, TG49:1, TG49:2, TG49:3, TG49:4, TG50:1, TG50:2, TG50:3, TG50:4, TG51:1, TG51:2, TG51:3, TG51:4, TG52:3, TG52:5, TG53:2, TG53:3, TG53:4, TG54:5, TG54:6 and TG54:7. In some embodiments, the FSLMs comprise five or more of TG45:1, TG46:1, TG46:3, TG47:1, TG47:2, TG48:0, TG48:1, TG48:2, TG49:1, TG49:2, TG49:3, TG49:4, TG50:1, TG50:2, TG50:3, TG50:4, TG51:1, TG51:2, TG51:3, TG51:4, TG52:3, TG52:5, TG53:2, TG53:3, TG53:4, TG54:5, TG54:6 and TG54:7. In some embodiments, the FSLMs comprise ten or more of TG45:1, TG46:1, TG46:3, TG47:1, TG47:2, TG48:0, TG48:1, TG48:2, TG49:1, TG49:2, TG49:3, TG49:4, TG50:1, TG50:2, TG50:3, TG50:4, TG51:1, TG51:2, TG51:3, TG51:4, TG52:3, TG52:5, TG53:2, TG53:3, TG53:4, TG54:5, TG54:6 and TG54:7. In some embodiments, the FSLMs comprise fifteen or more of TG45:1, TG46:1, TG46:3, TG47:1, TG47:2, TG48:0, TG48:1, TG48:2, TG49:1, TG49:2, TG49:3, TG49:4, TG50:1, TG50:2, TG50:3, TG50:4, TG51:1, TG51:2, TG51:3, TG51:4, TG52:3, TG52:5, TG53:2, TG53:3, TG53:4, TG54:5, TG54:6 and TG54:7. In some embodiments, the FSLMs comprise twenty or more of TG45:1, TG46:1, TG46:3, TG47:1, TG47:2, TG48:0, TG48:1, TG48:2, TG49:1, TG49:2, TG49:3, TG49:4, TG50:1, TG50:2, TG50:3, TG50:4, TG51:1, TG51:2, TG51:3, TG51:4, TG52:3, TG52:5, TG53:2, TG53:3, TG53:4, TG54:5, TG54:6 and TG54:7. In some embodiments, the FSLMs comprise twenty-five or more of TG45:1, TG46:1, TG46:3, TG47:1, TG47:2, TG48:0, TG48:1, TG48:2, TG49:1, TG49:2, TG49:3, TG49:4, TG50:1, TG50:2, TG50:3, TG50:4, TG51:1, TG51:2, TG51:3, TG51:4, TG52:3, TG52:5, TG53:2, TG53:3, TG53:4, TG54:5, TG54:6 and TG54:7.

In some embodiments, the FSLMs comprise one or more of cholesteryl esters (CEs), such as 16:0 (CE16:0) and cholesteryl ester 20:5 (CE20:5). In some embodiments, the FSLMs comprise one or more of ether phospholipids, e.g., one or more ether diacylglycerophosphocholines (PCO-), one or more phosphatidylethanolamine ethers (PEO-) or one or more phosphatidylcholine ethers, such as phosphatidylcholine ether PC(O-17:0/20:4). In some embodiments, the FSLMs comprise one or more of cholesteryl ester CE16:0, CE20:5 and 0-17:0/20:4. In some embodiments, the FSLMs comprise two or more of cholesteryl ester CE16:0, CE20:5 and 0-17:0/20:4. In some embodiments, the FSLMs comprise cholesteryl ester CE16:0, CE20:5 and 0-17:0/20:4.

In some embodiments, the efficacy of FXN replacement therapy in a subject can be determined, evaluated, and/or monitored based on the analysis of one or more FSLM lipid profiles before and after administration or initiation of FXN replacement therapy in the subject. Based on the results of the FSLM lipid profile analysis, adjustments can be made to the FXN replacement therapy in a subject to, e.g., initiate, increase, decrease or cease FXN replacement therapy in the subject.

### Definitions

Unless defined otherwise, all technical and scientific terms used herein have the meaning commonly understood by a person skilled in the art to which this disclosure belongs. The following references, the entire disclosures of which are incorporated herein by reference, provide one of skill with a general definition of many of the terms (unless defined otherwise herein) used in this disclosure: Singleton et al., Dictionary of Microbiology and Molecular Biology (2^{nd} ed. 1994); The Cambridge Dictionary of Science and Technology (Walker ed., 1988); The Glossary of Genetics, 5^{th} Ed., R. Rieger et al. (eds.), Springer Verlag (1991); and Hale & Marham, the Harper Collins Dictionary of Biology (1991). Generally, the procedures of molecular biology methods described or inherent herein and the like are common methods used in the art. Such standard techniques can be found in reference manuals such as for example Sambrook et al., (2000, Molecular Cloning—A Laboratory Manual, Third Edition, Cold Spring Harbor Laboratories); and Ausubel et al., (1994, Current Protocols in Molecular Biology, John Wiley & Sons, New-York).

The following terms may have meanings ascribed to them below, unless specified otherwise. However, it should be understood that other meanings that are known or understood by those having ordinary skill in the art are also possible, and within the scope of the present disclosure.

As used herein, the singular forms “a”, “and”, and “the” include plural references unless the context clearly dictates otherwise. All technical and scientific terms used herein have the same meaning.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein can be modified by the term about.

As used herein, the term “marker” or “biomarker” is a biological molecule, or a panel of biological molecules, whose lipid level is correlated, e.g., either positively or negatively, with FXN levels. In some embodiments, a “marker” or a “biomarker” is an expressed gene, whose lipid level may be measured by measuring levels of the corresponding mRNA or a protein.

As used herein, a marker or biomarker of the disclosure whose respective levels are positively or negatively correlated to frataxin (FXN) levels in a cell or a sample obtained from a subject is referred to as a “Frataxin-sensitive lipid marker” or “FSLM”.

In some embodiments, an FSLM is a marker or a biomarker, e.g., a lipid, that is present in different amounts in samples obtained from a healthy subject as compared to a sample obtained from an FXN-deficient subject, e.g., a subject with FRDA. In further embodiments, an FSLM is contrary regulated by FXN gene ablation or deficiency, followed by FXN replacement therapy. For example, an FSLM may be a lipid, e.g., a triglyceride, wherein the three acyl groups in the triglyceride molecule contain less than 56 carbon atoms, e.g., TG53:2, that is present at a higher level in a sample obtained from an FXN-deficient subject as compared to a sample obtained from a healthy subject, and levels of which decrease in a sample from an FXN-deficient subject following FXN replacement therapy. Alternatively, for example, an FSLM may be PCO-, e.g., PC(−16:0/20:3), that is present at a lower level in a sample obtained an FXN-deficient subject as compared to a sample obtained from a healthy subject, and levels of which increase in a sample from an FXN-deficient subject following FXN replacement therapy.

In some embodiments, the FSLMs of the present disclosure are contrary regulated by FXN gene ablation or deficiency, followed by FXN protein replacement. Thus, in some embodiments, the FSLMs of the present disclosure are both associated with FXN deficiency in a subject and conversely associated with FXN replacement. An FSLM of the disclosure can be used to detect and/or monitor (e.g., serve as a surrogate for) FXN levels in a sample, e.g., a cell or tissue sample. In some embodiments, the FSLMs comprise one or more triglycerides (e.g., one or more triglycerides, wherein the three acyl groups in each triglyceride molecule contain 45 carbons to 58 carbons, or wherein the three acyl groups in each triglyceride molecule contain less than 56 carbons, or wherein the three acyl groups in each triglyceride molecule contain 56 or more carbons), ether phospholipids (e.g., PCO- and PEO-), phosphatidylcholines (PCs), cholesteryl esters (CEs); and diglycerides (DGs).

The term “control sample” or “control,” as used herein, refers to any clinically relevant comparative sample, or comparative lipid profile, including, for example, a sample from an FXN healthy subject (i.e., a subject with a normal FXN level), a normal FXN lipid profile, a sample obtained from an FXN deficient subject (i.e., a subject completely or partially lacking FXN lipid), a baseline FXN(−) lipid profile, or a sample obtained from an FXN-deficient subject following administration of FXN replacement therapy, or an FXN replacement lipid profile. A control sample can also be a sample from a subject from an earlier time point, e.g., prior to treatment with FXN replacement therapy. A control sample can be a purified sample, and/or a lipid provided with a kit. Such control samples can be diluted, for example, in a dilution series to allow for quantitative measurement of levels of analytes, e.g., markers, in test samples. A control sample may include a sample derived from one or more subjects. A control sample may also be a sample taken at an earlier time point from the subject to be assessed. For example, the control sample could be a sample taken from the subject to be assessed before treatment with FXN replacement therapy. The control sample may also be a sample from an animal model, or from a tissue or cell line derived from the animal model of a mitochondrial disease such as FRDA. The level of one or more FSLMs (e.g., 1, 2, 3, 4, 5, 6, 7, 8, or 9 or more FSLMs) in a control sample consists of a group of measurements that may be determined, e.g., based on any appropriate statistical measurement, such as, for example, measures of central tendency including average, median, or modal values. In one embodiment, “different from a control” is preferably statistically significantly different from a control.

As used herein, “changed, altered, increased or decreased” is understood as having a level of the one or more FSLM to be detected at a level that is statistically different, e.g., increased or decreased, as compared to a control sample or to a predetermined threshold value, e.g., from an FXN healthy subject (i.e., a subject with a normal FXN level), or a sample from an FXN deficient subject (e.g., a subject completely or partially lacking FXN lipid or having a reduced level of FXN lipid as compared to a normal control subject). Changed, altered, increased or decreased, as compared to control or threshold value, can also include a difference in the rate of change of the level of one or more FSLMs obtained in a series of at least two subject samples obtained over time. Determination of statistical significance is within the ability of those skilled in the art and can include any acceptable means for determining and/or measuring statistical significance, such as, for example, the number of standard deviations from the mean that constitute a positive or negative result, an increase in the detected level of an FSLM in a sample versus a control, wherein the increase is above some threshold value, or a decrease in the detected level of an FSLM in a sample versus a control, wherein the decrease is below some threshold value.

As used herein, “detecting”, “detection”, “determining”, and the like are understood to refer to identification of the presence and/or level of one or more FSLMs, such as one or more triglycerides (e.g., one or more triglycerides, wherein the three acyl groups in each triglyceride molecule contain 45 carbons to 55 carbons, or wherein the three acyl groups in each triglyceride molecule contain less than 56 carbons, CE16:0, CE20:5 and PC(O-17:0/20:4.

As used herein, the terms “FXN deficient patient” or “FXN deficient subject”, used interchangeably herein, refer to a subject who has been determined to have an FXN deficiency, e.g., been diagnosed with Friedreich's Ataxia (FRDA). In some embodiments, an FXN deficient subject has a reduced level of FXN lipid or activity, e.g., partially or completely lacking FXN expression or activity, as compared to a normal control subject (e.g., has less than 70%, 60%, 50%, 40%, 30%, 20%, or 10% of the FXN expression level or activity of a normal control subject). In some embodiments, an FXN deficient subject has not yet received FXN replacement therapy and is, therefore, therapy naive. In some embodiments, an FXN deficient subject is scheduled to receive FXN replacement therapy. In some embodiments, an FXN deficient subject is currently undergoing FXN replacement therapy. In some embodiments, an FXN deficient subject has already undergone an FXN replacement therapy. In some embodiments, an FXN deficient subject who is undergoing or has already undergone FXN replacement therapy has FXN levels that are partially or completely restored to FXN levels of a healthy subject.

Certain diseases result in FXN deficiencies in subjects, including mitochondrial diseases such as Friedreich's Ataxia (FRDA). As used herein, the term “FXN replacement therapy” refers to replacement of frataxin in a subject which results in increased level or activity of frataxin in the subject. The FXN replacement therapy may be carried out by FXN protein delivery or through delivery of a nucleic acid encoding FXN to a subject. FXN protein delivery to the subject can include delivery of FXN protein or delivery of a FXN fusion protein. As used herein, the term “FXN fusion protein” refers to full length FXN or a fragment of FXN fused to a full length or a fragment of a different protein, or to a peptide. In some embodiments, an FXN fusion protein comprises full-length hFXN (SEQ ID NO: 1) or mature hFXN (SEQ ID NO: 2), as described herein. In some embodiments, the FXN protein or fragment thereof is fused to a cell penetrating peptide (CPP). In some embodiments, the CPP is an HIV-TAT polypeptide. In some embodiments, FXN replacement therapy comprises administering to an FXN-deficient subject an FXN fusion protein comprising or consisting of SEQ ID NO: 12.

As used herein, the terms “disorders”, “diseases”, and “abnormal state” are used inclusively and refer to any deviation from the normal structure or function of any part, organ, or system of the body (or any combination thereof). A specific disease is manifested by characteristic symptoms and signs, including biological, chemical, and physical changes, and is often associated with a variety of other factors including, but not limited to, demographic, environmental, employment, genetic, and medically historical factors. An early stage disease state includes a state wherein one or more physical symptoms are not yet detectable. Certain characteristic signs, symptoms, and related factors can be quantitated through a variety of methods to yield important diagnostic information.

As used herein, the term “mitochondrial disease” refers to a disease which is the result of either inherited or spontaneous mutations in mtDNA or nDNA which leads to altered functions of the proteins or RNA molecules that normally reside in mitochondria, which decreases the functions of the mitochondria to induce diseases of various types in, for example, the central nervous system, skeletal muscles, heart, eyes, liver, kidneys, large intestine (colon), small intestine, internal ear and pancreas; as well as blood, skin and endocrine glands. In one non-limiting embodiment, the mitochondrial disease is Friedreich's Ataxia (FRDA).

As used herein, a sample obtained at an “earlier time point” is a sample that was obtained at a sufficient time in the past such that clinically relevant information could be obtained in the sample from the earlier time point as compared to the later time point. In certain embodiments, an earlier time point is at least 1, 2, 3, 4, 5, 6, 7, 8, 9, 10, 11, 12, 13, 14, 15, 16, 17, 18, 19, 20, 21, 22, or 23 hours, or 1, 2, 3, 4, 5, 6, or 7 days earlier. In some embodiments, an earlier time point is at least one, two, three or four weeks earlier. In certain embodiments, an earlier time point is at least six weeks earlier. In certain embodiments, an earlier time point is at least two months earlier. In certain embodiments, an earlier time point is at least three months earlier. In certain embodiments, an earlier time point is at least six months earlier. In certain embodiments, an earlier time point is at least nine months earlier. In certain embodiments, an earlier time point is at least one year earlier. Multiple subject samples (e.g., 3, 4, 5, 6, 7, or more) can be obtained at regular or irregular intervals over time and analyzed for trends in changes in FSLM levels. Appropriate intervals for testing for a particular subject can be determined by one of skill in the art based on ordinary considerations.

The term “lipid profile” as used herein, refers to a set of data obtained as a result of measuring levels of one or more lipids, such as one or more triglycerides (e.g., one or more triglycerides, wherein the three acyl groups in each triglyceride molecule contain 45 carbons to 55 carbons, or wherein the three acyl groups in each triglyceride molecule contain less than 56 carbons, ether phospholipids, such as PCO- or PEC, e.g., PC(O-17:0/20:4), cholesteryl esters, such as, CE16:0, CE20:5 and CE14:1, and diglycerides, such as DE(18:1/18:2), in a sample, e.g., a sample obtained from a subject. The term “lipid profile” may refer to the raw data, e.g., m/z values and/or retention times, obtained as a result of LC/MS analysis, or to the normalized lipid values. Lipid profiles may be determined by any convenient means for measuring a level of a lipid, such as LC/MS, and other techniques known to a person skilled in the art or described herein. Lipid profiles enable analysis of differential lipid levels between two or more samples, between samples and control, as well as between samples and thresholds. A lipid profile can also be determined by any means known to a person skilled in the art or described herein for measuring the level of a lipid, e.g., mass spectrometry, such as LC/MS, etc.

As referred to herein, the term “FXN lipid profile” includes any one of the following three FXN lipid profiles: a normal FXN lipid profile, a baseline FXN(−) lipid profile, or an FXN replacement lipid profile. In some embodiments, the baseline FXN(−) lipid profile can be used as a control. In some embodiments, the normal FXN lipid profile can be used as a control.

In some embodiments, the term “FXN lipid profile” for one or more FSLMs refers to the lipid level of the one or more FSLMs or to a value or set of values indicative of the lipid level of the one or more FSLMs. In some embodiments, an FXN lipid profile comprises a feature vector of values indicative of level of the one or more FSLMs. In some embodiments, “determining an FXN lipid profile” for one or more FSLMs comprises detecting the level of the one or more FSLMs. In some embodiments, “determining an FXN lipid profile” for one or more FSLMs comprises determining a lipid feature vector of values indicative of the level of the one or more FSLMs.

As referred to herein, the term “normal FXN lipid profile” refers to a lipid profile of one or more FSLMs in a sample obtained from a normal, healthy subject or subjects (i.e., a subject that is not FXN deficient). In some embodiments, a “normal FXN lipid profile” also encompasses an average of multiple, e.g., two or more, normal FXN lipid profiles (e.g., from two or more subjects). As referred to herein, the term “baseline FXN(−) lipid profile” refers to the lipid profile of one or more FSLMs in a sample from an FXN deficient subject prior to treatment with an FXN replacement therapy. In some embodiments, the term “baseline FXN(−) lipid profile encompasses an average of multiple, e.g., two or more, baseline FXN(−) lipid profiles (e.g., from two more subjects).

An average FXN lipid profile, e.g., an average normal FXN lipid profile or an average baseline FXN(−) lipid profile, may be determined by methods known in the art, e.g., by determining the level of one or more FSLMs in a sample obtained from each of two or more subjects, e.g., normal subjects or FXN-deficient subjects, and then calculating an average lipid level of the one or more FSLMs.

As referred to herein, the term “reference FXN lipid profile” encompasses a “normal FXN lipid profile” and a “baseline FXN(−) lipid profile”, i.e., can be either one. The reference lipid profile, e.g., reference normal FXN lipid profile or reference baseline FXN(−) lipid profile, can be used as a control, e.g., for comparing to an FXN replacement lipid profile to evaluate response of a subject to an FXN replacement therapy.

As referred to herein, the term “FXN replacement lipid profile” refers to the lipid profile for one or more FSLMs in a sample obtained from an FXN-deficient subject subsequent to administration of at least one dose of an FXN replacement therapy.

As used herein, the term “treatment with FXN replacement therapy” refers to administration to a subject of at least one dose of an FXN replacement therapy. The terms “following treatment with FXN replacement therapy”, “subsequent to treatment with FXN replacement therapy”, “following administration of FXN replacement therapy” or “subsequent to administration of FXN replacement therapy”, as used herein, refers to at least 1 day, e.g., at least 2, 3, 4, 5, 6, 7, 8, 9, 10, 11, 12, 13, 14, 15, 16, 17, 18, 19, 20, 25, 30, 35, 40, or more days after administration of a dose of an FXN replacement therapy.

As referred to herein, the term “evaluate response of a subject to FXN replacement therapy” encompasses evaluating efficacy of FXN replacement therapy by (a) determining an FXN replacement lipid profile in a sample from an FXN deficient subject following treatment with FXN replacement therapy; (b) comparing the FXN replacement lipid profile with a reference FXN lipid profile; and (c) using the comparison in step (b) to evaluate or determine efficacy of the FXN replacement therapy.

In some embodiments, the reference FXN lipid profile is a baseline FXN(−) lipid profile, i.e., the lipid profile of one or more FSLMs in a sample obtained from an FXN deficient subject prior to treatment with FXN replacement therapy. In some embodiments, a difference between the FXN replacement lipid profile and the baseline FXN(−) lipid profile is indicative of the efficacy of or response to the FXN replacement therapy.

In some embodiments, the reference FXN lipid profile is a normal FXN lipid profile, i.e., the lipid profile of one or more FSLMs in a sample obtained from a normal subject. In some embodiments, a comparison between the FXN replacement lipid profile and the normal FXN lipid profile, e.g., a similarity between the two profiles, is indicative of the efficacy of or response to the FXN replacement therapy.

A “higher level of lipid”, “higher lipid level”, “higher level”, “increased level,” and the like of an FSLM refers to a lipid level in a test sample that is greater than the standard error of the assay employed to assess lipid level, and is preferably at least 25% more, at least 50% more, at least 75% more, at least two, at least three, at least four, at least five, at least six, at least seven, at least eight, at least nine, or at least ten times the lipid level of the FSLM in a control sample (e.g., a sample obtained from a healthy subject, a sample obtained from an FXN deficient subject, or a sample obtained from a subject following FXN replacement therapy) and preferably, the average lipid level of the FSLM or FSLMs in several control samples.

The term “including” is used herein to mean, and is used interchangeably with, the phrase “including but not limited to.”

As used herein, “one or more” is understood as encompassing each value 1, 2, 3, 4, 5, 6, 7, 8, 9, 10, and any value greater than 10.

The term “or” is used inclusively herein to mean, and is used interchangeably with, the term “and/or,” unless context clearly indicates otherwise.

As used herein, “patient” or “subject” can mean either a human or non-human animal, preferably a mammal. By “subject” is meant any animal, including horses, dogs, cats, pigs, goats, rabbits, hamsters, monkeys, guinea pigs, rats, mice, lizards, snakes, sheep, cattle, fish, and birds. A human subject may be referred to as a patient.

As used herein, a “reference level” of an FSLM may be an absolute or relative amount or concentration of the FSLM, a presence or absence of the FSLM, a range of amount or concentration of the FSLM, a minimum and/or maximum amount or concentration of the FSLM, a mean amount or concentration of the FSLM, and/or a median amount or concentration of the FSLM; and, in addition, “reference levels” of combinations of FSLMs may also be ratios of absolute or relative amounts or concentrations of two or more FSLMs with respect to each other. Appropriate positive and negative reference levels of FSLMs for a particular disease state, phenotype, or lack thereof may be determined by measuring levels of desired FSLMs in one or more appropriate subjects, and such reference levels may be tailored to specific populations of subjects (e.g., a reference level may be age-matched so that comparisons may be made between FSLM levels in samples from subjects of a certain age and reference levels for a particular disease state, phenotype, or lack thereof in a certain age group). Such reference levels may also be tailored to specific techniques that are used to measure levels of FSLMs in biological samples (e.g., LC-MS, GC-MS, etc.), where the levels of FSLMs may differ based on the specific technique that is used.

As used herein, “sample” or “biological sample” includes a specimen or culture obtained from any source. In some embodiments, a sample includes any specimen or culture that comprises cells in which FXN lipid profile may be analyzed. In some embodiments, a sample includes any specimen or culture from a subject deficient in FXN or a subject being treated with FXN replacement therapy. For example, biological samples can be obtained from a solid tissue sample, preferably a buccal sample, alternatively a skin biopsy sample, skin strip, hair follicle, muscle biopsy sample, or a body fluid sample such as blood (including any blood product, such as whole blood, plasma, serum, or specific types of cells of the blood, e.g., platelets), urine, saliva, or seminal fluid. In one embodiment, the biological sample is a buccal sample. In another embodiment, the biological sample is a skin sample, e.g., a skin biopsy sample or a skin strip. In one embodiment, the biological sample is a platelet sample. Alternatively, a sample can comprise exosomes which may be harvested in order to be tested for FSLMs.

The term “such as” is used herein to mean, and is used interchangeably, with the phrase “such as but not limited to.”

The term “triglyceride”, which may be used herein interchangeably with the term “triacylglycerol”, refers to a molecule represented by Formula I:

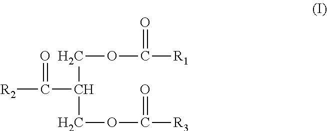

wherein each of R₁, R₂ and R₃ is independently a hydrocarbon chain that may comprise one or more double bonds. As will be readily understood by one of ordinary skill in the art, a term used in reference to specific triglycerides, such as“TG50:4”, refers to triglycerides of Formula I in which R₁, R₂ and R₃ together contain a total of 50 carbons and 4 double bonds. Similarly, the term ““TG51:1” refers to a triglyceride of Formula I in which R₁, R₂ and R₃ together contain a total of 51 carbons and 1 double bond, etc. All other references to specific triglycerides listed below are understood accordingly.

It will also be understood by one of ordinary skill in the art that each term used in reference to specific triglycerides as listed below may encompass more than one triglyceride species. For example, the term “TG50:4” may encompass more than one triglyceride species in which R₁, R₂ and R₃ together contain a total of 56 carbons and 6 double bonds e.g., TG(18:3/16:0/16:1), and TG(18:2/18:2/14:0). Additionally, the term “TG(18:3/16:0/16:1)” encompasses triglycerides in which the positions of the double bonds in R₁ and R₃ may vary.

In some embodiments, the term “triglyceride” refers to a group of lipids that encompasses TG45:1, TG46:1, TG46:3, TG47:1, TG47:2, TG48:0, TG48:1, TG48:2, TG48:3, TG49:1, TG49:2, TG49:3, TG49:4, TG50:1, TG50:2, TG50:3, TG50:4, TG50:5, TG51:1, TG51:2, TG51:3, TG51:4, TG52:2, TG52:3, TG52:4, TG52:5, TG52:6, TG53:2, TG53:3, TG53:4, TG53:5, TG54:4, TG54:5, TG54:6, TG54:7, TG56:4, TG56:5, TG56:6, TG56:7, TG56:8, TG56:9, TG56:10, TG58:5, TG58:6, TG58:7, TG58:8, TG58:11. In some embodiments, the term “triglyceride” refers to a group of triglycerides, wherein the three acyl groups in each triglyceride molecule contain less than 56 carbons and/or wherein the three acyl groups in each triglyceride molecule contain 7 or less unsaturations, e.g., TG45:1, TG46:1, TG46:3, TG47:1, TG47:2, TG48:0, TG48:1, TG48:2, TG48:3, TG49:1, TG49:2, TG49:3, TG49:4, TG50:1, TG50:2, TG50:3, TG50:4, TG50:5, TG51:1, TG51:2, TG51:3, TG51:4, TG52:2, TG52:3, TG52:4, TG52:5, TG52:6, TG53:2, TG53:3, TG53:4, TG53:5, TG54:4, TG54:5, TG54:6 and TG54:7.

In some embodiments, the term “triglyceride” also encompasses epoxy-TGs, i.e., triglycerides in which one or more double bonds, if present, has been oxidized to form an epoxide.

As used herein, the term “unsaturation”, as used herein, refers to a double bond present in a triglyceride. In some embodiments, the language “three acyl groups in each triglyceride molecule contain 7 unsaturations or less” means that the three acyl groups in each triglyceride molecule contain a total of 7, or less than 7, double bonds. In some embodiments, the language “three acyl groups in each triglyceride molecule contain more than 7 unsaturations” means that the three acyl groups in each triglyceride molecule contain more than 7 double bonds. In some embodiments, the language “three acyl groups in each triglyceride molecule contain 6 or more unsaturations” means that the three acyl groups in each triglyceride molecule contain 6 or more double bonds.

As used herein, the term “ether phospholipid” refers to a group of lipids that encompasses ether diacylglycerophosphocholines (PCO-) and phosphatidylethanolamine ethers (PEO-).

The term “ether diacylglycerophosphocholine”, which may be used interchangeably with the term “PCO-”, the term “ether phosphatidylcholine”, the term “phosphatidylcholine ether” or the term “PC ether”, refers to a molecule represented by Formula (II):

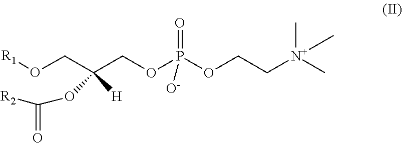

wherein each of R₁ and R₂ is independently a hydrocarbon chain that may comprise one or more double bonds. The term “ether diacylglycerophosphocholines” encompasses, but is not limited to, 1-alkyl, 2-acylglycerophosphocholines representing group GP0102 of the LIPID MAPS® Lipid Classification System (http://lipidmaps.org). The term “ether diacyglycerophosphocholines” also encompasses PC(O-16:0/14:0), PC(O-16:0/18:2), PC(O-16:0/20:3), PC(O-16:0/20:4), PC(O-16:0/22:6), PC(O-17:0/20:4), PC(O-18:0/18:1), PC(O-18:0/22:6), PC(O-18:0/18:2), PC(O-18:1/18:2), PC(O-18:1/20:4), PC(O-18:1/20:5), PC(O-18:1/22:6), PC(O-(20:0/22:6), PC(O-20:1/22.6), PC(O-20:2/20:4), PC(O-22:1/22:6), PC(O-22.2/20:4), PC(O-(24:1/22:6), PC(O-24:2/20:4), PCO-34:2, PCO-36:3, PCO-38:3, PCO-40:2, PCO-40:6, PCO-44:7 and PCO-46.8.

As will be readily understood by one of ordinary skill in the art, the term “PC(O-18:0/18:1)”, for example, refers to a molecule of Formula (II) in which R₁ contains a total of 18 carbons and no double bonds and R₂ contains a total of 18 carbons and 1 double bond. As will also be readily understood by one of ordinary skill in the art, the term “PCO-40:6”, for example, refers to a molecule of Formula (II) in which R₁ and R₂ together contain a total of 40 carbons and a total of 6 double bonds. All other references to specific triglycerides listed above are understood accordingly. It will also be understood by one of ordinary skill in the art that each term used in reference to PCO- as listed above may encompass more than one PCO- species. For example, the term “PCO-40:6” may encompass PCO- species in which the number of carbons and the positions of the double bonds in R₁ and R₂ vary. For example, the term PCO-(18:0/18:1) may encompass PCO- species in which the position of the double bond in R₂ varies.

The term “phosphatidylethanolamine ether”, which may be used interchangeably with the term “ether phosphatidylethanolamine”, the term “PEO-” or the term “PE ether”, refers to a molecule represented by Formula (III):

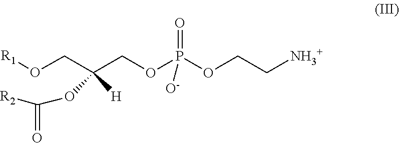

The term “phosphatidylethanolamine ethers” encompasses 1-alkyl, 2-acylglycerophosphoethanolamines, which represent group GP0202 of the LIPID MAPS® Lipid Classification System (http://lipidmaps.org). Exemplary PEO- species include, e.g., PEO-38:5, PEO-36:0, PEO-38:0, PEO-40:0, PEO-34:1, PEO-38:6, etc. It will also be understood by one of ordinary skill in the art that each term used in reference to PEO- as listed above may encompass more than one PEO- species. For example, the term PEO-38:6 may encompass PEO- species in which the number of carbons and the positions of the double bonds in R₁ and R₂ vary.

The term “phosphatidylcholine”, which may be used interchangeably with the term “PC”, encompasses lipids of the structure comprising a glycerol backbone covalently bonded to two fatty acids and a phosphocholine molecule. The fatty acids can be of variable length, hydroxylated, and contain double bonds. In some embodiments, the term “phosphatidylcholines” encompasses the following species: PC(15:0/20:3), PC(15:0/22:6), PC(16:0/14:0), PC(16:0/22:4), PC(16:1/16:0), PC (16:1/20:4), PC(16:1/22:5), PC(17:0/20:5), PC(17:1/20:4), PC(18:0/20:3), PC(18:0/22:4), PC(18:1/20:3), PC(18:1/24:1), PC(18:2/18:2), PC(18:2/18:3), PC(18:2/20:5), PC(20:4/15:0), PC(20:4/20:0), PC(40:6), and PC(42:7).

As will be readily understood by one of ordinary skill in the art, the term “PC(18:1/20:3)”, for example, refers to a phosphatidylcholine in which one fatty acid contains a total of 18 carbons and 1 double bond, and the second fatty acid contains a total of 20 carbons and 3 double bonds. As will also be readily understood by one of ordinary skill in the art, the term “PC(42:7)”, for example, refers to a phosphatidylcholine containing a total of 42 carbon atoms in its two fatty acids and a total of 7 double bonds. All other references to specific PCs listed above are understood accordingly. It will also be understood by one of ordinary skill in the art that each term used in reference to PCs as listed above may encompass more than one PC species.

The term “cholesteryl ester”, which may be used interchangeably with the term “CE”, encompasses lipids of the structure comprising cholesterol covalently bonded to a fatty acid via an ester bond. The ester bond is formed between the carboxylate group of the fatty acid and the hydroxyl group of cholesterol. In some embodiments, the term “cholesteryl ester” encompasses CE14:1, CE16:0 and CE20:5. As will be readily understood by one of ordinary skill in the art, the term “CE14:1”, for example, refers to a cholesteryl ester containing 14 carbon atoms in its fatty acid and one double bond. All other references to specific PCs listed above are understood accordingly. It will also be understood by one of ordinary skill in the art that each term used in reference to PC as listed above may encompass more than one PC species. For example, the term “CE14:1” may encompass species in which the position of the double bond in the fatty acid varies.

The term “diglyceride”, which may be used interchangeably with the term “DGs”, encompasses lipids of the structure comprising two fatty acid chains covalently bonded to a glycerol molecule through ester linkages. The term “diglyceride” encompasses 1,2-diacylglycerols and 1,3-diacylglycerols. In some embodiments, the term “diglycerides” encompasses DG(18:1/18:2), in which one fatty acid contains a total of 18 carbons and 1 double bond, and the second fatty acid contains a total of 18 carbons and 2 double bonds. It will also be understood by one of ordinary skill in the art that each term used in reference to DGs, e.g., DG(18:1/18:2) may encompass more than one DG species. For example, the term “DG(18:1) may encompass DG species in which the locations of one or more double bonds vary.

As used herein, the term “progression of FXN deficiency” refers to worsening of a condition of an FXN-deficient subject over time. This term encompasses an increase in severity and/or duration of existing symptoms of FXN deficiency and/or appearance of one or more new symptoms of FXN deficiency in an FXN-deficient subject.

As used herein, “internal standard” is understood as a chemical substance that is added in a known amount directly to each sample containing an analyte. The amount of analyte present is then determined relative to the internal standard as a calibrant. In some embodiments, the internal standard is an isotopically labeled internal standard, and is an isotopically labeled version of the analyte molecule. The mass spectrometric signal produced by the analyte differs from the mass spectrometric signal produced by the isotopically labeled standard, the difference being dependent on the type and the number of the isotope atoms incorporated into the isotopically labeled version of the analyte. In some embodiments, isotopically labeled version of one or more of TG45:1, TG46:1, TG46:3, TG47:1, TG47:2, TG48:0, TG48:1, TG48:2, TG49:1, TG49:2, TG49:3, TG49:4, TG50:1, TG50:2, TG50:3, TG50:4, TG51:1, TG51:2, TG51:3, TG51:4, TG52:3, TG52:5, TG53:2, TG53:3, TG53:4, TG54:5, TG54:6, TG54:7, TG56:6, TG56:7, TG56:8, TG56:9, TG56:10, TG58:8, TG58:11, CE16:0, CE20:5, PC(O-16:0/18:2), PC(O-16:0/22:6), PC(O-18:0/18:2), PC(O-18:1/20:5), PCO-36:3, PCO-34:2, PCO-40:2, PCO-44:7 and PC(O-17:0/20:4) may function as an internal standard.

As used herein, the term “mass spectrometry” or “MS” refers to an analytical technique to identify compounds by their mass. MS refers to methods of filtering, detecting, and measuring ions based on their mass-to-charge ratio, or “m/z”. MS technology generally includes (1) ionizing the compounds to form charged compounds; and (2) detecting the molecular weight of the charged compounds and calculating a mass-to-charge ratio. The compounds may be ionized and detected by any suitable means. A “mass spectrometer” generally includes an ionizer and an ion detector. In general, one or more molecules of interest are ionized, and the ions are subsequently introduced into a mass spectrometric instrument where, due to a combination of magnetic and electric fields, the ions follow a path in space that is dependent upon mass (“m”) and charge (“z”).

As used herein, the term “operating in negative ion mode” refers to those mass spectrometry methods where negative ions are generated and detected. The term “operating in positive ion mode” as used herein, refers to those mass spectrometry methods where positive ions are generated and detected.

As used herein, the term “ionization” or “ionizing” refers to the process of generating an analyte ion having a net electrical charge equal to one or more electron units. Negative ions are those having a net negative charge of one or more electron units, while positive ions are those having a net positive charge of one or more electron units.

Any compositions or methods provided herein can be combined with one or more of any of the other compositions and methods provided herein.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50.

Reference will now be made in detail to exemplary embodiments of the disclosure. While the disclosure will be described in conjunction with the exemplary embodiments, it will be understood that it is not intended to limit the disclosure to those embodiments. To the contrary, it is intended to cover alternatives, modifications, and equivalents as may be included within the spirit and scope of the disclosure as defined by the appended claims.

### FSLMs of the Disclosure

The present disclosure provides a set of markers, also referred to herein as FXN-sensitive lipid markers (FSLMs), whose respective levels are positively or negatively correlated to frataxin (FXN) levels in a cell or a subject (e.g., a sample from a subject). In some embodiments, the FSLMs of the present disclosure are contrary regulated by FXN gene ablation or deficiency, followed by FXN protein replacement. Thus, said FSLMs of the present disclosure are both associated with FXN deficiency in a subject and conversely associated with FXN replacement. All of the FSLMs disclosed herein were found to be sensitive to FXN levels and are considered markers of FXN replacement therapy. Therefore, these FSLMs can be used to evaluate and/or monitor progression of FXN deficiency in a subject, as described herein. These FSLMs can also be used to evaluate and/or monitor an FXN replacement therapy, e.g., evaluate, monitor, or determine efficacy of an FXN replacement therapy, in a subject, as described herein.

For example, in one aspect, the present disclosure provides a method for determining, evaluating, and/or monitoring the effectiveness of FXN replacement therapy comprising determining: (i) a baseline FXN(−) lipid profile for one or more FSLMs in a sample from an FXN deficient subject prior to treatment with FXN replacement therapy; and (ii) determining a subject FXN replacement lipid profile for the one or more FSLMs in a sample from an FXN deficient subject undergoing FXN replacement therapy or subsequent to treatment with FXN replacement therapy; comparing the subject FXN replacement lipid profile with the baseline FXN(−) lipid profile; and using the results of the comparison to determine, evaluate, or monitor effectiveness of the FXN replacement therapy. Based on the results of the FSLM lipid profile analysis, adjustments can be made to the FXN replacement therapy in the subject to, e.g., initiate, increase (e.g., increase dose and/or frequency of administration), decrease (e.g., decrease dose and/or frequency of administration) or cease FXN replacement therapy in the subject.

Another aspect of the disclosure relates to providing a method for identifying one or more FSLMs, which are markers whose levels is sensitive to FXN levels in a cell of a subject, e.g., an FXN deficient subject. The method comprises determining the lipid profile in a sample from a healthy subject, having normal FXN levels, referred to herein as a normal FXN lipid profile; determining the lipid profile in a sample from a subject having deficient FXN levels, referred to herein as a baseline FXN(−) lipid profile; and comparing the normal FXN lipid profile with the baseline FXN(−) lipid profile; wherein the lipid markers whose level is altered in the baseline FXN(−) lipid profile compared to the normal FXN lipid profile are identified as FSLMs. Additionally, or alternatively, the method for determining FSLMs may comprise a comparison between the lipid profiles obtained from a sample from an FXN deficient subject receiving FXN replacement therapy (baseline FXN(−) lipid profile) and the lipid profiles obtained from a sample from the FXN deficient subject during or after receiving FXN replacement therapy. The lipid profile from a sample obtained from an FXN deficient subject during or after FXN replacement therapy is also referred to herein as an FXN replacement lipid profile. In embodiments, the lipids whose level is altered in the FXN replacement lipid profile as compared to the baseline FXN(−) lipid profile, are identified as FSLMs.

In some embodiments, the FSLMs of the disclosure comprise any combination of one or more TGs (e.g., one or more triglycerides, wherein the three acyl groups in each triglyceride molecule contain 45 carbons to 55 carbons, or wherein the three acyl groups in each triglyceride molecule contain less than 56 carbons, or wherein the three acyl groups in each triglyceride molecule contain 7 or less unsaturations), cholesteryl esters, e.g., CE16:0 or CE20:5; and ether phospholipids, e.g., PCO- or PEO, including phosphatidylcholine ethers, such as PC(O-17:0/20:4, phosphatidylcholines (PCs) and diglycerides (DGs). In some embodiments, the FSLMs of the disclosure comprise any combination of one or more of TG45:1, TG46:1, TG46:3, TG47:1, TG47:2, TG48:0, TG48:1, TG48:2, TG49:1, TG49:2, TG49:3, TG49:4, TG50:1, TG50:2, TG50:3, TG50:4, TG51:1, TG51:2, TG51:3, TG51:4, TG52:3, TG52:5, TG53:2, TG53:3, TG53:4, TG54:5, TG54:6, TG54:7, CE16:0, CE20:5, PC(O-16:0/18:2), PC(O-16:0/22:6), PC(O-18:0/18:2), PC(O-18:1/20:5), PCO-36:3, PCO-34:2, PCO-40:2, PCO-44:7 and PC(O-17:0/20:4). In some embodiments, the FSLMs of the disclosure comprise any combination of one or more of TG45:1, TG46:1, TG46:3, TG47:1, TG47:2, TG48:0, TG48:1, TG48:2, TG48:3, TG49:1, TG49:2, TG49:3, TG49:4, TG50:1, TG50:2, TG50:3, TG50:4, TG50:5, TG51:1, TG51:2, TG51:3, TG51:4, TG52:2, TG52:3, TG52:4, TG52:5, TG52:6, TG53:2, TG53:3, TG53:4, TG53:5, TG54:4, TG54:5, TG54:6, TG54:7, PC(O-16:0/14:0), PC(O-16:0/18:2), PC(O-16:0/20:3), PC(O-16:0/20:4), PC(O-16:0/22:6), PC(O-17:0/20:4), PC(O-18:0/18:1), PC(O-18:0/22:6), PC(O-18:0/18:2), PC(O-18:1/18:2), PC(O-18:1/20:4), PC(O-18:1/20:5), PC(O-18:1/22:6), PC(O-(20:0/22:6), PC(O-20:1/22.6), PC(O-20:2/20:4), PC(O-22:2/20:4), PC(O-22:1/22:6), PC(O-22:2/20:4), PC(O-(24:1/22:6), PC(O-24:2/20:4), PCO-34:2, PCO-36:3, PCO-34:2, PCO-38:3, PCO-40:2, PCO-40:6, PCO-44:7, PCO-46.8, PC(15:0/20:3), PC(15:0/22:6), PC(16:0/14:0), PC(16:0/22:4), PC(16:1/16:0), PC(16:1/20:4), PC(16:1/22.5), PC(17:0/20:5), PC(18:0/20:3), PC(18:0/22:4), PC(18:1/20:3), PC(18:2/18:2), PC(20:4/15:0), PC40:6, PC42:7, CE16:0, CE20:5, CE14:1 and DG18:1/18:2. In some embodiments of the present disclosure, other markers known in the art for measuring FXN lipid or efficacy of FXN replacement therapy can be used in connection with the methods of the present disclosure.

In some embodiments, the one or more (e.g., 1, 2, 3, 4, 5, 6, 7, 8, 9, 10, or more) FSLMs is selected, e.g., from TG45:1, TG46:1, TG46:3, TG47:1, TG47:2, TG48:0, TG48:1, TG48:2, TG48:3, TG49:1, TG49:2, TG49:3, TG49:4, TG50:1, TG50:2, TG50:3, TG50:4, TG50:5, TG51:1, TG51:2, TG51:3, TG51:4, TG52:2, TG52:3, TG52:4, TG52:5, TG52:6, TG53:2, TG53:3, TG53:4, TG53:5, TG54:4, TG54:5, TG54:6, TG54:7, PC(O-16:0/14:0), PC(O-16:0/18:2), PC(O-16:0/20:3), PC(O-16:0/20:4), PC(O-16:0/22:6), PC(O-17:0/20:4), PC(O-18:0/18:1), PC(O-18:0/22:6), PC(O-18:0/18:2), PC(O-18:1/18:2), PC(O-18:1/20:4), PC(O-18:1/20:5), PC(O-18:1/22:6), PC(O-(20:0/22:6), PC(O-20:1/22.6), PC(O-20:2/20:4), PC(O-22:2/20:4), PC(O-22:1/22:6), PC(O-22:2/20:4), PC(O-(24:1/22:6), PC(O-24:2/20:4), PCO-34:2, PCO-36:3, PCO-34:2, PCO-38:3, PCO-40:2, PCO-40:6, PCO-44:7, PCO-46.8, PC(15:0/20:3), PC(15:0/22:6), PC(16:0/14:0), PC(16:0/22:4), PC(16:1/16:0), PC(16:1/20:4), PC(16:1/22.5), PC(17:0/20:5), PC(18:0/20:3), PC(18:0/22:4), PC(18:1/20:3), PC(18:2/18:2), PC(20:4/15:0), PC40:6, PC42:7, PC(17:1/20:4), PC(18:2/18:3), PC(18:1/24:1), PC(18:2/20:5), PC(20:4/20:0), CE14:1, CE16:0, CE20:5 and DG18:1/18:2.

In some embodiments, the term “one or more FSLMs” is intended to mean that one or more (e.g., 1, 2, 3, 4, 5, 6, 7, 8, 9, 10, or more) FSLMs is selected, e.g., from the following groups: (a) triglycerides (TGs), wherein the three acyl groups in each triglyceride molecule contain less than 56 carbons and/or wherein the three acyl groups in each triglyceride molecule contain 7 or less unsaturations; (b) ether phospholipids (e.g., PCO- and PEO-); (c) phosphatidylcholines (PCs); (d) cholesteryl esters (CEs); and (e) diglycerides (DGs). In some embodiments, the one or more FSLMs may be selected from the group consisting of TG45:1, TG46:1, TG46:3, TG47:1, TG47:2, TG48:0, TG48:1, TG48:2, TG48:3, TG49:1, TG49:2, TG49:3, TG49:4, TG50:1, TG50:2, TG50:3, TG50:4, TG50:5, TG51:1, TG51:2, TG51:3, TG51:4, TG52:2, TG52:3, TG52:4, TG52:5, TG52:6, TG53:2, TG53:3, TG53:4, TG53:5, TG54:4, TG54:5, TG54:6, TG54:7, PC(O-16:0/14:0), PC(O-16:0/18:2), PC(O-16:0/20:3), PC(O-16:0/20:4), PC(O-16:0/22:6), PC(O-17:0/20:4), PC(O-18:0/18:1), PC(O-18:0/22:6), PC(O-18:0/18:2), PC(O-18:1/18:2), PC(O-18:1/20:4), PC(O-18:1/20:5), PC(O-18:1/22:6), PC(O-(20:0/22:6), PC(O-20:1/22.6), PC(O-20:2/20:4), PC(O-22:2/20:4), PC(O-22:1/22:6), PC(O-22:2/20:4), PC(O-(24:1/22:6), PC(O-24:2/20:4), PCO-34:2, PCO-36:3, PCO-34:2, PCO-38:3, PCO-40:2, PCO-40:6, PCO-44:7, PCO-46.8, PC(15:0/20:3), PC(15:0/22:6), PC(16:0/14:0), PC(16:0/22:4), PC(16:1/16:0), PC(16:1/20:4), PC(16:1/22.5), PC(17:0/20:5), PC(18:0/20:3), PC(18:0/22:4), PC(18:1/20:3), PC(18:2/18:2), PC(20:4/15:0), PC40:6, PC42:7, PC(17:1/20:4), PC(18:2/18:3), PC(18:1/24:1), PC(18:2/20:5) and PC(20:4/20:0), CE14:1, CE16:0, CE20:5 and DG18:1/18:2.

Methods provided herein include one or any combination of, e.g., 1, 2, 3, 4, 5, 6, 7, 8, 9, 10, or more FSLMs selected from one or more TGs wherein the three acyl groups in each TG molecule contain less than 56 carbon atoms, and/or wherein the three acyl groups in each TG molecule contain 7 or less unsaturations, cholesteryl esters, e.g., CE16:0 or CE20:5, and ether phospholipids, e.g., PCO- or PEO, including phosphatidylcholine ethers, such as PC(O-17:0/20:4).

In some embodiments, the one or more FSLMs of the disclosure comprise one or more (e.g., 1, 2, 3, 4, 5, 6, 7, 8, 9, 10, or more) lipid markers selected from one or more TGs (e.g., one or more triglycerides, wherein the three acyl groups in each triglyceride molecule contain less than 56 carbon atoms, or wherein the three acyl groups in each TG molecule contain 7 or less unsaturations, cholesteryl esters, e.g., CE16:0 or CE20:5 and ether phospholipids, e.g., PCO- or PEO, including phosphatidylcholine ethers, such as PC(O-17:0/20:4.

In some embodiments, the one or more FSLMs comprise one or any combination of one or more TGs, wherein the three acyl groups in each TG molecule contain less than 56 carbon atoms. In some embodiments, the one or more FSLMs comprise one or any combination of one or more TGs, wherein the three acyl groups in each TG molecule contain 7 or less unsaturations. In some embodiments, the one or more FSLMs comprise one or any combination of CE16:0, CE20:5, PC(O-16:0/18:2), PC(O-16:0/22:6), PC(O-18:0/18:2), PC(O-18:1/20:5), PCO-36:3, PCO-34:2, PCO-40:2, PCO-44:7 and PC(O-17:0/20:4). In some embodiments, the one or more FSLMs comprise one or any combination of one or more TGs, wherein the three acyl groups in each TG molecule contain less than 56 carbon atoms, CE16:0, CE20:5, PC(O-16:0/18:2), PC(O-16:0/22:6), PC(O-18:0/18:2), PC(O-18:1/20:5), PCO-36:3, PCO-34:2, PCO-40:2, PCO-44:7 and PC(O-17:0/20:4). In some embodiments, the one or more FSLMs comprise one or any combination of one or more TGs, wherein the three acyl groups in each TG molecule contain 7 or less unsaturations, CE16:0, CE20:5, PC(O-16:0/18:2), PC(O-16:0/22:6), PC(O-18:0/18:2), PC(O-18:1/20:5), PCO-36:3, PCO-34:2, PCO-40:2, PCO-44:7 and PC(O-17:0/20:4).

In some embodiments, the one or more FSLMs comprise one or any combination of TG45:1, TG46:1, TG46:3, TG47:1, TG47:2, TG48:0, TG48:1, TG48:2, TG49:1, TG49:2, TG49:3, TG49:4, TG50:1, TG50:2, TG50:3, TG50:4, TG51:1, TG51:2, TG51:3, TG51:4, TG52:3, TG52:5, TG53:2, TG53:3, TG53:4, TG54:5, TG54:6 and TG54:7. In some embodiments, the one or more FSLMs comprise one or any combination of TG45:1, TG46:1, TG46:3, TG47:1, TG47:2, TG48:0, TG48:1, TG48:2, TG48:3, TG49:1, TG49:2, TG49:3, TG49:4, TG50:1, TG50:2, TG50:3, TG50:4, TG50:5, TG51:1, TG51:2, TG51:3, TG51:4, TG52:2, TG52:3, TG52:4, TG52:5, TG52:6, TG53:2, TG53:3, TG53:4, TG53:5, TG54:4, TG54:5, TG54:6 and TG54:7.

In some embodiments, the one or more FSLMs comprise TG45:1. In some embodiments, the one or more FSLMs comprise TG46:1. In some embodiments, the one or more FSLMs comprise TG46:3. In some embodiments, the one or more FSLMs comprise TG47:1. In some embodiments, the one or more FSLMs comprise TG47:2. In some embodiments, the one or more FSLMs comprise TG48:0. In some embodiments, the one or more FSLMs comprise TG48:1. In some embodiments, the one or more FSLMs comprise TG48:2. In some embodiments, the one or more FSLMs comprise TG48:1. In some embodiments, the one or more FSLMs comprise TG48:3. In some embodiments, the one or more FSLMs comprise TG49:1. In some embodiments, the one or more FSLMs comprise TG49:2. In some embodiments, the one or more FSLMs comprise TG49:3. In some embodiments, the one or more FSLMs comprise TG49:4. In some embodiments, the one or more FSLMs comprise TG50:1. In some embodiments, the one or more FSLMs comprise TG50:2. In some embodiments, the one or more FSLMs comprise TG50:3. In some embodiments, the one or more FSLMs comprise TG50:4. In some embodiments, the one or more FSLMs comprise TG50:5. In some embodiments, the one or more FSLMs comprise TG51:1. In some embodiments, the one or more FSLMs comprise TG51:2. In some embodiments, the one or more FSLMs comprise TG51:3. In some embodiments, the one or more FSLMs comprise TG51:4. In some embodiments, the one or more FSLMs comprise TG52:2. In some embodiments, the one or more FSLMs comprise TG52:3. In some embodiments, the one or more FSLMs comprise TG52:4. In some embodiments, the one or more FSLMs comprise TG52:5. In some embodiments, the one or more FSLMs comprise TG52:6. In some embodiments, the one or more FSLMs comprise TG53:2. In some embodiments, the one or more FSLMs comprise TG53:3. In some embodiments, the one or more FSLMs comprise TG53:4. In some embodiments, the one or more FSLMs comprise TG53:5. In some embodiments, the one or more FSLMs comprise TG54:4. In some embodiments, the one or more FSLMs comprise TG54:5. In some embodiments, the one or more FSLMs comprise TG54:6. In some embodiments, the one or more FSLMs comprise TG54:7.

In some embodiments, the one or more FSLMs comprise one or any combination of TG45:1, TG46:1, TG46:3, TG47:1, TG47:2, TG48:0, TG48:1, TG48:2, TG48:3, TG49:1, TG49:2, TG49:3, TG49:4, TG50:1, TG50:2, TG50:3, TG50:4, TG50:5, TG51:1, TG51:2, TG51:3, TG51:4, TG52:2, TG52:3, TG52:4, TG52:5, TG52:6, TG53:2, TG53:3, TG53:4, TG53:5, TG54:4, TG54:5, TG54:6, TG54:7, PC(O-16:0/14:0), PC(O-16:0/18:2), PC(O-16:0/20:3), PC(O-16:0/20:4), PC(O-16:0/22:6), PC(O-17:0/20:4), PC(O-18:0/18:1), PC(O-18:0/22:6), PC(O-18:0/18:2), PC(O-18:1/18:2), PC(O-18:1/20:4), PC(O-18:1/20:5), PC(O-18:1/22:6), PC(O-(20:0/22:6), PC(O-20:1/22.6), PC(O-20:2/20:4), PC(O-22:2/20:4), PC(O-22:1/22:6), PC(O-22:2/20:4), PC(O-(24:1/22:6), PC(O-24:2/20:4), PCO-34:2, PCO-36:3, PCO-34:2, PCO-38:3, PCO-40:2, PCO-40:6, and PCO-44:7, PCO-46.8, PC(15:0/20:3), PC(15:0/22:6), PC(16:0/14:0), PC(16:0/22:4), PC(16:1/16:0), PC(16:1/20:4), PC(16:1/22.5), PC(17:0/20:5), PC(18:0/20:3), PC(18:0/22:4), PC(18:1/20:3), PC(18:2/18:2), PC(20:4/15:0), PC40:6, PC42:7, PC(17:1/20:4), PC(18:2/18:3), PC(18:1/24:1), PC(18:2/20:5) and PC(20:4/20:0), CE14:1, CE16:0 and CE20:5 and DG18:1/18:2.

In some embodiments, the one or more FSLMs comprise 2 or more, e.g., 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, or 25 or more, of TG45:1, TG46:1, TG46:3, TG47:1, TG47:2, TG48:0, TG48:1, TG48:2, TG48:3, TG49:1, TG49:2, TG49:3, TG49:4, TG50:1, TG50:2, TG50:3, TG50:4, TG50:5, TG51:1, TG51:2, TG51:3, TG51:4, TG52:2, TG52:3, TG52:4, TG52:5, TG52:6, TG53:2, TG53:3, TG53:4, TG53:5, TG54:4, TG54:5, TG54:6, TG54:7, PC(O-16:0/14:0), PC(O-16:0/18:2), PC(O-16:0/20:3), PC(O-16:0/20:4), PC(O-16:0/22:6), PC(O-17:0/20:4), PC(O-18:0/18:1), PC(O-18:0/22:6), PC(O-18:0/18:2), PC(O-18:1/18:2), PC(O-18:1/20:4), PC(O-18:1/20:5), PC(O-18:1/22:6), PC(O-(20:0/22:6), PC(O-20:1/22.6), PC(O-20:2/20:4), PC(O-22:2/20:4), PC(O-22:1/22:6), PC(O-22:2/20:4), PC(O-(24:1/22:6), PC(O-24:2/20:4), PCO-34:2, PCO-36:3, PCO-34:2, PCO-38:3, PCO-40:2, PCO-40:6, and PCO-44:7, PCO-46.8, PC(15:0/20:3), PC(15:0/22:6), PC(16:0/14:0), PC(16:0/22:4), PC(16:1/16:0), PC(16:1/20:4), PC(16:1/22.5), PC(17:0/20:5), PC(18:0/20:3), PC(18:0/22:4), PC(18:1/20:3), PC(18:2/18:2), PC(20:4/15:0), PC40:6, PC42:7, PC(17:1/20:4), PC(18:2/18:3), PC(18:1/24:1), PC(18:2/20:5) and PC(20:4/20:0), CE14:1, CE16:0 and CE20:5 and DG18:1/18:2, in any combination.

In some embodiments, the one or more FSLMs comprise CE16:0. In some embodiments, the one or more FSLMs comprise CE20:5. In some embodiments, the one or more FSLMs comprise PC(O-16:0/18:2). In some embodiments, the one or more FSLMs comprise PC(O-16:0/22:6). In some embodiments, the one or more FSLMs comprise PC(O-18:0/18:2). In some embodiments, the one or more FSLMs comprise PC(O-18:1/20:5). In some embodiments, the one or more FSLMs comprise PCO-36:3. In some embodiments, the one or more FSLMs comprise PCO-34:2. In some embodiments, the one or more FSLMs comprise PCO-40:2. In some embodiments, the one or more FSLMs comprise PCO-44:7. In some embodiments, the one or more FSLMs comprise PC(O-17:0/20:4).

In some embodiments, the one or more FSLMs comprise one or more of TG45:1, TG46:1, TG46:3, TG47:1, TG47:2, TG48:0, TG48:1, TG48:2, TG48:3, TG49:1, TG49:2, TG49:3, TG49:4, TG50:1, TG50:2, TG50:3, TG50:4, TG50:5, TG51:1, TG51:2, TG51:3, TG51:4, TG52:2, TG52:3, TG52:4, TG52:5, TG52:6, TG53:2, TG53:3, TG53:4, TG53:5, TG54:4, TG54:5, TG54:6 and TG54:7. In some embodiments, the one or more FSLMs comprise two or more of TG45:1, TG46:1, TG46:3, TG47:1, TG47:2, TG48:0, TG48:1, TG48:2, TG48:3, TG49:1, TG49:2, TG49:3, TG49:4, TG50:1, TG50:2, TG50:3, TG50:4, TG50:5, TG51:1, TG51:2, TG51:3, TG51:4, TG52:2, TG52:3, TG52:4, TG52:5, TG52:6, TG53:2, TG53:3, TG53:4, TG53:5, TG54:4, TG54:5, TG54:6 and TG54:7. In some embodiments, the one or more FSLMs comprise three or more of TG45:1, TG46:1, TG46:3, TG47:1, TG47:2, TG48:0, TG48:1, TG48:2, TG48:3, TG49:1, TG49:2, TG49:3, TG49:4, TG50:1, TG50:2, TG50:3, TG50:4, TG50:5, TG51:1, TG51:2, TG51:3, TG51:4, TG52:2, TG52:3, TG52:4, TG52:5, TG52:6, TG53:2, TG53:3, TG53:4, TG53:5, TG54:4, TG54:5, TG54:6 and TG54:7. In some embodiments, the one or more FSLMs comprise four or more of TG45:1, TG46:1, TG46:3, TG47:1, TG47:2, TG48:0, TG48:1, TG48:2, TG48:3, TG49:1, TG49:2, TG49:3, TG49:4, TG50:1, TG50:2, TG50:3, TG50:4, TG50:5, TG51:1, TG51:2, TG51:3, TG51:4, TG52:2, TG52:3, TG52:4, TG52:5, TG52:6, TG53:2, TG53:3, TG53:4, TG53:5, TG54:4, TG54:5, TG54:6 and TG54:7. In some embodiments, the one or more FSLMs comprise five or more of TG45:1, TG46:1, TG46:3, TG47:1, TG47:2, TG48:0, TG48:1, TG48:2, TG48:3, TG49:1, TG49:2, TG49:3, TG49:4, TG50:1, TG50:2, TG50:3, TG50:4, TG50:5, TG51:1, TG51:2, TG51:3, TG51:4, TG52:2, TG52:3, TG52:4, TG52:5, TG52:6, TG53:2, TG53:3, TG53:4, TG53:5, TG54:4, TG54:5, TG54:6 and TG54:7. In some embodiments, the one or more FSLMs comprise six or more of TG45:1, TG46:1, TG46:3, TG47:1, TG47:2, TG48:0, TG48:1, TG48:2, TG48:3, TG49:1, TG49:2, TG49:3, TG49:4, TG50:1, TG50:2, TG50:3, TG50:4, TG50:5, TG51:1, TG51:2, TG51:3, TG51:4, TG52:2, TG52:3, TG52:4, TG52:5, TG52:6, TG53:2, TG53:3, TG53:4, TG53:5, TG54:4, TG54:5, TG54:6 and TG54:7. In some embodiments, the one or more FSLMs comprise seven or more of TG45:1, TG46:1, TG46:3, TG47:1, TG47:2, TG48:0, TG48:1, TG48:2, TG48:3, TG49:1, TG49:2, TG49:3, TG49:4, TG50:1, TG50:2, TG50:3, TG50:4, TG50:5, TG51:1, TG51:2, TG51:3, TG51:4, TG52:2, TG52:3, TG52:4, TG52:5, TG52:6, TG53:2, TG53:3, TG53:4, TG53:5, TG54:4, TG54:5, TG54:6 and TG54:7. In some embodiments, the one or more FSLMs comprise eight or more of TG45:1, TG46:1, TG46:3, TG47:1, TG47:2, TG48:0, TG48:1, TG48:2, TG48:3, TG49:1, TG49:2, TG49:3, TG49:4, TG50:1, TG50:2, TG50:3, TG50:4, TG50:5, TG51:1, TG51:2, TG51:3, TG51:4, TG52:2, TG52:3, TG52:4, TG52:5, TG52:6, TG53:2, TG53:3, TG53:4, TG53:5, TG54:4, TG54:5, TG54:6 and TG54:7. In some embodiments, the one or more FSLMs comprise nine or more of TG45:1, TG46:1, TG46:3, TG47:1, TG47:2, TG48:0, TG48:1, TG48:2, TG48:3, TG49:1, TG49:2, TG49:3, TG49:4, TG50:1, TG50:2, TG50:3, TG50:4, TG50:5, TG51:1, TG51:2, TG51:3, TG51:4, TG52:2, TG52:3, TG52:4, TG52:5, TG52:6, TG53:2, TG53:3, TG53:4, TG53:5, TG54:4, TG54:5, TG54:6 and TG54:7. In some embodiments, the one or more FSLMs comprise ten or more of TG45:1, TG46:1, TG46:3, TG47:1, TG47:2, TG48:0, TG48:1, TG48:2, TG48:3, TG49:1, TG49:2, TG49:3, TG49:4, TG50:1, TG50:2, TG50:3, TG50:4, TG50:5, TG51:1, TG51:2, TG51:3, TG51:4, TG52:2, TG52:3, TG52:4, TG52:5, TG52:6, TG53:2, TG53:3, TG53:4, TG53:5, TG54:4, TG54:5, TG54:6 and TG54:7. In some embodiments, the one or more FSLMs comprise eleven or more of TG45:1, TG46:1, TG46:3, TG47:1, TG47:2, TG48:0, TG48:1, TG48:2, TG48:3, TG49:1, TG49:2, TG49:3, TG49:4, TG50:1, TG50:2, TG50:3, TG50:4, TG50:5, TG51:1, TG51:2, TG51:3, TG51:4, TG52:2, TG52:3, TG52:4, TG52:5, TG52:6, TG53:2, TG53:3, TG53:4, TG53:5, TG54:4, TG54:5, TG54:6 and TG54:7. In some embodiments, the one or more FSLMs comprise twelve or more of TG45:1, TG46:1, TG46:3, TG47:1, TG47:2, TG48:0, TG48:1, TG48:2, TG48:3, TG49:1, TG49:2, TG49:3, TG49:4, TG50:1, TG50:2, TG50:3, TG50:4, TG50:5, TG51:1, TG51:2, TG51:3, TG51:4, TG52:2, TG52:3, TG52:4, TG52:5, TG52:6, TG53:2, TG53:3, TG53:4, TG53:5, TG54:4, TG54:5, TG54:6 and TG54:7. In some embodiments, the one or more FSLMs comprise thirteen or more of TG45:1, TG46:1, TG46:3, TG47:1, TG47:2, TG48:0, TG48:1, TG48:2, TG48:3, TG49:1, TG49:2, TG49:3, TG49:4, TG50:1, TG50:2, TG50:3, TG50:4, TG50:5, TG51:1, TG51:2, TG51:3, TG51:4, TG52:2, TG52:3, TG52:4, TG52:5, TG52:6, TG53:2, TG53:3, TG53:4, TG53:5, TG54:4, TG54:5, TG54:6 and TG54:7. In some embodiments, the one or more FSLMs comprise fourteen or more of TG45:1, TG46:1, TG46:3, TG47:1, TG47:2, TG48:0, TG48:1, TG48:2, TG48:3, TG49:1, TG49:2, TG49:3, TG49:4, TG50:1, TG50:2, TG50:3, TG50:4, TG50:5, TG51:1, TG51:2, TG51:3, TG51:4, TG52:2, TG52:3, TG52:4, TG52:5, TG52:6, TG53:2, TG53:3, TG53:4, TG53:5, TG54:4, TG54:5, TG54:6 and TG54:7. In some embodiments, the one or more FSLMs comprise fifteen or more of TG45:1, TG46:1, TG46:3, TG47:1, TG47:2, TG48:0, TG48:1, TG48:2, TG48:3, TG49:1, TG49:2, TG49:3, TG49:4, TG50:1, TG50:2, TG50:3, TG50:4, TG50:5, TG51:1, TG51:2, TG51:3, TG51:4, TG52:2, TG52:3, TG52:4, TG52:5, TG52:6, TG53:2, TG53:3, TG53:4, TG53:5, TG54:4, TG54:5, TG54:6 and TG54:7. In some embodiments, the one or more FSLMs comprise sixteen or more of TG45:1, TG46:1, TG46:3, TG47:1, TG47:2, TG48:0, TG48:1, TG48:2, TG48:3, TG49:1, TG49:2, TG49:3, TG49:4, TG50:1, TG50:2, TG50:3, TG50:4, TG50:5, TG51:1, TG51:2, TG51:3, TG51:4, TG52:2, TG52:3, TG52:4, TG52:5, TG52:6, TG53:2, TG53:3, TG53:4, TG53:5, TG54:4, TG54:5, TG54:6 and TG54:7. In some embodiments, the one or more FSLMs comprise seventeen or more of TG45:1, TG46:1, TG46:3, TG47:1, TG47:2, TG48:0, TG48:1, TG48:2, TG48:3, TG49:1, TG49:2, TG49:3, TG49:4, TG50:1, TG50:2, TG50:3, TG50:4, TG50:5, TG51:1, TG51:2, TG51:3, TG51:4, TG52:2, TG52:3, TG52:4, TG52:5, TG52:6, TG53:2, TG53:3, TG53:4, TG53:5, TG54:4, TG54:5, TG54:6 and TG54:7. In some embodiments, the one or more FSLMs comprise eighteen or more of TG45:1, TG46:1, TG46:3, TG47:1, TG47:2, TG48:0, TG48:1, TG48:2, TG48:3, TG49:1, TG49:2, TG49:3, TG49:4, TG50:1, TG50:2, TG50:3, TG50:4, TG50:5, TG51:1, TG51:2, TG51:3, TG51:4, TG52:2, TG52:3, TG52:4, TG52:5, TG52:6, TG53:2, TG53:3, TG53:4, TG53:5, TG54:4, TG54:5, TG54:6 and TG54:7. In some embodiments, the one or more FSLMs comprise nineteen or more of TG45:1, TG46:1, TG46:3, TG47:1, TG47:2, TG48:0, TG48:1, TG48:2, TG48:3, TG49:1, TG49:2, TG49:3, TG49:4, TG50:1, TG50:2, TG50:3, TG50:4, TG50:5, TG51:1, TG51:2, TG51:3, TG51:4, TG52:2, TG52:3, TG52:4, TG52:5, TG52:6, TG53:2, TG53:3, TG53:4, TG53:5, TG54:4, TG54:5, TG54:6 and TG54:7. In some embodiments, the one or more FSLMs comprise twenty or more of TG45:1, TG46:1, TG46:3, TG47:1, TG47:2, TG48:0, TG48:1, TG48:2, TG48:3, TG49:1, TG49:2, TG49:3, TG49:4, TG50:1, TG50:2, TG50:3, TG50:4, TG50:5, TG51:1, TG51:2, TG51:3, TG51:4, TG52:2, TG52:3, TG52:4, TG52:5, TG52:6, TG53:2, TG53:3, TG53:4, TG53:5, TG54:4, TG54:5, TG54:6 and TG54:7. In some embodiments, the one or more FSLMs comprise twenty-one or more of TG45:1, TG46:1, TG46:3, TG47:1, TG47:2, TG48:0, TG48:1, TG48:2, TG48:3, TG49:1, TG49:2, TG49:3, TG49:4, TG50:1, TG50:2, TG50:3, TG50:4, TG50:5, TG51:1, TG51:2, TG51:3, TG51:4, TG52:2, TG52:3, TG52:4, TG52:5, TG52:6, TG53:2, TG53:3, TG53:4, TG53:5, TG54:4, TG54:5, TG54:6 and TG54:7. In some embodiments, the one or more FSLMs comprise twenty-two or more of TG45:1, TG46:1, TG46:3, TG47:1, TG47:2, TG48:0, TG48:1, TG48:2, TG48:3, TG49:1, TG49:2, TG49:3, TG49:4, TG50:1, TG50:2, TG50:3, TG50:4, TG50:5, TG51:1, TG51:2, TG51:3, TG51:4, TG52:2, TG52:3, TG52:4, TG52:5, TG52:6, TG53:2, TG53:3, TG53:4, TG53:5, TG54:4, TG54:5, TG54:6 and TG54:7. In some embodiments, the one or more FSLMs comprise twenty-three or more of TG45:1, TG46:1, TG46:3, TG47:1, TG47:2, TG48:0, TG48:1, TG48:2, TG48:3, TG49:1, TG49:2, TG49:3, TG49:4, TG50:1, TG50:2, TG50:3, TG50:4, TG50:5, TG51:1, TG51:2, TG51:3, TG51:4, TG52:2, TG52:3, TG52:4, TG52:5, TG52:6, TG53:2, TG53:3, TG53:4, TG53:5, TG54:4, TG54:5, TG54:6 and TG54:7. In some embodiments, the one or more FSLMs comprise twenty-four or more of TG45:1, TG46:1, TG46:3, TG47:1, TG47:2, TG48:0, TG48:1, TG48:2, TG48:3, TG49:1, TG49:2, TG49:3, TG49:4, TG50:1, TG50:2, TG50:3, TG50:4, TG50:5, TG51:1, TG51:2, TG51:3, TG51:4, TG52:2, TG52:3, TG52:4, TG52:5, TG52:6, TG53:2, TG53:3, TG53:4, TG53:5, TG54:4, TG54:5, TG54:6 and TG54:7. In some embodiments, the one or more FSLMs comprise twenty-five or more of TG45:1, TG46:1, TG46:3, TG47:1, TG47:2, TG48:0, TG48:1, TG48:2, TG48:3, TG49:1, TG49:2, TG49:3, TG49:4, TG50:1, TG50:2, TG50:3, TG50:4, TG50:5, TG51:1, TG51:2, TG51:3, TG51:4, TG52:2, TG52:3, TG52:4, TG52:5, TG52:6, TG53:2, TG53:3, TG53:4, TG53:5, TG54:4, TG54:5, TG54:6 and TG54:7. In some embodiments, the one or more FSLMs comprise twenty-six or more of TG45:1, TG46:1, TG46:3, TG47:1, TG47:2, TG48:0, TG48:1, TG48:2, TG48:3, TG49:1, TG49:2, TG49:3, TG49:4, TG50:1, TG50:2, TG50:3, TG50:4, TG50:5, TG51:1, TG51:2, TG51:3, TG51:4, TG52:2, TG52:3, TG52:4, TG52:5, TG52:6, TG53:2, TG53:3, TG53:4, TG53:5, TG54:4, TG54:5, TG54:6 and TG54:7. In some embodiments, the one or more FSLMs comprise twenty-seven or more of TG45:1, TG46:1, TG46:3, TG47:1, TG47:2, TG48:0, TG48:1, TG48:2, TG48:3, TG49:1, TG49:2, TG49:3, TG49:4, TG50:1, TG50:2, TG50:3, TG50:4, TG50:5, TG51:1, TG51:2, TG51:3, TG51:4, TG52:2, TG52:3, TG52:4, TG52:5, TG52:6, TG53:2, TG53:3, TG53:4, TG53:5, TG54:4, TG54:5, TG54:6 and TG54:7. In some embodiments, the one or more FSLMs comprise TG45:1, TG46:1, TG46:3, TG47:1, TG47:2, TG48:0, TG48:1, TG48:2, TG48:3, TG49:1, TG49:2, TG49:3, TG49:4, TG50:1, TG50:2, TG50:3, TG50:4, TG50:5, TG51:1, TG51:2, TG51:3, TG51:4, TG52:2, TG52:3, TG52:4, TG52:5, TG52:6, TG53:2, TG53:3, TG53:4, TG53:5, TG54:4, TG54:5, TG54:6 and TG54:7.

In some embodiments, the one or more FSLMs comprise one or more of CE16:0, CE20:5, PC(O-16:0/18:2), PC(O-16:0/22:6), PC(O-18:0/18:2), PC(O-18:1/20:5), PCO-36:3, PCO-34:2, PCO-40:2, PCO-44:7 and PC(O-17:0/20:4). In some embodiments, the one or more FSLMs comprise two or more of CE16:0, CE20:5, PC(O-16:0/18:2), PC(O-16:0/22:6), PC(O-18:0/18:2), PC(O-18:1/20:5), PCO-36:3, PCO-34:2, PCO-40:2, PCO-44:7 and PC(O-17:0/20:4). In some embodiments, the one or more FSLMs comprise three or more of CE16:0, CE20:5, PC(O-16:0/18:2), PC(O-16:0/22:6), PC(O-18:0/18:2), PC(O-18:1/20:5), PCO-36:3, PCO-34:2, PCO-40:2, PCO-44:7 and PC(O-17:0/20:4). In some embodiments, the one or more FSLMs comprise four or more of CE16:0, CE20:5, PC(O-16:0/18:2), PC(O-16:0/22:6), PC(O-18:0/18:2), PC(O-18:1/20:5), PCO-36:3, PCO-34:2, PCO-40:2, PCO-44:7 and PC(O-17:0/20:4). In some embodiments, the one or more FSLMs comprise five or more of CE16:0, CE20:5, PC(O-16:0/18:2), PC(O-16:0/22:6), PC(O-18:0/18:2), PC(O-18:1/20:5), PCO-36:3, PCO-34:2, PCO-40:2, PCO-44:7 and PC(O-17:0/20:4). In some embodiments, the one or more FSLMs comprise six or more of CE16:0, CE20:5, PC(O-16:0/18:2), PC(O-16:0/22:6), PC(O-18:0/18:2), PC(O-18:1/20:5), PCO-36:3, PCO-34:2, PCO-40:2, PCO-44:7 and PC(O-17:0/20:4). In some embodiments, the one or more FSLMs comprise seven or more of CE16:0, CE20:5, PC(O-16:0/18:2), PC(O-16:0/22:6), PC(O-18:0/18:2), PC(O-18:1/20:5), PCO-36:3, PCO-34:2, PCO-40:2, PCO-44:7 and PC(O-17:0/20:4). In some embodiments, the one or more FSLMs comprise eight or more of CE16:0, CE20:5, PC(O-16:0/18:2), PC(O-16:0/22:6), PC(O-18:0/18:2), PC(O-18:1/20:5), PCO-36:3, PCO-34:2, PCO-40:2, PCO-44:7 and PC(O-17:0/20:4). In some embodiments, the one or more FSLMs comprise nine or more of CE16:0, CE20:5, PC(O-16:0/18:2), PC(O-16:0/22:6), PC(O-18:0/18:2), PC(O-18:1/20:5), PCO-36:3, PCO-34:2, PCO-40:2, PCO-44:7 and PC(O-17:0/20:4). In some embodiments, the one or more FSLMs comprise ten or more of CE16:0, CE20:5, PC(O-16:0/18:2), PC(O-16:0/22:6), PC(O-18:0/18:2), PC(O-18:1/20:5), PCO-36:3, PCO-34:2, PCO-40:2, PCO-44:7 and PC(O-17:0/20:4). In some embodiments, the one or more FSLMs comprise CE16:0, CE20:5, PC(O-16:0/18:2), PC(O-16:0/22:6), PC(O-18:0/18:2), PC(O-18:1/20:5), PCO-36:3, PCO-34:2, PCO-40:2, PCO-44:7 and PC(O-17:0/20:4).

In some embodiments, the one or more FSLMs comprise CE16:0. In some embodiments, the one or more FSLMs comprise CE20:5. In some embodiments, the one or more FSLMs comprise PC(O-16:0/18:2). In some embodiments, the one or more FSLMs comprise PC(O-16:0/22:6). In some embodiments, the one or more FSLMs comprise PC(O-18:0/18:2). In some embodiments, the one or more FSLMs comprise PC(O-18:1/20:5). In some embodiments, the one or more FSLMs comprise PCO-36:3. In some embodiments, the one or more FSLMs comprise PCO-34:2. In some embodiments, the one or more FSLMs comprise PCO-40:2. In some embodiments, the one or more FSLMs comprise PCO-44:7. In some embodiments, the one or more FSLMs comprise PC(O-17:0/20:4). In some embodiments, the one or more FSLMs comprise CE16:0 and CE20:5. In some embodiments, the one or more FSLMs comprise CE16:0 and PC(O-17:0/20:4). In some embodiments, the one or more FSLMs comprise CE20:5 and PC(O-17:0/20:4). In some embodiments, the one or more FSLMs comprise CE16:0, CE20:5 and PC(O-17:0/20:4).

By way of example, an FXN lipid profile may be determined through the measurement of lipid levels of at least one or any combination of more than one FSLM. Hereinafter a lipid profile may also be referred to as a signature.

In one embodiment of the disclosure, a baseline FXN(−) lipid profile comprises altered levels of at least one or any combination of more than one FSLM, such as one or more triglycerides (e.g., one or more triglycerides, wherein the three acyl groups in each triglyceride molecule contain 45 carbons to 55 carbons, or wherein the three acyl groups in each triglyceride molecule contain less than 56 carbons), ether phospholipids, e.g., PCO- and PEO-, phosphatidylcholines (PCs), cholesteryl esters (CEs) and diglycerides (DGs), e.g., CE16:0, CE20:5, PC(O-16:0/18:2), PC(O-16:0/22:6), PC(O-18:0/18:2), PC(O-18:1/20:5), PCO-36:3, PCO-34:2, PCO-40:2, PCO-44:7 and PC(O-17:0/20:4.

In one embodiment of the disclosure, a baseline FXN(−) lipid profile may comprise the downregulated lipid levels of at least one of CE16:0, CE20:5, PC(O-16:0/18:2), PC(O-16:0/22:6), PC(O-18:0/18:2), PC(O-18:1/20:5), PCO-36:3, PCO-34:2, PCO-40:2, PCO-44:7 and PC(O-17:0/20:4), or any combination thereof. In one embodiment of the disclosure, a baseline FXN(−) lipid profile may comprise the downregulated lipid levels of at least one of PC(O-16:0/14:0), PC(O-16:0/18:2), PC(O-16:0/20:3), PC(O-16:0/20:4), PC(O-16:0/22:6), PC(O-17:0/20:4), PC(O-18:0/18:1), PC(O-18:0/22:6), PC(O-18:0/18:2), PC(O-18:1/18:2), PC(O-18:1/20:4), PC(O-18:1/20:5), PC(O-18:1/22:6), PC(O-(20:0/22:6), PC(O-20:1/22.6), PC(O-20:2/20:4), PC(O-22:2/20:4), PC(O-22:1/22:6), PC(O-22:2/20:4), PC(O-(24:1/22:6), PC(O-24:2/20:4), PCO-34:2, PCO-36:3, PCO-34:2, PCO-38:3, PCO-40:2, PCO-40:6, PCO-44:7, PCO-46.8, PC(17:1/20:4), PC(18:2/18:3), PC(18:1/24:1), PC(18:2/20:5), PC(20:4/20:0), CE16:0 and CE20:5. A measure of effectiveness of FXN replacement therapy may be indicated by a pattern of upregulation of levels of any one or more of these FSLMs.

In one embodiment of the disclosure, a baseline FXN(−) lipid profile may comprise the upregulated lipid levels of at least one of TG45:1, TG46:1, TG46:3, TG47:1, TG47:2, TG48:0, TG48:1, TG48:2, TG49:1, TG49:2, TG49:3, TG49:4, TG50:1, TG50:2, TG50:3, TG50:4, TG51:1, TG51:2, TG51:3, TG51:4, TG52:3, TG52:5, TG53:2, TG53:3, TG53:4, TG54:5, TG54:6 and TG54:7, or any combination thereof. In one embodiment of the disclosure, a baseline FXN(−) lipid profile may comprise the upregulated lipid levels of at least one of TG45:1, TG46:1, TG46:3, TG47:1, TG47:2, TG48:0, TG48:1, TG48:2, TG48:3, TG49:1, TG49:2, TG49:3, TG49:4, TG50:1, TG50:2, TG50:3, TG50:4, TG50:5, TG51:1, TG51:2, TG51:3, TG51:4, TG52:2, TG52:3, TG52:4, TG52:5, TG52:6, TG53:2, TG53:3, TG53:4, TG53:5, TG54:4, TG54:5, TG54:6, TG54:7, PC(15:0/20:3), PC(15:0/22:6), PC(16:0/14:0), PC(16:0/22:4), PC(16:1/16:0), PC(16:1/20:4), PC(16:1/22.5), PC(17:0/20:5), PC(18:0/20:3), PC(18:0/22:4), PC(18:1/20:3), PC(18:2/18:2), PC(20:4/15:0), PC40:6, PC42:7, DG18:1/18:2, and CE14:1. A measure of effectiveness of FXN replacement therapy may be indicated by a pattern of downregulation of levels of any one or more of these FSLMs.

In one embodiment, an FXN replacement lipid profile comprises the reversed lipid levels of a baseline FXN(−) lipid profile.

In another embodiment, an FXN replacement lipid profile for use as an indicator of FXN replacement treatment effectiveness may comprise one or any combination of TG45:1, TG46:1, TG46:3, TG47:1, TG47:2, TG48:0, TG48:1, TG48:2, TG49:1, TG49:2, TG49:3, TG49:4, TG50:1, TG50:2, TG50:3, TG50:4, TG51:1, TG51:2, TG51:3, TG51:4, TG52:3, TG52:5, TG53:2, TG53:3, TG53:4, TG54:5, TG54:6, TG54:7, CE16:0, CE20:5, PC(O-16:0/18:2), PC(O-16:0/22:6), PC(O-18:0/18:2), PC(O-18:1/20:5), PCO-36:3, PCO-34:2, PCO-40:2, PCO-44:7 and PC(O-17:0/20:4). In another embodiment, an FXN replacement lipid profile for use as an indicator of FXN replacement treatment effectiveness may comprise one or any combination of TG45:1, TG46:1, TG46:3, TG47:1, TG47:2, TG48:0, TG48:1, TG48:2, TG48:3, TG49:1, TG49:2, TG49:3, TG49:4, TG50:1, TG50:2, TG50:3, TG50:4, TG50:5, TG51:1, TG51:2, TG51:3, TG51:4, TG52:2, TG52:3, TG52:4, TG52:5, TG52:6, TG53:2, TG53:3, TG53:4, TG53:5, TG54:4, TG54:5, TG54:6, TG54:7, PC(O-16:0/14:0), PC(O-16:0/18:2), PC(O-16:0/20:3), PC(O-16:0/20:4), PC(O-16:0/22:6), PC(O-17:0/20:4), PC(O-18:0/18:1), PC(O-18:0/22:6), PC(O-18:0/18:2), PC(O-18:1/18:2), PC(O-18:1/20:4), PC(O-18:1/20:5), PC(O-18:1/22:6), PC(O-(20:0/22:6), PC(O-20:1/22.6), PC(O-20:2/20:4), PC(O-22:2/20:4), PC(O-22:1/22:6), PC(O-22:2/20:4), PC(O-(24:1/22:6), PC(O-24:2/20:4), PCO-34:2, PCO-36:3, PCO-34:2, PCO-38:3, PCO-40:2, PCO-40:6, PCO-44:7, PCO-46.8, PC(15:0/20:3), PC(15:0/22:6), PC(16:0/14:0), PC(16:0/22:4), PC(16:1/16:0), PC(16:1/20:4), PC(16:1/22.5), PC(17:0/20:5), PC(18:0/20:3), PC(18:0/22:4), PC(18:1/20:3), PC(18:2/18:2), PC(20:4/15:0), PC40:6, PC42:7, PC(17:1/20:4), PC(18:2/18:3), PC(18:1/24:1), PC(18:2/20:5), PC(20:4/20:0), CE14:1, CE16:0, CE20:5 and DG18:1/18:2.

In some embodiments, an FXN replacement lipid profile is characterized by the contrary regulation of FSLMs, which is defined by any FSLMs that were downregulated in FXN depletion conditions, e.g., in a subject with FRDA, that become upregulated following FXN replacement therapy; and the reverse is also valid, such that any FSLMs that were upregulated in FXN depletion conditions, e.g., in a subject with FRDA, become downregulated following FXN replacement therapy. Accordingly, detection of altered lipid of one or more FSLMs in a sample following FXN replacement therapy allows for monitoring of efficacy of the FXN replacement therapy in a subject. For example, in one embodiment, a lack of altered lipid of one or more FSLMs in a sample following FXN replacement therapy indicates that the FXN replacement therapy may not have been successful and/or that increased FXN replacement therapy may be needed. Likewise, in another embodiment, altered lipid of one or more FSLMs in a sample following FXN replacement therapy indicates that FXN replacement therapy was successful.

As referred to herein, lipid feature vectors are a set of values that characterize a lipid profile. Lipid feature vectors may comprise a set of n FSLMs, n being the number of different lipids whose levels were measured in a sample. By way of example, n may be all triglycerides (e.g., one or more triglycerides, wherein the three acyl groups in each triglyceride molecule contain 45 carbons to 55 carbons, or wherein the three acyl groups in each triglyceride molecule contain less than 56 carbons; or wherein the three acyl groups in each triglyceride molecule contain 7 or less unsaturations), CE16:0, CE20:5, PC(O-16:0/18:2), PC(O-16:0/22:6), PC(O-18:0/18:2), PC(O-18:1/20:5), PCO-36:3, PCO-34:2, PCO-40:2, PCO-44:7 and PC(O-17:0/20:4). Alternatively, n may be at least one, two, or three, or four, or five, or six, or any number of triglycerides, wherein the three acyl groups in each triglyceride molecule contain less than 56 carbon atoms and/or 7 or less unsaturations (e.g., TG45:1, TG46:1, TG46:3, TG47:1, TG47:2, TG48:0, TG48:1, TG48:2, TG48:3, TG49:1, TG49:2, TG49:3, TG49:4, TG50:1, TG50:2, TG50:3, TG50:4, TG50:5, TG51:1, TG51:2, TG51:3, TG51:4, TG52:2, TG52:3, TG52:4, TG52:5, TG52:6, TG53:2, TG53:3, TG53:4, TG53:5, TG54:4, TG54:5, TG54:6, TG54:7).

In one embodiment, a set of FSLMs may comprise at least one or any combination of more than one FSLM, e.g., triglycerides, CE16:0, CE20:5, PC(O-16:0/18:2), PC(O-16:0/22:6), PC(O-18:0/18:2), PC(O-18:1/20:5), PCO-36:3, PCO-34:2, PCO-40:2, PCO-44:7 and PC(O-17:0/20:4). In one embodiment, one or more of FSLMs comprise one or more of TG45:1, TG46:1, TG46:3, TG47:1, TG47:2, TG48:0, TG48:1, TG48:2, TG48:3, TG49:1, TG49:2, TG49:3, TG49:4, TG50:1, TG50:2, TG50:3, TG50:4, TG50:5, TG51:1, TG51:2, TG51:3, TG51:4, TG52:2, TG52:3, TG52:4, TG52:5, TG52:6, TG53:2, TG53:3, TG53:4, TG53:5, TG54:4, TG54:5, TG54:6, TG54:7, PC(O-16:0/14:0), PC(O-16:0/18:2), PC(O-16:0/20:3), PC(O-16:0/20:4), PC(O-16:0/22:6), PC(O-17:0/20:4), PC(O-18:0/18:1), PC(O-18:0/22:6), PC(O-18:0/18:2), PC(O-18:1/18:2), PC(O-18:1/20:4), PC(O-18:1/20:5), PC(O-18:1/22:6), PC(O-(20:0/22:6), PC(O-20:1/22.6), PC(O-20:2/20:4), PC(O-22:2/20:4), PC(O-22:1/22:6), PC(O-22:2/20:4), PC(O-(24:1/22:6), PC(O-24:2/20:4), PCO-34:2, PCO-36:3, PCO-34:2, PCO-38:3, PCO-40:2, PCO-40:6, PCO-44:7, PCO-46.8, PC(15:0/20:3), PC(15:0/22:6), PC(16:0/14:0), PC(16:0/22:4), PC(16:1/16:0), PC(16:1/20:4), PC(16:1/22.5), PC(17:0/20:5), PC(18:0/20:3), PC(18:0/22:4), PC(18:1/20:3), PC(18:2/18:2), PC(20:4/15:0), PC40:6, PC42:7, PC(17:1/20:4), PC(18:2/18:3), PC(18:1/24:1), PC(18:2/20:5), PC(20:4/20:0), CE14:1, CE16:0, CE20:5 and DG18:1/18:2, in any combination.

In one embodiment, methods provided by the present disclosure comprise determining an FXN lipid profile for the one or more FSLMs described above. In one embodiment, the one or more FSLMs comprise CE16:0, CE20:5, PC(O-16:0/18:2), PC(O-16:0/22:6), PC(O-18:0/18:2), PC(O-18:1/20:5), PCO-36:3, PCO-34:2, PCO-40:2, PCO-44:7 or PC(O-17:0/20:4). In one embodiment, the one or more FSLMs comprise one or more of TG45:1, TG46:1, TG46:3, TG47:1, TG47:2, TG48:0, TG48:1, TG48:2, TG48:3, TG49:1, TG49:2, TG49:3, TG49:4, TG50:1, TG50:2, TG50:3, TG50:4, TG50:5, TG51:1, TG51:2, TG51:3, TG51:4, TG52:2, TG52:3, TG52:4, TG52:5, TG52:6, TG53:2, TG53:3, TG53:4, TG53:5, TG54:4, TG54:5, TG54:6, TG54:7, PC(O-16:0/14:0), PC(O-16:0/18:2), PC(O-16:0/20:3), PC(O-16:0/20:4), PC(O-16:0/22:6), PC(O-17:0/20:4), PC(O-18:0/18:1), PC(O-18:0/22:6), PC(O-18:0/18:2), PC(O-18:1/18:2), PC(O-18:1/20:4), PC(O-18:1/20:5), PC(O-18:1/22:6), PC(O-(20:0/22:6), PC(O-20:1/22.6), PC(O-20:2/20:4), PC(O-22:2/20:4), PC(O-22:1/22:6), PC(O-22:2/20:4), PC(O-(24:1/22:6), PC(O-24:2/20:4), PCO-34:2, PCO-36:3, PCO-34:2, PCO-38:3, PCO-40:2, PCO-40:6, PCO-44:7, PCO-46.8, PC(15:0/20:3), PC(15:0/22:6), PC(16:0/14:0), PC(16:0/22:4), PC(16:1/16:0), PC(16:1/20:4), PC(16:1/22.5), PC(17:0/20:5), PC(18:0/20:3), PC(18:0/22:4), PC(18:1/20:3), PC(18:2/18:2), PC(20:4/15:0), PC40:6, PC42:7, PC(17:1/20:4), PC(18:2/18:3), PC(18:1/24:1), PC(18:2/20:5), PC(20:4/20:0), CE14:1, CE16:0, CE20:5 and DG18:1/18:2.

By way of example, a normal FXN lipid profile, obtained from samples of healthy subjects, may be comprised of lipid levels of a set of FSLMs, and may be represented by and referred to as a normal FXN lipid feature vector. As described in the following examples, FSLMs when measured in FXN deficient samples, e.g., samples from subject with FRDA, may present lipid levels that are different from the levels of lipid of FSLMs in healthy subjects, and thus may be represented by and referred to as a deficient FXN lipid feature vector. In one embodiment, the difference between a deficient FXN lipid feature vector and a normal FXN lipid feature vector may be detected and quantified by the distance between the two lipid feature vectors. In an alternative scenario, lipid levels of FSLMs from a sample from an FXN deficient subject, e.g., subject with FRDA, following FXN replacement treatment may present yet different lipid levels, and may be represented by and referred to as an FXN replacement lipid feature vector. As for the previous two lipid feature vectors, the difference between an FXN replacement lipid feature vector and either a normal FXN lipid feature vector or a deficient FXN lipid feature vector may be detected and quantified by the distance between the replacement FXN lipid feature vector and the normal FXN lipid feature vector or the deficient FXN lipid feature vector.

As such, having a sample from an FXN deficient subject obtained prior to treatment and a sample obtained post-FXN replacement treatment, a first FXN lipid feature vector may be determined for the FXN replacement lipid profile and a second FXN lipid feature vector may be determined for the baseline FXN(−) lipid profile; wherein determining a distance, or scalar product, between the first and the second lipid feature vectors may be used for determining effectiveness of the FXN replacement therapy. In an embodiment of the disclosure, a third lipid feature vector may be determined for the normal FXN lipid profile, the normal lipid profile being established for the FSLMs in a sample from a healthy subject. In an embodiment, the distance between the second (baseline FXN(−) lipid profile) and third (normal FXN lipid profile) FXN lipid feature vectors may be determined. In another embodiment, the distance between the first (FXN replacement lipid profile) and third (normal FXN lipid profile) FXN lipid feature vectors may be determined, and may be used for determining effectiveness of the FXN replacement therapy. In an embodiment, the distance between the first and third lipid feature vectors may be normalized to the distance between the second and third lipid feature vectors, and the resulting normalized distance may be used to determine effectiveness of the FXN replacement therapy. In an embodiment, the resulting normalized distance may be a value ranging from 0 (zero) to 1 (one), wherein the smaller the value (closest to zero) the more effective the therapy.

The biomarkers of the disclosure, such as one or more triglycerides (e.g., one or more of triglycerides (TGs), wherein the three acyl groups in each triglyceride molecule contain less than 56 carbons and/or wherein the three acyl groups in each triglyceride molecule contain 7 or less unsaturations; ether phospholipids (e.g., PCO- and PEO-), phosphatidylcholines (PCs), cholesteryl esters (CEs); and diglycerides (DGs), are correlated with FXN levels in a subject. Accordingly, in one aspect, the present disclosure provides methods for using, measuring, detecting, quantifying, and the like of one or more FSLMs, such as one or more of triglycerides (TGs), wherein the three acyl groups in each triglyceride molecule contain less than 56 carbons and/or wherein the three acyl groups in each triglyceride molecule contain 7 or less unsaturations; ether phospholipids (e.g., PCO- and PEO-), phosphatidylcholines (PCs), cholesteryl esters (CEs); and diglycerides (DGs), for determining and/or monitoring the FXN status in a subject or for determining, evaluating, and/or monitoring FXN replacement therapy in a subject.

In another aspect, the present disclosure relates to using, measuring, detecting, quantifying, and the like of one or more of the FSLMs, such as one or more of triglycerides (TGs), wherein the three acyl groups in each triglyceride molecule contain less than 56 carbons and/or wherein the three acyl groups in each triglyceride molecule contain 7 or less unsaturations; ether phospholipids (e.g., PCO- and PEO-), phosphatidylcholines (PCs), cholesteryl esters (CEs); and diglycerides (DGs) as a surrogate for measuring FXN expression levels.

In addition, in another embodiment, the FSLMs may be used in combination with one or more additional markers for a mitochondrial disease, e.g., FRDA. Other markers that may be used in combination with the one or more FSLMs, such as one or more of triglycerides (TGs), wherein the three acyl groups in each triglyceride molecule contain less than 56 carbons and/or wherein the three acyl groups in each triglyceride molecule contain 7 or less unsaturations; ether phospholipids (e.g., PCO- and PEO-), phosphatidylcholines (PCs), cholesteryl esters (CEs); and diglycerides (DGs), include any measurable characteristic described herein that reflects in a quantitative or qualitative manner the physiological state of an organism, e.g., whether the organism has a mitochondrial disease, e.g., FRDA. The physiological state of an organism is inclusive of any disease or non-disease state, e.g., a subject having a mitochondrial disease, e.g., FRDA, or a subject who is otherwise healthy. The FSLMs of the disclosure that may be used in combination with one or more additional markers include characteristics that can be objectively measured and evaluated as indicators of normal processes, pathogenic processes, or pharmacologic responses to a therapeutic intervention. Such combination markers can be clinical parameters (e.g., age, performance status), laboratory measures (e.g., molecular markers), or genetic or other molecular determinants. In other embodiments, the present disclosure also involves the analysis and consideration of any clinical and/or subject-related health data, for example, data obtained from an Electronic Medical Record (e.g., collection of electronic health information about individual subjects or populations relating to various types of data, such as, demographics, medical history, medication and allergies, immunization status, laboratory test results, radiology images, vital signs, personal statistics like age and weight, and billing information).

The present disclosure also contemplates the use of particular combinations of the FSLMs disclosed herein, e.g., one or more of triglycerides, wherein the three acyl group in each triglyceride molecule contain less than 56 carbons and/or wherein the three acyl groups in each triglyceride molecule contain 7 or less unsaturations, ether phospholipids, phosphatidylcholines (PCs), cholesteryl esters (CEs) and diglycerides (DGs) (e.g., TG45:1, TG46:1, TG46:3, TG47:1, TG47:2, TG48:0, TG48:1, TG48:2, TG48:3, TG49:1, TG49:2, TG49:3, TG49:4, TG50:1, TG50:2, TG50:3, TG50:4, TG50:5, TG51:1, TG51:2, TG51:3, TG51:4, TG52:2, TG52:3, TG52:4, TG52:5, TG52:6, TG53:2, TG53:3, TG53:4, TG53:5, TG54:4, TG54:5, TG54:6, TG54:7, PC(O-16:0/14:0), PC(O-16:0/18:2), PC(O-16:0/20:3), PC(O-16:0/20:4), PC(O-16:0/22:6), PC(O-17:0/20:4), PC(O-18:0/18:1), PC(O-18:0/22:6), PC(O-18:0/18:2), PC(O-18:1/18:2), PC(O-18:1/20:4), PC(O-18:1/20:5), PC(O-18:1/22:6), PC(O-(20:0/22:6), PC(O-20:1/22.6), PC(O-20:2/20:4), PC(O-22:2/20:4), PC(O-22:1/22:6), PC(O-22:2/20:4), PC(O-(24:1/22:6), PC(O-24:2/20:4), PCO-34:2, PCO-36:3, PCO-34:2, PCO-38:3, PCO-40:2, PCO-40:6, PCO-44:7, PCO-46.8, PC(15:0/20:3), PC(15:0/22:6), PC(16:0/14:0), PC(16:0/22:4), PC(16:1/16:0), PC(16:1/20:4), PC(16:1/22.5), PC(17:0/20:5), PC(18:0/20:3), PC(18:0/22:4), PC(18:1/20:3), PC(18:2/18:2), PC(20:4/15:0), PC40:6, PC42:7, PC(17:1/20:4), PC(18:2/18:3), PC(18:1/24:1), PC(18:2/20:5), PC(20:4/20:0), CE14:1, CE16:0, CE20:5 and DG18:1/18:2), in any combination. In one embodiment, the disclosure contemplates FSLM sets with at least two (2) members, which may include any two of TG45:1, TG46:1, TG46:3, TG47:1, TG47:2, TG48:0, TG48:1, TG48:2, TG48:3, TG49:1, TG49:2, TG49:3, TG49:4, TG50:1, TG50:2, TG50:3, TG50:4, TG50:5, TG51:1, TG51:2, TG51:3, TG51:4, TG52:2, TG52:3, TG52:4, TG52:5, TG52:6, TG53:2, TG53:3, TG53:4, TG53:5, TG54:4, TG54:5, TG54:6, TG54:7, PC(O-16:0/14:0), PC(O-16:0/18:2), PC(O-16:0/20:3), PC(O-16:0/20:4), PC(O-16:0/22:6), PC(O-17:0/20:4), PC(O-18:0/18:1), PC(O-18:0/22:6), PC(O-18:0/18:2), PC(O-18:1/18:2), PC(O-18:1/20:4), PC(O-18:1/20:5), PC(O-18:1/22:6), PC(O-(20:0/22:6), PC(O-20:1/22.6), PC(O-20:2/20:4), PC(O-22:2/20:4), PC(O-22:1/22:6), PC(O-22:2/20:4), PC(O-(24:1/22:6), PC(O-24:2/20:4), PCO-34:2, PCO-36:3, PCO-34:2, PCO-38:3, PCO-40:2, PCO-40:6, PCO-44:7, PCO-46.8, PC(15:0/20:3), PC(15:0/22:6), PC(16:0/14:0), PC(16:0/22:4), PC(16:1/16:0), PC(16:1/20:4), PC(16:1/22.5), PC(17:0/20:5), PC(18:0/20:3), PC(18:0/22:4), PC(18:1/20:3), PC(18:2/18:2), PC(20:4/15:0), PC40:6, PC42:7, PC(17:1/20:4), PC(18:2/18:3), PC(18:1/24:1), PC(18:2/20:5), PC(20:4/20:0), CE14:1, CE16:0, CE20:5 and DG18:1/18:2). In another embodiment, the disclosure contemplates FSLM sets with at least three (3) members, which may include any three of TG45:1, TG46:1, TG46:3, TG47:1, TG47:2, TG48:0, TG48:1, TG48:2, TG48:3, TG49:1, TG49:2, TG49:3, TG49:4, TG50:1, TG50:2, TG50:3, TG50:4, TG50:5, TG51:1, TG51:2, TG51:3, TG51:4, TG52:2, TG52:3, TG52:4, TG52:5, TG52:6, TG53:2, TG53:3, TG53:4, TG53:5, TG54:4, TG54:5, TG54:6, TG54:7, PC(O-16:0/14:0), PC(O-16:0/18:2), PC(O-16:0/20:3), PC(O-16:0/20:4), PC(O-16:0/22:6), PC(O-17:0/20:4), PC(O-18:0/18:1), PC(O-18:0/22:6), PC(O-18:0/18:2), PC(O-18:1/18:2), PC(O-18:1/20:4), PC(O-18:1/20:5), PC(O-18:1/22:6), PC(O-(20:0/22:6), PC(O-20:1/22.6), PC(O-20:2/20:4), PC(O-22:2/20:4), PC(O-22:1/22:6), PC(O-22:2/20:4), PC(O-(24:1/22:6), PC(O-24:2/20:4), PCO-34:2, PCO-36:3, PCO-34:2, PCO-38:3, PCO-40:2, PCO-40:6, PCO-44:7, PCO-46.8, PC(15:0/20:3), PC(15:0/22:6), PC(16:0/14:0), PC(16:0/22:4), PC(16:1/16:0), PC(16:1/20:4), PC(16:1/22.5), PC(17:0/20:5), PC(18:0/20:3), PC(18:0/22:4), PC(18:1/20:3), PC(18:2/18:2), PC(20:4/15:0), PC40:6, PC42:7, PC(17:1/20:4), PC(18:2/18:3), PC(18:1/24:1), PC(18:2/20:5), PC(20:4/20:0), CE14:1, CE16:0, CE20:5 and DG18:1/18:2. In another embodiment, the disclosure contemplates FSLM sets with at least four (4) members, which may include any four of TG45:1, TG46:1, TG46:3, TG47:1, TG47:2, TG48:0, TG48:1, TG48:2, TG48:3, TG49:1, TG49:2, TG49:3, TG49:4, TG50:1, TG50:2, TG50:3, TG50:4, TG50:5, TG51:1, TG51:2, TG51:3, TG51:4, TG52:2, TG52:3, TG52:4, TG52:5, TG52:6, TG53:2, TG53:3, TG53:4, TG53:5, TG54:4, TG54:5, TG54:6, TG54:7, PC(O-16:0/14:0), PC(O-16:0/18:2), PC(O-16:0/20:3), PC(O-16:0/20:4), PC(O-16:0/22:6), PC(O-17:0/20:4), PC(O-18:0/18:1), PC(O-18:0/22:6), PC(O-18:0/18:2), PC(O-18:1/18:2), PC(O-18:1/20:4), PC(O-18:1/20:5), PC(O-18:1/22:6), PC(O-(20:0/22:6), PC(O-20:1/22.6), PC(O-20:2/20:4), PC(O-22:2/20:4), PC(O-22:1/22:6), PC(O-22:2/20:4), PC(O-(24:1/22:6), PC(O-24:2/20:4), PCO-34:2, PCO-36:3, PCO-34:2, PCO-38:3, PCO-40:2, PCO-40:6, PCO-44:7, PCO-46.8, PC(15:0/20:3), PC(15:0/22:6), PC(16:0/14:0), PC(16:0/22:4), PC(16:1/16:0), PC(16:1/20:4), PC(16:1/22.5), PC(17:0/20:5), PC(18:0/20:3), PC(18:0/22:4), PC(18:1/20:3), PC(18:2/18:2), PC(20:4/15:0), PC40:6, PC42:7, PC(17:1/20:4), PC(18:2/18:3), PC(18:1/24:1), PC(18:2/20:5), PC(20:4/20:0), CE14:1, CE16:0, CE20:5 and DG18:1/18:2. In another embodiment, the disclosure contemplates FSLM sets with at least five (5) members, which may include any five of TG45:1, TG46:1, TG46:3, TG47:1, TG47:2, TG48:0, TG48:1, TG48:2, TG48:3, TG49:1, TG49:2, TG49:3, TG49:4, TG50:1, TG50:2, TG50:3, TG50:4, TG50:5, TG51:1, TG51:2, TG51:3, TG51:4, TG52:2, TG52:3, TG52:4, TG52:5, TG52:6, TG53:2, TG53:3, TG53:4, TG53:5, TG54:4, TG54:5, TG54:6, TG54:7, PC(O-16:0/14:0), PC(O-16:0/18:2), PC(O-16:0/20:3), PC(O-16:0/20:4), PC(O-16:0/22:6), PC(O-17:0/20:4), PC(O-18:0/18:1), PC(O-18:0/22:6), PC(O-18:0/18:2), PC(O-18:1/18:2), PC(O-18:1/20:4), PC(O-18:1/20:5), PC(O-18:1/22:6), PC(O-(20:0/22:6), PC(O-20:1/22.6), PC(O-20:2/20:4), PC(O-22:2/20:4), PC(O-22:1/22:6), PC(O-22:2/20:4), PC(O-(24:1/22:6), PC(O-24:2/20:4), PCO-34:2, PCO-36:3, PCO-34:2, PCO-38:3, PCO-40:2, PCO-40:6, PCO-44:7, PCO-46.8, PC(15:0/20:3), PC(15:0/22:6), PC(16:0/14:0), PC(16:0/22:4), PC(16:1/16:0), PC(16:1/20:4), PC(16:1/22.5), PC(17:0/20:5), PC(18:0/20:3), PC(18:0/22:4), PC(18:1/20:3), PC(18:2/18:2), PC(20:4/15:0), PC40:6, PC42:7, PC(17:1/20:4), PC(18:2/18:3), PC(18:1/24:1), PC(18:2/20:5), PC(20:4/20:0), CE14:1, CE16:0, CE20:5 and DG18:1/18:2. In another embodiment, the disclosure contemplates FSLM sets with at least six (6) members, which may include any six of TG45:1, TG46:1, TG46:3, TG47:1, TG47:2, TG48:0, TG48:1, TG48:2, TG48:3, TG49:1, TG49:2, TG49:3, TG49:4, TG50:1, TG50:2, TG50:3, TG50:4, TG50:5, TG51:1, TG51:2, TG51:3, TG51:4, TG52:2, TG52:3, TG52:4, TG52:5, TG52:6, TG53:2, TG53:3, TG53:4, TG53:5, TG54:4, TG54:5, TG54:6, TG54:7, PC(O-16:0/14:0), PC(O-16:0/18:2), PC(O-16:0/20:3), PC(O-16:0/20:4), PC(O-16:0/22:6), PC(O-17:0/20:4), PC(O-18:0/18:1), PC(O-18:0/22:6), PC(O-18:0/18:2), PC(O-18:1/18:2), PC(O-18:1/20:4), PC(O-18:1/20:5), PC(O-18:1/22:6), PC(O-(20:0/22:6), PC(O-20:1/22.6), PC(O-20:2/20:4), PC(O-22:2/20:4), PC(O-22:1/22:6), PC(O-22:2/20:4), PC(O-(24:1/22:6), PC(O-24:2/20:4), PCO-34:2, PCO-36:3, PCO-34:2, PCO-38:3, PCO-40:2, PCO-40:6, PCO-44:7, PCO-46.8, PC(15:0/20:3), PC(15:0/22:6), PC(16:0/14:0), PC(16:0/22:4), PC(16:1/16:0), PC(16:1/20:4), PC(16:1/22.5), PC(17:0/20:5), PC(18:0/20:3), PC(18:0/22:4), PC(18:1/20:3), PC(18:2/18:2), PC(20:4/15:0), PC40:6, PC42:7, PC(17:1/20:4), PC(18:2/18:3), PC(18:1/24:1), PC(18:2/20:5), PC(20:4/20:0), CE14:1, CE16:0, CE20:5 and DG18:1/18:2. In another embodiment, the disclosure contemplates FSLM sets with at least seven (7) members, which may include any seven of TG45:1, TG46:1, TG46:3, TG47:1, TG47:2, TG48:0, TG48:1, TG48:2, TG48:3, TG49:1, TG49:2, TG49:3, TG49:4, TG50:1, TG50:2, TG50:3, TG50:4, TG50:5, TG51:1, TG51:2, TG51:3, TG51:4, TG52:2, TG52:3, TG52:4, TG52:5, TG52:6, TG53:2, TG53:3, TG53:4, TG53:5, TG54:4, TG54:5, TG54:6, TG54:7, PC(O-16:0/14:0), PC(O-16:0/18:2), PC(O-16:0/20:3), PC(O-16:0/20:4), PC(O-16:0/22:6), PC(O-17:0/20:4), PC(O-18:0/18:1), PC(O-18:0/22:6), PC(O-18:0/18:2), PC(O-18:1/18:2), PC(O-18:1/20:4), PC(O-18:1/20:5), PC(O-18:1/22:6), PC(O-(20:0/22:6), PC(O-20:1/22.6), PC(O-20:2/20:4), PC(O-22:2/20:4), PC(O-22:1/22:6), PC(O-22:2/20:4), PC(O-(24:1/22:6), PC(O-24:2/20:4), PCO-34:2, PCO-36:3, PCO-34:2, PCO-38:3, PCO-40:2, PCO-40:6, PCO-44:7, PCO-46.8, PC(15:0/20:3), PC(15:0/22:6), PC(16:0/14:0), PC(16:0/22:4), PC(16:1/16:0), PC(16:1/20:4), PC(16:1/22.5), PC(17:0/20:5), PC(18:0/20:3), PC(18:0/22:4), PC(18:1/20:3), PC(18:2/18:2), PC(20:4/15:0), PC40:6, PC42:7, PC(17:1/20:4), PC(18:2/18:3), PC(18:1/24:1), PC(18:2/20:5), PC(20:4/20:0), CE14:1, CE16:0, CE20:5 and DG18:1/18:2. In another embodiment, the disclosure contemplates FSLM sets with at least eight (8) members, which may include any eight of TG45:1, TG46:1, TG46:3, TG47:1, TG47:2, TG48:0, TG48:1, TG48:2, TG48:3, TG49:1, TG49:2, TG49:3, TG49:4, TG50:1, TG50:2, TG50:3, TG50:4, TG50:5, TG51:1, TG51:2, TG51:3, TG51:4, TG52:2, TG52:3, TG52:4, TG52:5, TG52:6, TG53:2, TG53:3, TG53:4, TG53:5, TG54:4, TG54:5, TG54:6, TG54:7, PC(O-16:0/14:0), PC(O-16:0/18:2), PC(O-16:0/20:3), PC(O-16:0/20:4), PC(O-16:0/22:6), PC(O-17:0/20:4), PC(O-18:0/18:1), PC(O-18:0/22:6), PC(O-18:0/18:2), PC(O-18:1/18:2), PC(O-18:1/20:4), PC(O-18:1/20:5), PC(O-18:1/22:6), PC(O-(20:0/22:6), PC(O-20:1/22.6), PC(O-20:2/20:4), PC(O-22:2/20:4), PC(O-22:1/22:6), PC(O-22:2/20:4), PC(O-(24:1/22:6), PC(O-24:2/20:4), PCO-34:2, PCO-36:3, PCO-34:2, PCO-38:3, PCO-40:2, PCO-40:6, PCO-44:7, PCO-46.8, PC(15:0/20:3), PC(15:0/22:6), PC(16:0/14:0), PC(16:0/22:4), PC(16:1/16:0), PC(16:1/20:4), PC(16:1/22.5), PC(17:0/20:5), PC(18:0/20:3), PC(18:0/22:4), PC(18:1/20:3), PC(18:2/18:2), PC(20:4/15:0), PC40:6, PC42:7, PC(17:1/20:4), PC(18:2/18:3), PC(18:1/24:1), PC(18:2/20:5), PC(20:4/20:0), CE14:1, CE16:0, CE20:5 and DG18:1/18:2. In another embodiment, the disclosure contemplates FSLM sets with at least nine (9) members, which may include any nine of TG45:1, TG46:1, TG46:3, TG47:1, TG47:2, TG48:0, TG48:1, TG48:2, TG48:3, TG49:1, TG49:2, TG49:3, TG49:4, TG50:1, TG50:2, TG50:3, TG50:4, TG50:5, TG51:1, TG51:2, TG51:3, TG51:4, TG52:2, TG52:3, TG52:4, TG52:5, TG52:6, TG53:2, TG53:3, TG53:4, TG53:5, TG54:4, TG54:5, TG54:6, TG54:7, PC(O-16:0/14:0), PC(O-16:0/18:2), PC(O-16:0/20:3), PC(O-16:0/20:4), PC(O-16:0/22:6), PC(O-17:0/20:4), PC(O-18:0/18:1), PC(O-18:0/22:6), PC(O-18:0/18:2), PC(O-18:1/18:2), PC(O-18:1/20:4), PC(O-18:1/20:5), PC(O-18:1/22:6), PC(O-(20:0/22:6), PC(O-20:1/22.6), PC(O-20:2/20:4), PC(O-22:2/20:4), PC(O-22:1/22:6), PC(O-22:2/20:4), PC(O-(24:1/22:6), PC(O-24:2/20:4), PCO-34:2, PCO-36:3, PCO-34:2, PCO-38:3, PCO-40:2, PCO-40:6, PCO-44:7, PCO-46.8, PC(15:0/20:3), PC(15:0/22:6), PC(16:0/14:0), PC(16:0/22:4), PC(16:1/16:0), PC(16:1/20:4), PC(16:1/22.5), PC(17:0/20:5), PC(18:0/20:3), PC(18:0/22:4), PC(18:1/20:3), PC(18:2/18:2), PC(20:4/15:0), PC40:6, PC42:7, PC(17:1/20:4), PC(18:2/18:3), PC(18:1/24:1), PC(18:2/20:5), PC(20:4/20:0), CE14:1, CE16:0, CE20:5 and DG18:1/18:2. In another embodiment, the disclosure contemplates FSLM sets with at least ten (10) members, which may include any ten of TG45:1, TG46:1, TG46:3, TG47:1, TG47:2, TG48:0, TG48:1, TG48:2, TG48:3, TG49:1, TG49:2, TG49:3, TG49:4, TG50:1, TG50:2, TG50:3, TG50:4, TG50:5, TG51:1, TG51:2, TG51:3, TG51:4, TG52:2, TG52:3, TG52:4, TG52:5, TG52:6, TG53:2, TG53:3, TG53:4, TG53:5, TG54:4, TG54:5, TG54:6, TG54:7, PC(O-16:0/14:0), PC(O-16:0/18:2), PC(O-16:0/20:3), PC(O-16:0/20:4), PC(O-16:0/22:6), PC(O-17:0/20:4), PC(O-18:0/18:1), PC(O-18:0/22:6), PC(O-18:0/18:2), PC(O-18:1/18:2), PC(O-18:1/20:4), PC(O-18:1/20:5), PC(O-18:1/22:6), PC(O-(20:0/22:6), PC(O-20:1/22.6), PC(O-20:2/20:4), PC(O-22:2/20:4), PC(O-22:1/22:6), PC(O-22:2/20:4), PC(O-(24:1/22:6), PC(O-24:2/20:4), PCO-34:2, PCO-36:3, PCO-34:2, PCO-38:3, PCO-40:2, PCO-40:6, PCO-44:7, PCO-46.8, PC(15:0/20:3), PC(15:0/22:6), PC(16:0/14:0), PC(16:0/22:4), PC(16:1/16:0), PC(16:1/20:4), PC(16:1/22.5), PC(17:0/20:5), PC(18:0/20:3), PC(18:0/22:4), PC(18:1/20:3), PC(18:2/18:2), PC(20:4/15:0), PC40:6, PC42:7, PC(17:1/20:4), PC(18:2/18:3), PC(18:1/24:1), PC(18:2/20:5), PC(20:4/20:0), CE14:1, CE16:0, CE20:5 and DG18:1/18:2. In another embodiment, the disclosure contemplates FSLM sets with at least eleven (11) members, which may include any eleven of TG45:1, TG46:1, TG46:3, TG47:1, TG47:2, TG48:0, TG48:1, TG48:2, TG48:3, TG49:1, TG49:2, TG49:3, TG49:4, TG50:1, TG50:2, TG50:3, TG50:4, TG50:5, TG51:1, TG51:2, TG51:3, TG51:4, TG52:2, TG52:3, TG52:4, TG52:5, TG52:6, TG53:2, TG53:3, TG53:4, TG53:5, TG54:4, TG54:5, TG54:6, TG54:7, PC(O-16:0/14:0), PC(O-16:0/18:2), PC(O-16:0/20:3), PC(O-16:0/20:4), PC(O-16:0/22:6), PC(O-17:0/20:4), PC(O-18:0/18:1), PC(O-18:0/22:6), PC(O-18:0/18:2), PC(O-18:1/18:2), PC(O-18:1/20:4), PC(O-18:1/20:5), PC(O-18:1/22:6), PC(O-(20:0/22:6), PC(O-20:1/22.6), PC(O-20:2/20:4), PC(O-22:2/20:4), PC(O-22:1/22:6), PC(O-22:2/20:4), PC(O-(24:1/22:6), PC(O-24:2/20:4), PCO-34:2, PCO-36:3, PCO-34:2, PCO-38:3, PCO-40:2, PCO-40:6, PCO-44:7, PCO-46.8, PC(15:0/20:3), PC(15:0/22:6), PC(16:0/14:0), PC(16:0/22:4), PC(16:1/16:0), PC(16:1/20:4), PC(16:1/22.5), PC(17:0/20:5), PC(18:0/20:3), PC(18:0/22:4), PC(18:1/20:3), PC(18:2/18:2), PC(20:4/15:0), PC40:6, PC42:7, PC(17:1/20:4), PC(18:2/18:3), PC(18:1/24:1), PC(18:2/20:5), PC(20:4/20:0), CE14:1, CE16:0, CE20:5 and DG18:1/18:2. In another embodiment, the disclosure contemplates FSLM sets with at least twelve (12) members, which may include any twelve of TG45:1, TG46:1, TG46:3, TG47:1, TG47:2, TG48:0, TG48:1, TG48:2, TG48:3, TG49:1, TG49:2, TG49:3, TG49:4, TG50:1, TG50:2, TG50:3, TG50:4, TG50:5, TG51:1, TG51:2, TG51:3, TG51:4, TG52:2, TG52:3, TG52:4, TG52:5, TG52:6, TG53:2, TG53:3, TG53:4, TG53:5, TG54:4, TG54:5, TG54:6, TG54:7, PC(O-16:0/14:0), PC(O-16:0/18:2), PC(O-16:0/20:3), PC(O-16:0/20:4), PC(O-16:0/22:6), PC(O-17:0/20:4), PC(O-18:0/18:1), PC(O-18:0/22:6), PC(O-18:0/18:2), PC(O-18:1/18:2), PC(O-18:1/20:4), PC(O-18:1/20:5), PC(O-18:1/22:6), PC(O-(20:0/22:6), PC(O-20:1/22.6), PC(O-20:2/20:4), PC(O-22:2/20:4), PC(O-22:1/22:6), PC(O-22:2/20:4), PC(O-(24:1/22:6), PC(O-24:2/20:4), PCO-34:2, PCO-36:3, PCO-34:2, PCO-38:3, PCO-40:2, PCO-40:6, PCO-44:7, PCO-46.8, PC(15:0/20:3), PC(15:0/22:6), PC(16:0/14:0), PC(16:0/22:4), PC(16:1/16:0), PC(16:1/20:4), PC(16:1/22.5), PC(17:0/20:5), PC(18:0/20:3), PC(18:0/22:4), PC(18:1/20:3), PC(18:2/18:2), PC(20:4/15:0), PC40:6, PC42:7, PC(17:1/20:4), PC(18:2/18:3), PC(18:1/24:1), PC(18:2/20:5), PC(20:4/20:0), CE14:1, CE16:0, CE20:5 and DG18:1/18:2.

In other embodiments, the disclosure contemplates an FSLM set comprising at least 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 70, 80, 90, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, or 138 of the FSLMs, such as TG45:1, TG46:1, TG46:3, TG47:1, TG47:2, TG48:0, TG48:1, TG48:2, TG48:3, TG49:1, TG49:2, TG49:3, TG49:4, TG50:1, TG50:2, TG50:3, TG50:4, TG50:5, TG51:1, TG51:2, TG51:3, TG51:4, TG52:2, TG52:3, TG52:4, TG52:5, TG52:6, TG53:2, TG53:3, TG53:4, TG53:5, TG54:4, TG54:5, TG54:6, TG54:7, PC(O-16:0/14:0), PC(O-16:0/18:2), PC(O-16:0/20:3), PC(O-16:0/20:4), PC(O-16:0/22:6), PC(O-17:0/20:4), PC(O-18:0/18:1), PC(O-18:0/22:6), PC(O-18:0/18:2), PC(O-18:1/18:2), PC(O-18:1/20:4), PC(O-18:1/20:5), PC(O-18:1/22:6), PC(O-(20:0/22:6), PC(O-20:1/22.6), PC(O-20:2/20:4), PC(O-22:2/20:4), PC(O-22:1/22:6), PC(O-22:2/20:4), PC(O-(24:1/22:6), PC(O-24:2/20:4), PCO-34:2, PCO-36:3, PCO-34:2, PCO-38:3, PCO-40:2, PCO-40:6, PCO-44:7, PCO-46.8, PC(15:0/20:3), PC(15:0/22:6), PC(16:0/14:0), PC(16:0/22:4), PC(16:1/16:0), PC(16:1/20:4), PC(16:1/22.5), PC(17:0/20:5), PC(18:0/20:3), PC(18:0/22:4), PC(18:1/20:3), PC(18:2/18:2), PC(20:4/15:0), PC40:6, PC42:7, PC(17:1/20:4), PC(18:2/18:3), PC(18:1/24:1), PC(18:2/20:5), PC(20:4/20:0), CE14:1, CE16:0, CE20:5 and DG18:1/18:2.

In certain embodiments, the level of one or more FSLMs is increased following treatment of a subject with an FXN replacement therapy, e.g., a subject deficient in FXN. In some embodiments, the one or more FSLMs is selected from the group consisting of cholesteryl esters, e.g., CE16:0 or CE20:5 and ether phospholipids, e.g., PCO-, PEO-, including phosphatidylcholine ethers, such as PC(O-17:0/20:4). In some embodiments, the one or more FSLMs include one or more of PC(O-16:0/14:0), PC(O-16:0/18:2), PC(O-16:0/20:3), PC(O-16:0/20:4), PC(O-16:0/22:6), PC(O-17:0/20:4), PC(O-18:0/18:1), PC(O-18:0/22:6), PC(O-18:0/18:2), PC(O-18:1/18:2), PC(O-18:1/20:4), PC(O-18:1/20:5), PC(O-18:1/22:6), PC(O-(20:0/22:6), PC(O-20:1/22.6), PC(O-20:2/20:4), PC(O-22:2/20:4), PC(O-22:1/22:6), PC(O-22:2/20:4), PC(O-(24:1/22:6), PC(O-24:2/20:4), PCO-34:2, PCO-36:3, PCO-34:2, PCO-38:3, PCO-40:2, PCO-40:6, PCO-44:7, PCO-46.8, PC(17:1/20:4), PC(18:2/18:3), PC(18:1/24:1), PC(18:2/20:5), PC(20:4/20:0), CE16:0 and CE20:5.

In certain embodiments, the level of one or more FSLMs is decreased following treatment of a subject with FXN replacement, e.g., a subject deficient in FXN. In some embodiments, the one or more FSLM is selected from the group consisting of triglycerides, wherein the three acyl groups in each triglyceride molecule contain less than 56 carbon atoms, e.g., TG45:1, TG46:1, TG46:3, TG47:1, TG47:2, TG48:0, TG48:1, TG48:2, TG48:3, TG49:1, TG49:2, TG49:3, TG49:4, TG50:1, TG50:2, TG50:3, TG50:4, TG50:5, TG51:1, TG51:2, TG51:3, TG51:4, TG52:2, TG52:3, TG52:4, TG52:5, TG52:6, TG53:2, TG53:3, TG53:4, TG53:5, TG54:4, TG54:5, TG54:6, TG54:7. In some embodiments, the FSLMs are selected from the group consisting of TG45:1, TG46:1, TG46:3, TG47:1, TG47:2, TG48:0, TG48:1, TG48:2, TG48:3, TG49:1, TG49:2, TG49:3, TG49:4, TG50:1, TG50:2, TG50:3, TG50:4, TG50:5, TG51:1, TG51:2, TG51:3, TG51:4, TG52:2, TG52:3, TG52:4, TG52:5, TG52:6, TG53:2, TG53:3, TG53:4, TG53:5, TG54:4, TG54:5, TG54:6, TG54:7, PC(15:0/20:3), PC(15:0/22:6), PC(16:0/14:0), PC(16:0/22:4), PC(16:1/16:0), PC(16:1/20:4), PC(16:1/22.5), PC(17:0/20:5), PC(18:0/20:3), PC(18:0/22:4), PC(18:1/20:3), PC(18:2/18:2), PC(20:4/15:0), PC40:6, PC42:7, DG18:1/18:2 and CE14:1.

In another aspect, the present disclosure provides for the identification of a “diagnostic lipid signature” or “diagnostic lipid profile” based on the levels of the FSLMs of the disclosure in a biological sample, that correlates with FXN in the sample. The “levels of the FSLMs” can refer to the lipid level of an FSLM in a biological sample.

In certain embodiments, the diagnostic signature is obtained by (1) detecting the level of at least 1, 2, 3, 4, 5, 6, 7, 8, 9, 10 or more FSLMs, such as TG45:1, TG46:1, TG46:3, TG47:1, TG47:2, TG48:0, TG48:1, TG48:2, TG48:3, TG49:1, TG49:2, TG49:3, TG49:4, TG50:1, TG50:2, TG50:3, TG50:4, TG50:5, TG51:1, TG51:2, TG51:3, TG51:4, TG52:2, TG52:3, TG52:4, TG52:5, TG52:6, TG53:2, TG53:3, TG53:4, TG53:5, TG54:4, TG54:5, TG54:6, TG54:7, PC(O-16:0/14:0), PC(O-16:0/18:2), PC(O-16:0/20:3), PC(O-16:0/20:4), PC(O-16:0/22:6), PC(O-17:0/20:4), PC(O-18:0/18:1), PC(O-18:0/22:6), PC(O-18:0/18:2), PC(O-18:1/18:2), PC(O-18:1/20:4), PC(O-18:1/20:5), PC(O-18:1/22:6), PC(O-(20:0/22:6), PC(O-20:1/22.6), PC(O-20:2/20:4), PC(O-22:2/20:4), PC(O-22:1/22:6), PC(O-22:2/20:4), PC(O-(24:1/22:6), PC(O-24:2/20:4), PCO-34:2, PCO-36:3, PCO-34:2, PCO-38:3, PCO-40:2, PCO-40:6, PCO-44:7, PCO-46.8, PC(15:0/20:3), PC(15:0/22:6), PC(16:0/14:0), PC(16:0/22:4), PC(16:1/16:0), PC(16:1/20:4), PC(16:1/22.5), PC(17:0/20:5), PC(18:0/20:3), PC(18:0/22:4), PC(18:1/20:3), PC(18:2/18:2), PC(20:4/15:0), PC40:6, PC42:7, PC(17:1/20:4), PC(18:2/18:3), PC(18:1/24:1), PC(18:2/20:5), PC(20:4/20:0), CE14:1, CE16:0, CE20:5 and DG18:1/18:2, in a biological sample from a subject receiving FXN replacement therapy; (2) comparing the levels of the at least 1, 2, 3, 4, 5, 6, 7, 8, 9, 10 or more FSLMs, to the levels of the same FSLMs from a control sample, such as a baseline FXN(−) lipid profile; and (3) determining if the at least 1, 2, 3, 4, 5, 6, 7, 8, 9, 10 or more FSLMs detected in the biological sample are above or below the levels of the FSLMs in the control (e.g., baseline FXN(−) lipid profile). If the at least 1, 2, 3, 4, 5, 6, 7, 8, 9, 10 or more FSLMs are above or below the control (e.g., baseline FXN(−) lipid profile), then the diagnostic signature is indicative of the effectiveness of FXN replacement therapy.

In accordance with various embodiments, algorithms may be employed to predict whether or not a biological sample from a subject comprises FXN, or to evaluate or monitor whether the subject has effectively received FXN replacement therapy. The skilled artisan will appreciate that an algorithm can be any computation, formula, statistical survey, nomogram, look-up Tables, decision tree method, or computer program which processes a set of input variables (e.g., number of markers (n) which have been detected at a level exceeding some threshold level, or number of markers (n) which have been detected at a level below some threshold level) through a number of well-defined successive steps to eventually produce a score or “output.” Any suitable algorithm-whether computer-based or manual-based (e.g., look-up Tables)—is contemplated herein.

The disclosure provides for the use of various combinations and sub-combinations of FSLMs. For example, one or more of FSLMs, such as triglycerides, wherein the three acyl groups in each triglyceride molecule contain less than 56 carbons and/or wherein the three acyl groups in each triglyceride molecule contain 7 or less unsaturations (e.g., TG45:1, TG46:1, TG46:3, TG47:1, TG47:2, TG48:0, TG48:1, TG48:2, TG48:3, TG49:1, TG49:2, TG49:3, TG49:4, TG50:1, TG50:2, TG50:3, TG50:4, TG50:5, TG51:1, TG51:2, TG51:3, TG51:4, TG52:2, TG52:3, TG52:4, TG52:5, TG52:6, TG53:2, TG53:3, TG53:4, TG53:5, TG54:4, TG54:5, TG54:6, TG54:7), may be used alone or in combination with one or more of ether phospholipids (e.g., PCO- and PEO-), phosphatidylcholines (PCs), cholesteryl esters (CEs); and diglycerides (DGs), in any combination. It is understood that any single FSLM or combination of the FSLMs provided herein can be used in the disclosure unless clearly indicated otherwise.

### Samples

The present disclosure may be practiced with any suitable biological sample that potentially contains, expresses, includes, a detectable FSLM. For example, the biological sample may be obtained from a solid tissue sample, such as a skin biopsy sample, muscle biopsy sample, preferably a buccal sample, or a body fluid sample such as blood (including any blood product, such as whole blood, plasma, serum, or specific types of cells of the blood), urine, saliva, or seminal fluid.

In some embodiments, a sample which may be used for measuring an FXN lipid profile in the context of the present disclosure may be selected from the group consisting of a buccal sample, a skin sample, a hair follicle and a muscle biopsy sample. In one embodiment, the sample is a buccal sample. In another embodiment, the sample is a skin sample.

In some embodiments, a sample which may be used for measuring an FXN lipid profile in the context of the present disclosure may be a blood sample or a blood-derived sample, e.g., a whole plasma sample, a serum sample or a platelet sample. In one embodiment, the sample is a plasma sample. In another embodiment, the sample is a platelet sample.

In some embodiments, a sample which may be used for measuring an FXN lipid profile may be obtained from an FXN deficient subject prior to administration of FXN replacement therapy, during administration of FXN replacement therapy or after administration of FXN replacement therapy. In some embodiments, a sample may be obtained from an FXN deficient subject at least 5 days following the last administration of the FXN replacement therapy. In some embodiments, a sample may be obtained from an FXN deficient subject at least 10 days following the last administration of the FXN replacement therapy. In some embodiments, a sample may be obtained from an FXN deficient subject at least 15 days following the last administration of the FXN replacement therapy. In some embodiments, a sample may be obtained from an FXN deficient subject 5-25 days, 10-40 days or 15 to 45 days following the last administration of the FXN replacement therapy, e.g., 5 to 15 days, 10 to 25 days, 15 to 20 days, 20 to 35 days or 25 to 45 days after the last administration of the FXN replacement therapy. In some embodiments, a sample may be obtained from an FXN deficient subject 20 to 25 days, e.g., 20, 21, 22, 23 or 25 days, after the last administration of the FXN replacement therapy. In some embodiments, a sample may be obtained from an FXN deficient subject at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 days after the last administration of the FXN replacement therapy.

Any commercial device or system for isolating and/or obtaining tissue and/or blood or other biological products, and/or for processing said materials prior to conducting a detection reaction is contemplated.

Detection and/or Measurement of FSLMs

Various methodologies may be utilized for measuring the distance between lipid feature vectors. Once the data is normalized, the distance may be achieved for example by calculating the mean squared error, which may be extracted from the difference in the levels of each gene measured in two different profiles, such as baseline FXN(−) and FXN replacement for example. Alternatively, the distance may be achieved by calculating a correlation coefficient or applying a t-test.

The present disclosure contemplates any suitable means, techniques, and/or procedures for detecting and/or measuring the FSLMs of the disclosure. For example, levels of FSLMs in a sample may be measured using liquid chromatography-mass spectrometry (LC-MS), an analytical chemistry technique that combines the physical separation capabilities of liquid chromatography with the mass analysis capabilities of mass spectrometry. LC-MS is a powerful technique that has very high sensitivity, making it useful for the separation, general detection and potential identification of chemicals of particular masses in the presence of other chemicals (i.e., in complex mixtures). LC-MS includes targeted LC-MS and untargeted LC-MS. Targeted LC-MS can be performed when standards required for the quantification of the target analytes are available. Alternatively, surrogate analytes can be used to facilitate quantification of the target analytes if their standards are not commercially available. Examples of surrogate analytes include but are not limited to stable isotope-labeled standards of the target analytes, such as stable-isotope-labeled analogues of TG45:1, TG46:1, TG46:3, TG47:1, TG47:2, TG48:0, TG48:1, TG48:2, TG49:1, TG49:2, TG49:3, TG49:4, TG50:1, TG50:2, TG50:3, TG50:4, TG51:1, TG51:2, TG51:3, TG51:4, TG52:3, TG52:5, TG53:2, TG53:3, TG53:4, TG54:5, TG54:6, TG54:7 CE16:0, CE20:5, PC(O-16:0/18:2), PC(O-16:0/22:6), PC(O-18:0/18:2), PC(O-18:1/20:5), PCO-36:3, PCO-34:2, PCO-40:2, PCO-44:7 or PC(O-17:0/20:4.

Exemplary workflow that may be used to detect and measure levels of lipids in the context of the present disclosure are described, e.g., in Forest et al., J. of Proteome Research 2018, 17(11):3657-3670 and Wu et al., Advanced Drug Delivery Reviews 2020, 159:294-307, the entire contents of each of which are hereby incorporated herein by reference.

### Frataxin Replacement Therapy

In some aspects, the methodology provided in the present disclosure refers to the determination of a lipid profile associated with FXN replacement therapy. FXN replacement therapy involves the administration of a therapeutic compound capable of increasing levels of FXN in a subject in need thereof, e.g., an FXN replacement therapeutic, to a subject in need thereof. A number of alternatives for delivery of exogenous FXN may be envisioned. The FXN replacement therapeutic may be provided by FXN protein delivery or through delivery of a nucleic acid capable of increasing FXN levels in a subject, e.g., a nucleic acid encoding FXN. FXN protein delivery may be delivery of full length FXN or delivery of a FXN fusion protein.

In some embodiments, the FXN replacement therapy comprises administration to a subject in need thereof a nucleic acid capable of increasing levels of FXN in the subject. In some embodiments, the nucleic acid may be a nucleic acid encoding FXN, e.g., FXN mRNA. In some embodiments, the nucleic acid may be an antisense oligonucleotide, e.g., an antisense oligonucleotide that activates expression of FXN protein as described, e.g., in Li et al., Nucleic Acid Ther. 2018, 28(1): 23-33 or in Mikaeili et al., Scientific Reports 2018, 8:17217, the entire contents of each of which are hereby incorporated herein by reference. In some embodiments, the nucleic acid may be siRNA capable of activating expression of FXN protein as described, e.g., in Shen et al., Bioorganic & Medicinal Chemistry Letters 2018, 28(17):2850-2855, the entire contents of which are hereby incorporated herein by reference.

In some embodiments, the FXN replacement therapy comprises administration of an FXN fusion protein. As used herein, the term “FXN fusion protein” refers to FXN or a fragment of FXN fused to a full length or a fragment of a different protein, or to a peptide. In some embodiments, an FXN fusion protein comprises a polypeptide that comprises FXN, e.g., full-length hFXN (SEQ ID NO: 1) or mature hFXN (SEQ ID NO: 2). The full-length hFXN protein (amino acids 1-210) has the amino acid sequence of SEQ ID NO: 1.

| | |
|---|---|
| SEQ ID NO: 1 | MWTLGRRAVAGLLASPSPAQAQTLTRVPRPAELAPLCGRRGLRTD |
| Full-length | IDATCTPRRASSNQRGLNQIWNVKKQSVYLMNLRKSGTLGHPGSL |
| hFXN | DETTYERLAEETLDSLAEFFEDLADKPYTFEDYDVSFGSGVLTVKL |
| hFXN₁₋₂₁₀ | GGDLGTYVINKQTPNKQIWLSSPSSGPKRYDWTGKNWVYSHDGV |
| | SLHELLAAELTKALKTKLDLSSLAYSGKDA |

In the mitochondrial matrix, the full-length hFXN protein it is cleaved at amino acid 81, resulting in the mature form of FXN, having 130 aa and a predicted molecular weight of 14.2 kDa (SEQ ID NO: 2).

| | |
|---|---|
| SEQ ID NO: 2 | SGTLGHPGSLDETTYERLAEETLDSLAEFFEDLADKPYTFEDYDVSF |
| Mature hFXN | GSGVLTVKLGGDLGTYVINKQTPNKQIWLSSPSSGPKRYDWTGKN |
| hFXN₈₁₋₂₁₀ | WVYSHDGVSLHELLAAELTKALKTKLDLSSLAYSGKDA |

The full-length hFXN (SEQ ID NO: 1) comprises mature hFXN (SEQ ID NO: 2) and a mitochondrial targeting sequence (MTS) having the amino acid sequence

(SEQ ID NO: 3)
MWTLGRRAVAGLLASPSPAQAQTLTRVPRPAELAPLCGRRGLRTDIDAT
CTPRRASSNQRGLNQIWNVKKQSVYLMNLRK.

In some embodiments, the FXN fusion protein also comprises a cell penetrating peptide (CPP). The term “cell penetrating peptide” or “CPP”, as used herein, refers to a short peptide sequence, typically between 5 and 30 amino acids long, that can facilitate cellular intake of various molecular cargo, such as proteins. Within the context of the present disclosure, a CPP present in an FXN fusion protein facilitates the delivery of the FXN fusion protein into a cell, e.g., a recipient cell. CPPs may be polycationic, i.e., have an amino acid composition that either contains a high relative abundance of positively charged amino acids, such as lysine or arginine. CCPs may also be amphipathic, i.e., have sequences that contain an alternating pattern of polar/charged amino acids and non-polar, hydrophobic amino acids. CPPs may also be hydrophobic, i.e., contain only apolar residues with low net charge, or have hydrophobic amino acid groups that are crucial for cellular uptake.

A CPP that may be comprised in the FXN fusion protein may be any CPP known to a person skilled in the art. For example, the CPP may be any CPP listed in the Database of Cell-Penetrating Peptides CPPsite 2.0, the entire contents of which are hereby incorporated herein by reference. For examples, a CPP useful in the context of the present disclosure may be a cell penetrating peptide derived from a protein selected from the group consisting of HIV Trans-Activator of Transcription peptide (HIV-TAT), galanin, mastoparan, transportan, penetratin, polyarginine, VP22, transportan, amphipathic peptides such as MAP, KALA, ppTG20, proline-rich peptides, MPG-derived peptides, Pep-1, and also loligomers, arginine-rich peptides and calcitonin-derived peptides.

In some embodiments, a CPP of the present disclosure comprises a TAT protein domain comprising amino acids 47-57 of the 86 amino acid full length HIV-TAT protein (which 11 amino acid peptide may also be referred to herein as “HIV-TAT”; SEQ ID NO: 4). In one embodiment, the CPP consists of HIV-TAT (SEQ ID NO: 4). In some embodiments, the CPP comprises amino acids 47-57 of the 86 amino acid full length HIV-TAT protein with a methionine added at the amino terminus for initiation (12 AA; “HIV-TAT+M”): MYGRKKRRQRRR (SEQ ID NO: 5). Table 1 below lists amino acid sequences of exemplary CPPs.

TABLE 1
Exemplary CPPs and corresponding sequences

| SEQ ID NO. | CPP | Amino Acid Sequence |
|---|---|---|
| 4 | HIV-TAT | YGRKKRRQRRR |
| 5 | HIV-TAT + M | MYGRKKRRQRRR |
| 6 | Galanin | GWTLNSAGYLLGPHAVGNHRSFSDKNGLTS |
| 7 | Mastoparan | INLKALAALAKKIL-NH₂ |
| 8 | Transportan | GWTLNSAGYLLGKINLKALAALAKKIL |
| 9 | Penetratin | RQIKIWFQNRRMKWKK |
| 10 | Polyarginine | RRRRRRRRR |
| 11 | VP22 | DAATATRGRSAASRPTERPRAPARSASRPRRPVE |

In some embodiments, the CPP comprised in the FXN fusion protein is HIV-TAT (SEQ ID NO: 4). In some embodiments, the CPP comprised in the FXN fusion protein is HIV-TAT+M (SEQ ID NO: 5). In some embodiments, the FXN fusion protein comprises full-length FXN, e.g., SEQ ID NO: 1, and HIV-TAT, e.g., SEQ ID NO: 4, as CPP. In some embodiments, the FXN fusion protein comprises full-length FXN, e.g., SEQ ID NO: 1, and HIV-TAT+M, e.g., SEQ ID NO: 5, as CPP.

In some embodiments, in a FXN fusion protein of the present disclosure, the CPP may be fused together with the FXN, e.g., full-length FXN, via a linker to form a single polypeptide chain. Examples of FXN fusion proteins include TAT-FXN fusion proteins, where TAT or a fragment of TAT may be directly or indirectly (through a linker) linked to either the N- or the C-terminus of FXN. In one specific example, the linker may comprise the amino acid sequence GG.

In some aspects, the CPP, e.g., HIV-TAT, that is present in an FXN fusion protein of the present disclosure facilitates delivery of the FXN fusion protein into a cell, e.g., a cell that may be present in vitro, ex vivo, or in a subject. Once inside the cell, the FXN fusion protein may be processed by cellular machinery to remove the CPP, e.g., HIV-TAT, from the FXN.

One specific example of a TAT-FXN fusion protein is referred to as an exemplary FXN fusion protein. The exemplary FXN fusion protein is a 24.9 kDa fusion protein currently under investigation as an FXN replacement therapy to restore functional levels of FXN in the mitochondria of subjects with FRDA. The exemplary FXN fusion protein includes the HIV-TAT peptide linked to the N-terminus of the full-length hFXN protein. The mechanism of action of the exemplary FXN fusion protein relies on the cell-penetrating ability of the HIV-TAT peptide to deliver the exemplary FXN fusion protein into cells and the subsequent processing into mature hFXN after translocation into the mitochondria. The exemplary FXN fusion protein is described, e.g., in US 2021/0047378, the entire contents of which are hereby incorporated herein by reference. The exemplary FXN fusion protein comprises the following amino acid sequence (224 amino acids):

(SEQ ID NO: 12)
MYGRKKRRQRRRGGMWTLGRRAVAGLLASPSPAQAQTLTRVPRPAELAP
LCGRRGLRTDIDATCTPRRASSNQRGLNQIWNVKKQSVYLMNLRKSGTL
GHPGSLDETTYERLAEETLDSLAEFFEDLADKPYTFEDYDVSFGSGVLT
VKLGGDLGTYVINKQTPNKQIWLSSPSSGPKRYDWTGKNWVYSHDGVSL
HELLAAELTKALKTKLDLSSLAYSGKDA.

FXN replacement may also be delivered by viral gene replacement, which may utilize retroviral, lentiviral, and adeno-associated viral vectors, as well as adenoviruses. Alternatively, FXN replacement therapy may be achieved by upregulation of endogenous mutant FXN gene, which depending on the number of GAA repeats is expressed in varying levels in carriers of the mutant FXN allele.

### FSLM Applications

### Methods for Evaluating Efficacy of a FXN Replacement Therapy

In some aspects, the present disclosure provides methods for evaluating and/or monitoring efficacy of FXN replacement therapy in a subject. The disclosure further provides methods for determining whether a subject is in need of FXN replacement therapy or a change in FXN replacement therapy, e.g., determining whether FXN replacement therapy should be initiated, increased, decreased or ceased in a subject. In some embodiments, the methods are carried out by the subject using a sample obtained from the same subject or as a point of care test, and results can be assessed by the subject or by a physician.

In some embodiments, the present disclosure provides methods for evaluating efficacy of a frataxin replacement therapy, the method comprising: (a) determining a baseline FSLM(−) lipid profile for one or more FXN-sensitive lipid markers (FSLMs) in a sample obtained from an FXN deficient subject prior to administration of the FXN replacement therapy; (b) determining an FXN replacement lipid profile for the one or more FXN-sensitive lipid markers (FSLMs) in a sample obtained from the FXN deficient subject following administration of the FXN replacement therapy; (c) comparing the FXN replacement lipid profile determined in step (b) with the baseline FXN(−) lipid profile determined in step (a); and (d) determining efficacy of the FXN replacement therapy based on the comparison in step (c).

In some embodiments, comparing the FXN replacement lipid profile with the baseline FXN(−) lipid profile comprises comparing the amount of one or more FSLMs in the FXN replacement lipid profile with the amount of lipid of the corresponding one or more FSLMs in the baseline FXN(−) lipid profile.

In some embodiments, the present disclosure also provides a method for evaluating efficacy of a frataxin (FXN) replacement therapy, the method comprising: (a) determining an FXN replacement lipid profile for one or more FXN-sensitive lipid markers (FSLMs) in a sample obtained from an FXN deficient subject following administration of an FXN replacement therapy; (b) comparing the subject FXN replacement lipid profile determined in step (a) with a reference FXN lipid profile for the one or more FSLMs; and (c) determining efficacy of the FXN replacement therapy based on the comparison in step (b). In some embodiments, the reference FXN lipid profile is a baseline FXN(−) lipid profile for the one or more FSLMs. In some embodiments, the baseline FXN(−) lipid profile for the one or more FSLMs is determined in a sample obtained from an FXN deficient subject prior to administration of an FXN replacement therapy.

In some embodiments, comparing the FXN replacement lipid profile with the reference FXN lipid profile comprises comparing the amount of one or more FSLMs in the FXN replacement lipid profile with the amount of lipid of the corresponding one or more FSLMs in the reference FXN lipid profile.

In some embodiments, the one or more FSLMs comprise one or any combination of one or more TGs, ether phospholipids (e.g., PCO- or PEO-), PCs, DGs or CEs as described herein. For example, the one or more FSLMs may comprise one or more TGs, wherein the three acyl groups in each TG molecule contain less than 56 carbon atoms, e.g., one or more of TG45:1, TG46:1, TG46:3, TG47:1, TG47:2, TG48:0, TG48:1, TG48:2, TG48:3, TG49:1, TG49:2, TG49:3, TG49:4, TG50:1, TG50:2, TG50:3, TG50:4, TG50:5, TG51:1, TG51:2, TG51:3, TG51:4, TG52:2, TG52:3, TG52:4, TG52:5, TG52:6, TG53:2, TG53:3, TG53:4, TG53:5, TG54:4, TG54:5, TG54:6 and TG54:7

In certain embodiments, a decrease in the amount of one or more TGs, wherein the three acyl groups in each TG molecule contain less than 56 carbon atoms (e.g., TG45:1, TG46:1, TG46:3, TG47:1, TG47:2, TG48:0, TG48:1, TG48:2, TG48:3, TG49:1, TG49:2, TG49:3, TG49:4, TG50:1, TG50:2, TG50:3, TG50:4, TG50:5, TG51:1, TG51:2, TG51:3, TG51:4, TG52:2, TG52:3, TG52:4, TG52:5, TG52:6, TG53:2, TG53:3, TG53:4, TG53:5, TG54:4, TG54:5, TG54:6 and TG54:7), in a FXN replacement lipid profile, as compared to a reference FXN lipid profile, e.g., a baseline FXN(−) lipid profile, is an indication that the FXN replacement therapy is effective.

In certain embodiments, a decrease in the amount of one or more TGs, wherein the three acyl groups in each TG molecule contain less than 56 carbon atoms (e.g., TG45:1, TG46:1, TG46:3, TG47:1, TG47:2, TG48:0, TG48:1, TG48:2, TG48:3, TG49:1, TG49:2, TG49:3, TG49:4, TG50:1, TG50:2, TG50:3, TG50:4, TG50:5, TG51:1, TG51:2, TG51:3, TG51:4, TG52:2, TG52:3, TG52:4, TG52:5, TG52:6, TG53:2, TG53:3, TG53:4, TG53:5, TG54:4, TG54:5, TG54:6 and TG54:7) in a sample obtained from an FXN-deficient subject following administration of FXN replacement therapy, as compared to the amount of the one or more FSLMs in a reference sample, e.g., a sample obtained from the FXN-deficient subject prior to administration of the FXN replacement therapy, is an indication that the FXN replacement therapy is effective.

In certain embodiments, a decrease in the amount of one or more TGs, wherein the three acyl groups in each TG molecule contain 7 or less unsaturations (e.g., TG45:1, TG46:1, TG46:3, TG47:1, TG47:2, TG48:0, TG48:1, TG48:2, TG48:3, TG49:1, TG49:2, TG49:3, TG49:4, TG50:1, TG50:2, TG50:3, TG50:4, TG50:5, TG51:1, TG51:2, TG51:3, TG51:4, TG52:2, TG52:3, TG52:4, TG52:5, TG52:6, TG53:2, TG53:3, TG53:4, TG53:5, TG54:4, TG54:5, TG54:6 and TG54:7), in a FXN replacement lipid profile, as compared to a reference FXN lipid profile, e.g., a baseline FXN(−) lipid profile, is an indication that the FXN replacement therapy is effective.

In certain embodiments, a decrease in the amount of TGs, wherein the three acyl groups in each TG molecule contain 7 or less unsaturations (e.g., TG45:1, TG46:1, TG46:3, TG47:1, TG47:2, TG48:0, TG48:1, TG48:2, TG48:3, TG49:1, TG49:2, TG49:3, TG49:4, TG50:1, TG50:2, TG50:3, TG50:4, TG50:5, TG51:1, TG51:2, TG51:3, TG51:4, TG52:2, TG52:3, TG52:4, TG52:5, TG52:6, TG53:2, TG53:3, TG53:4, TG53:5, TG54:4, TG54:5, TG54:6 and TG54:7) in a sample obtained from an FXN-deficient subject following administration of FXN replacement therapy, as compared to the amount of the one or more FSLMs in a reference sample, e.g., a sample obtained from the FXN-deficient subject prior to administration of the FXN replacement therapy, is an indication that the FXN replacement therapy is effective.

Specifically, in some embodiments, a decrease in the amount of TG45:1 in a sample obtained from an FXN deficient subject following administration of FXN replacement therapy, as compared to the amount of TG45:1 in a sample obtained from the subject prior to administration of the FXN replacement therapy, is an indication that the FXN replacement therapy is effective.

Specifically, in some embodiments, a decrease in the amount of TG46:1 in a sample obtained from an FXN deficient subject following administration of FXN replacement therapy, as compared to the amount of TG46:1 in a sample obtained from the subject prior to administration of the FXN replacement therapy, is an indication that the FXN replacement therapy is effective.

Specifically, in some embodiments, a decrease in the amount of TG46:3 in a sample obtained from an FXN deficient subject following administration of FXN replacement therapy, as compared to the amount of TG46:3 in a sample obtained from the subject prior to administration of the FXN replacement therapy, is an indication that the FXN replacement therapy is effective.

Specifically, in some embodiments, a decrease in the amount of TG47:1 in a sample obtained from an FXN deficient subject following administration of FXN replacement therapy, as compared to the amount of TG47:1 in a sample obtained from the subject prior to administration of the FXN replacement therapy, is an indication that the FXN replacement therapy is effective.

Specifically, in some embodiments, a decrease in the amount of TG47:2 in a sample obtained from an FXN deficient subject following administration of FXN replacement therapy, as compared to the amount of TG47:2 in a sample obtained from the subject prior to administration of the FXN replacement therapy, is an indication that the FXN replacement therapy is effective.

Specifically, in some embodiments, a decrease in the amount of TG48:0 in a sample obtained from an FXN deficient subject following administration of FXN replacement therapy, as compared to the amount of TG48:0 in a sample obtained from the subject prior to administration of the FXN replacement therapy, is an indication that the FXN replacement therapy is effective.

Specifically, in some embodiments, a decrease in the amount of TG48:1 in a sample obtained from an FXN deficient subject following administration of FXN replacement therapy, as compared to the amount of TG48:1 in a sample obtained from the subject prior to administration of the FXN replacement therapy, is an indication that the FXN replacement therapy is effective.

Specifically, in some embodiments, a decrease in the amount of TG48:2 in a sample obtained from an FXN deficient subject following administration of FXN replacement therapy, as compared to the amount of TG48:2 in a sample obtained from the subject prior to administration of the FXN replacement therapy, is an indication that the FXN replacement therapy is effective.

Specifically, in some embodiments, a decrease in the amount of TG48:3 in a sample obtained from an FXN deficient subject following administration of FXN replacement therapy, as compared to the amount of TG48:3 in a sample obtained from the subject prior to administration of the FXN replacement therapy, is an indication that the FXN replacement therapy is effective.

Specifically, in some embodiments, a decrease in the amount of TG49:1 in a sample obtained from an FXN deficient subject following administration of FXN replacement therapy, as compared to the amount of TG49:1 in a sample obtained from the subject prior to administration of the FXN replacement therapy, is an indication that the FXN replacement therapy is effective.

Specifically, in some embodiments, a decrease in the amount of TG49:2 in a sample obtained from an FXN deficient subject following administration of FXN replacement therapy, as compared to the amount of TG49:2 in a sample obtained from the subject prior to administration of the FXN replacement therapy, is an indication that the FXN replacement therapy is effective.

Specifically, in some embodiments, a decrease in the amount of TG49:3 in a sample obtained from an FXN deficient subject following administration of FXN replacement therapy, as compared to the amount of TG49:3 in a sample obtained from the subject prior to administration of the FXN replacement therapy, is an indication that the FXN replacement therapy is effective.

Specifically, in some embodiments, a decrease in the amount of TG49:4 in a sample obtained from an FXN deficient subject following administration of FXN replacement therapy, as compared to the amount of TG49:4 in a sample obtained from the subject prior to administration of the FXN replacement therapy, is an indication that the FXN replacement therapy is effective.

Specifically, in some embodiments, a decrease in the amount of TG50:1 in a sample obtained from an FXN deficient subject following administration of FXN replacement therapy, as compared to the amount of TG50:1 in a sample obtained from the subject prior to administration of the FXN replacement therapy, is an indication that the FXN replacement therapy is effective.

Specifically, in some embodiments, a decrease in the amount of TG50:2 in a sample obtained from an FXN deficient subject following administration of FXN replacement therapy, as compared to the amount of TG50:2 in a sample obtained from the subject prior to administration of the FXN replacement therapy, is an indication that the FXN replacement therapy is effective.

Specifically, in some embodiments, a decrease in the amount of TG50:3 in a sample obtained from an FXN deficient subject following administration of FXN replacement therapy, as compared to the amount of TG50:3 in a sample obtained from the subject prior to administration of the FXN replacement therapy, is an indication that the FXN replacement therapy is effective.

Specifically, in some embodiments, a decrease in the amount of TG50:4 in a sample obtained from an FXN deficient subject following administration of FXN replacement therapy, as compared to the amount of TG50:4 in a sample obtained from the subject prior to administration of the FXN replacement therapy, is an indication that the FXN replacement therapy is effective.

Specifically, in some embodiments, a decrease in the amount of TG50:5 in a sample obtained from an FXN deficient subject following administration of FXN replacement therapy, as compared to the amount of TG50:5 in a sample obtained from the subject prior to administration of the FXN replacement therapy, is an indication that the FXN replacement therapy is effective.

Specifically, in some embodiments, a decrease in the amount of TG51:1 in a sample obtained from an FXN deficient subject following administration of FXN replacement therapy, as compared to the amount of TG51:1 in a sample obtained from the subject prior to administration of the FXN replacement therapy, is an indication that the FXN replacement therapy is effective.

Specifically, in some embodiments, a decrease in the amount of TG51:2 in a sample obtained from an FXN deficient subject following administration of FXN replacement therapy, as compared to the amount of TG51:2 in a sample obtained from the subject prior to administration of the FXN replacement therapy, is an indication that the FXN replacement therapy is effective.

Specifically, in some embodiments, a decrease in the amount of TG51:3 in a sample obtained from an FXN deficient subject following administration of FXN replacement therapy, as compared to the amount of TG51:3 in a sample obtained from the subject prior to administration of the FXN replacement therapy, is an indication that the FXN replacement therapy is effective.

Specifically, in some embodiments, a decrease in the amount of TG51:4 in a sample obtained from an FXN deficient subject following administration of FXN replacement therapy, as compared to the amount of TG51:4 in a sample obtained from the subject prior to administration of the FXN replacement therapy, is an indication that the FXN replacement therapy is effective.

Specifically, in some embodiments, a decrease in the amount of TG52:2 in a sample obtained from an FXN deficient subject following administration of FXN replacement therapy, as compared to the amount of TG52:2 in a sample obtained from the subject prior to administration of the FXN replacement therapy, is an indication that the FXN replacement therapy is effective.

Specifically, in some embodiments, a decrease in the amount of TG52:3 in a sample obtained from an FXN deficient subject following administration of FXN replacement therapy, as compared to the amount of TG52:3 in a sample obtained from the subject prior to administration of the FXN replacement therapy, is an indication that the FXN replacement therapy is effective.

Specifically, in some embodiments, a decrease in the amount of TG52:4 in a sample obtained from an FXN deficient subject following administration of FXN replacement therapy, as compared to the amount of TG52:4 in a sample obtained from the subject prior to administration of the FXN replacement therapy, is an indication that the FXN replacement therapy is effective.

Specifically, in some embodiments, a decrease in the amount of TG52:5 in a sample obtained from an FXN deficient subject following administration of FXN replacement therapy, as compared to the amount of TG52:5 in a sample obtained from the subject prior to administration of the FXN replacement therapy, is an indication that the FXN replacement therapy is effective.

Specifically, in some embodiments, a decrease in the amount of TG52:6 in a sample obtained from an FXN deficient subject following administration of FXN replacement therapy, as compared to the amount of TG52:6 in a sample obtained from the subject prior to administration of the FXN replacement therapy, is an indication that the FXN replacement therapy is effective.

Specifically, in some embodiments, a decrease in the amount of TG53:2 in a sample obtained from an FXN deficient subject following administration of FXN replacement therapy, as compared to the amount of TG53:2 in a sample obtained from the subject prior to administration of the FXN replacement therapy, is an indication that the FXN replacement therapy is effective.

Specifically, in some embodiments, a decrease in the amount of TG53:3 in a sample obtained from an FXN deficient subject following administration of FXN replacement therapy, as compared to the amount of TG53:3 in a sample obtained from the subject prior to administration of the FXN replacement therapy, is an indication that the FXN replacement therapy is effective.

Specifically, in some embodiments, a decrease in the amount of TG53:4 in a sample obtained from an FXN deficient subject following administration of FXN replacement therapy, as compared to the amount of TG53:4 in a sample obtained from the subject prior to administration of the FXN replacement therapy, is an indication that the FXN replacement therapy is effective.

Specifically, in some embodiments, a decrease in the amount of TG53:5 in a sample obtained from an FXN deficient subject following administration of FXN replacement therapy, as compared to the amount of TG53:5 in a sample obtained from the subject prior to administration of the FXN replacement therapy, is an indication that the FXN replacement therapy is effective.

Specifically, in some embodiments, a decrease in the amount of TG54:4 in a sample obtained from an FXN deficient subject following administration of FXN replacement therapy, as compared to the amount of TG54:4 in a sample obtained from the subject prior to administration of the FXN replacement therapy, is an indication that the FXN replacement therapy is effective.

Specifically, in some embodiments, a decrease in the amount of TG54:5 in a sample obtained from an FXN deficient subject following administration of FXN replacement therapy, as compared to the amount of TG54:5 in a sample obtained from the subject prior to administration of the FXN replacement therapy, is an indication that the FXN replacement therapy is effective.

Specifically, in some embodiments, a decrease in the amount of TG54:6 in a sample obtained from an FXN deficient subject following administration of FXN replacement therapy, as compared to the amount of TG54:6 in a sample obtained from the subject prior to administration of the FXN replacement therapy, is an indication that the FXN replacement therapy is effective.

Specifically, in some embodiments, a decrease in the amount of TG54:7 in a sample obtained from an FXN deficient subject following administration of FXN replacement therapy, as compared to the amount of TG54:7 in a sample obtained from the subject prior to administration of the FXN replacement therapy, is an indication that the FXN replacement therapy is effective.

In certain embodiments, an increase in the amount of one or more PCO-, e.g., one or more of PC(O-16:0/14:0), PC(O-16:0/18:2), PC(O-16:0/20:3), PC(O-16:0/20:4), PC(O-16:0/22:6), PC(O-17:0/20:4), PC(O-18:0/18:1), PC(O-18:0/22:6), PC(O-18:0/18:2), PC(O-18:1/18:2), PC(O-18:1/20:4), PC(O-18:1/20:5), PC(O-18:1/22:6), PC(O-(20:0/22:6), PC(O-20:1/22.6), PC(O-20:2/20:4), PC(O-22:2/20:4), PC(O-22:1/22:6), PC(O-22:2/20:4), PC(O-(24:1/22:6), PC(O-24:2/20:4), PCO-34:2, PCO-36:3, PCO-34:2, PCO-38:3, PCO-40:2, PCO-40:6, PCO-44:7 and PCO-46.8, in a FXN replacement lipid profile, as compared to a reference FXN lipid profile, e.g., a baseline FXN(−) lipid profile, is an indication that the FXN replacement therapy is effective.

In certain embodiments, an increase in the amount of one or more PCO-, e.g., one or more of PC(O-16:0/14:0), PC(O-16:0/18:2), PC(O-16:0/20:3), PC(O-16:0/20:4), PC(O-16:0/22:6), PC(O-17:0/20:4), PC(O-18:0/18:1), PC(O-18:0/22:6), PC(O-18:0/18:2), PC(O-18:1/18:2), PC(O-18:1/20:4), PC(O-18:1/20:5), PC(O-18:1/22:6), PC(O-(20:0/22:6), PC(O-20:1/22.6), PC(O-20:2/20:4), PC(O-22:2/20:4), PC(O-22:1/22:6), PC(O-22:2/20:4), PC(O-(24:1/22:6), PC(O-24:2/20:4), PCO-34:2, PCO-36:3, PCO-34:2, PCO-38:3, PCO-40:2, PCO-40:6, PCO-44:7 and PCO-46.8, in a sample obtained from an FXN-deficient subject following administration of FXN replacement therapy, as compared to the amount of the one or more FSLMs in a reference sample, e.g., a sample obtained from the FXN-deficient subject prior to administration of the FXN replacement therapy, is an indication that the FXN replacement therapy is effective.

In some embodiments, an increase in the amount of PC(O-16:0/14:0) in a sample obtained from an FXN deficient subject following administration of FXN replacement therapy, as compared to the amount of PC(O-16:0/14:0) in a sample obtained from the subject prior to administration of the FXN replacement therapy, is an indication that the FXN replacement therapy is effective.

In some embodiments, an increase in the amount of PC(O-16:0/18:2) in a sample obtained from an FXN deficient subject following administration of FXN replacement therapy, as compared to the amount of PC(O-16:0/18:2) in a sample obtained from the subject prior to administration of the FXN replacement therapy, is an indication that the FXN replacement therapy is effective.

In some embodiments, an increase in the amount of PC(O-16:0/20:3) in a sample obtained from an FXN deficient subject following administration of FXN replacement therapy, as compared to the amount of PC(O-16:0/20:3) in a sample obtained from the subject prior to administration of the FXN replacement therapy, is an indication that the FXN replacement therapy is effective.

In some embodiments, an increase in the amount of PC(O-16:0/20:4) in a sample obtained from an FXN deficient subject following administration of FXN replacement therapy, as compared to the amount of PC(O-16:0/20:4) in a sample obtained from the subject prior to administration of the FXN replacement therapy, is an indication that the FXN replacement therapy is effective.

In some embodiments, an increase in the amount of PC(O-16:0/22:6) in a sample obtained from an FXN deficient subject following administration of FXN replacement therapy, as compared to the amount of PC(O-16:0/22:6) in a sample obtained from the subject prior to administration of the FXN replacement therapy, is an indication that the FXN replacement therapy is effective.

In some embodiments, an increase in the amount of PC(O-17:0/20:4) in a sample obtained from an FXN deficient subject following administration of FXN replacement therapy, as compared to the amount of PC(O-17:0/20:4) in a sample obtained from the subject prior to administration of the FXN replacement therapy, is an indication that the FXN replacement therapy is effective.

In some embodiments, an increase in the amount of PC(O-18:0/18:1) in a sample obtained from an FXN deficient subject following administration of FXN replacement therapy, as compared to the amount of PC(O-18:0/18:2) in a sample obtained from the subject prior to administration of the FXN replacement therapy, is an indication that the FXN replacement therapy is effective.

In some embodiments, an increase in the amount of PC(O-18:0/18:2) in a sample obtained from an FXN deficient subject following administration of FXN replacement therapy, as compared to the amount of PC(O-18:0/18:2) in a sample obtained from the subject prior to administration of the FXN replacement therapy, is an indication that the FXN replacement therapy is effective.

In some embodiments, an increase in the amount of PC(O-18:0/22:6) in a sample obtained from an FXN deficient subject following administration of FXN replacement therapy, as compared to the amount of PC(O-18:0/22:6) in a sample obtained from the subject prior to administration of the FXN replacement therapy, is an indication that the FXN replacement therapy is effective.

In some embodiments, an increase in the amount of PC(O-18:1/18:2) in a sample obtained from an FXN deficient subject following administration of FXN replacement therapy, as compared to the amount of PC(O-18:1/18:2) in a sample obtained from the subject prior to administration of the FXN replacement therapy, is an indication that the FXN replacement therapy is effective.

In some embodiments, an increase in the amount of PC(O-18:1/20:4) in a sample obtained from an FXN deficient subject following administration of FXN replacement therapy, as compared to the amount of PC(O-18:1/20:4) in a sample obtained from the subject prior to administration of the FXN replacement therapy, is an indication that the FXN replacement therapy is effective.

In some embodiments, an increase in the amount of PC(O-18:1/20:5) in a sample obtained from an FXN deficient subject following administration of FXN replacement therapy, as compared to the amount of PC(O-18:1/20:5) in a sample obtained from the subject prior to administration of the FXN replacement therapy, is an indication that the FXN replacement therapy is effective.

In some embodiments, an increase in the amount of PC(O-18:1/22:6) in a sample obtained from an FXN deficient subject following administration of FXN replacement therapy, as compared to the amount of PC(O-18:1/22:6) in a sample obtained from the subject prior to administration of the FXN replacement therapy, is an indication that the FXN replacement therapy is effective.

In some embodiments, an increase in the amount of PC(O-(20:0/22:6) in a sample obtained from an FXN deficient subject following administration of FXN replacement therapy, as compared to the amount of PC(O-(20:0/22:6) in a sample obtained from the subject prior to administration of the FXN replacement therapy, is an indication that the FXN replacement therapy is effective.

In some embodiments, an increase in the amount of PC(O-20:1/22.6) in a sample obtained from an FXN deficient subject following administration of FXN replacement therapy, as compared to the amount of PC(O-20:1/22.6) in a sample obtained from the subject prior to administration of the FXN replacement therapy, is an indication that the FXN replacement therapy is effective.

In some embodiments, an increase in the amount of PC(O-20:2/20:4) in a sample obtained from an FXN deficient subject following administration of FXN replacement therapy, as compared to the amount of PC(O-20:2/20:4) in a sample obtained from the subject prior to administration of the FXN replacement therapy, is an indication that the FXN replacement therapy is effective.

In some embodiments, an increase in the amount of PC(O-22:2/20:4) in a sample obtained from an FXN deficient subject following administration of FXN replacement therapy, as compared to the amount of PC(O-22:2/20:4) in a sample obtained from the subject prior to administration of the FXN replacement therapy, is an indication that the FXN replacement therapy is effective.

In some embodiments, an increase in the amount of PC(O-22:1/22:6) in a sample obtained from an FXN deficient subject following administration of FXN replacement therapy, as compared to the amount of PC(O-22:1/22:6) in a sample obtained from the subject prior to administration of the FXN replacement therapy, is an indication that the FXN replacement therapy is effective.

In some embodiments, an increase in the amount of PC(O-22:2/20:4) in a sample obtained from an FXN deficient subject following administration of FXN replacement therapy, as compared to the amount of PC(O-22:2/20:4) in a sample obtained from the subject prior to administration of the FXN replacement therapy, is an indication that the FXN replacement therapy is effective.

In some embodiments, an increase in the amount of PC(O-(24:1/22:6) in a sample obtained from an FXN deficient subject following administration of FXN replacement therapy, as compared to the amount of PC(O-(24:1/22:6) in a sample obtained from the subject prior to administration of the FXN replacement therapy, is an indication that the FXN replacement therapy is effective.

In some embodiments, an increase in the amount of PC(O-24:2/20:4) in a sample obtained from an FXN deficient subject following administration of FXN replacement therapy, as compared to the amount of PC(O-24:2/20:4) in a sample obtained from the subject prior to administration of the FXN replacement therapy, is an indication that the FXN replacement therapy is effective.

In some embodiments, an increase in the amount of PCO-34:2 in a sample obtained from an FXN deficient subject following administration of FXN replacement therapy, as compared to the amount of PCO-34:2 in a sample obtained from the subject prior to administration of the FXN replacement therapy, is an indication that the FXN replacement therapy is effective.

In some embodiments, an increase in the amount of PCO-36:3 in a sample obtained from an FXN deficient subject following administration of FXN replacement therapy, as compared to the amount of PCO-36:3 in a sample obtained from the subject prior to administration of the FXN replacement therapy, is an indication that the FXN replacement therapy is effective.

In some embodiments, an increase in the amount of PCO-34:2 in a sample obtained from an FXN deficient subject following administration of FXN replacement therapy, as compared to the amount of PCO-34:2 in a sample obtained from the subject prior to administration of the FXN replacement therapy, is an indication that the FXN replacement therapy is effective.

In some embodiments, an increase in the amount of PCO-38:3 in a sample obtained from an FXN deficient subject following administration of FXN replacement therapy, as compared to the amount of PCO-38:3 in a sample obtained from the subject prior to administration of the FXN replacement therapy, is an indication that the FXN replacement therapy is effective.

In some embodiments, an increase in the amount of PCO-40:2 in a sample obtained from an FXN deficient subject following administration of FXN replacement therapy, as compared to the amount of PCO-40:2 in a sample obtained from the subject prior to administration of the FXN replacement therapy, is an indication that the FXN replacement therapy is effective.

In some embodiments, an increase in the amount of PCO-40:6 in a sample obtained from an FXN deficient subject following administration of FXN replacement therapy, as compared to the amount of PCO-40:6 in a sample obtained from the subject prior to administration of the FXN replacement therapy, is an indication that the FXN replacement therapy is effective.

In some embodiments, an increase in the amount of PCO-44:7 in a sample obtained from an FXN deficient subject following administration of FXN replacement therapy, as compared to the amount of PCO-44:7 in a sample obtained from the subject prior to administration of the FXN replacement therapy, is an indication that the FXN replacement therapy is effective.

amountamount

In some embodiments, an increase in the amount of PCO-46:8 in a sample obtained from an FXN deficient subject following administration of FXN replacement therapy, as compared to the amount of PCO-46.8 in a sample obtained from the subject prior to administration of the FXN replacement therapy, is an indication that the FXN replacement therapy is effective.

In certain embodiments, a decrease in the amount of one or more PCs selected from the group consisting of PC(15:0/20:3), PC(15:0/22:6), PC(16:0/14:0), PC(16:0/22:4), PC(16:1/16:0), PC(16:1/20:4), PC(16:1/22.5), PC(17:0/20:5), PC(18:0/20:3), PC(18:0/22:4), PC(18:1/20:3), PC(18:2/18:2), PC(20:4/15:0), PC40:6, PC42:7, in a FXN replacement lipid profile, as compared to a reference FXN lipid profile, e.g., a baseline FXN(−) lipid profile, is an indication that the FXN replacement therapy is effective.

In certain embodiments, a decrease in the amount of one or more PCs selected from the group consisting of PC(15:0/20:3), PC(15:0/22:6), PC(16:0/14:0), PC(16:0/22:4), PC(16:1/16:0), PC(16:1/20:4), PC(16:1/22.5), PC(17:0/20:5), PC(18:0/20:3), PC(18:0/22:4), PC(18:1/20:3), PC(18:2/18:2), PC(20:4/15:0), PC40:6, PC42:7, in a sample obtained from an FXN-deficient subject following administration of FXN replacement therapy, as compared to the amount of the one or more FSLMs in a reference sample, e.g., a sample obtained from the FXN-deficient subject prior to administration of the FXN replacement therapy, is an indication that the FXN replacement therapy is effective.

In some embodiments, a decrease in the amount of PC(15:0/20:3) in a sample obtained from an FXN deficient subject following administration of FXN replacement therapy, as compared to the amount of PC(15:0/20:3) in a sample obtained from the subject prior to administration of the FXN replacement therapy, is an indication that the FXN replacement therapy is effective.

In some embodiments, a decrease in the amount of PC(15:0/22:6) in a sample obtained from an FXN deficient subject following administration of FXN replacement therapy, as compared to the amount of PC(15:0/22:6) in a sample obtained from the subject prior to administration of the FXN replacement therapy, is an indication that the FXN replacement therapy is effective.

In some embodiments, a decrease in the amount of PC(16:0/14:0) in a sample obtained from an FXN deficient subject following administration of FXN replacement therapy, as compared to the amount of PC(16:0/14:0) in a sample obtained from the subject prior to administration of the FXN replacement therapy, is an indication that the FXN replacement therapy is effective.

In some embodiments, a decrease in the amount of PC(16:0/22:4) in a sample obtained from an FXN deficient subject following administration of FXN replacement therapy, as compared to the amount of PC(16:0/22:4) in a sample obtained from the subject prior to administration of the FXN replacement therapy, is an indication that the FXN replacement therapy is effective.

In some embodiments, a decrease in the amount of PC(16:1/16:0) in a sample obtained from an FXN deficient subject following administration of FXN replacement therapy, as compared to the amount of PC(16:1/16:0) in a sample obtained from the subject prior to administration of the FXN replacement therapy, is an indication that the FXN replacement therapy is effective.

In some embodiments, a decrease in the amount of PC(16:1/20:4) in a sample obtained from an FXN deficient subject following administration of FXN replacement therapy, as compared to the amount of PC(16:1/20:4) in a sample obtained from the subject prior to administration of the FXN replacement therapy, is an indication that the FXN replacement therapy is effective.

In some embodiments, a decrease in the amount of PC(16:1/22.5) in a sample obtained from an FXN deficient subject following administration of FXN replacement therapy, as compared to the amount of PC(16:1/22.5) in a sample obtained from the subject prior to administration of the FXN replacement therapy, is an indication that the FXN replacement therapy is effective.

In some embodiments, a decrease in the amount of PC(17:0/20:5) in a sample obtained from an FXN deficient subject following administration of FXN replacement therapy, as compared to the amount of PC(17:0/20:5) in a sample obtained from the subject prior to administration of the FXN replacement therapy, is an indication that the FXN replacement therapy is effective.

In some embodiments, a decrease in the amount of PC(18:0/20:3) in a sample obtained from an FXN deficient subject following administration of FXN replacement therapy, as compared to the amount of PC(18:0/20:3) in a sample obtained from the subject prior to administration of the FXN replacement therapy, is an indication that the FXN replacement therapy is effective.

In some embodiments, a decrease in the amount of PC(18:0/22:4) in a sample obtained from an FXN deficient subject following administration of FXN replacement therapy, as compared to the amount of PC(18:0/22:4) in a sample obtained from the subject prior to administration of the FXN replacement therapy, is an indication that the FXN replacement therapy is effective.

In some embodiments, a decrease in the amount of PC(18:1/20:3) in a sample obtained from an FXN deficient subject following administration of FXN replacement therapy, as compared to the amount of PC(18:1/20:3) in a sample obtained from the subject prior to administration of the FXN replacement therapy, is an indication that the FXN replacement therapy is effective.

In some embodiments, a decrease in the amount of PC(18:2/18:2) in a sample obtained from an FXN deficient subject following administration of FXN replacement therapy, as compared to the amount of PC(18:2/18:2) in a sample obtained from the subject prior to administration of the FXN replacement therapy, is an indication that the FXN replacement therapy is effective.

In some embodiments, a decrease in the amount of PC(20:4/15:0) in a sample obtained from an FXN deficient subject following administration of FXN replacement therapy, as compared to the amount of PC(20:4/15:0) in a sample obtained from the subject prior to administration of the FXN replacement therapy, is an indication that the FXN replacement therapy is effective.

In some embodiments, a decrease in the amount of PC40:6 in a sample obtained from an FXN deficient subject following administration of FXN replacement therapy, as compared to the amount of PC40:6 in a sample obtained from the subject prior to administration of the FXN replacement therapy, is an indication that the FXN replacement therapy is effective.

In some embodiments, a decrease in the amount of PC42:7 in a sample obtained from an FXN deficient subject following administration of FXN replacement therapy, as compared to the amount of PC42:7 in a sample obtained from the subject prior to administration of the FXN replacement therapy, is an indication that the FXN replacement therapy is effective.

In certain embodiments, an increase in the amount of one or more PCs selected from the group consisting of PC(17:1/20:4), PC(18:2/18:3), PC(18:1/24:1), PC(18:2/20:5) and PC(20:4/20:0), in a FXN replacement lipid profile, as compared to a reference FXN lipid profile, e.g., a baseline FXN(−) lipid profile, is an indication that the FXN replacement therapy is effective.

In certain embodiments, an increase in the amount of one or more PCs selected from the group consisting of PC(17:1/20:4), PC(18:2/18:3), PC(18:1/24:1), PC(18:2/20:5) and PC(20:4/20:0), in a sample obtained from an FXN-deficient subject following administration of FXN replacement therapy, as compared to the amount of the one or more FSLMs in a reference sample, e.g., a sample obtained from the FXN-deficient subject prior to administration of the FXN replacement therapy, is an indication that the FXN replacement therapy is effective.

In some embodiments, an increase in the amount of PC(17:1/20:4) in a sample obtained from an FXN deficient subject following administration of FXN replacement therapy, as compared to the amount of PC(17:1/20:4) in a sample obtained from the subject prior to administration of the FXN replacement therapy, is an indication that the FXN replacement therapy is effective.

In some embodiments, an increase in the amount of PC(18:2/18:3) in a sample obtained from an FXN deficient subject following administration of FXN replacement therapy, as compared to the amount of PC(18:2/18:3) in a sample obtained from the subject prior to administration of the FXN replacement therapy, is an indication that the FXN replacement therapy is effective.

In some embodiments, an increase in the amount of PC(18:1/24:1) in a sample obtained from an FXN deficient subject following administration of FXN replacement therapy, as compared to the amount of PC(18:1/24:1) in a sample obtained from the subject prior to administration of the FXN replacement therapy, is an indication that the FXN replacement therapy is effective.

In some embodiments, an increase in the amount of PC(18:2/20:5) in a sample obtained from an FXN deficient subject following administration of FXN replacement therapy, as compared to the amount of PC(18:2/20:5) in a sample obtained from the subject prior to administration of the FXN replacement therapy, is an indication that the FXN replacement therapy is effective.

In some embodiments, an increase in the amount of PC(20:4/20:0) in a sample obtained from an FXN deficient subject following administration of FXN replacement therapy, as compared to the amount of PC(20:4/20:0) in a sample obtained from the subject prior to administration of the FXN replacement therapy, is an indication that the FXN replacement therapy is effective.

In some embodiments, a decrease in the amount of DG(18:1/18:2) in a sample obtained from an FXN deficient subject following administration of FXN replacement therapy, as compared to the amount of DG(18:1/18:2) in a sample obtained from the subject prior to administration of the FXN replacement therapy, is an indication that the FXN replacement therapy is effective.

In some embodiments, a decrease in the amount of CE14:1 in a sample obtained from an FXN deficient subject following administration of FXN replacement therapy, as compared to the amount of CE14:1 in a sample obtained from the subject prior to administration of the FXN replacement therapy, is an indication that the FXN replacement therapy is effective.

In some embodiments, an increase in the amount of CE16:0 in a sample obtained from an FXN deficient subject following administration of FXN replacement therapy, as compared to the amount of CE16:0 in a sample obtained from the subject prior to administration of the FXN replacement therapy, is an indication that the FXN replacement therapy is effective.

In some embodiments, an increase in the amount of CE20:5 in a sample obtained from an FXN deficient subject following administration of FXN replacement therapy, as compared to the amount of CE20:5 in a sample obtained from the subject prior to administration of the FXN replacement therapy, is an indication that the FXN replacement therapy is effective.

In certain embodiments of the methods provided herein, lack of an increase or decrease in the amount of one or more FSLMs, e.g., one or any combination of one or more TGs, ether phospholipids (e.g., PCO- or PEO-), PCs, DGs or CEs as described herein, in a sample obtained from an FXN-deficient subject following administration of an FXN replacement therapy, as compared to the amount of the one or more FSLMs in a control sample, e.g., a sample from the FXN-deficient subject prior to administration of the FXN replacement therapy, is an indication that the FXN replacement therapy is ineffective, e.g., at the current dose, and should be modified. For example, the FXN replacement therapy may be modified by increasing the dose and/or administration frequency of the FXN replacement therapy.

In certain embodiments, the methods provided herein may also include monitoring a subject being administered FXN replacement therapy. In some embodiments, lack of an increase or decrease in the detected amount of one or more FSLMs, e.g., one or any combination of one or more TGs, ether phospholipids (e.g., PCO- or PEO-), PCs, DGs or CEs as described herein, in a second sample obtained from a subject after administration of FXN replacement therapy, as compared to the amount of the one or more FSLMs in a first sample obtained from the subject before administration of FXN replacement therapy, is an indication that the FXN replacement therapy is not efficacious and/or the subject is not responsive FXN replacement therapy. The method may further include the step of adjusting the FXN replacement therapy, e.g., by increasing the dose and/or administration frequency of the FXN replacement therapy.

In other embodiments, an increased or decreased lipid amount of one or more FSLMs, e.g., one or any combination of one or more TGs, ether phospholipids (e.g., PCO- or PEO-), PCs, DGs or CEs as described herein, in a second sample obtained from a subject after administration of FXN replacement therapy, as compared to the amount of the one or more FSLMs in a first sample obtained from the subject before administration of FXN replacement therapy, is an indication that the FXN replacement therapy is efficacious and/or the subject is responsive to the FXN replacement therapy. The method may further include the step of adjusting the FXN replacement therapy, e.g., by decreasing the dose and/or administration frequency of the FXN replacement therapy, or ceasing the therapy.

amount

In certain embodiments, the amount of one or more TGs, wherein the three acyl groups in each TG molecule contain less than 56 carbon atoms, or contain 7 or less unsaturations, is decreased in an FXN deficient subject following treatment with an FXN replacement. In some embodiments, the one or more TGs are selected from the group consisting of TG45:1, TG46:1, TG46:3, TG47:1, TG47:2, TG48:0, TG48:1, TG48:2, TG48:3, TG49:1, TG49:2, TG49:3, TG49:4, TG50:1, TG50:2, TG50:3, TG50:4, TG50:5, TG51:1, TG51:2, TG51:3, TG51:4, TG52:2, TG52:3, TG52:4, TG52:5, TG52:6, TG53:2, TG53:3, TG53:4, TG53:5, TG54:4, TG54:5, TG54:6 and TG54:7.

In some embodiments, the amount of TG45:1 is decreased following treatment of an FXN deficient subject with FXN replacement therapy. In some embodiments, the amount of TG46:1 is decreased following treatment of an FXN deficient subject with FXN replacement therapy. In some embodiments, the amount of TG46:3 is decreased following treatment of an FXN deficient subject with FXN replacement therapy. In some embodiments, the amount of TG47:1 is decreased following treatment of an FXN deficient subject with FXN replacement therapy. In some embodiments, the amount of TG47:2 is decreased following treatment of an FXN deficient subject with FXN replacement therapy. In some embodiments, the amount of TG48:0 is decreased following treatment of an FXN deficient subject with FXN replacement therapy. In some embodiments, the amount of TG48:1 is decreased following treatment of an FXN deficient subject with FXN replacement therapy. In some embodiments, the amount of TG48:2 is decreased following treatment of an FXN deficient subject with FXN replacement therapy. In some embodiments, the amount of TG48:3 is decreased following treatment of an FXN deficient subject with FXN replacement therapy. In some embodiments, the amount of TG49:1 is decreased following treatment of an FXN deficient subject with FXN replacement therapy. In some embodiments, the amount of TG49:2 is decreased following treatment of an FXN deficient subject with FXN replacement therapy. In some embodiments, the amount of TG49:3 is decreased following treatment of an FXN deficient subject with FXN replacement therapy. In some embodiments, the amount of TG49:4 is decreased following treatment of an FXN deficient subject with FXN replacement therapy. In some embodiments, the amount of TG50:1 is decreased following treatment of an FXN deficient subject with FXN replacement therapy. In some embodiments, the amount of TG50:2 is decreased following treatment of an FXN deficient subject with FXN replacement therapy. In some embodiments, the amount of TG50:3 is decreased following treatment of an FXN deficient subject with FXN replacement therapy. In some embodiments, the amount of TG50:4 is decreased following treatment of an FXN deficient subject with FXN replacement therapy. In some embodiments, the amount of TG50:5 is decreased following treatment of an FXN deficient subject with FXN replacement therapy. In some embodiments, the amount of TG51:1 is decreased following treatment of an FXN deficient subject with FXN replacement therapy. In some embodiments, the amount of TG51:2 is decreased following treatment of an FXN deficient subject with FXN replacement therapy. In some embodiments, the amount of TG51:3 is decreased following treatment of an FXN deficient subject with FXN replacement therapy. In some embodiments, the amount of TG51:4 is decreased following treatment of an FXN deficient subject with FXN replacement therapy. In some embodiments, the amount of TG52:2 is decreased following treatment of an FXN deficient subject with FXN replacement therapy. In some embodiments, the amount of TG52:3 is decreased following treatment of an FXN deficient subject with FXN replacement therapy. In some embodiments, the amount of TG52:4 is decreased following treatment of an FXN deficient subject with FXN replacement therapy. In some embodiments, the amount of TG52:5 is decreased following treatment of an FXN deficient subject with FXN replacement therapy. In some embodiments, the amount of TG52:6 is decreased following treatment of an FXN deficient subject with FXN replacement therapy. In some embodiments, the amount of TG53:2 is decreased following treatment of an FXN deficient subject with FXN replacement therapy. In some embodiments, the amount of TG53:3 is decreased following treatment of an FXN deficient subject with FXN replacement therapy. In some embodiments, the amount of TG53:4 is decreased following treatment of an FXN deficient subject with FXN replacement therapy. In some embodiments, the amount of TG53:5 is decreased following treatment of an FXN deficient subject with FXN replacement therapy. In some embodiments, the amount of TG54:4 is decreased following treatment of an FXN deficient subject with FXN replacement therapy. In some embodiments, the amount of TG54:5 is decreased following treatment of an FXN deficient subject with FXN replacement therapy. In some embodiments, the amount of TG54:6 is decreased following treatment of an FXN deficient subject with FXN replacement therapy. In some embodiments, the amount of TG54:7 is decreased following treatment of an FXN deficient subject with FXN replacement therapy.

In certain embodiments, the amount of one or more PCO- is increased in an FXN deficient subject following treatment with an FXN replacement therapy. In some embodiments, the one or more PCO- are selected from the group consisting of PC(O-16:0/14:0), PC(O-16:0/18:2), PC(O-16:0/20:3), PC(O-16:0/20:4), PC(O-16:0/22:6), PC(O-17:0/20:4), PC(O-18:0/18:1), PC(O-18:0/22:6), PC(O-18:0/18:2), PC(O-18:1/18:2), PC(O-18:1/20:4), PC(O-18:1/20:5), PC(O-18:1/22:6), PC(O-(20:0/22:6), PC(O-20:1/22.6), PC(O-20:2/20:4), PC(O-22:2/20:4), PC(O-22:1/22:6), PC(O-22:2/20:4), PC(O-(24:1/22:6), PC(O-24:2/20:4), PCO-34:2, PCO-36:3, PCO-34:2, PCO-38:3, PCO-40:2, PCO-40:6, and PCO-44:7 and PCO-46.8.

In some embodiments, the amount of PC(O-16:0/14:0) is increased following treatment of an FXN deficient subject with FXN replacement therapy. In some embodiments, the amount of PC(O-16:0/18:2) is increased following treatment of an FXN deficient subject with FXN replacement therapy. In some embodiments, the amount of PC(O-16:0/20:3) is increased following treatment of an FXN deficient subject with FXN replacement therapy. In some embodiments, the amount of PC(O-16:0/20:4) is increased following treatment of an FXN deficient subject with FXN replacement therapy. In some embodiments, the amount of PC(O-16:0/22:6) is increased following treatment of an FXN deficient subject with FXN replacement therapy. In some embodiments, the amount of PC(O-17:0/20:4) is increased following treatment of an FXN deficient subject with FXN replacement therapy. In some embodiments, the amount of PC(O-18:0/18:1) is increased following treatment of an FXN deficient subject with FXN replacement therapy. In some embodiments, the amount of PC(O-18:0/22:6) is increased following treatment of an FXN deficient subject with FXN replacement therapy. In some embodiments, the amount of PC(O-18:0/18:2) is increased following treatment of an FXN deficient subject with FXN replacement therapy. In some embodiments, the amount of PC(O-18:1/18:2) is increased following treatment of an FXN deficient subject with FXN replacement therapy. In some embodiments, the amount of PC(O-18:1/20:4) is increased following treatment of an FXN deficient subject with FXN replacement therapy. In some embodiments, the amount of PC(O-18:1/20:5) is increased following treatment of an FXN deficient subject with FXN replacement therapy. In some embodiments, the amount of PC(O-18:1/22:6) is increased following treatment of an FXN deficient subject with FXN replacement therapy. In some embodiments, the amount of PC(O-(20:0/22:6) is increased following treatment of an FXN deficient subject with FXN replacement therapy. In some embodiments, the amount of PC(O-20:1/22.6) is increased following treatment of an FXN deficient subject with FXN replacement therapy. In some embodiments, the amount of PC(O-20:2/20:4) is increased following treatment of an FXN deficient subject with FXN replacement therapy. In some embodiments, the amount of PC(O-22:2/20:4) is increased following treatment of an FXN deficient subject with FXN replacement therapy. In some embodiments, the amount of PC(O-22:1/22:6) is increased following treatment of an FXN deficient subject with FXN replacement therapy. In some embodiments, the amount of PC(O-22:2/20:4) is increased following treatment of an FXN deficient subject with FXN replacement therapy. In some embodiments, the amount of PC(O-(24:1/22:6) is increased following treatment of an FXN deficient subject with FXN replacement therapy. In some embodiments, the amount of PCO-34:2 is increased following treatment of an FXN deficient subject with FXN replacement therapy. In some embodiments, the amount of PCO-36:3 is increased following treatment of an FXN deficient subject with FXN replacement therapy. In some embodiments, the amount of PCO-34:2 is increased following treatment of an FXN deficient subject with FXN replacement therapy. In some embodiments, the amount of PCO-38:3 is increased following treatment of an FXN deficient subject with FXN replacement therapy. In some embodiments, the amount of PCO-40:2 is increased following treatment of an FXN deficient subject with FXN replacement therapy. In some embodiments, the amount of PCO-40:6 is increased following treatment of an FXN deficient subject with FXN replacement therapy. In some embodiments, the amount of PCO-44:7 is increased following treatment of an FXN deficient subject with FXN replacement therapy. In some embodiments, the amount of PCO-46.8 is increased following treatment of an FXN deficient subject with FXN replacement therapy.

In certain embodiments, the amount of one or more PCs selected from the group consisting of PC(15:0/20:3), PC(15:0/22:6), PC(16:0/14:0), PC(16:0/22:4), PC(16:1/16:0), PC(16:1/20:4), PC(16:1/22.5), PC(17:0/20:5), PC(18:0/20:3), PC(18:0/22:4), PC(18:1/20:3), PC(18:2/18:2), PC(20:4/15:0), PC40:6, PC42:7 is decreased in an FXN deficient subject following treatment with an FXN replacement.

In some embodiments, the amount of PC(15:0/20:3) is decreased following treatment of an FXN deficient subject with FXN replacement therapy. In some embodiments, the amount of PC(15:0/22:6) is decreased following treatment of an FXN deficient subject with FXN replacement therapy. In some embodiments, the amount of PC(16:0/14:0) is decreased following treatment of an FXN deficient subject with FXN replacement therapy. In some embodiments, the amount of PC(16:0/22:4) is decreased following treatment of an FXN deficient subject with FXN replacement therapy. In some embodiments, the amount of PC(16:1/16:0) is decreased following treatment of an FXN deficient subject with FXN replacement therapy. In some embodiments, the amount of PC(16:1/20:4) is decreased following treatment of an FXN deficient subject with FXN replacement therapy. In some embodiments, the amount of PC(16:1/22.5) is decreased following treatment of an FXN deficient subject with FXN replacement therapy. In some embodiments, the amount of PC(17:0/20:5) is decreased following treatment of an FXN deficient subject with FXN replacement therapy. In some embodiments, the amount of PC(18:0/20:3) is decreased following treatment of an FXN deficient subject with FXN replacement therapy. In some embodiments, the amount of PC(18:0/22:4) is decreased following treatment of an FXN deficient subject with FXN replacement therapy. In some embodiments, the amount of PC(18:1/20:3) is decreased following treatment of an FXN deficient subject with FXN replacement therapy. In some embodiments, the amount of PC(18:2/18:2) is decreased following treatment of an FXN deficient subject with FXN replacement therapy. In some embodiments, the amount of PC(20:4/15:0) is decreased following treatment of an FXN deficient subject with FXN replacement therapy. In some embodiments, the amount of PC40:6 is decreased following treatment of an FXN deficient subject with FXN replacement therapy. In some embodiments, the amount of PC42:7 is decreased following treatment of an FXN deficient subject with FXN replacement therapy.

In certain embodiments, the amount of one or more PCs selected from the group consisting of PC(17:1/20:4), PC(18:2/18:3), PC(18:1/24:1), PC(18:2/20:5) and PC(20:4/20:0) is increased in an FXN deficient subject following treatment with an FXN replacement.

In some embodiments, the amount of PC(17:1/20:4) is increased following treatment of an FXN deficient subject with FXN replacement therapy. In some embodiments, the amount of PC(18:2/18:3) is increased following treatment of an FXN deficient subject with FXN replacement therapy. In some embodiments, the amount of PC(18:1/24:1) is increased following treatment of an FXN deficient subject with FXN replacement therapy. In some embodiments, the amount of PC(18:2/20:5) is increased following treatment of an FXN deficient subject with FXN replacement therapy. In some embodiments, the amount of PC(20:4/20:0) is increased following treatment of an FXN deficient subject with FXN replacement therapy.

In some embodiments, the amount of DG(18:1/18:2) is decreased following treatment of an FXN deficient subject with FXN replacement therapy.

In some embodiments, the amount of CE14:1 is decreased following treatment of an FXN deficient subject with FXN replacement therapy.

In some embodiments, the amount of CE16:0 is increased following treatment of an FXN deficient subject with FXN replacement therapy.

In some embodiments, the amount of CE20:5 is increased following treatment of an FXN deficient subject with FXN replacement therapy.

Methods for Monitoring Treatment with FXN Replacement Therapy

In some aspects, the present disclosure also provides a method of monitoring treatment of a subject with a frataxin (FXN) replacement therapy, the method comprising: (a) determining a first FXN replacement lipid profile for one or more FXN-sensitive lipid markers (FSLMs) in a first sample obtained from an FXN deficient subject at a first time point following administration of an FXN replacement therapy to the subject, (b) determining a second FXN replacement lipid profile for the one or more FXN-sensitive lipid markers (FSLMs) in a second sample obtained from the subject at a second time point that is later than the first time point; and (c) comparing the second FXN replacement lipid profile with the first FXN replacement lipid profile; thereby monitoring treatment of the subject with the FXN replacement therapy.

In some embodiments, comparing the second FXN replacement lipid profile with the first FXN replacement lipid profile comprises comparing the amount of one or more FSLMs in the second FXN replacement lipid profile with the amount of the corresponding one or more FSLMs in the first FXN replacement lipid profile.

In some embodiments, the one or more FSLMs comprise one or any combination of one or more TGs, ether phospholipids (e.g., PCO- or PEO-), PCs, DGs or CEs as described herein. For example, the one or more FSLMs may comprise one or more TGs, wherein the three acyl groups in each TG molecule contain less than 56 carbon atoms, e.g., one or more of TG45:1, TG46:1, TG46:3, TG47:1, TG47:2, TG48:0, TG48:1, TG48:2, TG48:3, TG49:1, TG49:2, TG49:3, TG49:4, TG50:1, TG50:2, TG50:3, TG50:4, TG50:5, TG51:1, TG51:2, TG51:3, TG51:4, TG52:2, TG52:3, TG52:4, TG52:5, TG52:6, TG53:2, TG53:3, TG53:4, TG53:5, TG54:4, TG54:5, TG54:6 and TG54:7

In certain embodiments, a decrease in the amount of one or more of TGs, wherein the three acyl groups in each TG molecule contain less than 56 carbon atoms (e.g., TG45:1, TG46:1, TG46:3, TG47:1, TG47:2, TG48:0, TG48:1, TG48:2, TG48:3, TG49:1, TG49:2, TG49:3, TG49:4, TG50:1, TG50:2, TG50:3, TG50:4, TG50:5, TG51:1, TG51:2, TG51:3, TG51:4, TG52:2, TG52:3, TG52:4, TG52:5, TG52:6, TG53:2, TG53:3, TG53:4, TG53:5, TG54:4, TG54:5, TG54:6 and TG54:7), in the second FXN replacement lipid profile, as compared to the first FXN replacement lipid profile, is an indication that the FXN replacement therapy is effective.

In certain embodiments, a decrease in the amount of one or more TGs, wherein the three acyl groups in each TG molecule contain 7 or less unsaturations (e.g., TG45:1, TG46:1, TG46:3, TG47:1, TG47:2, TG48:0, TG48:1, TG48:2, TG48:3, TG49:1, TG49:2, TG49:3, TG49:4, TG50:1, TG50:2, TG50:3, TG50:4, TG50:5, TG51:1, TG51:2, TG51:3, TG51:4, TG52:2, TG52:3, TG52:4, TG52:5, TG52:6, TG53:2, TG53:3, TG53:4, TG53:5, TG54:4, TG54:5, TG54:6 and TG54:7), in the second FXN replacement lipid profile, as compared to the first FXN replacement lipid profile, is an indication that the FXN replacement therapy is effective. In certain embodiments, lack of a decrease in the amount of one or more TGs, wherein the three acyl groups in each TG molecule contain 7 or less unsaturations (e.g., TG45:1, TG46:1, TG46:3, TG47:1, TG47:2, TG48:0, TG48:1, TG48:2, TG48:3, TG49:1, TG49:2, TG49:3, TG49:4, TG50:1, TG50:2, TG50:3, TG50:4, TG50:5, TG51:1, TG51:2, TG51:3, TG51:4, TG52:2, TG52:3, TG52:4, TG52:5, TG52:6, TG53:2, TG53:3, TG53:4, TG53:5, TG54:4, TG54:5, TG54:6 and TG54:7), in the second FXN replacement lipid profile, as compared to the first FXN replacement lipid profile, is an indication that the FXN replacement therapy is not effective.

In certain embodiments, an increase in the amount of one or more PCO-, e.g., one or more of PC(O-16:0/14:0), PC(O-16:0/18:2), PC(O-16:0/20:3), PC(O-16:0/20:4), PC(O-16:0/22:6), PC(O-17:0/20:4), PC(O-18:0/18:1), PC(O-18:0/22:6), PC(O-18:0/18:2), PC(O-18:1/18:2), PC(O-18:1/20:4), PC(O-18:1/20:5), PC(O-18:1/22:6), PC(O-(20:0/22:6), PC(O-20:1/22.6), PC(O-20:2/20:4), PC(O-22:2/20:4), PC(O-22:1/22:6), PC(O-22:2/20:4), PC(O-(24:1/22:6), PC(O-24:2/20:4), PCO-34:2, PCO-36:3, PCO-34:2, PCO-38:3, PCO-40:2, PCO-40:6, PCO-44:7 and PCO-46.8, in the second FXN replacement lipid profile, as compared to the first FXN replacement lipid profile, is an indication that the FXN replacement therapy is effective. In certain embodiments, lack an increase in the amount of one or more PCO-, e.g., one or more of PC(O-16:0/14:0), PC(O-16:0/18:2), PC(O-16:0/20:3), PC(O-16:0/20:4), PC(O-16:0/22:6), PC(O-17:0/20:4), PC(O-18:0/18:1), PC(O-18:0/22:6), PC(O-18:0/18:2), PC(O-18:1/18:2), PC(O-18:1/20:4), PC(O-18:1/20:5), PC(O-18:1/22:6), PC(O-(20:0/22:6), PC(O-20:1/22.6), PC(O-20:2/20:4), PC(O-22:2/20:4), PC(O-22:1/22:6), PC(O-22:2/20:4), PC(O-(24:1/22:6), PC(O-24:2/20:4), PCO-34:2, PCO-36:3, PCO-34:2, PCO-38:3, PCO-40:2, PCO-40:6, PCO-44:7 and PCO-46.8, in the second FXN replacement lipid profile, as compared to the first FXN replacement lipid profile, is an indication that the FXN replacement therapy is not effective.

In certain embodiments, a decrease in the amount of one or more PCs selected from the group consisting of PC(15:0/20:3), PC(15:0/22:6), PC(16:0/14:0), PC(16:0/22:4), PC(16:1/16:0), PC(16:1/20:4), PC(16:1/22.5), PC(17:0/20:5), PC(18:0/20:3), PC(18:0/22:4), PC(18:1/20:3), PC(18:2/18:2), PC(20:4/15:0), PC40:6, PC42:7, in the second FXN replacement lipid profile, as compared to the first FXN replacement lipid profile, is an indication that the FXN replacement therapy is effective. In certain embodiments, lack of a decrease in the amount of one or more PCs selected from the group consisting of PC(15:0/20:3), PC(15:0/22:6), PC(16:0/14:0), PC(16:0/22:4), PC(16:1/16:0), PC(16:1/20:4), PC(16:1/22.5), PC(17:0/20:5), PC(18:0/20:3), PC(18:0/22:4), PC(18:1/20:3), PC(18:2/18:2), PC(20:4/15:0), PC40:6, PC42:7, in the second FXN replacement lipid profile, as compared to the first FXN replacement lipid profile, is an indication that the FXN replacement therapy is not effective.

In certain embodiments, an increase in the amount of one or more PCs selected from the group consisting of PC(17:1/20:4), PC(18:2/18:3), PC(18:1/24:1), PC(18:2/20:5) and PC(20:4/20:0), in the second FXN replacement lipid profile, as compared to the first FXN replacement lipid profile, is an indication that the FXN replacement therapy is effective. In certain embodiments, lack of an increase in the amount of one or more PCs selected from the group consisting of PC(17:1/20:4), PC(18:2/18:3), PC(18:1/24:1), PC(18:2/20:5) and PC(20:4/20:0), in the second FXN replacement lipid profile, as compared to the first FXN replacement lipid profile, is an indication that the FXN replacement therapy is not effective.

In some embodiments, a decrease in the amount of DG(18:1/18:2) in the second FXN replacement lipid profile, as compared to the first FXN replacement lipid profile, is an indication that the FXN replacement therapy is effective. In some embodiments, lack of a decrease in the amount of DG(18:1/18:2) in the second FXN replacement lipid profile, as compared to the first FXN replacement lipid profile, is an indication that the FXN replacement therapy is not effective.

In some embodiments, a decrease in the amount of CE14:1 in the second FXN replacement lipid profile, as compared to the first FXN replacement lipid profile, is an indication that the FXN replacement therapy is effective. In some embodiments, lack of a decrease in the amount of CE14:1 in the second FXN replacement lipid profile, as compared to the first FXN replacement lipid profile, is an indication that the FXN replacement therapy is not effective.

In some embodiments, an increase in the amount of CE16:0 in the second FXN replacement lipid profile, as compared to the first FXN replacement lipid profile, is an indication that the FXN replacement therapy is effective. In some embodiments, lack of an increase in the amount of CE16:0 in the second FXN replacement lipid profile, as compared to the first FXN replacement lipid profile, is an indication that the FXN replacement therapy is not effective.

In some embodiments, an increase in the amount of CE20:5 in the second FXN replacement lipid profile, as compared to the first FXN replacement lipid profile, is an indication that the FXN replacement therapy is effective. In some embodiments, lack of an increase in the amount of CE20:5 in the second FXN replacement lipid profile, as compared to the first FXN replacement lipid profile, is an indication that the FXN replacement therapy is not effective.

In some embodiments, the method for monitoring treatment with FXN replacement therapy further comprises making a determination, or making a recommendation to a healthcare provider, to maintain the FXN replacement therapy regimen based on the comparison in step (c). In some embodiments, the method further comprises making a determination, or making a recommendation to a healthcare provider, to alter the FXN replacement therapy regimen based on the comparison in step (c). In some embodiments, the method further comprises making a determination to maintain the dose and/or administration frequency, increase the dose and/or administration frequency, or decrease the dose and/or administration frequency of the FXN replacement therapy based on the comparison in step (c). In some embodiments, the method further comprises making a recommendation, e.g., to a healthcare provider, to maintain the dose and/or administration frequency, increase the dose and/or administration frequency, or decrease the dose and/or administration frequency of the FXN replacement therapy based on the comparison in step (c).

For example, in some embodiments, the method comprises continuing to administer the FXN replacement therapy regimen (e.g., without changing the regimen, e.g., maintaining the dose and/or administration frequency) to the subject based on the comparison in step (c). In some embodiments, the method further comprises altering the FXN replacement therapy regimen based on the comparison in step (c). In some embodiments, the method further comprises administering an altered FXN replacement therapy regimen based on the comparison in step (c). In some embodiments, administering an altered FXN replacement therapy regimen comprises administering an increased dose and/or administration frequency, or administering a decreased dose and/or administration frequency of the FXN replacement therapy.

For example, the method for monitoring treatment of a subject with FXN replacement therapy may further comprise the step of continuing administering the FXN replacement therapy to the subject without adjustments, or decreasing the dose and/or administration frequency of the FXN replacement therapy if the FXN replacement therapy is determined to be effective. In other embodiments, the method for monitoring treatment of a subject with FXN replacement therapy may further comprise a step of adjusting FXN replacement therapy by increasing the dose and/or administration frequency of the FXN replacement therapy if the FXN replacement therapy is determined to be not effective.

It will be appreciated that following administration of FXN replacement therapy to an FXN deficient subject, the level of FXN achieved in the subject may reach a desired or target level of FXN, e.g., similar to levels of FXN present in a normal, healthy subject, or similar to levels of FXN present in hFXN heterozygotes. In such instances, it is beneficial for the dose and/or frequency of administration of the FXN replacement therapy to then be maintained. Further, when such a desired level of FXN in the subject is achieved and maintained constant over time, it is beneficial for the dose and/or frequency of administration of the FXN replacement therapy to be maintained. It will be further appreciated that following administration of FXN replacement therapy to an FXN deficient subject, the level of FXN achieved in the subject may be higher than the desired or target level of FXN, e.g., higher than the level of FXN present in a normal, healthy subject, or higher than the level of FXN present in hFXN heterozygotes. In such instances, it is beneficial for the dose and/or frequency of administration of the FXN replacement therapy to be decreased. Alternatively, the level of FXN achieved in the subject may be lower than the desired or target level of FXN, e.g., lower than the level of FXN present in a normal, healthy subject, or lower than the level of FXN present in hFXN heterozygotes. In such instances, it is beneficial for the dose and/or frequency of administration of the FXN replacement therapy to be increased.

Accordingly, a decrease in the amount of one or more TGs, wherein the three acyl groups in each TG molecule contain 7 or less unsaturations (e.g., TG45:1, TG46:1, TG46:3, TG47:1, TG47:2, TG48:0, TG48:1, TG48:2, TG48:3, TG49:1, TG49:2, TG49:3, TG49:4, TG50:1, TG50:2, TG50:3, TG50:4, TG50:5, TG51:1, TG51:2, TG51:3, TG51:4, TG52:2, TG52:3, TG52:4, TG52:5, TG52:6, TG53:2, TG53:3, TG53:4, TG53:5, TG54:4, TG54:5, TG54:6 and TG54:7), in the second FXN replacement lipid profile, as compared to the first FXN replacement lipid profile, is an indication that the dose and/or administration schedule of the FXN replacement therapy should be maintained or decreased.

In some embodiments, lack of a decrease in the amount of one or more TGs, wherein the three acyl groups in each TG molecule contain 7 or less unsaturations (e.g., TG45:1, TG46:1, TG46:3, TG47:1, TG47:2, TG48:0, TG48:1, TG48:2, TG48:3, TG49:1, TG49:2, TG49:3, TG49:4, TG50:1, TG50:2, TG50:3, TG50:4, TG50:5, TG51:1, TG51:2, TG51:3, TG51:4, TG52:2, TG52:3, TG52:4, TG52:5, TG52:6, TG53:2, TG53:3, TG53:4, TG53:5, TG54:4, TG54:5, TG54:6 and TG54:7), in the second FXN replacement lipid profile, as compared to the first FXN replacement lipid profile, is an indication that the dose and/or administration schedule of the FXN replacement therapy should be maintained or increased.

In certain embodiments, an increase in the amount of one or more PCO-, e.g., one or more of PC(O-16:0/14:0), PC(O-16:0/18:2), PC(O-16:0/20:3), PC(O-16:0/20:4), PC(O-16:0/22:6), PC(O-17:0/20:4), PC(O-18:0/18:1), PC(O-18:0/22:6), PC(O-18:0/18:2), PC(O-18:1/18:2), PC(O-18:1/20:4), PC(O-18:1/20:5), PC(O-18:1/22:6), PC(O-(20:0/22:6), PC(O-20:1/22.6), PC(O-20:2/20:4), PC(O-22:2/20:4), PC(O-22:1/22:6), PC(O-22:2/20:4), PC(O-(24:1/22:6), PC(O-24:2/20:4), PCO-34:2, PCO-36:3, PCO-34:2, PCO-38:3, PCO-40:2, PCO-40:6, PCO-44:7 and PCO-46.8, is an indication that the dose and/or administration schedule of the FXN replacement therapy should be maintained or decreased.

In certain embodiments, lack of an increase in the amount of one or more PCO-, e.g., one or more of PC(O-16:0/14:0), PC(O-16:0/18:2), PC(O-16:0/20:3), PC(O-16:0/20:4), PC(O-16:0/22:6), PC(O-17:0/20:4), PC(O-18:0/18:1), PC(O-18:0/22:6), PC(O-18:0/18:2), PC(O-18:1/18:2), PC(O-18:1/20:4), PC(O-18:1/20:5), PC(O-18:1/22:6), PC(O-(20:0/22:6), PC(O-20:1/22.6), PC(O-20:2/20:4), PC(O-22:2/20:4), PC(O-22:1/22:6), PC(O-22:2/20:4), PC(O-(24:1/22:6), PC(O-24:2/20:4), PCO-34:2, PCO-36:3, PCO-34:2, PCO-38:3, PCO-40:2, PCO-40:6, PCO-44:7 and PCO-46.8, is an indication that the dose and/or administration schedule of the FXN replacement therapy should be maintained or increased.

In certain embodiments, a decrease in the amount of one or more PCs selected from the group consisting of PC(15:0/20:3), PC(15:0/22:6), PC(16:0/14:0), PC(16:0/22:4), PC(16:1/16:0), PC(16:1/20:4), PC(16:1/22.5), PC(17:0/20:5), PC(18:0/20:3), PC(18:0/22:4), PC(18:1/20:3), PC(18:2/18:2), PC(20:4/15:0), PC40:6, PC42:7, is an indication that the dose and/or administration schedule of the FXN replacement therapy should be maintained or decreased.

In certain embodiments, lack a decrease in the amount of one or more PCs selected from the group consisting of PC(15:0/20:3), PC(15:0/22:6), PC(16:0/14:0), PC(16:0/22:4), PC(16:1/16:0), PC(16:1/20:4), PC(16:1/22.5), PC(17:0/20:5), PC(18:0/20:3), PC(18:0/22:4), PC(18:1/20:3), PC(18:2/18:2), PC(20:4/15:0), PC40:6, PC42:7, is an indication that the dose and/or administration schedule of the FXN replacement therapy should be maintained or increased.

In certain embodiments, an increase in the amount of one or more PCs selected from the group consisting of PC(17:1/20:4), PC(18:2/18:3), PC(18:1/24:1), PC(18:2/20:5) and PC(20:4/20:0), in the second FXN replacement lipid profile, as compared to the first FXN replacement lipid profile, is an indication that the dose and/or administration schedule of the FXN replacement therapy should be maintained or decreased.

In certain embodiments, lack of an increase in the amount of one or more PCs selected from the group consisting of PC(17:1/20:4), PC(18:2/18:3), PC(18:1/24:1), PC(18:2/20:5) and PC(20:4/20:0), in the second FXN replacement lipid profile, as compared to the first FXN replacement lipid profile, is an indication that the dose and/or administration schedule of the FXN replacement therapy should be maintained or increased.

In some embodiments, a decrease in the amount of DG(18:1/18:2) in a sample obtained from an FXN deficient subject following administration of FXN replacement therapy, as compared to the amount of DG(18:1/18:2) in the second FXN replacement lipid profile, as compared to the first FXN replacement lipid profile, is an indication that the dose and/or administration schedule of the FXN replacement therapy should be maintained or decreased.

In some embodiments, lack of a decrease in the amount of DG(18:1/18:2) in a sample obtained from an FXN deficient subject following administration of FXN replacement therapy, as compared to the amount of DG(18:1/18:2) in the second FXN replacement lipid profile, as compared to the first FXN replacement lipid profile, is an indication that the dose and/or administration schedule of the FXN replacement therapy should be maintained or increased.

In some embodiments, a decrease in the amount of CE14:1 in a sample in the second FXN replacement lipid profile, as compared to the first FXN replacement lipid profile, is an indication that the dose and/or administration schedule of the FXN replacement therapy should be maintained or decreased.

In some embodiments, lack of a decrease in the amount of CE14:1 in the second FXN replacement lipid profile, as compared to the first FXN replacement lipid profile, is an indication that the dose and/or administration schedule of the FXN replacement therapy should be maintained or increased.

In some embodiments, an increase in the amount of CE16:0 in the second FXN replacement lipid profile, as compared to the first FXN replacement lipid profile, is an indication that the dose and/or administration schedule of the FXN replacement therapy should be maintained or decreased.

In some embodiments, lack of an increase in the amount of CE16:0 in the second FXN replacement lipid profile, as compared to the first FXN replacement lipid profile, is an indication that the dose and/or administration schedule of the FXN replacement therapy should be maintained or increased.

In some embodiments, an increase in the amount of CE20:5 in the second FXN replacement lipid profile, as compared to the first FXN replacement lipid profile, is an indication that the dose and/or administration schedule of the FXN replacement therapy should be maintained or decreased.

In some embodiments, lack of an increase in the amount of CE20:5 in the second FXN replacement lipid profile, as compared to the first FXN replacement lipid profile, is an indication that the dose and/or administration schedule of the FXN replacement therapy should be maintained or increased.

Methods of the present disclosure for evaluating efficacy of a frataxin replacement therapy or for detecting and/or measuring one or more FSLM of the present disclosure, e.g., TGs, ether phospholipids (e.g., PCO- or PEO-), PCs, DGs or CEs as described herein, involve detection and/or quantification of one or more FSLMs using mass spectrometry (MS). During analysis using MS, an FSLM may be ionized and thereby converted into an ionized FSLM (e.g., a positively charged ionized FSLM or a negatively charged ionized FSLM). Ionization of FSLM makes it possible to detect the ionized FSLM by MS by identifying its corresponding m/z value. In some embodiments, one or more FSLMs may be detected and/or quantified using tandem mass spectrometry (MS/MS). During analysis using MS/MS, an FSLM may be ionized and subsequently fragmented in a mass spectrometer, thereby forming an ionized FSLM fragment (e.g., a positively charged ionized FSLM fragment or a negatively charged ionized FSLM fragment). Ionization and fragmentation of FSLM makes it possible to detect and identify the ionized FSLM by MS/MS by identifying m/z of FSLM fragments. Thus, the method of the disclosure transforms the FSLMs of the disclosure, e.g., TGs, ether phospholipids (e.g., PCO- or PEO-), PCs, DGs or CEs, to an ionized FSLM or an ionized FSLM fragment. Forming an ionized FSLM or an ionized FSLM fragment is required in order to identify, detect and/or quantify the presence of the FSLM of interest and necessarily changes the physical characteristics and properties of the FSLM of interest as a result of conducting the methods of the disclosure.

An exemplary method for detecting the presence or absence of a lipid, or determining an amount of a lipid, e.g., FSLM, in a biological sample involves obtaining a biological sample from a subject and subjecting the biological sample to liquid chromatography and mass spectrometry (LC/MS). It is understood that the methods provided herein for detecting a lipid or determining an amount of a lipid in a biological sample includes the steps to perform the assay.

In certain embodiments, all FSLMs are detected using the same method. In certain embodiments, all FSLMs are detected using the same biological sample (e.g., same body fluid or tissue). In certain embodiments, different FSLMs are detected using various methods. In certain embodiments, FSLMs are detected in different biological samples. In some embodiments, a biological sample is a solid tissue sample, preferably a buccal sample, alternatively a skin biopsy sample, skin strip, hair follicle, muscle biopsy sample, or a body fluid sample such as blood (including any blood product, such as whole blood, plasma, serum, or specific types of cells of the blood), urine, saliva, or seminal fluid. In one embodiment, the biological sample is a skin sample. In one embodiment, the biological sample is a buccal sample. In one embodiment, the biological sample is a blood-derived sample, e.g., a plasma sample or a platelet sample.

FSLM levels can be detected based on the absolute level or a normalized or relative level. Detection of absolute FSLM levels may be preferable when monitoring the treatment of a subject or in determining if there is a change in the FXN status of a subject. For example, the levels of one or more FSLMs can be monitored in a subject undergoing treatment with an FXN replacement therapy, e.g., at regular intervals, such as a monthly intervals. A modulation in the levels of one or more FSLMs can be monitored over time to observe trends in changes of the FSLM levels. Levels of the FSLMs of the disclosure in the subject may be higher than the levels of those FSLMs in a normal sample, but may be lower than the prior levels, thus indicating a lack of efficacy of the FXN replacement therapy in the subject. Changes, or not, in FSLM levels may be more relevant to treatment decisions for the subject than FSLM levels present in the population. Rapid changes in FSLM levels in a subject may be indicative of an abnormal FXN levels, even if the FSLMs are within normal ranges for the population.

As an alternative to making determinations based on the absolute level of the FSLM, determinations may be based on the normalized level of the FSLM. FSLM levels are normalized by correcting the absolute level of an FSLM by comparing its level to the level of a lipid that is not an FSLM, e.g., a lipid that is not sensitive to FXN levels. This normalization allows the comparison of the FSLM level in one sample, e.g., a sample from an FXN deficient subject, to another sample, e.g., a normal sample, or between samples from different sources.

Any one of the FXN lipid profiles described herein may be part of one or more algorithms which may be used to analyze the FXN lipid profile of a sample and determine whether the sample represents a sample of a normal subject, a sample from a subject prior to FXN replacement therapy or a sample from a subject after FXN replacement therapy. The one or more algorithms may be used to analyze a sample from a subject treated with an FXN-replacement therapy and determine whether the subject has reacted effectively to the treatment, and therefore expresses a profile characteristic of an FXN replacement lipid profile or not. Thus an algorithm for analyzing the lipid profile of a sample may use any one of a baseline FXN(−) lipid profile, an FXN replacement lipid profile, or a normal FXN lipid profile, or a combination of profiles. Where a sample having FXN signature lipid patterns consistent with baseline FXN(−) lipid profile represents lack of effectiveness of FXN replacement therapy; and the sample having FXN lipid profile consistent with FXN replacement lipid profile and/or normal FXN lipid profile represents effectiveness of FXN replacement therapy. In an embodiment, a classifier may be applied to FXN lipid profiles obtained from subject samples in order to obtain information about the samples, for example to characterize the status of the FXN lipid profile, or to define whether the subject was administered FXN replacement therapy or not. Alternatively or additionally, a classifier may be applied for evaluating whether the FXN lipid profile of the subject sample reached a certain threshold necessary for FXN replacement treatment to be considered effective.

### Methods of for Treating FXN Deficiency

Provided in the disclosure is also a method of treatment of a subject suffering from a mitochondrial disease having FXN deficiency, the method comprising determining an FXN lipid profile in a sample from the subject, and comparing the FXN lipid profile obtained from the sample with at least one of a normal FXN lipid profile, a baseline FXN(−) lipid profile, or an FXN replacement lipid profile. The sample may be further classified as having a normal FXN, a baseline FXN(−) or an FXN replacement profile. Using the results of the comparison of the sample FXN profile with the FXN profiles described herein, a therapy regime using FXN replacement therapy may be initiated, paused or ceased. Alternatively, an FXN replacement therapy dosage regime may be modified, e.g., increased or decreased. In one embodiment, the method further comprises obtaining or providing a sample from a subject, e.g., an FXN-deficient subject, such as a subject with FRDA.

In some embodiments, the present disclosure provides a method for treating an FXN deficiency, the method comprising: (a) determining an FXN lipid profile in a sample obtained from an FXN deficient subject for one or more FXN-sensitive lipid markers (FSLMs), (b) comparing the FXN lipid profile of the sample with at least one other lipid profile selected from the group consisting of normal FXN lipid profile for the one or more FSLMs, baseline FXN(−) lipid profile for the one or more FSLMs, and FXN replacement lipid profile for the one or more FSLMs, (c) classifying the FXN lipid profile determined in step (a) as corresponding to a normal FXN lipid profile, baseline FXN(−) lipid profile or an FXN replacement lipid profile, and (d) maintaining, initiating or modulating an FXN replacement therapy based on the classification of the FXN lipid profile of the sample.

In some embodiments, modulating an FXN replacement therapy comprises increasing the dosage, decreasing the dosage, increasing the administration frequency, decreasing the administration frequency, or any combination thereof, of the FXN replacement therapy.

In some embodiments, the one or more FSLMs comprise one or any combination of one or more TGs, ether phospholipids (e.g., PCO- or PEO-), PCs, DGs or CEs as described herein.

In some embodiments, when there is an increase in the amount of one or more of TGs, wherein the three acyl groups in each TG molecule contain less than 56 carbon atoms or contain 7 or less unsaturations (e.g., TG45:1, TG46:1, TG46:3, TG47:1, TG47:2, TG48:0, TG48:1, TG48:2, TG48:3, TG49:1, TG49:2, TG49:3, TG49:4, TG50:1, TG50:2, TG50:3, TG50:4, TG50:5, TG51:1, TG51:2, TG51:3, TG51:4, TG52:2, TG52:3, TG52:4, TG52:5, TG52:6, TG53:2, TG53:3, TG53:4, TG53:5, TG54:4, TG54:5, TG54:6 and TG54:7) in the sample obtained from the FXN deficient subject as compared to the amount in the normal FXN lipid profile, the FXN lipid profile determined in step (a) is classified as corresponding to a baseline FXN(−) lipid profile.

In some embodiments, when there is a decrease in the amount of one or more PCO-, (e.g., one or more of PC(O-16:0/14:0), PC(O-16:0/18:2), PC(O-16:0/20:3), PC(O-16:0/20:4), PC(O-16:0/22:6), PC(O-17:0/20:4), PC(O-18:0/18:1), PC(O-18:0/22:6), PC(O-18:0/18:2), PC(O-18:1/18:2), PC(O-18:1/20:4), PC(O-18:1/20:5), PC(O-18:1/22:6), PC(O-(20:0/22:6), PC(O-20:1/22.6), PC(O-20:2/20:4), PC(O-22:2/20:4), PC(O-22:1/22:6), PC(O-22:2/20:4), PC(O-(24:1/22:6), PC(O-24:2/20:4), PCO-34:2, PCO-36:3, PCO-34:2, PCO-38:3, PCO-40:2, PCO-40:6, PCO-44:7 and PCO-46.8) in the sample obtained from the FXN deficient subject as compared to the amount in the normal FXN lipid profile, the FXN lipid profile determined in step (a) is classified as corresponding to a baseline FXN(−) lipid profile.

In some embodiments, when there is an increase in the amount of one or more PCs selected from the group consisting of PC(15:0/20:3), PC(15:0/22:6), PC(16:0/14:0), PC(16:0/22:4), PC(16:1/16:0), PC(16:1/20:4), PC(16:1/22.5), PC(17:0/20:5), PC(18:0/20:3), PC(18:0/22:4), PC(18:1/20:3), PC(18:2/18:2), PC(20:4/15:0), PC40:6, PC42:7 in the sample obtained from the FXN deficient subject as compared to the amount in the normal FXN lipid profile, the FXN lipid profile determined in step (a) is classified as corresponding to a baseline FXN(−) lipid profile.

In some embodiments, when there is a decrease in the amount of one or more PCs selected from the group consisting of PC(17:1/20:4), PC(18:2/18:3), PC(18:1/24:1), PC(18:2/20:5) and PC(20:4/20:0) in the sample obtained from the FXN deficient subject as compared to the amount in the normal FXN lipid profile, the FXN lipid profile determined in step (a) is classified as corresponding to a baseline FXN(−) lipid profile.

In some embodiments, when there is an increase in the amount of DG(18:1/18:2) in the sample obtained from the FXN deficient subject as compared to the amount in the normal FXN lipid profile, the FXN lipid profile determined in step (a) is classified as corresponding to a baseline FXN(−) lipid profile.

In some embodiments, when there is an increase in the amount of CE14:1 in the sample obtained from the FXN deficient subject as compared to the amount in the normal FXN lipid profile, the FXN lipid profile determined in step (a) is classified as corresponding to a baseline FXN(−) lipid profile.

In some embodiments, when there is a decrease in the amount of CE16:0 in the sample obtained from the FXN deficient subject as compared to the amount in the normal FXN lipid profile, the FXN lipid profile determined in step (a) is classified as corresponding to a baseline FXN(−) lipid profile.

In some embodiments, when there is a decrease in the amount of CE20:5 in the sample obtained from the FXN deficient subject as compared to the amount in the normal FXN lipid profile, the FXN lipid profile determined in step (a) is classified as corresponding to a baseline FXN(−) lipid profile.

In some embodiments, when the FXN lipid profile in the sample is classified as a baseline FXN(−) lipid profile, administration of an FXN replacement therapy is initiated in the FXN deficient subject. In some embodiments, when the FXN lipid profile in the sample is classified as a baseline FXN(−) lipid profile, and the FXN deficient subject is already undergoing FXN replacement therapy, the FXN replacement therapy regiment is altered, e.g., the dose and/or administration frequency of the FXN replacement therapy is increased.

In some embodiments, when there is a lack of change in the amount of any one of TGs, ether phospholipids (e.g., PCO- or PEO-), PCs, DGs or CEs as described herein in the sample obtained from the FXN deficient subject as compared to the amount in the normal FXN lipid profile, the FXN lipid profile determined in step (a) is classified as corresponding to a normal FXN lipid profile.

In some embodiments, when the FXN lipid profile in the sample is classified as a normal FXN lipid profile, administration of an FXN replacement therapy is not initiated in the FXN deficient subject. In some embodiments, when the FXN lipid profile in the sample is classified as a normal FXN lipid profile, and the FXN deficient subject is already undergoing FXN replacement therapy, the FXN replacement therapy regimen is maintained (i.e., not changed), e.g., the dose and/or administration frequency of the FXN replacement therapy is maintained.

In some aspects, the present disclosure also provides a method of treating an FXN deficiency in a subject, the method comprising: (a) determining an FXN lipid profile for one or more FSLMs in a sample from an FXN deficient subject; and (b) recommending to a healthcare provider to administer an FXN replacement therapy to the subject based on the subject FXN lipid profile determined in step (a). In some aspects, the present disclosure also provides a method of treating an FXN deficiency in a subject, the method comprising: (a) obtaining an FXN lipid profile for one or more FSLMs in a sample obtained from an FXN deficient subject; and (b) administering an FXN replacement therapy to the subject based on the subject FXN lipid profile.

In some embodiments, the method further comprises comparing the FXN lipid profile for the one or more FSGMs in the sample with at least one other lipid profile selected from the group consisting of normal FXN lipid profile for the one or more FSGMs, baseline FXN(−) lipid profile for the one or more FSGMs, and FXN replacement lipid profile for the one or more FSGMs. In some embodiments, the method further comprises classifying the FXN lipid profile for the one or more FSGMs in the sample as corresponding to a normal FXN lipid profile for the one or more FSGMs, baseline FXN(−) lipid profile for the one or more FSGMs, or FXN replacement lipid profile for the one or more FSGMs. In some embodiments, the one or more FSLMs comprise one or any combination of one or more TGs, ether phospholipids (e.g., PCO- or PEO-), PCs, DGs or CEs as described herein.

In some embodiments, when there is an increase in the amount of one or more of TGs, wherein the three acyl groups in each TG molecule contain less than 56 carbon atoms or contain 7 or less unsaturations (e.g., TG45:1, TG46:1, TG46:3, TG47:1, TG47:2, TG48:0, TG48:1, TG48:2, TG48:3, TG49:1, TG49:2, TG49:3, TG49:4, TG50:1, TG50:2, TG50:3, TG50:4, TG50:5, TG51:1, TG51:2, TG51:3, TG51:4, TG52:2, TG52:3, TG52:4, TG52:5, TG52:6, TG53:2, TG53:3, TG53:4, TG53:5, TG54:4, TG54:5, TG54:6 and TG54:7) in the sample obtained from the FXN deficient subject as compared to the amount in the normal FXN lipid profile, then an FXN replacement therapy is administered to the FXN deficient subject, or a recommendation to a healthcare provider is made to administer an FXN replacement therapy to the FXN deficient subject. In some embodiments, when there is an increase in the amount of one or more of TGs, wherein the three acyl groups in each TG molecule contain less than 56 carbon atoms or contain 7 or less unsaturations (e.g., TG45:1, TG46:1, TG46:3, TG47:1, TG47:2, TG48:0, TG48:1, TG48:2, TG48:3, TG49:1, TG49:2, TG49:3, TG49:4, TG50:1, TG50:2, TG50:3, TG50:4, TG50:5, TG51:1, TG51:2, TG51:3, TG51:4, TG52:2, TG52:3, TG52:4, TG52:5, TG52:6, TG53:2, TG53:3, TG53:4, TG53:5, TG54:4, TG54:5, TG54:6 and TG54:7) in the sample obtained from the FXN deficient subject as compared to the amount in the normal FXN lipid profile, and the FXN deficient subject is undergoing an FXN replacement therapy, then the FXN replacement therapy regimen is altered (e.g., the dosage and/or administration frequency is increased), or a recommendation to a healthcare provider is made to alter the FXN replacement therapy regimen.

In some embodiments, when there is lack of an increase in the amount of one or more of TGs, wherein the three acyl groups in each TG molecule contain less than 56 carbon atoms or contain 7 or less unsaturations (e.g., TG45:1, TG46:1, TG46:3, TG47:1, TG47:2, TG48:0, TG48:1, TG48:2, TG48:3, TG49:1, TG49:2, TG49:3, TG49:4, TG50:1, TG50:2, TG50:3, TG50:4, TG50:5, TG51:1, TG51:2, TG51:3, TG51:4, TG52:2, TG52:3, TG52:4, TG52:5, TG52:6, TG53:2, TG53:3, TG53:4, TG53:5, TG54:4, TG54:5, TG54:6 and TG54:7) in the sample obtained from the FXN deficient subject as compared to the amount in the normal FXN lipid profile, then an FXN replacement therapy is not administered to the FXN deficient subject, or a recommendation to a healthcare provider is made to not administer an FXN replacement therapy to the subject. In some embodiments, when there is a lack of an increase in the amount of one or more of TGs, wherein the three acyl groups in each TG molecule contain less than 56 carbon atoms or contain 7 or less unsaturations (e.g., TG45:1, TG46:1, TG46:3, TG47:1, TG47:2, TG48:0, TG48:1, TG48:2, TG48:3, TG49:1, TG49:2, TG49:3, TG49:4, TG50:1, TG50:2, TG50:3, TG50:4, TG50:5, TG51:1, TG51:2, TG51:3, TG51:4, TG52:2, TG52:3, TG52:4, TG52:5, TG52:6, TG53:2, TG53:3, TG53:4, TG53:5, TG54:4, TG54:5, TG54:6 and TG54:7) in the sample obtained from the FXN deficient subject as compared to the amount in the normal FXN lipid profile, and wherein the FXN deficient subject is undergoing an FXN replacement therapy, the FXN replacement therapy regimen is maintained (i.e., not changed), or a recommendation to a healthcare provider is made to maintain the FXN replacement therapy regimen.

In some embodiments, when there is a decrease in the amount of one or more PCO-, (e.g., one or more of PC(O-16:0/14:0), PC(O-16:0/18:2), PC(O-16:0/20:3), PC(O-16:0/20:4), PC(O-16:0/22:6), PC(O-17:0/20:4), PC(O-18:0/18:1), PC(O-18:0/22:6), PC(O-18:0/18:2), PC(O-18:1/18:2), PC(O-18:1/20:4), PC(O-18:1/20:5), PC(O-18:1/22:6), PC(O-(20:0/22:6), PC(O-20:1/22.6), PC(O-20:2/20:4), PC(O-22:2/20:4), PC(O-22:1/22:6), PC(O-22:2/20:4), PC(O-(24:1/22:6), PC(O-24:2/20:4), PCO-34:2, PCO-36:3, PCO-34:2, PCO-38:3, PCO-40:2, PCO-40:6, PCO-44:7 and PCO-46.8) in the sample obtained from the FXN deficient subject as compared to the amount in the normal FXN lipid profile, then an FXN replacement therapy is administered to the FXN deficient subject, or a recommendation to a healthcare provider is made to administer an FXN replacement therapy to the FXN deficient subject. In some embodiments, when there is a decrease in the amount of one or more PCO-, (e.g., one or more of PC(O-16:0/14:0), PC(O-16:0/18:2), PC(O-16:0/20:3), PC(O-16:0/20:4), PC(O-16:0/22:6), PC(O-17:0/20:4), PC(O-18:0/18:1), PC(O-18:0/22:6), PC(O-18:0/18:2), PC(O-18:1/18:2), PC(O-18:1/20:4), PC(O-18:1/20:5), PC(O-18:1/22:6), PC(O-(20:0/22:6), PC(O-20:1/22.6), PC(O-20:2/20:4), PC(O-22:2/20:4), PC(O-22:1/22:6), PC(O-22:2/20:4), PC(O-(24:1/22:6), PC(O-24:2/20:4), PCO-34:2, PCO-36:3, PCO-34:2, PCO-38:3, PCO-40:2, PCO-40:6, PCO-44:7 and PCO-46.8) in the sample obtained from the FXN deficient subject as compared to the amount in the normal FXN lipid profile, and the FXN deficient subject is undergoing an FXN replacement therapy, then the FXN replacement therapy regimen is altered (e.g., the dosage and/or administration frequency is increased), or a recommendation to a healthcare provider is made to alter the FXN replacement therapy regimen.

In some embodiments, when there is lack of a decrease in the amount of one or more PCO-, (e.g., one or more of PC(O-16:0/14:0), PC(O-16:0/18:2), PC(O-16:0/20:3), PC(O-16:0/20:4), PC(O-16:0/22:6), PC(O-17:0/20:4), PC(O-18:0/18:1), PC(O-18:0/22:6), PC(O-18:0/18:2), PC(O-18:1/18:2), PC(O-18:1/20:4), PC(O-18:1/20:5), PC(O-18:1/22:6), PC(O-(20:0/22:6), PC(O-20:1/22.6), PC(O-20:2/20:4), PC(O-22:2/20:4), PC(O-22:1/22:6), PC(O-22:2/20:4), PC(O-(24:1/22:6), PC(O-24:2/20:4), PCO-34:2, PCO-36:3, PCO-34:2, PCO-38:3, PCO-40:2, PCO-40:6, PCO-44:7 and PCO-46.8) in the sample obtained from the FXN deficient subject as compared to the amount in the normal FXN lipid profile, then an FXN replacement therapy is not administered to the FXN deficient subject, or a recommendation to a healthcare provider is made to not administer an FXN replacement therapy to the subject. In some embodiments, when there is a lack of a decrease in the amount of one or more PCO-, (e.g., one or more of PC(O-16:0/14:0), PC(O-16:0/18:2), PC(O-16:0/20:3), PC(O-16:0/20:4), PC(O-16:0/22:6), PC(O-17:0/20:4), PC(O-18:0/18:1), PC(O-18:0/22:6), PC(O-18:0/18:2), PC(O-18:1/18:2), PC(O-18:1/20:4), PC(O-18:1/20:5), PC(O-18:1/22:6), PC(O-(20:0/22:6), PC(O-20:1/22.6), PC(O-20:2/20:4), PC(O-22:2/20:4), PC(O-22:1/22:6), PC(O-22:2/20:4), PC(O-(24:1/22:6), PC(O-24:2/20:4), PCO-34:2, PCO-36:3, PCO-34:2, PCO-38:3, PCO-40:2, PCO-40:6, PCO-44:7 and PCO-46.8) in the sample obtained from the FXN deficient subject as compared to the amount in the normal FXN lipid profile, and wherein the FXN deficient subject is undergoing an FXN replacement therapy, the FXN replacement therapy regimen is maintained (i.e., not changed), or a recommendation to a healthcare provider is made to maintain the FXN replacement therapy regimen.

In some embodiments, when there is an increase in the amount of one or more PCs selected from the group consisting of PC(15:0/20:3), PC(15:0/22:6), PC(16:0/14:0), PC(16:0/22:4), PC(16:1/16:0), PC(16:1/20:4), PC(16:1/22.5), PC(17:0/20:5), PC(18:0/20:3), PC(18:0/22:4), PC(18:1/20:3), PC(18:2/18:2), PC(20:4/15:0), PC40:6, PC42:7 in the sample obtained from the FXN deficient subject as compared to the amount in the normal FXN lipid profile, then an FXN replacement therapy is administered to the FXN deficient subject, or a recommendation to a healthcare provider is made to administer an FXN replacement therapy to the FXN deficient subject. In some embodiments, when there is an increase in the amount of one or more PCs selected from the group consisting of PC(15:0/20:3), PC(15:0/22:6), PC(16:0/14:0), PC(16:0/22:4), PC(16:1/16:0), PC(16:1/20:4), PC(16:1/22.5), PC(17:0/20:5), PC(18:0/20:3), PC(18:0/22:4), PC(18:1/20:3), PC(18:2/18:2), PC(20:4/15:0), PC40:6, PC42:7 in the sample obtained from the FXN deficient subject as compared to the amount in the normal FXN lipid profile, and the FXN deficient subject is undergoing an FXN replacement therapy, then the FXN replacement therapy regimen is altered (e.g., the dosage and/or administration frequency is increased), or a recommendation to a healthcare provider is made to alter the FXN replacement therapy regimen.

In some embodiments, when there is lack of an increase in the amount of one or more PCs selected from the group consisting of PC(15:0/20:3), PC(15:0/22:6), PC(16:0/14:0), PC(16:0/22:4), PC(16:1/16:0), PC(16:1/20:4), PC(16:1/22.5), PC(17:0/20:5), PC(18:0/20:3), PC(18:0/22:4), PC(18:1/20:3), PC(18:2/18:2), PC(20:4/15:0), PC40:6, PC42:7 in the sample obtained from the FXN deficient subject as compared to the amount in the normal FXN lipid profile, then an FXN replacement therapy is not administered to the FXN deficient subject, or a recommendation to a healthcare provider is made to not administer an FXN replacement therapy to the subject. In some embodiments, when there is a lack of an increase in the amount of one or more PCs selected from the group consisting of PC(15:0/20:3), PC(15:0/22:6), PC(16:0/14:0), PC(16:0/22:4), PC(16:1/16:0), PC(16:1/20:4), PC(16:1/22.5), PC(17:0/20:5), PC(18:0/20:3), PC(18:0/22:4), PC(18:1/20:3), PC(18:2/18:2), PC(20:4/15:0), PC40:6, PC42:7 in the sample obtained from the FXN deficient subject as compared to the amount in the normal FXN lipid profile, and wherein the FXN deficient subject is undergoing an FXN replacement therapy, the FXN replacement therapy regimen is maintained (i.e., not changed), or a recommendation to a healthcare provider is made to maintain the FXN replacement therapy regimen.

In some embodiments, when there is a decrease in the amount of one or more PCs selected from the group consisting of PC(17:1/20:4), PC(18:2/18:3), PC(18:1/24:1), PC(18:2/20:5) and PC(20:4/20:0) in the sample obtained from the FXN deficient subject as compared to the amount in the normal FXN lipid profile, then an FXN replacement therapy is administered to the FXN deficient subject, or a recommendation to a healthcare provider is made to administer an FXN replacement therapy to the FXN deficient subject. In some embodiments, when there is a decrease in the amount of one or more PCs selected from the group consisting of PC(17:1/20:4), PC(18:2/18:3), PC(18:1/24:1), PC(18:2/20:5) and PC(20:4/20:0) in the sample obtained from the FXN deficient subject as compared to the amount in the normal FXN lipid profile, and the FXN deficient subject is undergoing an FXN replacement therapy, then the FXN replacement therapy regimen is altered (e.g., the dosage and/or administration frequency is increased), or a recommendation to a healthcare provider is made to alter the FXN replacement therapy regimen.

In some embodiments, when there is lack of a decrease in the amount of one or more PCs selected from the group consisting of PC(17:1/20:4), PC(18:2/18:3), PC(18:1/24:1), PC(18:2/20:5) and PC(20:4/20:0) in the sample obtained from the FXN deficient subject as compared to the amount in the normal FXN lipid profile, then an FXN replacement therapy is not administered to the FXN deficient subject, or a recommendation to a healthcare provider is made to not administer an FXN replacement therapy to the subject. In some embodiments, when there is a lack of a decrease in the amount of one or more PCs selected from the group consisting of PC(17:1/20:4), PC(18:2/18:3), PC(18:1/24:1), PC(18:2/20:5) and PC(20:4/20:0) in the sample obtained from the FXN deficient subject as compared to the amount in the normal FXN lipid profile, and wherein the FXN deficient subject is undergoing an FXN replacement therapy, the FXN replacement therapy regimen is maintained (i.e., not changed), or a recommendation to a healthcare provider is made to maintain the FXN replacement therapy regimen.

In some embodiments, when there is an increase in the amount of DG(18:1/18:2) in the sample obtained from the FXN deficient subject as compared to the amount in the normal FXN lipid profile, then an FXN replacement therapy is administered to the FXN deficient subject, or a recommendation to a healthcare provider is made to administer an FXN replacement therapy to the FXN deficient subject. In some embodiments, when there is an increase in the amount of DG(18:1/18:2) in the sample obtained from the FXN deficient subject as compared to the amount in the normal FXN lipid profile, and the FXN deficient subject is undergoing an FXN replacement therapy, then the FXN replacement therapy regimen is altered (e.g., the dosage and/or administration frequency is increased), or a recommendation to a healthcare provider is made to alter the FXN replacement therapy regimen.

In some embodiments, when there is lack of an increase in the amount of DG(18:1/18:2) in the sample obtained from the FXN deficient subject as compared to the amount in the normal FXN lipid profile, then an FXN replacement therapy is not administered to the FXN deficient subject, or a recommendation to a healthcare provider is made to not administer an FXN replacement therapy to the subject. In some embodiments, when there is a lack of a decrease in the amount of an increase in the amount of DG(18:1/18:2) in the sample obtained from the FXN deficient subject as compared to the amount in the normal FXN lipid profile, and wherein the FXN deficient subject is undergoing an FXN replacement therapy, the FXN replacement therapy regimen is maintained (i.e., not changed), or a recommendation to a healthcare provider is made to maintain the FXN replacement therapy regimen.

In some embodiments, when there is an increase in the amount of CE14:1 in the sample obtained from the FXN deficient subject as compared to the amount in the normal FXN lipid profile, then an FXN replacement therapy is administered to the FXN deficient subject, or a recommendation to a healthcare provider is made to administer an FXN replacement therapy to the FXN deficient subject. In some embodiments, when there is an increase in the amount of CE14:1 in the sample obtained from the FXN deficient subject as compared to the amount in the normal FXN lipid profile, and the FXN deficient subject is undergoing an FXN replacement therapy, then the FXN replacement therapy regimen is altered (e.g., the dosage and/or administration frequency is increased), or a recommendation to a healthcare provider is made to alter the FXN replacement therapy regimen.

In some embodiments, when there is lack of an increase in the amount of CE14:1 in the sample obtained from the FXN deficient subject as compared to the amount in the normal FXN lipid profile, then an FXN replacement therapy is not administered to the FXN deficient subject, or a recommendation to a healthcare provider is made to not administer an FXN replacement therapy to the subject. In some embodiments, when there is a lack of a decrease in the amount of an increase in the amount of CE14:1 in the sample obtained from the FXN deficient subject as compared to the amount in the normal FXN lipid profile, and wherein the FXN deficient subject is undergoing an FXN replacement therapy, the FXN replacement therapy regimen is maintained (i.e., not changed), or a recommendation to a healthcare provider is made to maintain the FXN replacement therapy regimen.

In some embodiments, when there is a decrease in the amount of CE16:0 in the sample obtained from the FXN deficient subject as compared to the amount in the normal FXN lipid profile, then an FXN replacement therapy is administered to the FXN deficient subject, or a recommendation to a healthcare provider is made to administer an FXN replacement therapy to the FXN deficient subject. In some embodiments, when there is a decrease in the amount of CE16:0 in the sample obtained from the FXN deficient subject as compared to the amount in the normal FXN lipid profile, and the FXN deficient subject is undergoing an FXN replacement therapy, then the FXN replacement therapy regimen is altered (e.g., the dosage and/or administration frequency is increased), or a recommendation to a healthcare provider is made to alter the FXN replacement therapy regimen.

In some embodiments, when there is lack of a decrease in the amount of CE16:0 in the sample obtained from the FXN deficient subject as compared to the amount in the normal FXN lipid profile, then an FXN replacement therapy is not administered to the FXN deficient subject, or a recommendation to a healthcare provider is made to not administer an FXN replacement therapy to the subject. In some embodiments, when there is a lack of a decrease in the amount of a decrease in the amount of CE16:0 in the sample obtained from the FXN deficient subject as compared to the amount in the normal FXN lipid profile, and wherein the FXN deficient subject is undergoing an FXN replacement therapy, the FXN replacement therapy regimen is maintained (i.e., not changed), or a recommendation to a healthcare provider is made to maintain the FXN replacement therapy regimen.

In some embodiments, when there is a decrease in the amount of CE20:5 in the sample obtained from the FXN deficient subject as compared to the amount in the normal FXN lipid profile, then an FXN replacement therapy is administered to the FXN deficient subject, or a recommendation to a healthcare provider is made to administer an FXN replacement therapy to the FXN deficient subject. In some embodiments, when there is a decrease in the amount of CE20:5 in the sample obtained from the FXN deficient subject as compared to the amount in the normal FXN lipid profile, and the FXN deficient subject is undergoing an FXN replacement therapy, then the FXN replacement therapy regimen is altered (e.g., the dosage and/or administration frequency is increased), or a recommendation to a healthcare provider is made to alter the FXN replacement therapy regimen.

In some embodiments, when there is lack of a decrease in the amount of CE20:5 in the sample obtained from the FXN deficient subject as compared to the amount in the normal FXN lipid profile, then an FXN replacement therapy is not administered to the FXN deficient subject, or a recommendation to a healthcare provider is made to not administer an FXN replacement therapy to the subject. In some embodiments, when there is a lack of a decrease in the amount of a decrease in the amount of CE20:5 in the sample obtained from the FXN deficient subject as compared to the amount in the normal FXN lipid profile, and wherein the FXN deficient subject is undergoing an FXN replacement therapy, the FXN replacement therapy regimen is maintained (i.e., not changed), or a recommendation to a healthcare provider is made to maintain the FXN replacement therapy regimen.

In other embodiments, the present disclosure also involves the analysis and consideration of any clinical and/or subject-related health data, for example, data obtained from an Electronic Medical Record (e.g., collection of electronic health information about individual subjects or populations relating to various types of data, such as, demographics, medical history, medication and allergies, immunization status, laboratory test results, radiology images, vital signs, personal statistics like age and weight, and billing information).

In certain embodiments the methods provided herein further comprise obtaining a biological sample from a subject suspected of having a mitochondrial disease, e.g., FRDA.

In certain embodiments the methods provided herein further comprise selecting a treatment regimen for the subject based on the level of the one or more FSLMs, such as triglycerides, cholesteryl esters, e.g., CE16:0, CE20:5, or ether phospholipids, e.g., PCO- or PEO-, including phosphatidylcholine ethers, such as PC(O-17:0/20:4). In certain embodiments, the treatment method is started, changed, revised, or maintained based on the results from the methods of the disclosure, e.g., when it is determined that the subject is responding to the treatment regimen, or when it is determined that the subject is not responding to the treatment regimen, or when it is determined that the subject is insufficiently responding to the treatment regimen. In certain embodiments, the treatment method is changed based on the results from the methods.

In certain embodiments of the diagnostic and monitoring methods provided herein, the method further comprises isolating a component of the biological sample.

In certain embodiments of the diagnostic and monitoring methods provided herein, the method further comprises concentrating a component of the biological sample.

### Methods for Monitoring FXN Deficiency

In some aspects, the present disclosure provides methods for evaluating FXN deficiency or monitoring or evaluating progression of FXN deficiency in a subject over time. In these methods the amount of one or more FSLMs described herein, e.g., one or more of TGs, PCO- or PCs described herein, in one or more samples obtained from a subject known or suspected of having an FXN deficiency is assessed. The samples may comprise one sample obtained from the subject that may be used for comparison to a reference sample, e.g., a sample obtained from a healthy subject. The samples may also comprise a first sample obtained from the subject at an earlier time point and a second sample obtained from the subject at a later time point. It is understood that the methods of the disclosure include obtaining and analyzing more than two samples (e.g., 3, 4, 5, 6, 7, 8, 9, or more samples) at regular or irregular intervals for assessment of FSLM levels. Pairwise comparisons can be made between consecutive or non-consecutive subject samples. Trends of FSLM levels and rates of change of FSLM levels can be analyzed for any two or more consecutive or non-consecutive subject samples.

Accordingly, the present disclosure provides a method for evaluating an FXN deficiency in a subject, comprising (a) determining an FXN lipid profile for one or more FSLMs in a sample obtained from the subject, e.g., an FXN deficient subject or a subject suspected of having an FXN deficiency; and (b) comparing the FXN replacement lipid profile determined in step (a) to a reference FXN lipid profile for the one or more FSGMs; and making a determination about the FXN deficiency of the subject based on the comparison in step (b). In some embodiments, the reference FXN lipid profile is a normal FXN lipid profile. In some embodiments, a determination as to the severity of the FXN deficiency is made. In some embodiments, a determination as to the necessity to administer an FXN replacement therapy is made.

In some embodiments, the one or more FSLMs comprise one or any combination of one or more TGs, ether phospholipids (e.g., PCO- or PEO-), PCs, DGs or CEs as described herein. For example, the one or more FSLMs may comprise one or more TGs, wherein the three acyl groups in each TG molecule contain less than 56 carbon atoms, e.g., one or more of TG45:1, TG46:1, TG46:3, TG47:1, TG47:2, TG48:0, TG48:1, TG48:2, TG48:3, TG49:1, TG49:2, TG49:3, TG49:4, TG50:1, TG50:2, TG50:3, TG50:4, TG50:5, TG51:1, TG51:2, TG51:3, TG51:4, TG52:2, TG52:3, TG52:4, TG52:5, TG52:6, TG53:2, TG53:3, TG53:4, TG53:5, TG54:4, TG54:5, TG54:6 and TG54:7. In some embodiments, an increase in the amount of one or more TGs, wherein the three acyl groups in each TG molecule contain less than 56 carbon atoms (e.g., one or more of TG45:1, TG46:1, TG46:3, TG47:1, TG47:2, TG48:0, TG48:1, TG48:2, TG48:3, TG49:1, TG49:2, TG49:3, TG49:4, TG50:1, TG50:2, TG50:3, TG50:4, TG50:5, TG51:1, TG51:2, TG51:3, TG51:4, TG52:2, TG52:3, TG52:4, TG52:5, TG52:6, TG53:2, TG53:3, TG53:4, TG53:5, TG54:4, TG54:5, TG54:6 and TG54:7) in the subject FXN lipid profile as compared to the normal FXN lipid profile is indicative that the subject has FXN deficiency.

In some embodiments, the one or more FSLMs may comprise one or more TGs, wherein the three acyl groups in each TG molecule contain 7 or less unsaturations, e.g., one or more of TG45:1, TG46:1, TG46:3, TG47:1, TG47:2, TG48:0, TG48:1, TG48:2, TG48:3, TG49:1, TG49:2, TG49:3, TG49:4, TG50:1, TG50:2, TG50:3, TG50:4, TG50:5, TG51:1, TG51:2, TG51:3, TG51:4, TG52:2, TG52:3, TG52:4, TG52:5, TG52:6, TG53:2, TG53:3, TG53:4, TG53:5, TG54:4, TG54:5, TG54:6 and TG54:7. In some embodiments, an increase in the amount of one or more TGs, wherein the three acyl groups in each TG molecule contain 7 or less unsaturations, (e.g., one or more of TG45:1, TG46:1, TG46:3, TG47:1, TG47:2, TG48:0, TG48:1, TG48:2, TG48:3, TG49:1, TG49:2, TG49:3, TG49:4, TG50:1, TG50:2, TG50:3, TG50:4, TG50:5, TG51:1, TG51:2, TG51:3, TG51:4, TG52:2, TG52:3, TG52:4, TG52:5, TG52:6, TG53:2, TG53:3, TG53:4, TG53:5, TG54:4, TG54:5, TG54:6 and TG54:7) in the subject FXN lipid profile as compared to the normal FXN lipid profile is indicative that the subject has FXN deficiency.

In some embodiments, the one or more FSLMs comprise one or more PCO-, e.g., one or more of PC(O-16:0/14:0), PC(O-16:0/18:2), PC(O-16:0/20:3), PC(O-16:0/20:4), PC(O-16:0/22:6), PC(O-17:0/20:4), PC(O-18:0/18:1), PC(O-18:0/22:6), PC(O-18:0/18:2), PC(O-18:1/18:2), PC(O-18:1/20:4), PC(O-18:1/20:5), PC(O-18:1/22:6), PC(O-(20:0/22:6), PC(O-20:1/22.6), PC(O-20:2/20:4), PC(O-22:2/20:4), PC(O-22:1/22:6), PC(O-22:2/20:4), PC(O-(24:1/22:6), PC(O-24:2/20:4), PCO-34:2, PCO-36:3, PCO-34:2, PCO-38:3, PCO-40:2, PCO-40:6, PCO-44:7 and PCO-46.8. In some embodiments, a decrease in the amount of one or more PCO- (e.g., one or more of PC(O-16:0/14:0), PC(O-16:0/18:2), PC(O-16:0/20:3), PC(O-16:0/20:4), PC(O-16:0/22:6), PC(O-17:0/20:4), PC(O-18:0/18:1), PC(O-18:0/22:6), PC(O-18:0/18:2), PC(O-18:1/18:2), PC(O-18:1/20:4), PC(O-18:1/20:5), PC(O-18:1/22:6), PC(O-(20:0/22:6), PC(O-20:1/22.6), PC(O-20:2/20:4), PC(O-22:2/20:4), PC(O-22:1/22:6), PC(O-22:2/20:4), PC(O-(24:1/22:6), PC(O-24:2/20:4), PCO-34:2, PCO-36:3, PCO-34:2, PCO-38:3, PCO-40:2, PCO-40:6, PCO-44:7 and PCO-46.8) in the subject FXN lipid profile as compared to the normal FXN lipid profile is indicative that the subject has FXN deficiency.

In some embodiments, the one or more FSLMs comprise one or more PCs, e.g., one or more of PC(15:0/20:3), PC(15:0/22:6), PC(16:0/14:0), PC(16:0/22:4), PC(16:1/16:0), PC(16:1/20:4), PC(16:1/22.5), PC(17:0/20:5), PC(18:0/20:3), PC(18:0/22:4), PC(18:1/20:3), PC(18:2/18:2), PC(20:4/15:0), PC40:6, PC42:7, PC(17:1/20:4), PC(18:2/18:3), PC(18:1/24:1), PC(18:2/20:5) and PC(20:4/20:0), PC(17:1/20:4), PC(18:2/18:3), PC(18:1/24:1), PC(18:2/20:5) and PC(20:4/20:0). In some embodiments, an increase in the amount of one or more PCs selected from the group consisting of PC(15:0/20:3), PC(15:0/22:6), PC(16:0/14:0), PC(16:0/22:4), PC(16:1/16:0), PC(16:1/20:4), PC(16:1/22.5), PC(17:0/20:5), PC(18:0/20:3), PC(18:0/22:4), PC(18:1/20:3), PC(18:2/18:2), PC(20:4/15:0), PC40:6, PC42:7 in the subject FXN lipid profile as compared to the normal FXN lipid profile is indicative that the subject has FXN deficiency. In some embodiments, a decrease in the amount of one or more PCs selected from the group consisting of PC(17:1/20:4), PC(18:2/18:3), PC(18:1/24:1), PC(18:2/20:5) and PC(20:4/20:0) in the subject FXN lipid profile as compared to the normal FXN lipid profile is indicative that the subject has FXN deficiency.

In some embodiments, the one or more FSLMs comprise one or more diglycerides, e.g., DG(18:1/18:2). In some embodiments, an increase in the amount of one or more DG, e.g., DG(18:1/18:2), in the subject FXN lipid profile as compared to the normal FXN lipid profile is indicative that the subject has FXN deficiency.

In some embodiments, an increase in the amount of CE14:1 in the subject FXN lipid profile as compared to the normal FXN lipid profile is indicative that the subject has FXN deficiency.

In some embodiments, a decrease in the amount of CE16:0 and/or CE20:5 in the subject FXN lipid profile as compared to the normal FXN lipid profile is indicative that the subject has FXN deficiency.

The present disclosure also provides a method for monitoring progression of an FXN deficiency in a subject, the method comprising: (a) determining a first FXN lipid profile for one or more FSLMs in a first sample obtained from the subject, e.g., an FXN deficient subject or a subject suspected of having an FXN deficiency, at a first time point; (b) determining a second FXN lipid profile for the one or more FSLMs in a second sample obtained from the subject at a second time point that is later than the first time point; and (c) comparing the second FXN lipid profile with the first FXN lipid profile; thereby monitoring FXN deficiency in the subject.

In some embodiments, the one or more FSLMs comprise one or any combination of one or more TGs, ether phospholipids (e.g., PCO- or PEO-), PCs, DGs or CEs as described herein. For example, the one or more FSLMs may comprise one or more TGs, wherein the three acyl groups in each TG molecule contain less than 56 carbon atoms, e.g., one or more of TG45:1, TG46:1, TG46:3, TG47:1, TG47:2, TG48:0, TG48:1, TG48:2, TG48:3, TG49:1, TG49:2, TG49:3, TG49:4, TG50:1, TG50:2, TG50:3, TG50:4, TG50:5, TG51:1, TG51:2, TG51:3, TG51:4, TG52:2, TG52:3, TG52:4, TG52:5, TG52:6, TG53:2, TG53:3, TG53:4, TG53:5, TG54:4, TG54:5, TG54:6 and TG54:7.

In some embodiments, an increase in the amount of one or more TGs, wherein the three acyl groups in each TG molecule contain less than 56 carbon atoms (e.g., one or more of TG45:1, TG46:1, TG46:3, TG47:1, TG47:2, TG48:0, TG48:1, TG48:2, TG48:3, TG49:1, TG49:2, TG49:3, TG49:4, TG50:1, TG50:2, TG50:3, TG50:4, TG50:5, TG51:1, TG51:2, TG51:3, TG51:4, TG52:2, TG52:3, TG52:4, TG52:5, TG52:6, TG53:2, TG53:3, TG53:4, TG53:5, TG54:4, TG54:5, TG54:6 and TG54:7) in the second FXN lipid profile as compared to the first FXN lipid profile is indicative that the FXN deficiency has progressed. In some embodiments, a lack of an increase in the amount of one or more TGs, wherein the three acyl groups in each TG molecule contain less than 56 carbon atoms (e.g., one or more of TG45:1, TG46:1, TG46:3, TG47:1, TG47:2, TG48:0, TG48:1, TG48:2, TG48:3, TG49:1, TG49:2, TG49:3, TG49:4, TG50:1, TG50:2, TG50:3, TG50:4, TG50:5, TG51:1, TG51:2, TG51:3, TG51:4, TG52:2, TG52:3, TG52:4, TG52:5, TG52:6, TG53:2, TG53:3, TG53:4, TG53:5, TG54:4, TG54:5, TG54:6 and TG54:7) in the second FXN lipid profile as compared to the first FXN lipid profile is indicative that the FXN deficiency has not progressed.

In some embodiments, the one or more FSLMs comprise one or any combination of one or more TGs, wherein the three acyl groups in each TG molecule contain 7 or less unsaturations, e.g., one or more of TG45:1, TG46:1, TG46:3, TG47:1, TG47:2, TG48:0, TG48:1, TG48:2, TG48:3, TG49:1, TG49:2, TG49:3, TG49:4, TG50:1, TG50:2, TG50:3, TG50:4, TG50:5, TG51:1, TG51:2, TG51:3, TG51:4, TG52:2, TG52:3, TG52:4, TG52:5, TG52:6, TG53:2, TG53:3, TG53:4, TG53:5, TG54:4, TG54:5, TG54:6 and TG54:7. In some embodiments, an increase in the amount of one or more TGs, wherein the three acyl groups in each TG molecule contain 7 or less unsaturations, (e.g., one or more of TG45:1, TG46:1, TG46:3, TG47:1, TG47:2, TG48:0, TG48:1, TG48:2, TG48:3, TG49:1, TG49:2, TG49:3, TG49:4, TG50:1, TG50:2, TG50:3, TG50:4, TG50:5, TG51:1, TG51:2, TG51:3, TG51:4, TG52:2, TG52:3, TG52:4, TG52:5, TG52:6, TG53:2, TG53:3, TG53:4, TG53:5, TG54:4, TG54:5, TG54:6 and TG54:7) in the second FXN lipid profile as compared to the first FXN lipid profile is indicative that the FXN deficiency has progressed. In some embodiments, a lack of an increase in the amount of one or more TGs, wherein the three acyl groups in each TG molecule contain 7 or less unsaturations, (e.g., one or more of TG45:1, TG46:1, TG46:3, TG47:1, TG47:2, TG48:0, TG48:1, TG48:2, TG48:3, TG49:1, TG49:2, TG49:3, TG49:4, TG50:1, TG50:2, TG50:3, TG50:4, TG50:5, TG51:1, TG51:2, TG51:3, TG51:4, TG52:2, TG52:3, TG52:4, TG52:5, TG52:6, TG53:2, TG53:3, TG53:4, TG53:5, TG54:4, TG54:5, TG54:6 and TG54:7) in the second FXN lipid profile as compared to the first FXN lipid profile is indicative that the FXN deficiency has not progressed.

In some embodiments, the one or more FSLMs comprise one or more PCO-, e.g., one or more of PC(O-16:0/14:0), PC(O-16:0/18:2), PC(O-16:0/20:3), PC(O-16:0/20:4), PC(O-16:0/22:6), PC(O-17:0/20:4), PC(O-18:0/18:1), PC(O-18:0/22:6), PC(O-18:0/18:2), PC(O-18:1/18:2), PC(O-18:1/20:4), PC(O-18:1/20:5), PC(O-18:1/22:6), PC(O-(20:0/22:6), PC(O-20:1/22.6), PC(O-20:2/20:4), PC(O-22:2/20:4), PC(O-22:1/22:6), PC(O-22:2/20:4), PC(O-(24:1/22:6), PC(O-24:2/20:4), PCO-34:2, PCO-36:3, PCO-34:2, PCO-38:3, PCO-40:2, PCO-40:6, PCO-44:7 and PCO-46.8. In some embodiments, a decrease in the amount of one or more PCO- (e.g., one or more of PC(O-16:0/14:0), PC(O-16:0/18:2), PC(O-16:0/20:3), PC(O-16:0/20:4), PC(O-16:0/22:6), PC(O-17:0/20:4), PC(O-18:0/18:1), PC(O-18:0/22:6), PC(O-18:0/18:2), PC(O-18:1/18:2), PC(O-18:1/20:4), PC(O-18:1/20:5), PC(O-18:1/22:6), PC(O-(20:0/22:6), PC(O-20:1/22.6), PC(O-20:2/20:4), PC(O-22:2/20:4), PC(O-22:1/22:6), PC(O-22:2/20:4), PC(O-(24:1/22:6), PC(O-24:2/20:4), PCO-34:2, PCO-36:3, PCO-34:2, PCO-38:3, PCO-40:2, PCO-40:6, PCO-44:7 and PCO-46.8) in the second FXN lipid profile as compared to the first FXN lipid profile is indicative that the FXN deficiency has progressed. In some embodiments, lack of a decrease in the amount of one or more PCO- (e.g., one or more of PC(O-16:0/14:0), PC(O-16:0/18:2), PC(O-16:0/20:3), PC(O-16:0/20:4), PC(O-16:0/22:6), PC(O-17:0/20:4), PC(O-18:0/18:1), PC(O-18:0/22:6), PC(O-18:0/18:2), PC(O-18:1/18:2), PC(O-18:1/20:4), PC(O-18:1/20:5), PC(O-18:1/22:6), PC(O-(20:0/22:6), PC(O-20:1/22.6), PC(O-20:2/20:4), PC(O-22:2/20:4), PC(O-22:1/22:6), PC(O-22:2/20:4), PC(O-(24:1/22:6), PC(O-24:2/20:4), PCO-34:2, PCO-36:3, PCO-34:2, PCO-38:3, PCO-40:2, PCO-40:6, PCO-44:7 and PCO-46.8) in the second FXN lipid profile as compared to the first FXN lipid profile is indicative that the FXN deficiency has not progressed.

In some embodiments, the one or more FSLMs comprise one or more PCs, e.g., one or more of PC(15:0/20:3), PC(15:0/22:6), PC(16:0/14:0), PC(16:0/22:4), PC(16:1/16:0), PC(16:1/20:4), PC(16:1/22.5), PC(17:0/20:5), PC(18:0/20:3), PC(18:0/22:4), PC(18:1/20:3), PC(18:2/18:2), PC(20:4/15:0), PC40:6, PC42:7, PC(17:1/20:4), PC(18:2/18:3), PC(18:1/24:1), PC(18:2/20:5) and PC(20:4/20:0), PC(17:1/20:4), PC(18:2/18:3), PC(18:1/24:1), PC(18:2/20:5) and PC(20:4/20:0).

In some embodiments, an increase in the amount of one or more PCs selected from the group consisting of PC(15:0/20:3), PC(15:0/22:6), PC(16:0/14:0), PC(16:0/22:4), PC(16:1/16:0), PC(16:1/20:4), PC(16:1/22.5), PC(17:0/20:5), PC(18:0/20:3), PC(18:0/22:4), PC(18:1/20:3), PC(18:2/18:2), PC(20:4/15:0), PC40:6, PC42:7 in the second FXN lipid profile as compared to the first FXN lipid profile is indicative that the FXN deficiency has progressed. In some embodiments, lack of an increase in the amount of one or more PCs selected from the group consisting of PC(15:0/20:3), PC(15:0/22:6), PC(16:0/14:0), PC(16:0/22:4), PC(16:1/16:0), PC(16:1/20:4), PC(16:1/22.5), PC(17:0/20:5), PC(18:0/20:3), PC(18:0/22:4), PC(18:1/20:3), PC(18:2/18:2), PC(20:4/15:0), PC40:6, PC42:7 in the second FXN lipid profile as compared to the first FXN lipid profile is indicative that the FXN deficiency has not progressed.

In some embodiments, a decrease in the amount of one or more PCs selected from the group consisting of PC(17:1/20:4), PC(18:2/18:3), PC(18:1/24:1), PC(18:2/20:5) and PC(20:4/20:0) in the second FXN lipid profile as compared to the first FXN lipid profile is indicative that the FXN deficiency has progressed. In some embodiments, lack of a decrease in the amount of one or more PCs selected from the group consisting of PC(17:1/20:4), PC(18:2/18:3), PC(18:1/24:1), PC(18:2/20:5) and PC(20:4/20:0) in the second FXN lipid profile as compared to the first FXN lipid profile is indicative that the FXN deficiency has not progressed.

In some embodiments, the one or more FSLMs comprise one or more diglycerides, e.g., DG(18:1/18:2). In some embodiments, an increase in the amount of one or more DG, e.g., DG(18:1/18:2), in the second FXN lipid profile as compared to the first FXN lipid profile is indicative that the FXN deficiency has progressed. In some embodiments, lack of an increase in the amount of one or more DG, e.g., DG(18:1/18:2), in the second FXN lipid profile as compared to the first FXN lipid profile is indicative that the FXN deficiency has not progressed.

In some embodiments, an increase in the amount of CE14:1 in the second FXN lipid profile as compared to the first FXN lipid profile is indicative that the FXN deficiency has progressed. In some embodiments, lack of an increase in the amount of CE14:1 in the second FXN lipid profile as compared to the first FXN lipid profile is indicative that the FXN deficiency has not progressed.

In some embodiments, a decrease in the amount of CE16:0 and/or CE20:5 in the second FXN lipid profile as compared to the first FXN lipid profile is indicative that the FXN deficiency has progressed. In some embodiments, lack of a decrease in the amount of CE16:0 and/or CE20:5 in the second FXN lipid profile as compared to the first FXN lipid profile is indicative that the FXN deficiency has not progressed.

### Methods for Detecting FSLMs

In some aspects, the present disclosure also provides a method for detecting one or more frataxin-sensitive lipid markers (FSLMs) in a sample from a frataxin (FXN) deficient subject, the method comprising contacting the sample, or a portion thereof, with one or more reagents specific for detecting the level of each of the one or more FSLMs.

In some aspects, the present disclosure also provides a method for detecting one or more frataxin-sensitive lipid markers (FSLMs) in a sample from a frataxin (FXN) deficient subject, the method comprising subjecting the sample, or a portion thereof, to liquid chromatography and mass spectrometry (LC/MS).

In some embodiments, the FXN deficient subject has Friedreich's Ataxia. In some embodiments, the sample is obtained from the FXN deficient subject before the subject is administered FXN replacement therapy. In some embodiments, the sample is obtained from the FXN deficient subject after the subject is administered FXN replacement therapy. In some embodiments, the sample is obtained from the FXN deficient subject at different time points, e.g., 1, 2, 3, 4 or more time points, while the subject is being administered FXN replacement therapy.

In some embodiments, the method further comprises obtaining a sample from the FXN deficient subject. In some embodiments, the sample is selected from the group consisting of a buccal sample, a skin sample, a hair follicle or a blood-derived sample. In some embodiments, the sample is a blood-derived sample, e.g., a plasma sample.

In some embodiments, the one or more FSLMs comprise one or any combination of one or more TGs, ether phospholipids (e.g., PCO- or PEO-), PCs, DGs or CEs as described herein. For example, in some embodiments, the one or more FSLMs comprise one or more of TGs, wherein the three acyl groups in each TG molecule contain less than 56 carbon atoms or 7 or less unsaturations (e.g., TG45:1, TG46:1, TG46:3, TG47:1, TG47:2, TG48:0, TG48:1, TG48:2, TG48:3, TG49:1, TG49:2, TG49:3, TG49:4, TG50:1, TG50:2, TG50:3, TG50:4, TG50:5, TG51:1, TG51:2, TG51:3, TG51:4, TG52:2, TG52:3, TG52:4, TG52:5, TG52:6, TG53:2, TG53:3, TG53:4, TG53:5, TG54:4, TG54:5, TG54:6 and TG54:7). In some embodiments, the one or more FSLMs comprise TG48:1, TG48:2, TG49:1, TG49:2, TG49:4, TG50:1, TG50:3, TG51:1, TG51:2, TG51:3, TG52:3, TG53:2 and TG56:8.

In some embodiments, the one or more FSLMs comprise one or more PCO-, e.g., one or more of PC(O-16:0/14:0), PC(O-16:0/18:2), PC(O-16:0/20:3), PC(O-16:0/20:4), PC(O-16:0/22:6), PC(O-17:0/20:4), PC(O-18:0/18:1), PC(O-18:0/22:6), PC(O-18:0/18:2), PC(O-18:1/18:2), PC(O-18:1/20:4), PC(O-18:1/20:5), PC(O-18:1/22:6), PC(O-(20:0/22:6), PC(O-20:1/22.6), PC(O-20:2/20:4), PC(O-22:2/20:4), PC(O-22:1/22:6), PC(O-22:2/20:4), PC(O-(24:1/22:6), PC(O-24:2/20:4), PCO-34:2, PCO-36:3, PCO-34:2, PCO-38:3, PCO-40:2, PCO-40:6, PCO-44:7 and PCO-46.8. In some embodiments, the one or more FSLMs comprise PC(O-16:0/18:2), PC(O-16:0/20:3), PC(O-18:1/18:2). PC(O-22:2/20:4) and PC(O-24:2/20:4).

In some embodiments, the one or more FSLMs comprise one or more PCs selected from the group consisting of PC(15:0/20:3), PC(15:0/22:6), PC(16:0/14:0), PC(16:0/22:4), PC(16:1/16:0), PC(16:1/20:4), PC(16:1/22.5), PC(17:0/20:5), PC(18:0/20:3), PC(18:0/22:4), PC(18:1/20:3), PC(18:2/18:2), PC(20:4/15:0), PC40:6, PC42:7, PC(17:1/20:4), PC(18:2/18:3), PC(18:1/24:1), PC(18:2/20:5) and PC(20:4/20:0). In some embodiments, the one or more FSLMs comprise PC(15:0/22:6) and PC(16:0/14:0). In some embodiments, the one or more FSLMs comprise PC(17:1/20:4), PC(18:2/18:3), PC(18:1/24:1), PC(18:2/20:5) and PC(20:4/20:0). In some embodiments, the one or more FSLMs comprise PC(18:2/18:3).

In some embodiments, the one or more FSLMs comprise one or more of DGs (e.g., DG18:1/18:2) and/or one or more of CEs (e.g., CE14:1, CE 16:0 and CE20:5).

### Kits/Panels

The invention also provides compositions and kits for evaluating and monitoring effectiveness of FXN replacement therapy. These kits may include a reagent useful to facilitate detection and/or quantification by mass spectrometry of one or more FSLM of the disclosure, such as one or more triglycerides (TGs), wherein the three acyl groups in each triglyceride molecule contain less than 56 carbons and/or wherein the three acyl groups in each triglyceride molecule contain 7 or less unsaturations; ether phospholipids (e.g., PCO- and PEO-), phosphatidylcholines (PCs), cholesteryl esters (CEs); and diglycerides (DGs). In some embodiments, the kits include a reagent useful to facilitate the detection and/or quantification by mass spectrometry of one or more FSLMs selected from the group consisting of TG45:1, TG46:1, TG46:3, TG47:1, TG47:2, TG48:0, TG48:1, TG48:2, TG48:3, TG49:1, TG49:2, TG49:3, TG49:4, TG50:1, TG50:2, TG50:3, TG50:4, TG50:5, TG51:1, TG51:2, TG51:3, TG51:4, TG52:2, TG52:3, TG52:4, TG52:5, TG52:6, TG53:2, TG53:3, TG53:4, TG53:5, TG54:4, TG54:5, TG54:6, TG54:7, PC(O-16:0/14:0), PC(O-16:0/18:2), PC(O-16:0/20:3), PC(O-16:0/20:4), PC(O-16:0/22:6), PC(O-17:0/20:4), PC(O-18:0/18:1), PC(O-18:0/22:6), PC(O-18:0/18:2), PC(O-18:1/18:2), PC(O-18:1/20:4), PC(O-18:1/20:5), PC(O-18:1/22:6), PC(O-(20:0/22:6), PC(O-20:1/22.6), PC(O-20:2/20:4), PC(O-22:2/20:4), PC(O-22:1/22:6), PC(O-22:2/20:4), PC(O-(24:1/22:6), PC(O-24:2/20:4), PCO-34:2, PCO-36:3, PCO-34:2, PCO-38:3, PCO-40:2, PCO-40:6, PCO-44:7, PCO-46.8, PC(15:0/20:3), PC(15:0/22:6), PC(16:0/14:0), PC(16:0/22:4), PC(16:1/16:0), PC(16:1/20:4), PC(16:1/22.5), PC(17:0/20:5), PC(18:0/20:3), PC(18:0/22:4), PC(18:1/20:3), PC(18:2/18:2), PC(20:4/15:0), PC40:6, PC42:7, PC(17:1/20:4), PC(18:2/18:3), PC(18:1/24:1), PC(18:2/20:5), PC(20:4/20:0), CE14:1, CE16:0, CE20:5 and DG18:1/18:2.

In some embodiments, a kit of the invention comprises an isotopically labeled analog of one or more of TG45:1, TG46:1, TG46:3, TG47:1, TG47:2, TG48:0, TG48:1, TG48:2, TG48:3, TG49:1, TG49:2, TG49:3, TG49:4, TG50:1, TG50:2, TG50:3, TG50:4, TG50:5, TG51:1, TG51:2, TG51:3, TG51:4, TG52:2, TG52:3, TG52:4, TG52:5, TG52:6, TG53:2, TG53:3, TG53:4, TG53:5, TG54:4, TG54:5, TG54:6, TG54:7, PC(O-16:0/14:0), PC(O-16:0/18:2), PC(O-16:0/20:3), PC(O-16:0/20:4), PC(O-16:0/22:6), PC(O-17:0/20:4), PC(O-18:0/18:1), PC(O-18:0/22:6), PC(O-18:0/18:2), PC(O-18:1/18:2), PC(O-18:1/20:4), PC(O-18:1/20:5), PC(O-18:1/22:6), PC(O-(20:0/22:6), PC(O-20:1/22.6), PC(O-20:2/20:4), PC(O-22:2/20:4), PC(O-22:1/22:6), PC(O-22:2/20:4), PC(O-(24:1/22:6), PC(O-24:2/20:4), PCO-34:2, PCO-36:3, PCO-34:2, PCO-38:3, PCO-40:2, PCO-40:6, PCO-44:7, PCO-46.8, PC(15:0/20:3), PC(15:0/22:6), PC(16:0/14:0), PC(16:0/22:4), PC(16:1/16:0), PC(16:1/20:4), PC(16:1/22.5), PC(17:0/20:5), PC(18:0/20:3), PC(18:0/22:4), PC(18:1/20:3), PC(18:2/18:2), PC(20:4/15:0), PC40:6, PC42:7, PC(17:1/20:4), PC(18:2/18:3), PC(18:1/24:1), PC(18:2/20:5), PC(20:4/20:0), CE14:1, CE16:0, CE20:5 and DG18:1/18:2. In some embodiments, a kit of the invention comprises an isotopically labeled analog of one or more of TG45:1, TG46:1, TG46:3, TG47:1, TG47:2, TG48:0, TG48:1, TG48:2, TG48:3, TG49:1, TG49:2, TG49:3, TG49:4, TG50:1, TG50:2, TG50:3, TG50:4, TG50:5, TG51:1, TG51:2, TG51:3, TG51:4, TG52:2, TG52:3, TG52:4, TG52:5, TG52:6, TG53:2, TG53:3, TG53:4, TG53:5, TG54:4, TG54:5, TG54:6 and TG54:7.

In some embodiments, a kit of the invention comprises an isotopically labeled analog of one or more of PC(O-16:0/14:0), PC(O-16:0/18:2), PC(O-16:0/20:3), PC(O-16:0/20:4), PC(O-16:0/22:6), PC(O-17:0/20:4), PC(O-18:0/18:1), PC(O-18:0/22:6), PC(O-18:0/18:2), PC(O-18:1/18:2), PC(O-18:1/20:4), PC(O-18:1/20:5), PC(O-18:1/22:6), PC(O-(20:0/22:6), PC(O-20:1/22.6), PC(O-20:2/20:4), PC(O-22:2/20:4), PC(O-22:1/22:6), PC(O-22:2/20:4), PC(O-(24:1/22:6), PC(O-24:2/20:4), PCO-34:2, PCO-36:3, PCO-34:2, PCO-38:3, PCO-40:2, PCO-40:6, PCO-44:7 and PCO-46.8.

In some embodiments, a kit of the invention comprises an isotopically labeled analog of one or more of PC(15:0/20:3), PC(15:0/22:6), PC(16:0/14:0), PC(16:0/22:4), PC(16:1/16:0), PC(16:1/20:4), PC(16:1/22.5), PC(17:0/20:5), PC(18:0/20:3), PC(18:0/22:4), PC(18:1/20:3), PC(18:2/18:2), PC(20:4/15:0), PC40:6, PC42:7, PC(17:1/20:4), PC(18:2/18:3), PC(18:1/24:1), PC(18:2/20:5), PC(20:4/20:0), CE14:1, CE16:0, CE20:5 and DG18:1/18:2.

In some embodiments, the isotopically labeled analog may comprise one or more deuterium atoms. The isotopically labeled analogs described above may be used as internal standards during mass spectrometry to facilitate quantification of one or more FSLMs described above.

In some embodiments, kits of the present disclosure further comprise instructions for (a) determining a baseline FSLM(−) lipid profile for one or more FXN-sensitive lipid markers (FSLMs) in a sample obtained from an FXN deficient subject prior to administration of the FXN replacement therapy; (b) determining an FXN replacement lipid profile for the one or more FXN-sensitive lipid markers (FSLMs) in a sample obtained from the FXN deficient subject following administration of the FXN replacement therapy; (c) comparing the FXN replacement lipid profile determined in step (b) with the baseline FXN(−) lipid profile determined in step (a); and (d) determining efficacy of the FXN replacement therapy based on the comparison in step (c).

In some embodiments, kits of the present disclosure further comprise instructions for (a) determining an FXN replacement lipid profile for one or more FXN-sensitive lipid markers (FSLMs) in a sample obtained from an FXN deficient subject following administration of an FXN replacement therapy; (b) comparing the subject FXN replacement lipid profile determined in step (a) with a reference FXN lipid profile for the one or more FSLMs; and (c) determining efficacy of the FXN replacement therapy based on the comparison in step (b).

In some embodiments, kits of the present disclosure further comprise instructions for (a) determining a first FXN replacement lipid profile for one or more FXN-sensitive lipid markers (FSLMs) in a first sample obtained from an FXN deficient subject at a first time point following administration of an FXN replacement therapy to the subject, (b) determining a second FXN replacement lipid profile for the one or more FXN-sensitive lipid markers (FSLMs) in a second sample obtained from the subject at a second time point that is later than the first time point; and (c) comparing the second FXN replacement lipid profile with the first FXN replacement profile.

### EXAMPLES

### Example 1. Lipidomic Analysis of Samples from FRDA Subjects and Healthy Subjects

The goal of this experiment was to identify lipids that may be present in different amounts in a subject with Friedreich's Ataxia (FRDA) as compared to a healthy subject, for potential use as biomarkers. To this end, plasma samples were collected from 11 human subjects with Friedreich's Ataxia (FRDA) and 8 healthy controls (N) as shown in Table 2 below.

TABLE 2
Description of subjects used in the study

| Subject No. | Gender | Age at sample collection | Group |
|---|---|---|---|
| 1 | F | 18 | FRDA |
| 2 | F | 13 | FRDA |
| 3 | F | 30 | FRDA |
| 4 | F | 26 | FRDA |
| 5 | M | 34 | FRDA |
| 6 | M | 41 | FRDA |
| 7 | M | 23 | FRDA |
| 8 | M | 24 | FRDA |
| 9 | M | 18 | FRDA |
| 10 | M | 25 | FRDA |
| 11 | M | 21 | FRDA |
| 12 | F | 27 | N |
| 13 | F | 30 | N |
| 14* | F | 26 | N |
| 15 | M | 25 | N |
| 16 | M | 17 | N |
| 17 | M | 33 | N |
| 18 | M | 36 | N |
| 19 | M | 24 | N |

*Sample 14 was lost during sample preparation

### Sample Preparation and Analysis

Lipids were extracted from the collected samples and analyzed according to a validated protocol previously described in J. of Proteome Research 2018, 17(11):3657-3670, the entire contents of which are hereby incorporated herein by reference. Samples were split into 4 aliquots and stored at −80° C. The lipids in the samples were analyzed by liquid chromatography and mass spectrometry (LC/MS) using a Zorbax Eclipse plus C18 column and a LC-QTOF 6530 Agilent operating in positive and negative modes. The volume of injection was 1 μL, corresponding to 0.33 μL of plasma equivalent. Internal standards were spiked into the plasma samples before extraction and monitored during LC/MS analysis. The lipids used as internal standards were PS(12:0/12:0), PC(14:0/14:0), PG(15:0/15:0) and PC(19:0/19:0).

### Quality Control

Internal samples were spiked into the collected plasma samples before extraction and monitored by LC/MS. A plasma QC sample was injected at the beginning, in the middle and at the end of the LC/MS analysis sequence in positive mode and in negative mode for a total of 6 QC run analyses. Table 3 below shows the results of the QC runs for four different internal standards.

TABLE 3
Results of quality control analyses

| | Samples | | | | | | | |
|---|---|---|---|---|---|---|---|---|
| | Positive Mode | | | | Negative Mode | | | |
| | m/z | RT (min) | RSD | Mean | m/z | RT (min) | RSD | Mean |
| PS(12:0/12:0) | 624.389 | 12.22 | 12.5% | 2.13E+06 | 622.372 | 12.22 | 10.89% | 3.93E+06 |
| PC(14:0/14:0) | 678.508 | 17.78 | 11.66% | 2.36E+07 | 722.498 | 17.76 | 12.22% | 3.50E+06 |
| PG(15:0/15:0) | 712.514 | N/A | N/A | N/A | 693.471 | 18.55 | 11.67% | 2.27E+06 |
| PC(19:0/19:0) | 818.666 | 35.82 | 13.2% | 6.11E+07 | 862.654 | 35.80 | 11.17% | 7.68E+06 |

For the plasma QC samples, analyzed in triplicates, the median relative standard deviation (RSD) was 11.10% in the positive mode and 6.44% in the negative mode. The results of the quality control analysis indicate that the internal standards and the QC samples meet the analysis criteria in terms of variability and intensity of signal. It is noted that the signal for the internal standard PG(15:0/15:0) was missing in the positive ion mode likely due to an error during sample preparation.

### Data Processing

Raw MS data was normalized using cyclic loss algorithm using all MS scan signals measured. Imputation of missing values was done using Knn5. All steps of data processing are described in J. of Proteome Research 2018, 17(11):3657-3670. After filtering and corrections from the data processing, the result dataset contained 1938 MS signals (lipid features), ready for statistical analysis.

### Statistical Analysis

Exploratory principal component analysis (PCA) was performed, followed by comparison between the 2 groups using a linear regression approach on each feature. Correction for age and gender were applied. A Storey correction was used to estimate the False Discovery Rate (FDR). To identify features associated with FRDA, the following criteria was used for the FDR and fold-change (FC): FDR=25% (which corresponds to P value of <0.04) and FC>1.23 or <0.81.

### Annotation of Lipids

Annotation of lipids was carried out using MS/MS on features meeting the criteria of P-value and FC.

### Results

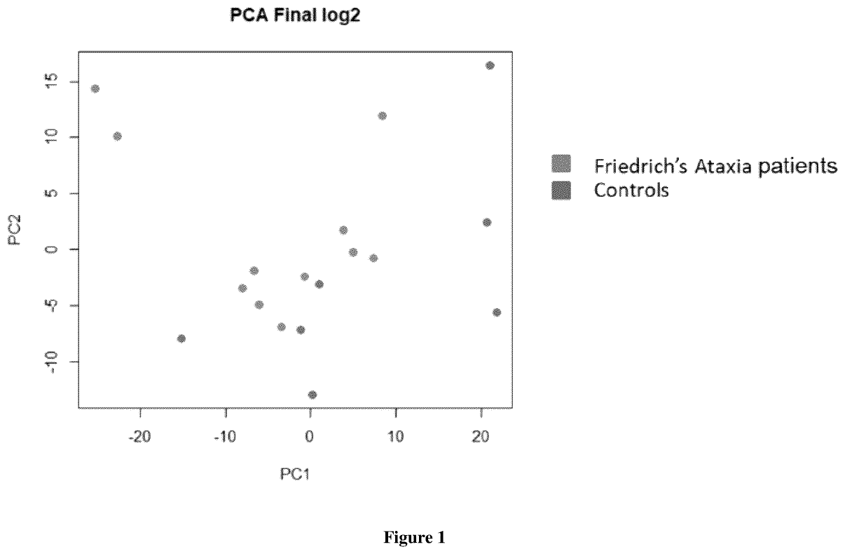

FIG. 1 is a scatter plot showing the results of principal component analysis (PCA) of all MS signals from all analyzed human plasma samples, with different colors indicating FRDA subjects and healthy subjects. The results shown in FIG. 1 indicate that there is a separation between samples from FRDA subjects and samples from healthy subjects.

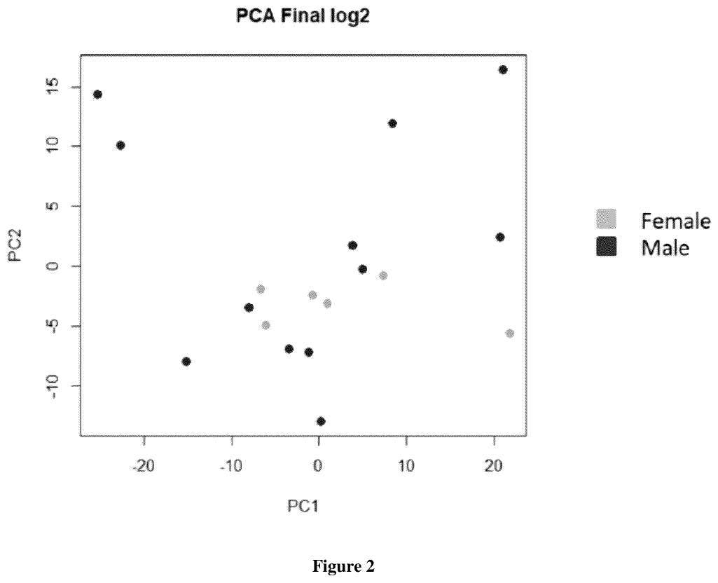

FIG. 2 is a scatter plot showing the results of PCA of all MS signals from all human plasma samples, with different colors indicating subjects of different genders. The results shown in FIG. 2 indicate that the results of the analysis are not impacted by gender of subjects. Nevertheless, correction for gender and age was done in statistical analysis.

### Comparison of FRDA Subjects and Control Subjects

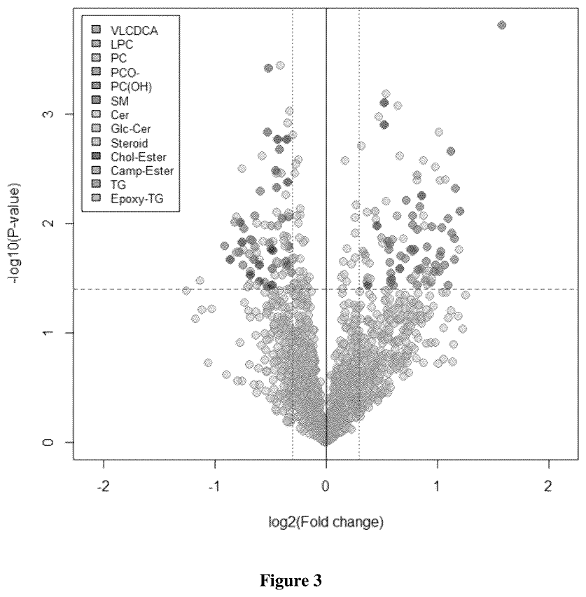

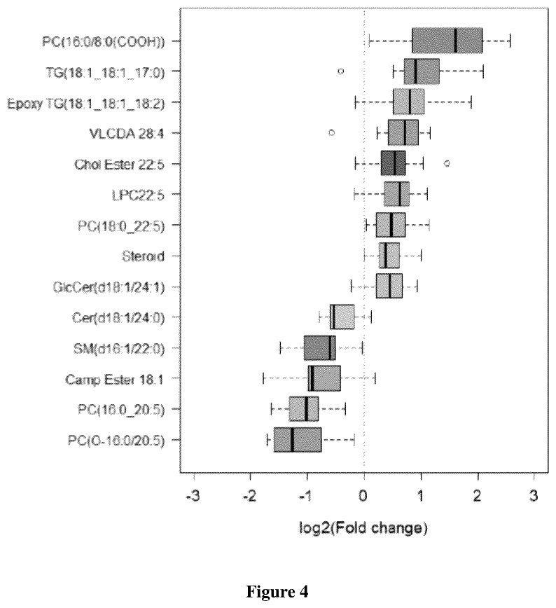

On all the 1938 features, 168 entities were found to discriminate between FRDA and healthy subjects with a p value <0.04 (corresponding to Q-value <0.25) and a fold change >1.23 or <0.81. These 168 entities were analyzed with 2 rounds of MS/MS, resulting in 111 annotations, of which 66 are unique lipids. Results are presented by the volcano plot (FIG. 3) and boxplots (FIG. 4) and are summarized in Table 4 below.

TABLE 4
Lipids found to discriminate between FRDA and healthy subjects.

| | | # of identified | # of identified |
|---|---|---|---|
| | | features that are | features that are |
| | | increased in | decreased in |
| Category | (Sub)class | FRDA subjects | FRDA subjects |
| Fatty acyls (FA) | Very-long-chain dicarboxylic acid | 2(2)* | |
| Glycerophospholipids | Monoacylglycerophophocholines | 1(1) | |
| (GP) | (LPC) | | |
| | Diacylglycerophosphocholines | 8(6) | 6(4) |
| | (PC) | | |
| | Ether | 1(1) | 7(7) |
| | Diacylglycerophosphocholines | | |
| | (PCO—) | | |
| | Oxidized | | 5(5) |
| | Diacylglycerophosphocholines | | |
| | (PC(OH)) | | |
| Sphingolipids (SP) | Sphingomyelins (SM) | | 13(12) |
| | Ceramides (Cer) | | 6(2) |
| | Neutral glycosphingolipids (Glc- | 1(1) | |
| | Cer) | | |
| Sterols Lipids (ST) | Stetroid conjugates | | 2(2) |
| | Cholesteryls esters (CE) | 7(3) | 12(3) |
| | Campesteryl esters | | 3(3) |
| Glycerolipids (GP) | Triacylglycerols (TG) | 33(15) | 2(2) |
| | Epoxy Triacylglycerols (TG) | 2(2) | |

| | | |
|---|---|---|
| Total | 55(31) | 56(35) |

*( ) number of unique lipids

FIG. 3 is a volcano plot that presents the comparison of human plasma samples from FRDA subjects vs. healthy controls. The different lipid subclasses are indicated by different colors. Gray dots represent lipids that are were not identified. The horizontal red line indicates the threshold p-value of 0.04, and the vertical red lines represent fold change of 1.23.

FIG. 4 is a boxplot showing log 2 fold change in the levels of the most significantly modulated lipids in human plasma samples obtained from FRDA subjects as compared to healthy subjects.

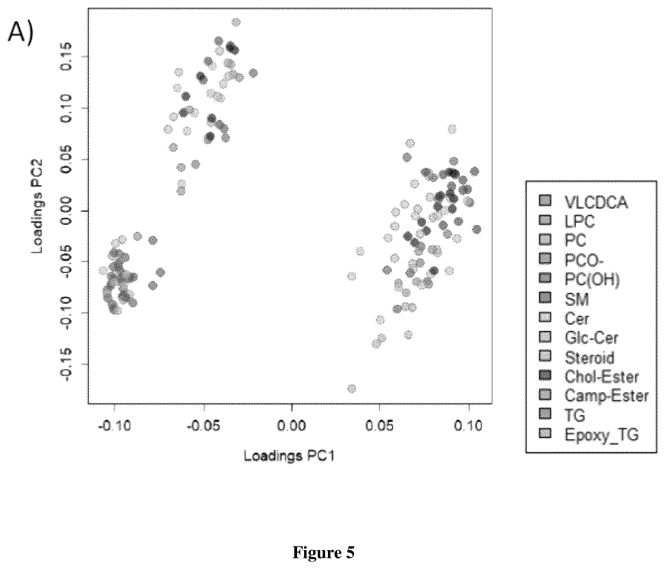

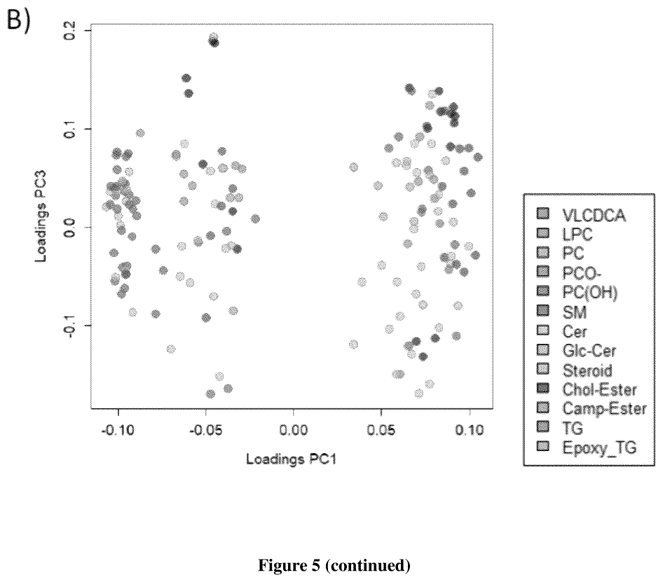

FIG. 5, panel A is a PCA loading plot of the 168 features discriminating FRDA subjects from healthy subjects with lipid subclasses for the 111 annotated features (66 unique lipids) using the color code for PC1 vs. PC2. FIG. 5, panel B is a PCA loading plot of the 168 features discriminating FRDA subjects from healthy subjects with lipid subclasses for the 111 annotated features (66 unique lipids) using the color code for PC1 vs. PC2 (panel A) and PC1 vs. PC3 (panel B). Lipids that are increased in human plasma samples from FRDA subjects as compared to healthy subjects are on the left side, while those that are decreased are on the right. Table 5 below shows lipids that were determined to be present at increased levels in FRDA subjects as compared to healthy controls.

TABLE 5
Lipids present at increased levels in FRDA subjects as compared to healthy controls.

| Sub(class) | ID | Fold change | P-value |
|---|---|---|---|
| Very-long-chain dicarboxylic acid | VLCDCA 28:1(OH)* | 1.50 | 0.029 |
| | VLCDCA 28:4* | 1.49 | 0.023 |
| Monoacylglycerophosphocholines (LPC) | LPC22:5 | 1.65 | 0.021 |
| Diacylglycerophosphocholines (PC) | PC(18:0_24:5) | 1.56 | 8.52E−03 |
| | PC(18:0_22:5) | 1.52 | 0.014 |
| | PC(18:0_22:4) | 1.28 | 0.019 |
| Ether Diacylglycerophosphocholines | PC(O-17:1/20:4) | 1.60 | 0.020 |
| (PCO—) | | | |
| Oxidized Diacylglycerophosphocholines | PC(16:0/8:0(COOH))* | 3.00 | 1.54E−04 |
| (PC(OH)) | PC(16:0/9:0(COOH)) Paz-PC* | 2.31 | 7.75E−03 |
| | PC(18:0/9:0(COOH))* | 2.18 | 2.21E−03 |
| | PC(18:0/8:0(COOH))* | 1.74 | 0.018 |
| | PC(16:0/18:3(OH))* | 1.47 | 0.015 |
| Neutral glycosphingolipids (Glc-Cer) | GlcCer(d18:1/24:1) | 1.42 | 0.014 |
| Steroid conjugates | Steroid conjugates* | 1.36 | 7.78E−03 |
| | Steroid conjugates* | 1.26 | 0.018 |
| Cholesteryls esters (CE) | CE22:5 | 1.82 | 0.006 |
| | CE17:0 | 1.44 | 7.78E−03 |
| | CE18:0 | 1.38 | 0.011 |
| Triacylglycerols (TG) | TG(18:1_18:1_17:0) | 2.24 | 4.82E−03 |
| | TG(18:1_18:1_20:3) | 2.24 | 0.022 |
| | TG(14:0_18:1_17:1) | 2.23 | 0.014 |
| | TG(16:0_17:1_18:1) | 2.10 | 0.024 |
| | TG48:3 | 2.05 | 0.018 |
| | TG(16:0_16:1_18:2) | 2.02 | 0.028 |
| | TG(18:1_18:1_17:0) | 1.99 | 0.024 |
| | TG(14:0_18:1_16:1) | 1.98 | 0.028 |
| | TG(18:2_16:1_15:0) | 1.87 | 0.022 |
| | TG(18:0_18:1_22:4) | 1.77 | 0.011 |
| | TG(18:2_18:1_17:1) | 1.77 | 0.037 |
| | TG(18:1_18:1_22:4) | 1.72 | 0.010 |
| | TG(18:1_18:2_20:3) | 1.70 | 0.032 |
| | TG54:3 | 1.50 | 0.032 |
| | TG(18:1_18:1_22:5) | 1.50 | 0.033 |
| Epoxy Triacylglycerols (Epoxy-TG) | Epoxy TG 16:0_18:1_18:2 (O) | 1.72 | 0.018 |
| | Epoxy TG 18:1_18:1_18:2 (O) | 1.77 | 3.62E−03 |

*annotation needs to be confirmed

It is noted that the lipid TG(18:1_18:1_17:0) is listed twice in Table 5 because it was identified based on two separate features during mass spectrometric analysis.

Table 6 below shows lipids that were determined to be present at decreased levels in FRDA subjects as compared to healthy controls.

TABLE 6
Lipids present at decreased levels in FRDA
subjects as compared to healthy controls.

| Sub(class) | ID | Fold change | P-value |
|---|---|---|---|
| Diacylglycerophosphocholines (PC) | PC(14:0_18:2) | 0.74 | 0.011 |
| | PC(18:0_20:5) | 0.63 | 0.028 |
| | PC(18:1_20:5) | 0.57 | 0.018 |
| | PC(16:0_20:5) | 0.57 | 9.84E−03 |
| Ether Diacylglycerophosphocholines | PC(O-20:2/20:4) | 0.80 | 0.028 |
| (PCO—) | PC(O-18:1/16:0) | 0.70 | 0.015 |
| | PCO-40:3 | 0.67 | 5.10E−03 |
| | PC(O-18:1/22:6) | 0.64 | 8.55E−03 |
| | PC(O-16:1/18:2) | 0.63 | 0.014 |
| | PC(P-18:0/20:5) | 0.60 | 0.024 |
| | PC(O-16:0/20:5) | 0.53 | 0.016 |
| Sphingomyelins (SM) | SM(d18:1/23:0) | 0.79 | 8.78E−03 |
| | SM(d18:1/24:0) | 0.78 | 0.022 |
| | SM(d18:2/23:0) | 0.76 | 9.11E−03 |
| | SM(d18:1/22:0) | 0.75 | 2.11E−03 |
| | SM(d18:2/22:0) | 0.74 | 4.77E−03 |
| | SM(d18:1/22:1) | 0.73 | 3.28E−03 |
| | SM(d18:2/24:0) | 0.72 | 0.026 |
| | SM(d18:1/21:0) | 0.70 | 3.81E−04 |
| | SM(d16:1/16:0) | 0.69 | 1.47E−03 |
| | SM(d16:1/22:0) | 0.64 | 0.021 |
| Ceramides (Cer) | Cer(d18:1/22:0) | 0.81 | 0.022 |
| | Cer(d18:1/24:0) | 0.77 | 0.013 |
| Neutral glycosphingolipids (Glc-Cer) | GlcCer(d18:1/24:1) | 1.42 | 0.014 |
| Cholesteryl esters (CE) | CE18:1 | 0.74 | 1.73E−03 |
| | CE14:0 | 0.71 | 0.017 |
| | CE20:5 | 0.59 | 0.015 |
| Campesteryl esters | 18:2 Campesteryl ester | 0.69 | 0.035 |
| | 18:3 Campesteryl ester | 0.64 | 0.016 |
| | 18:1 Campesteryl ester | 0.62 | 0.014 |
| Triacylglycerols (TG) | TG(18:2_20:5_20:4) | 0.60 | 0.011 |
| | TG60:8 | 0.58 | 0.018 |

### Conclusions

The experiment described in this example identified lipids that are modulated, e.g., present in increased or decreased amounts, in FRDA subjects as compared to healthy control subjects. The lipids identified as a result of this experiment may be used as biomarkers to monitor or evaluate effectiveness of frataxin (FXN) replacement therapy.

The increased amounts of oxidized PCs and epoxy TGs in FRDA subjects as compared to the amounts in healthy subjects are indicative of increased oxidative stress in FRDA subjects.

The absence of long chain acylcarnitines modulated in FRDA subjects is indicative of minimal perturbations of mitochondrial fatty acid metabolism. The decreased amounts of ether lipids (as precursors of plasmalogens) in FRDA subjects as compared to the amounts in healthy control subjects indicates perturbations in peroxisomal metabolism in FRDA subjects.

The levels of sphingolipids, such as sphingomyelins (SM) and ceramides (Cer) with very long chain fatty acid (≥20) in the sn2 position, are decreased in FRDA subjects as compared to healthy control subjects. Changes in the levels of sphingolipids have been reported in subjects with cardiovascular diseases and heart failure, and are predictive of adverse events (see, e.g., Laaksonen et al., Eur. Heart J. 2016, 37:1967 and Peterson et al., JAHA 2018, 7:e007931).

Many triacylglycerols (TGs) are increased in FRDA subjects as compared to healthy control subjects. These TGs have fatty acyl moieties with odd or branched chain, which is indicative of dysbiosis or altered lipogenesis.

### Example 2. Untargeted Lipidomic Analysis of Serum Samples from a Mouse Model of FRDA

The goal of this experiment was to determine if one or more lipids are modulated (i.e., levels of lipids are increased or decreased) in a mouse model of FRDA following administration of a frataxin (FXN) replacement therapeutic compound. To this end, plasma samples were collected from mice treated with FXN replacement therapeutic compound or vehicle control, and subjected to lipidomic analysis. The experiments used wild-type mice and FXN knock-out (FXN-KO) mice which represent a mouse model of FRDA. Specifically, the experiment utilized the groups of mice as described in the Table 7 below.

TABLE 7
Mice and conditions utilized in the experiment

| | No. of female | No. of male |
|---|---|---|
| Group | mice | mice |
| Wild-type mouse treated with | 2 | 5 |
| vehicle control (WTV) | | |
| Wild-type mouse treated with | 1 | 2 |
| article (i.e., FXN replacement | | |
| therapeutic compound) (WTTA) | | |
| Mutant (i.e., FXN-KO) mouse treated | 4 | 4 |
| with vehicle control (MV) | | |
| Mutant (i.e., FXN-KO) mouse treated | 4 | 1 |
| with article (i.e., FXN replacement | | |
| therapeutic compound) (MTA) | | |

### Sample Preparation and Analysis

Lipids were extracted from the collected samples and analyzed according to a validated protocol previously described in J. of Proteome Research 2018, 17(11):3657-3670, the entire contents of which are hereby incorporated herein by reference. Samples were split into 4 aliquots and stored at −80° C. The lipids in the samples were analyzed by liquid chromatography and mass spectrometry (LC/MS) using a Zorbax Eclipse plus C18 column and a LC-QTOF 6530 Agilent operating in positive and negative modes. The volume of injection was 1 μL, corresponding to 0.33 μL of plasma equivalent. Internal standards were spiked into the plasma samples before extraction and monitored during LC/MS analysis. The lipids used as internal standards were PS(12:0/12:0), PC(14:0/14:0), PG(15:0/15:0) and PC(19:0/19:0).

### Quality Control

Internal samples were spiked into the collected plasma samples before extraction and monitored by LC/MS. A plasma QC sample was injected at the beginning, in the middle and at the end of the LC/MS analysis sequence in positive mode and in negative mode for a total of 6 QC run analyses. Table 8 below shows the results of the QC runs for four different internal standards.

TABLE 8
Results of quality control analyses

| | Samples | | | | | |
|---|---|---|---|---|---|---|
| | Positive Mode | | | Negative Mode | | |
| | m/z | RT (min) | RSD | m/z | RT (min) | RSD |
| PS(12:0/12:0) | 624.389 | 12.23 | 9.43% | 622.372 | 12.23 | 13.20% |
| PC(14:0/14:0) | 678.508 | 17.81 | 7.80% | 722.498 | 17.82 | 13.90% |
| PG(15:0/15:0) | 712.514 | 18.69 | 6.24% | 693.471 | 18.7 | 9.83% |
| PC(19:0/19:0) | 818.666 | 35.87 | 9.00% | 862.654 | 35.86 | 7.60% |

The results of the quality control analysis indicate that the internal standards and the QC samples meet the analysis criteria.

### Data Processing

Raw MS data was normalized using cyclic loss algorithm using all MS scan signals measured. Imputation of missing values was done using Knn5. All steps of data processing are described in J. of Proteome Research 2018, 17(11):3657-3670.

### Statistical Analysis

The output text file containing the processed dataset was imported into Mass Professional Pro (MPP: version 12.6.1; Agilent Technologies Inc.) software. A t-test was performed between conditions (WTV, MV, MTA), and a threshold p-value of 0.005 was used. Correction for multiple testing was not considered because of the lack of power in the analysis.

### Annotation of Lipids

First, the resulting list from the data processing was searched in the in-house lipid database APHID, which contains 498 identifications of lipids done using MS/MS with the information on m/z values and retention times for these lipids using the LC-MS method used in the analysis. For the m/z not found in the APHID, a second search was conducted in METLIN, an open-access database which is based only on m/z values. To minimize false positive rate, the search was performed based on the consideration that the highest intensity ions are observed for the lipid (sub)classes as listed in Table 9 below.

TABLE 9
Lipid subclasses and the corresponding highest intensity ions observed

| | Subclass | Ion |
|---|---|---|
| Positive Ionization Mode | diacylglycerophosphocholines (PC) | [M + H]⁺ |
| | diacylglycerophosphoethanolamines | [M + H]⁺ |
| | (PE) | |
| | sphingomyelins (SM) | [M + H]⁺ |
| | fatty acids (FA) | [M + NH₄]⁺ |
| | phosphatidylinosinols (PI) | [M + NH₄]⁺ |
| | steryl esters (CE) | [M + NH₄]⁺ or |
| | | [M + Na]⁺ |
| | glycerolipids (GP) | [M + NH₄]⁺ or |
| | | [M + Na]⁺ |
| Negative Ionization | fatty acids (FA) | [M − H]⁻ |
| Mode | diacylglycerophosphoethanolamines | [M − H]⁻ |
| | (PE) | |
| | diacylglycerophosphoinositols (PI) | [M − H]⁻ |
| | diacylglycerophosphocholines (PC) | [M + HCOO]⁻ |
| | sphingomyelins (SM) | [M + HCOO]⁻ |
| | Ceramides (Cer) | [M + HCOO]⁻ |

At this step no MS/MS was done to confirm some identification or to identify acyl chain from TG and PC.

### Results

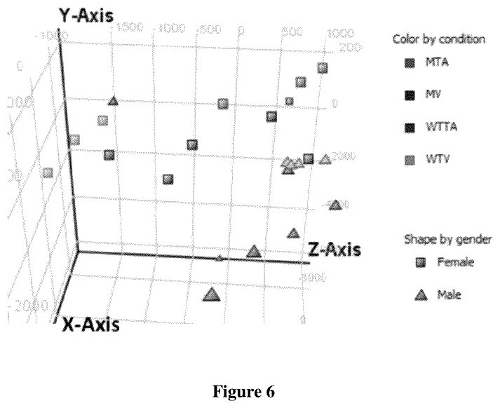

FIG. 6 is a scatter plot showing the results of PCA of all MS signals from all mouse plasma samples, with different colors indicating different conditions: WT mice treated with vehicle (WTV), WT mice treated with FXN replacement therapy (WTTA), FXN-KO mice treated with vehicle (MV) and FXN-KO mice treated with FXN replacement therapy (MTA). The shape of the dots indicate different genders. The PCA analysis clearly shows a difference between male vs. female within the same groups. The subsequent analysis was carried out only for female mice from the WTV, MV and MTA groups.

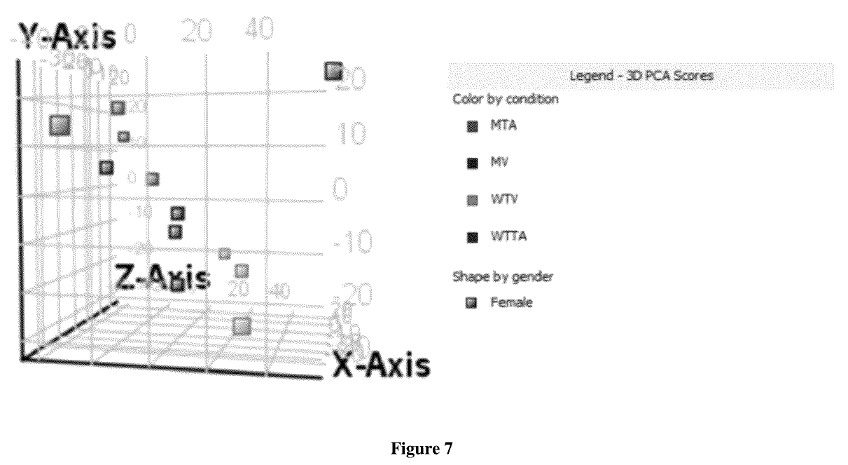

FIG. 7 is a scatter plot showing the results of PCA of all plasma samples from female mice with all MS signals, with different colors indicating different conditions: WTV, WTTA, MV and MTA. The PCA analysis shows separation between the 3 studied groups.

Comparison of MV Vs. WTV Samples from Female Mice (Effect of Mutation)

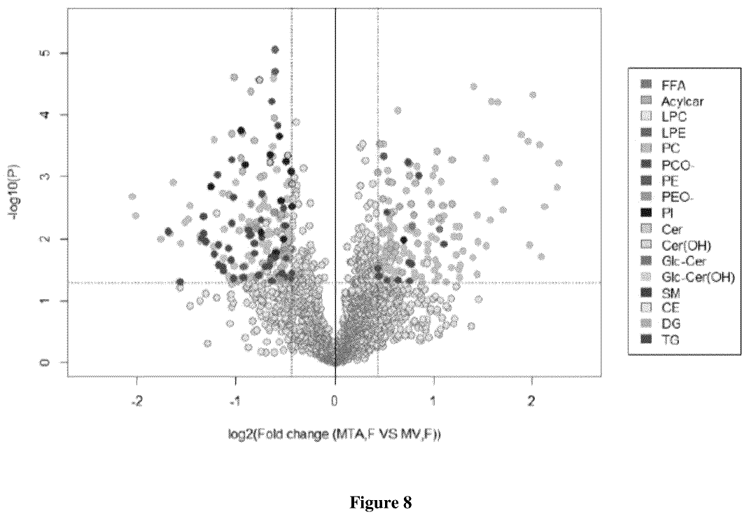

Of all the 2508 features, 356 entities discriminate MV vs. WTV with a p-value <0.05 and a fold change >1.35 between the two conditions. These 356 entities were searched for annotation in APHID and METLIN databases, resulting in 164 annotations. The results of the analyses showing lipids that are modulated in mutant mice as compared to wild-type mice treated with vehicle are presented in Table 10 below and in FIGS. 7 and 8.

TABLE 10
Lipids that are modulated in FXN-KO mice as compared to WT mice treated with vehicle.

| | | # of identified | # of identified |
|---|---|---|---|
| | | features that | features that |
| | | are increased | are decreased |
| | | in FXN-KO | in FXN-KO |
| Category | (Sub)class | mice | mice |
| Fatty acyls (FA) | Fatty acids and conjugates | 1 | |
| | Fatty acylcarnitines | 2(2*) | |
| Glycerophospholipids | Monoacylglycerophosphocholines | 6(1*) | 3(1*) |
| (GP) | (LPC) | | |
| | Monoacylglycerophosphoethanolamines | | 1 |
| | (LPE) | | |
| | Ether Monoacylglycerophosphocholines | | |
| | (LPCO—) | | |
| | Diacylglycerophosphocholines (PC) | 30(12*) | 29(10*) |
| | Diacylglycerophosphoethanolamines | | 2 |
| | (PE) | | |
| | Diacylglycerophosphoinositols (PI) | 1 | 12 |
| | Ether Diacylglycerophosphocholines | 5(1*) | 12(2*) |
| | (PCO—) | | |
| | Phosphatidylethanolamine ethers | | 7 |
| | (PEO—) | | |
| | Oxidized | | |
| | Diacylglycerophosphocholines | | |
| | (PC(OH)) | | |
| Sphingolipids (SP) | Sphingomyelins (SM) | 1 | 3(3*) |
| | Ceramides (Cer) | | 5(1*) |
| | Oxidized Ceramides (cer(OH)) | | 1 |
| | Neutral glycosphingolipids (Glc-Cer) | 1 | 1 |
| | Oxydized glycosphingolipids (Glc- | 1 | |
| | Cer(OH)) | | |
| Sterols Lipids (ST) | Steryls esters (CE) | 1 | 2 |
| Glycerolipids (GP) | Diacylglycerols (DG) | 1 | 2(1*) |
| | Triacylglycerols (TG) | 6 | 28 |
| Total | | 56 | 108 |

*No. of lipids identified using APHID database

FIG. 8 is a volcano plot presenting the comparison of plasma samples obtained from female MTA mice vs. female WTV mice. The different lipid subclasses are indicated by different colors, and gray dots represent lipids that were not identified. The horizontal red line indicates the threshold p-value of 0.05, and the vertical red line represents fold change of 1.35.

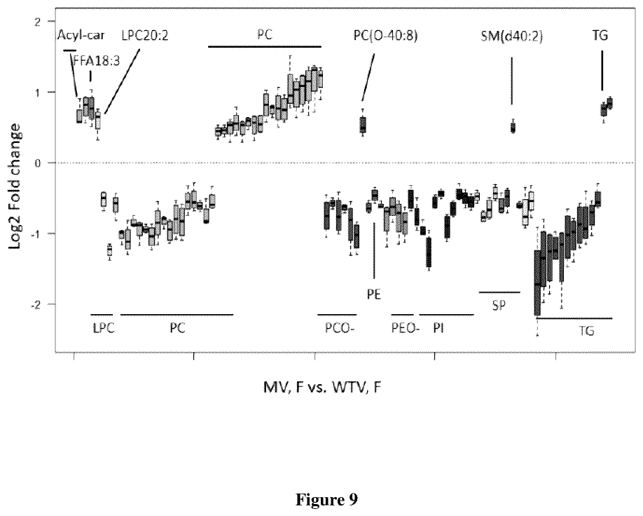

FIG. 9 is a panel of boxplots showing the most significantly modulated lipids (p<0.01) in plasma samples obtained from female MV mice vs. female WTV mice.

Comparison of MV vs. MTA samples from female mice (effect of treatment of mutant mice)

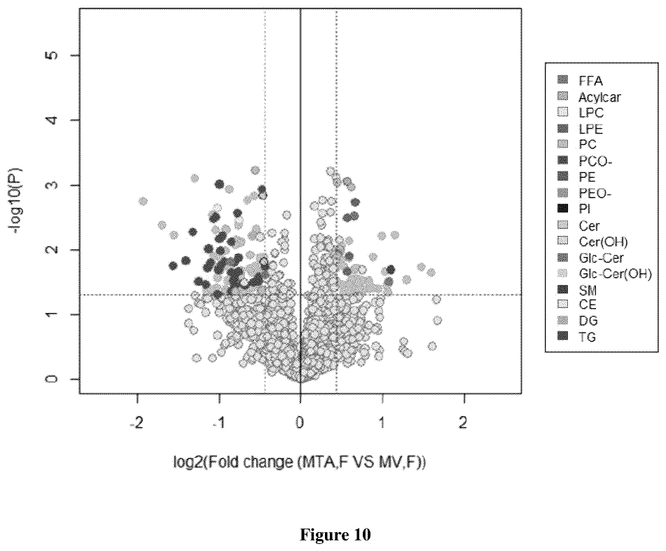

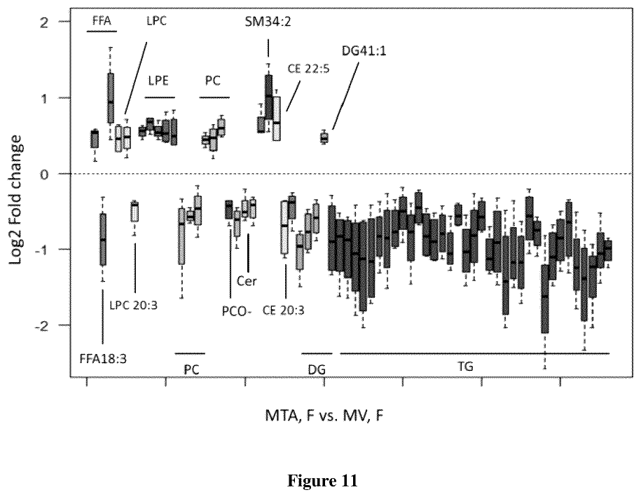

Of all the 2508 features, 167 entities discriminate MTA vs. MV with a p-value <0.05 and a fold change >1.35 between the two conditions. These 167 entities were searched for annotation in APHID and METLIN databases, resulting in 63 annotations. The results of the analyses showing lipids that are modulated in mutant mice treated with FXN replacement therapeutic compound (MTA) as compared to mutant mice treated with vehicle (MV) are presented in Table 11 below and in FIGS. 10 and 11.

TABLE 11
Lipids that are modulated in FXN-KO mice treated with FXN replacement
therapeutic compound as compared to FXN-KO mice treated with vehicle.

| | | # of identified | # of identified |
|---|---|---|---|
| | | features that | features that |
| | | are increased | are decreased |
| | | in FXN-KO | in FXN-KO |
| Category | (Sub)class | mice | mice |
| Fatty acyls (FA) | Fatty acids and conjugates | 2(1*) | 1 |
| Glycerophospholipids | Monoacylglycerophosphocholines | 2(2*) | 1 |
| (GP) | (LPC) | | |
| | Monoacylglycerophosphoethanolamines | 5(3*) | |
| | (LPE) | | |
| | Diacylglycerophosphocholines (PC) | 3(1*) | 3(1*) |
| | Diacylglycerophosphoethanolamines | | 1 |
| | (PE) | | |
| | Ether Diacylglycerophosphocholines | | 1 |
| | (PCO—) | | |
| Sphingolipids (SP) | Sphingomyelins (SM) | 1 | |
| | Ceramides (Cer) | | 2(1*) |
| | Neutral glycosphingolipids (Glc-Cer) | 1 | 1 |
| Sterols Lipids (ST) | Steryls esters (CE) | 1 | 1 |
| Glycerolipids (GP) | Diacylglycerols (DG) | 1 | 3 |
| | Triacylglycerols (TG) | | 36 |
| | Monoacylglycerols (MG) | | 1 |
| Total | | 15 | 48 |

*No. of lipids identified using APHID database

FIG. 10 is a volcano plot showing the comparison of plasma samples from female MTA mice vs. female WV. The different lipid subclasses are indicated by different colors, and gray dots represent lipids that were not identified. The horizontal red line indicates the threshold p-value of 0.05, and the vertical red line represents fold change of 1.35.

FIG. 11 shows boxplots of the 63 annotated lipids identified in plasma samples from female MTA mice vs. female MV mice.

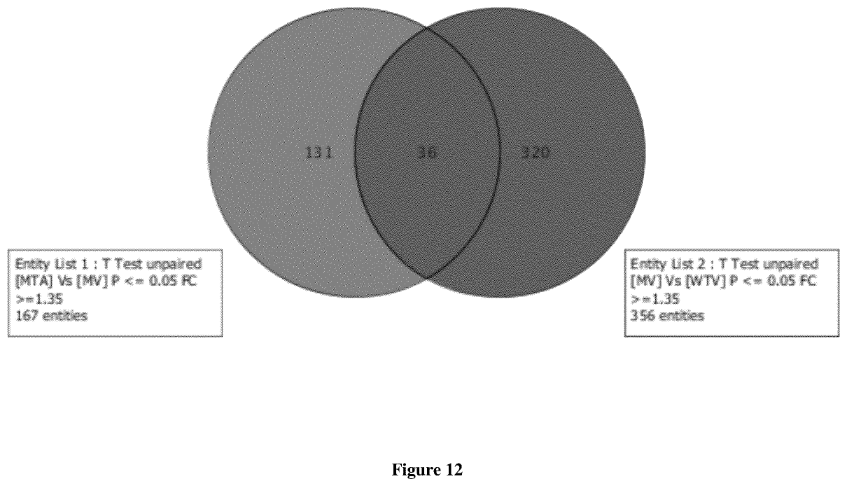

FIG. 12 is a Venn Diagram in which the red (left) circle represents 167 entities which discriminate between plasma samples obtained from MTA mice and MV mice, and the blue circle represents 356 entities which discriminate between plasma samples obtained from MV mice and WTV mice. The overlapping portion of the red and blue circles represents 36 entities which represent lipids that are modulated as a result of FXN-KO mutation and which are further modulated by FXN replacement therapy administered to the FXN-KO mice.

The list of 36 features common for the two comparisons (MTA vs. MV and MV vs. WTV) was screened and 4 features eluting during the dead volume plus 2 entities which were replicate features from a compound already in the list were filtered out. The final list contains 30 features which are listed in Table 12 below. In Table 12, the entity which is decreased in MV samples as compared to the WTV samples (showing the effect of FXN-KO mutation) and decreased in MTA samples as compared to MV samples (showing the effect of treatment of FXN-KO mice with the FXN replacement therapeutic compound) is shown in bold italic. Entities that are increased in MTA samples as compared to the MV samples (showing the effect of FXN-KO mutation) and decreased in MTA samples as compared to MV samples (showing the effect of treatment of FXN-KO mice with FXN replacement therapeutic compound) is shown in bold.

TABLE 12
Lipids and features that are simultaneously modulated in MTA vs. MV samples and MV vs. WTV samples.

| | | | [MV] Vs [WTV] | | | [MTA] Vs [MV] | | |
|---|---|---|---|---|---|---|---|---|
| | Compound | | | FC | | | FC | |
| Lipid ID | (mass DRT) | Adduct | p-value | (abs) | Regulation | p-value | (abs) | Regulation |
| DG(18:1/18:1) | pos637.5660@35.11 | NH4+ | 3.83E−02 | 1.38 | down | 2.30E−03 | 2.03 | down |
| unknown | neg: 684.5981@36.44 | | 1.00E−02 | 1.36 | down | 1.50E−03 | 1.48 | down |
| unknown | pos: 1523.2775@49.97 | | 4.33E−02 | 1.53 | down | 1.65E−02 | 2.12 | down |
| LPC20:3 | pos: 545.3489@8.71 | H+ | 6.20E−03 | 1.43 | down | 6.40E−03 | 1.41 | down |
| unknown | pos: 567.3159@6:70 | | 1.89E−02 | 1.82 | down | 1.20E−02 | 1.54 | down |
| unknown | pos: 589.3738@9.84 | | 1.25E−02 | 1.62 | down | 2.27E−02 | 1.51 | down |
| PC(P-34:4) | pos: 737.5352@19.05 | H+ | 1.00E−04 | 1.56 | down | 1.20E−03 | 1.39 | down |
| PE(36:0) | pos: 747.5733@22.88 | H+ | 3.00E−04 | 1.55 | down | 1.70E−03 | 1.58 | down |
| PC(40:7) | pos: 831.5737@25.40 | H+ | 7.10E−03 | 1.77 | down | 4.24E−02 | 1.39 | down |
| TG(50:5) | pos: 841.7204@43.58 | NH4+ | 2.10E−03 | 2.05 | down | 1.53E−02 | 1.9 | down |
| TG(51:2) | pos: 861.7842@58.38 | NH4+ | 2.68E−02 | 2.27 | down | 1.76E−02 | 2.95 | down |
| unknown | pos: 864.6560@46.20 | | 8.70E−03 | 1.6 | down | 4.25E−02 | 1.72 | down |
| TG(54:2) | pos: 875.7988@62.61 | NH4+ | 8.20E−03 | 2.52 | down | 1.48E−02 | 2.65 | down |
| unknown | pos: 876.7925@55.73 | | 2.74E−02 | 2.03 | down | 4.32E−02 | 2.23 | down |
| TG(54:7) | pos: 893.7427@46.51 | NH4+ | 1.12E−02 | 2.48 | down | 3.50E−02 | 2.24 | down |
| TG(54:8) | pos: 896.6927@43.62 | Na+ | 1.90E−03 | 1.68 | down | 1.03E−02 | 1.97 | down |
| TG(56:9) | pos: 917.7420@44.32 | NH4+ | 4.34E−02 | 2.04 | down | 4.89E−02 | 2.03 | down |
| TG(56:10) | pos: 920.6910@41.88 | Na+ | 7.30E−03 | 1.84 | down | 3.06E−02 | 1.78 | down |
| unknown | pos: 938.6841@44.74 | | 1.20E−03 | 2 | down | 8.70E−03 | 1.41 | up |
| unknown | pos: 278.2241@7.67 | | 6.30E−03 | 1.72 | up | 1.51E−02 | 1.81 | down |
| PC(46:6) | pos: 917.6898@35.97 | H+ | 1.65E−02 | 1.53 | up | 6.00E−04 | 1.47 | down |
| unknown | pos: 948.7571@64.29 | | 8.00E−03 | 1.39 | up | 7.80E−03 | 1.67 | down |
| TG(58:7) | pos: 949.8111@53.39 | NH4+ | 4.55E−02 | 1.55 | up | 6.80E−03 | 2 | down |
| TG(60:8) | pos: 980.7874@57.48 | Na+ | 2.98E−02 | 1.35 | up | 3.20E−02 | 1.5 | down |
| unknown | neg: 304.2391@11.76 | | 4.33E−02 | 1.42 | up | 1.44E−02 | 1.4 | up |
| unknown | pos: 409.3200@6.25 | | 1.82E−02 | 2.34 | up | 2.97E−02 | 1.78 | up |
| unknown | pos: 676.5400@36.17 | | 1.20E−03 | 3.07 | up | 4.54E−02 | 2.08 | up |
| unknown | pos: 704.5719@38.02 | | 3.04E−02 | 2.39 | up | 2.29E−02 | 3.01 | up |
| PC(40:6) | pos: 833.5946@24.98 | H+ | 7.10E−03 | 1.44 | up | 1.10E−03 | 1.51 | up |

The following lipids listed in Table 12 are modulated (increased or decreased) in MV vs. WTV (showing the effect of FXN-KO mutation) and normalize in MTA vs. MV (showing the effect of treatment with the FXN replacement therapeutic compound): pos:278:2241@7.67, PC(46:6), TG(60:8), pos:948.7571@64.29, TG(58:7), pos:938.6841@44.74. Other groups of lipids or features are up or down in MV vs. WTV, but changes are amplified with treatment with the FXN replacement therapeutic compound. This could suggest an adaptive mechanism that is targeted by the treatment.

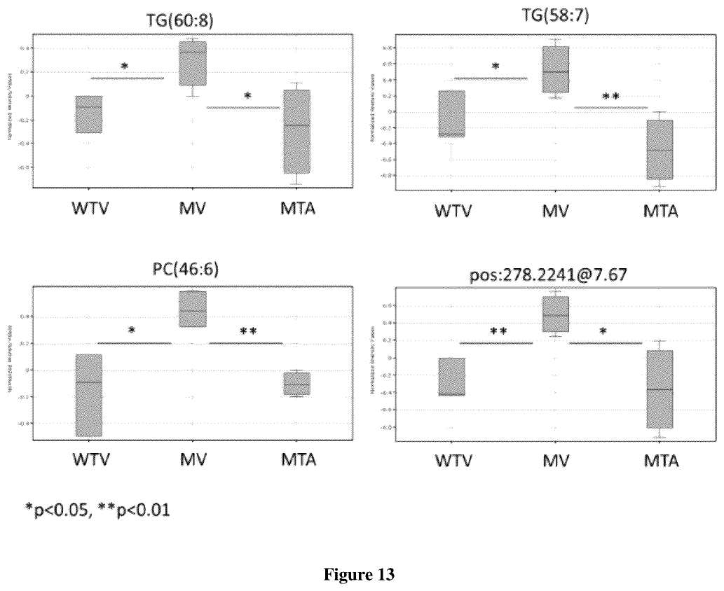

FIG. 13 is a series of boxplots of representative lipids or features that are increased in FXN-KO mice (MV) as compared to the WT mice (WTV) and that are decreased following treatment with FXN replacement therapy (MTA).

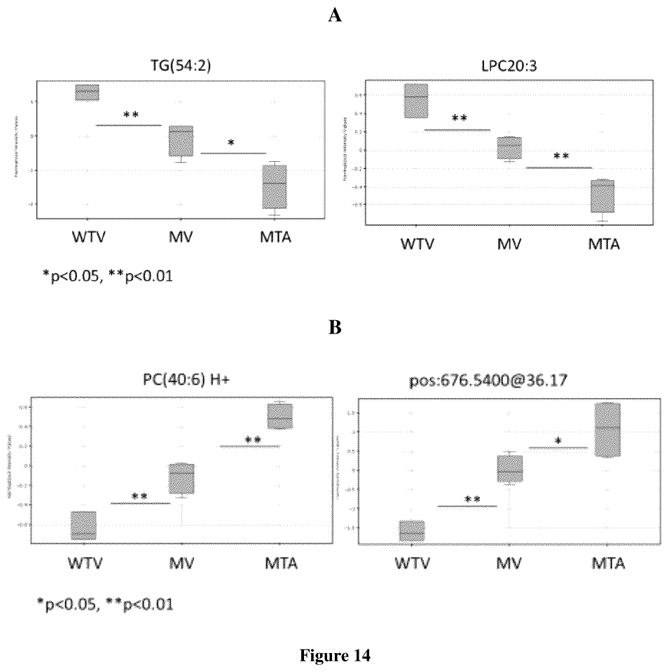

FIG. 14, panel A is a series of boxplots of representative lipids that are decreased in FXN-KO mice (MV) as compared to the WT mice (WTV) and that are further decreased following treatment with FXN replacement therapy (MTA). FIG. 14, panel B is a series of boxplots of representative lipids and features that are increased in FXN-KO mice treated with vehicle (MV) vs. WT mice treated with vehicle (WTV) and that are further increased following treatment with FXN replacement therapy (MTA).

The following lipid subclasses were investigated: triacylglycerols (TG), diacylglycerols (DG), phosphatidylcholines (PC) and lysophospholipids (LCP and LPE).

### Triacylglycerols

The following TGs are increased in FXN-KO mice and decrease following treatment with the FXN replacement therapeutic compound: TG(60:8), TG(59:9), TG (57:7), TG(58:7), TG(58:6), TG(56:9), TG(56:4), TG(54:5), TG(56:5), TG(56:3), TG(53:4), TG(54:2). In general, TGs decrease with the treatment with the FXN replacement therapeutic compound.

### Diacylglycerols

DGs decrease in FXN-KO mice and further decrease with treatment with the FXN replacement therapeutic compound.

### Phosphatidylcholines

The following PCs are modulated in the FXN-KO mice and are normalized following treatment with the FXN replacement therapeutic compound: PC(46:6), PC(41:6) and PC (44:5). However, for most PCs, changes associated with the FXN knockout are not modified or amplified with treatment.

### Lysophospholipids

For most lysophospholipids (LPC and LPE), treatment of FXN-KO mice with the FXN replacement therapeutic compound amplified the changes seen in the FXN-KO mice. Most LPC and LPE increase, except for LPC20:3 and LPC20:5, which decrease. It is noted that lysophospholipids are bioactive molecules, suggesting that treatment with the FXN replacement therapeutic compound is targeting an adaptive mechanism responsible for the formation of these bioactive lipids.

Fold Change Vs. Acyl Chain Length

The chain length of TGs and PCs that are modulated in the FXN-KO mice and normalize following treatment was investigated. To this end, “fold change” was plotted versus the number of carbons in the lipids using the list of 523 features.

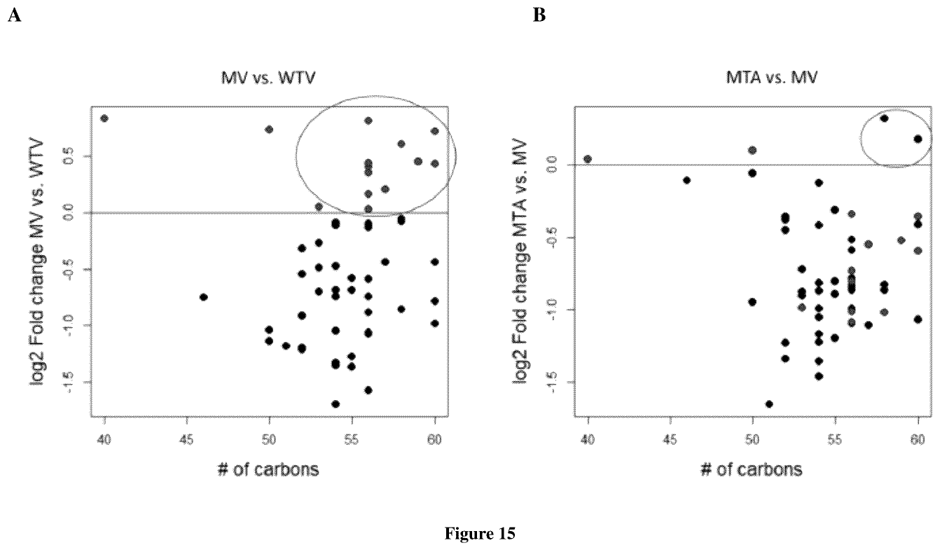

FIG. 15, panel A is a dot plot showing log 2 fold change in the levels of various triglycerides (TGs) vs. number of carbons in their acyl chain in FXN-KO mice treated with vehicle vs. wild-type mice treated with vehicle (MV vs. WTV). The circled dots represent TGs that are increased in FXN-KO mice versus wild-type mice, representing the effect of mutation. Of these TGs, many have an acyl chain length of >55. Some TGs that are increased in FXN-KO mice as compared to the wild-type mice that do not decrease following FXN replacement therapy have an acyl chain of ≤50.

FIG. 15, panel B is a dot plot showing log 2 fold change in the levels of various TGs vs. number of carbons in their acyl chain in FXN-KO mice treated with FXN replacement therapy or vehicle (MTA vs. MV). The circled dots represent TGs which were decreased in the FXN-KO mice as compared to the wild-type mice and were increased following treatment with the FXN replacement therapeutic compound. These TGs have long chain length (<55).

It is noted that very long acyl chains are formed by elongation of commonly found long acyl chain (C16 or palmitate; and C18 or stearate). They can be formed when the mitochondria is unable to oxidize long acyl chain. This could represent a mechanism of adaptation.

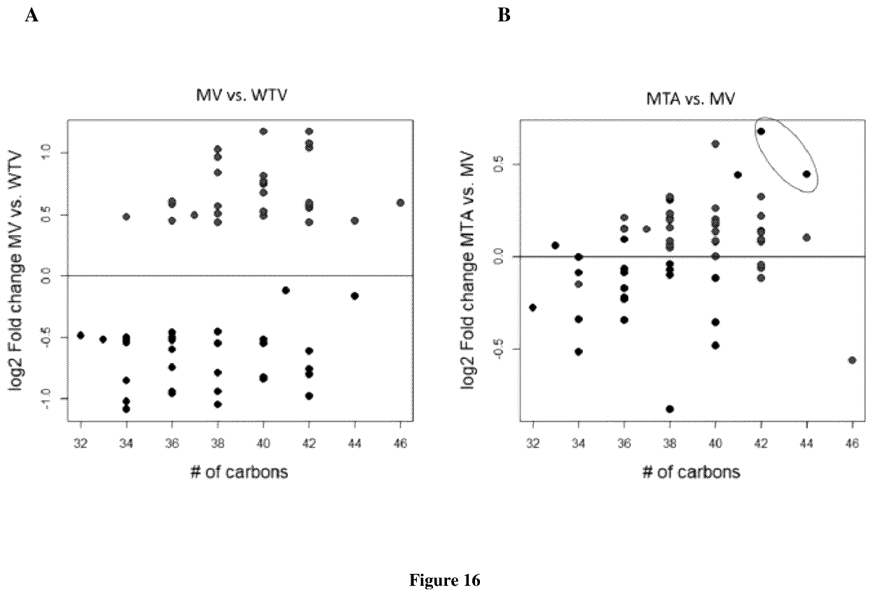

FIG. 16, panel A is a dot plot showing log 2 fold change in the levels of various phosphatidylcholines (PCs) vs. number of carbons in their acyl chain in FXN-KO mice treated with vehicle vs. wild-type mice treated with vehicle (MV vs. WTV).

FIG. 16, panel B is a dot plot showing log 2 fold change in the levels of various PCs versus their acyl chain length in FXN-KO mice treated with FXN replacement therapy or vehicle (MTA vs. MV). The circled dots represent two PCs that are decreased in FXN-KO mice and are increased following FXN replacement therapy.

In contrast to TGs, there is no trend observed for changes in PCs with very long vs. long acyl chains. The majority of PCs which are increased in FXN-KO mice are also increased following treatment with FXN replacement therapeutic compound. Only two PCs two PCs are decreased in FXN-KO mice and are increased following treatment with FXN replacement therapeutic compound, and both have very long chains.

### Example 3. Lipidomic Analysis of Samples from FRDA Subjects Treated with FXN Replacement Therapeutic Compound

The goal of this experiment was to identify lipids that are modulated (i.e., levels of lipids are increased or decreased) in subjects with Friedreich's Ataxia (FRDA) following treatment with FXN replacement therapy. To this end, plasma samples were collected from 27 human subjects with Friedreich's Ataxia (FRDA) who were administered placebo (7 subjects) or different doses of an exemplary FXN replacement therapeutic compound as follows: Group 1 received a dose of 25 mg (5 subjects), Group 2 received a dose of 50 mg (6 subjects) and Group 3 received a dose of 100 mg (7 subjects) of the exemplary FXN replacement therapeutic compound. The lipidomic analysis involved comparing samples from each FRDA patient obtained two days prior to the dose (day −2) and 15 days after the dose (day 15). The lipidomic analysis also involved comparing samples from each FRDA patient prior to the dose (day −2) to samples from healthy controls (30 subjects).

Lipids were extracted from the collected samples and analyzed according to a validated protocol previously described in J. of Proteome Research 2018, 17(11):3657-3670. This is a comprehensive label-free untargeted semi-quantitative high-resolution LC-QTOF-based workflow, which is optimized for coverage of polar and non-polar lipids, as well as resolution of their isomers. It enables measurement of reproducible >1,500 high-quality MS signals or features, defined by mass/charge (m/z) ratio, retention time and signal intensity from 100 μL of plasma or serum with a single instrument (0.33 μL of volume equivalent injected).

### Instrumentation:

LC-MS analyses were performed using a high-resolution LC-QTOF 6530 Agilent operating in positive and negative mode. Lipids were eluted on a Zorbax Eclipse plus C18 column.

### Sample Preparation

The samples were randomized and analyzed in batches to control for batch-to-batch variation. Lipids were extracted and analyzed according to the validated protocol described in J. of Proteome Research 2018, 17(11):3657-3670. Samples were split into 4 aliquots, and stored at −80° C. The volume of the injected sample was 1.4 μL in positive ionization mode and 2.1 μL in negative ionization mode, corresponding, respectively, to 0.47 μL and 0.70 μL of plasma equivalent. Samples were injected in the same order as batch extraction to allow a better correction.

### Quality Controls of Sample Analysis

Internal standards were spiked into the plasma samples before the extraction, and monitored during LC-MS analysis. A plasma QC sample was injected at the beginning, in the middle and at the end of the LC-MS sequence analysis in positive mode and in negative mode for a total of 5 QC run analysis. Table 13 below lists the results of quality control analyses.

TABLE 13
Results of quality control analyses

| | Samples | | | | | | | |
|---|---|---|---|---|---|---|---|---|
| | Positive mode | | | | Negative mode | | | |
| | m/z | RT (min) | RSD | Mean | m/z | RT (min) | RSD | Mean |
| PS(12:0/12:0) | 624.389 | 12.24 | 13.30 | 1758003 | 622.372 | 12.25 | 8.07 | 1072943 |
| PC(14:0/14:0) | 678.508 | 17.99 | 11.71 | 30615704 | 722.498 | 18.00 | 8.00 | 1991441 |
| PG(15:0/15:0) | 712.514 | 18.67 | 15.19 | 510287 | 693.471 | 18.68 | 8.41 | 1285095 |
| PC(19:0/19:0) | 818.666 | 36.14 | 14.84 | 46339181 | 862.654 | 36.13 | 8.85 | 3533302 |

The plasma QC samples (n=5) showed a median relative standard deviation (median RSD) of 17.87% in the positive mode and 11.66% in the negative mode. The internal standards and QC samples met criteria in term of variability and signal intensity.

### Data Processing

Raw data for each ionization mode was processed using the data processing workflow as previously described in J. of Proteome Research 2018, 17(11):3657-3670. Briefly, data processing included the following steps to ensure high quality data.

1) Filter of frequency: data was present at least 80% in one condition (example: Group 1 treated with the exemplary FXN replacement therapeutic compound). If a specific lipid was present in 80% of samples from a group in at least one condition, it was included in the analysis; otherwise it was excluded.

2) Normalization using cyclic loess algorithm using all MS scan signals present after filtering. This normalization, which is commonly used for microarray data analysis, was selected because it generates good alignment of signals, while allowing some variability in the distributions, and is, therefore, more flexible than other normalization algorithms such as Quantile.

3) Imputation of missing values using Knn. This step was found to be crucial to ensure robustness of subsequent statistical analyses, particularly with small datasets. Values are often not missing at random, and missing data can occur for different reasons. For imputation of missing values, we have selected k-nearest neighbor algorithm (k=5), which classifies the data based on similarities measures, and it was previously shown to work best with strongly correlated features, such as lipids.

4) Correction for batch of sample preparation (sample lipid extraction). Addition of this step was found to minimize variations due to sample processing, principally at the lipid extraction step. This is attributed to the inherent handling samples on several days by different persons.

The raw data corrected from positive ionization and negative ionization were merged together, producing a datafile of 3228 features (defined as signal intensity, mass/charge, and retention time).

### Statistical Analysis

A total of 3228 signals (lipid features) remained after data processing, of which ˜50% represents potential unique lipids; the remainder are dimers, fragments, adducts, contamination, etc. At this step, those signals (features) are not annotated/identified.

Exploratory principal component analysis (PCA) was achieved using PC1 vs. PC2 and PC1 vs. PC3 on the ratio data.

Comparison between samples at day 15 vs. day −2 was performed using paired t-test on each feature, for each group (dose). A Storey correction was used to estimate the False Discovery Rate (FDR, Q-value) in Group 3. A significant threshold was set at P-value=0.03, corresponding to a Q-value=0.05 for comparison of day 15 vs. day-2 of Group 3. The subjective threshold p-value=0.03 was used then for comparison in each group.

Pearson correlation was used for correlating frataxin levels and certain lipid classes (triglycerides (TGs) and cholesteryl esters (CEs)). The Pearson correlation corresponds to a linear relationship among the same pairs of variables in the population. A subjective high threshold p-value <0.2 was used to discern a trend among lipids selected.

Linear regression analysis was used to correlate each feature with frataxin levels. The following criteria were used as subjective threshold: p-value <0.05. Linear regression analysis enables the identification and characterization of relationships among multiple factors. For this analysis only 1 variable was used: frataxin level, so the p-values calculated for this regression analysis were the same as the Pearson correlation analysis. A more stringent threshold was used for this regression analysis because all features (>3000) were included, which is more a discovery-based analysis, compared to Pearson analysis, which was focused only on significant TGs and CEs.

Network analysis using Fruchterman-Reingold Method was used to determine positive correlation between associated features. Positive correlation between associated features was projected onto a 2-dimensional display using Fruchterman-Reingold layout algorithm (R package igraph: Csardi G, Nepusz T. The igraph software package for complex network research. InterJournal 2006; Complex Systems:1695). A starting point was given to the algorithm as the first two principal component analysis coordinates.

### Lipid Annotations

For lipid annotation, an in-house database was used. The database is composed of more than 450 lipids previously annotated by MS/MS in human plasma, using an algorithm to align mass and retention time. The database is principally composed of phospholipids and sphingolipids with their acyl chains and TGs with the sum of the 3 acyl chains. For the remaining lipids that were not annotated but were determined to be significantly different between day 15 and day −2, MS/MS analysis was performed to identify the lipid class and acyl chains when it was possible. Since acyl side chain positions were not validated with synthetic standards, phospholipids and glycerolipids were annotated along with the identified structure for the acyl side chains using the underscore convention for sn1 and sn2.

### Results

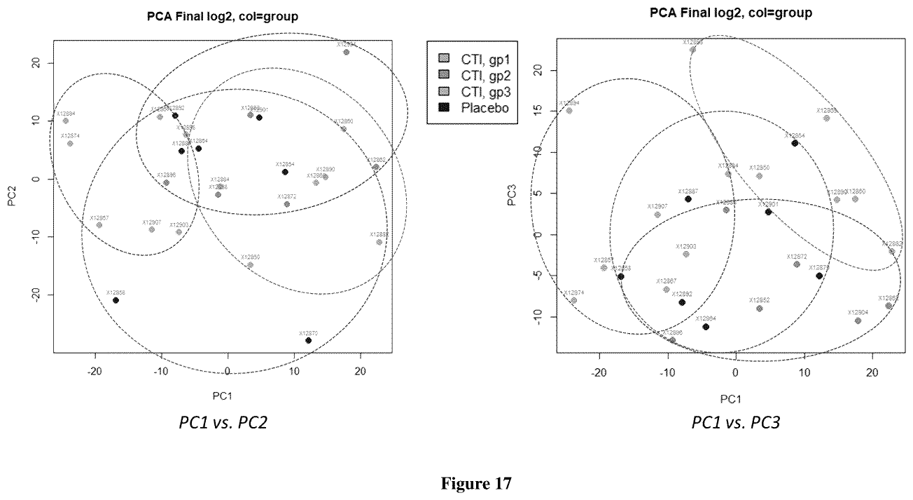

FIG. 17 is a scatter plot showing the results of Principal Component Analysis (PCA) of ratio of day 15/day −2 for all samples with MS signals, colored by group (Group 1, Group 2 and Group 3). The PCA analysis reveals a well-defined separation between Group 1 and Group 3 (subjects treated with 25 mg and 100 mg of the exemplary FXN replacement therapeutic compound).

Comparison Day 15 vs. Day −2

The comparisons are based on the intensity data of day 15 vs. day −2. A threshold p-value=0.03 was chosen based on the comparison of Group 3 (Paired t-test day 15 vs. day-2), corresponding to a Q-value=0.05. The same threshold was chosen in order to compare the group effect.

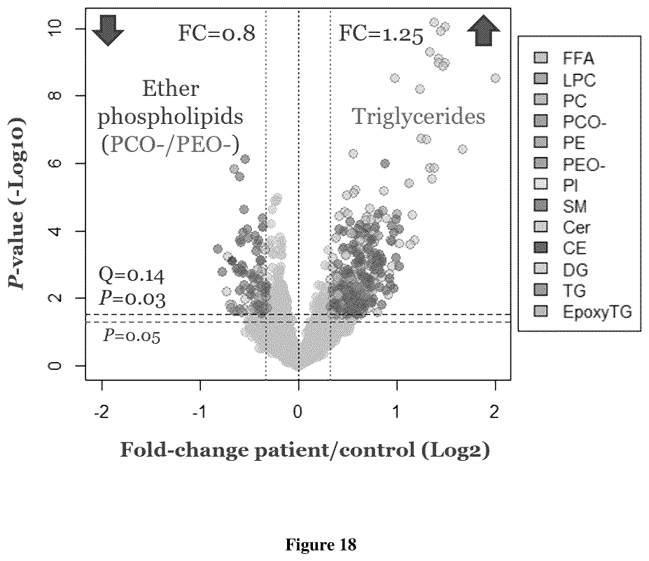

FIG. 18 is a volcano plot presenting the comparison for healthy human subjects vs. subjects with FRDA at day −2 (i.e., prior to treatment). The different lipid subclasses are indicated by different colors. Gray dots represent lipids that are were not identified. The horizontal lines indicates the threshold p-values of 0.03 and 0.05, and the vertical red lines represent fold change of 0.8 and 1.25

The results presented in FIG. 18 indicate that in FRDA subjects prior to treatment there are significant perturbations of plasma lipids from various subclasses as compared to healthy control subjects. Specifically, levels of TGs are increased in FRDA subjects as compared to healthy control subjects, while the levels of ether phospholipids, such as PCO- and PEO-, are decreased in FRDA subjects vs. healthy control subjects. The results presented in FIG. 18 are consistent with the results of Example 1 (see, e.g., Tables 5 and 6).

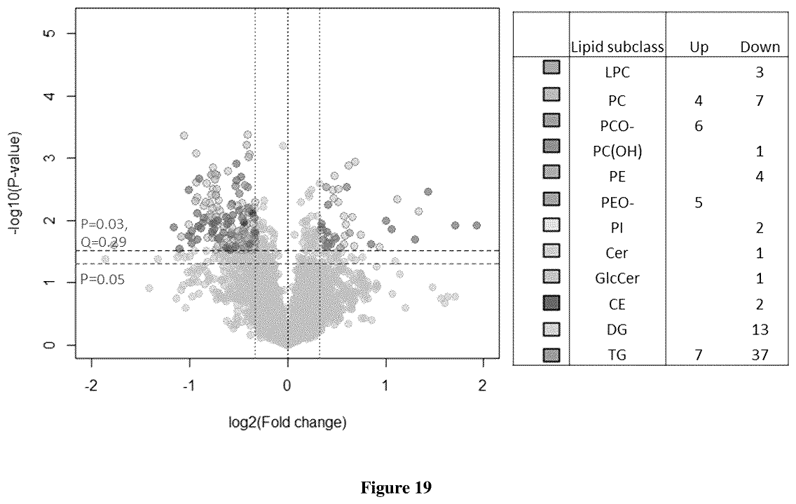

FIG. 19 is a volcano plot presenting the comparison for day 15 vs. day −2 for subjects who received placebo. Of all 3328 features, 200 discriminate day 15 vs. day −2 for subjects who received placebo, with a p-value <0.03 and a fold change of 1.25. From these 200 features, 93 were annotated to a lipid ID: 44 features (22 annotated) were increased, and 156 features (71 annotated) were decreased with placebo between day 15 and day −2.

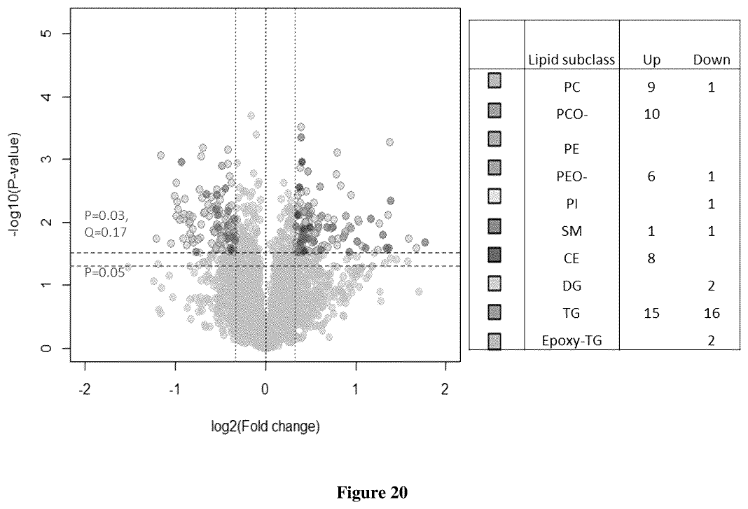

FIG. 20 is a volcano plot presenting the comparison for day 15 vs. day −2 for subjects of Group 1 (dosed with 25 mg of the exemplary FXN replacement therapeutic compound). Of all 3328 features, 237 discriminate day 15 vs. day-2, with a p-value <0.03 and a fold change of 1.25. From these 237 features, 73 were annotated to a lipid ID: 125 features (49 annotated) were increased, and 112 features (24 annotated) were decreased at day 15 vs. day −2 after dosing with 25 mg of the exemplary FXN replacement therapeutic compound.

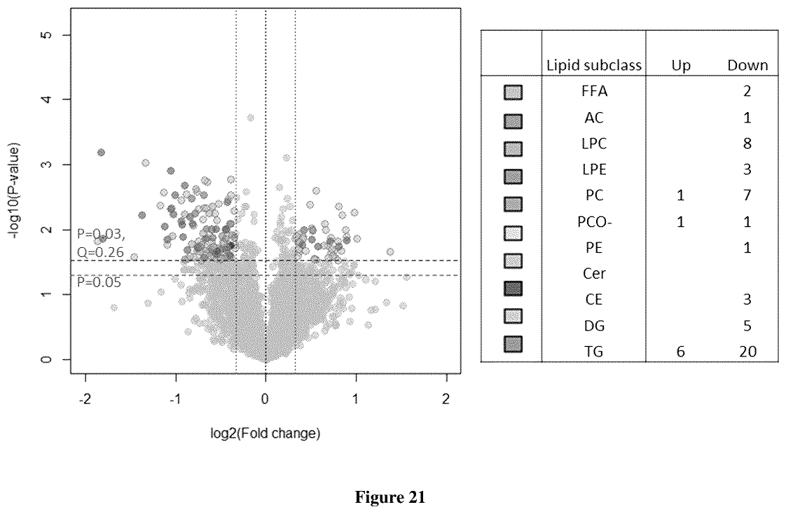

FIG. 21 is a volcano plot presenting the comparison for day 15 vs. day −2 for subjects of Group 2 (dosed with 50 mg of the exemplary FXN replacement therapeutic compound). Of all 3328 features, 169 discriminate day 15 vs. day-2, with a p-value <0.03 and a fold change of 1.25. From these 169 features, 59 were annotated to a lipid ID: 49 features (8 annotated) were increased, and 120 features (51 annotated) were decreased at day 15 vs. day −2 after dosing with 50 mg of the exemplary FXN replacement therapeutic compound.

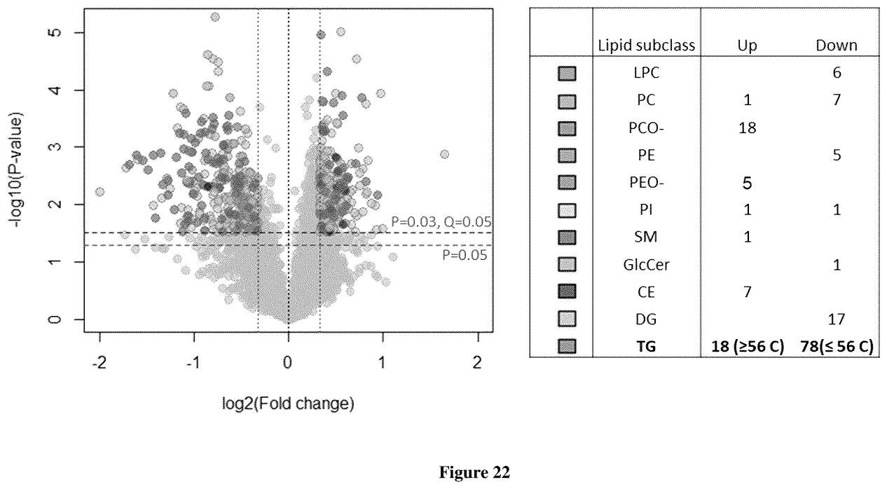

FIG. 22 is a volcano plot presenting the comparison for day 15 vs. day −2 for subjects of Group 3 (dosed with 100 mg of the exemplary FXN replacement therapeutic compound). Of all 3328 features, 506 discriminate day 15 vs. day-2, with a p-value <0.03 (Q-value<0.05) and a fold change of 1.25. From these 506 features, 166 were annotated to a lipid ID: 218 features (51 annotated) were increased, and 288 features (115 annotated) were decreased at day 15 vs. day −2 after dosing with 100 mg of the exemplary FXN replacement therapeutic compound. The effect of dosing with the exemplary FXN replacement therapeutic compound observed for Group 3 (506 significant features) is more pronounced than the effect observed for Groups 1, 2 and placebo (<250 significant features).

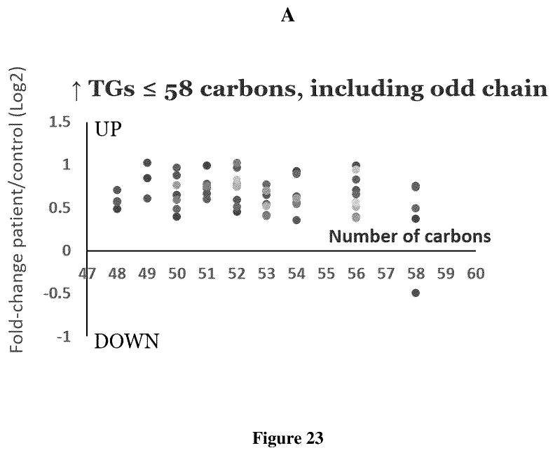

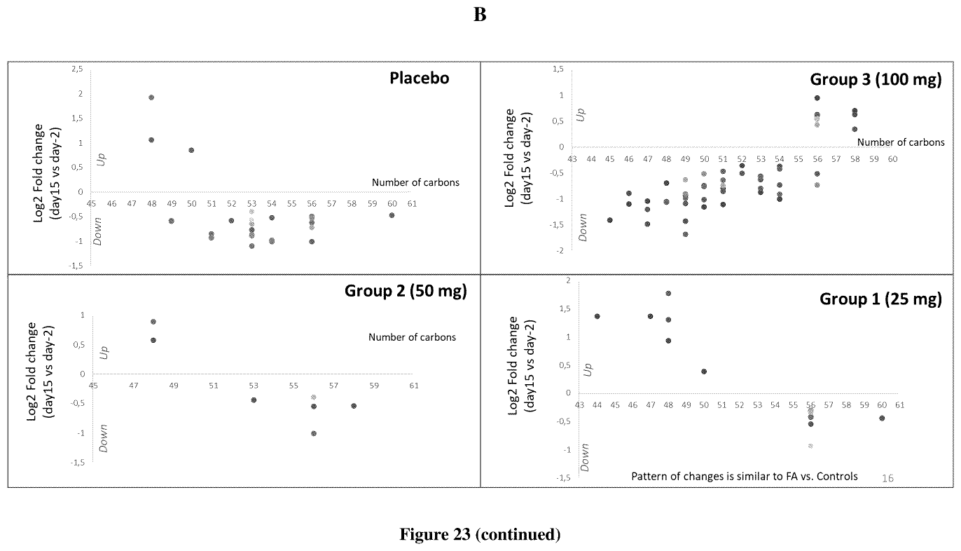

FIG. 23 is a series of dot plots showing log 2 fold change in the levels of TGs on day 15 vs. day −2 vs. the number of acyl chain carbons. Specifically, FIG. 23, Panel A is a dot plot showing log 2 fold change in the levels of TGs vs. the number of acyl chain carbons for samples from FRDA subjects at day −2 (prior to treatment) vs. healthy control subjects. FIG. 23, Panel B is a series of dot plots showing log 2 fold change in the levels of TGs on day 15 vs. day −2 vs. the number of acyl chain carbons for samples from subjects dosed with placebo, 100 mg (Group 3), 50 mg (Group 2) and 25 mg (Group 1) of the exemplary FXN replacement therapeutic compound. The TGs graphed in the dot plots are 49 unique TGs selected based on results for Group 3, with p-value<0.03, FC>1.25 or FC<0.8 for day 15 vs. day-2 for this group.

For Group 3, it was observed that TGs with a total number of acyl chain carbons <56 are decreased following dosing with the exemplary FXN replacement therapeutic compound, and those with a total number of acyl chain carbons ≥56 are increased following dosing with the exemplary FXN replacement therapeutic compound.

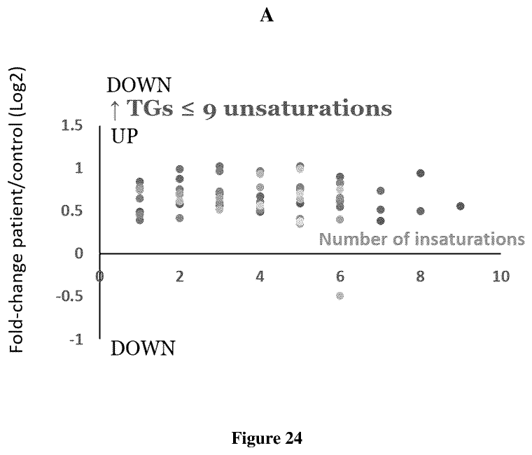

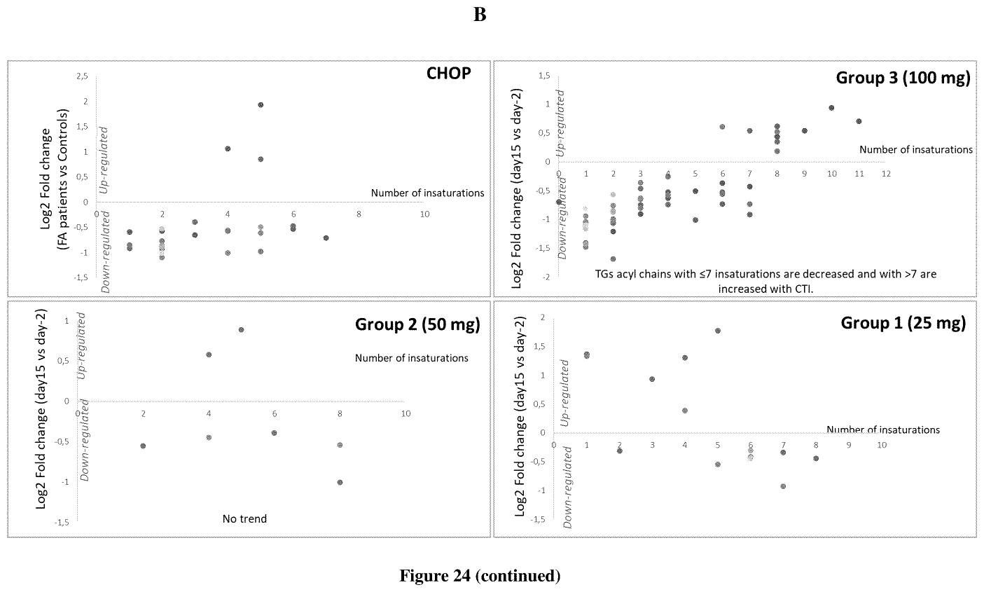

FIG. 24 is a series of dot plots showing log 2 fold change in the levels of TGs vs. the number of acyl chain unsaturations. Specifically, FIG. 24, Panel A is a dot plot showing log 2 fold change in the levels of TGs vs. the number of acyl chain unsaturations for samples from FRDA subjects at day −2 (prior to treatment) vs. healthy control subjects. FIG. 24, Panel B is a series of dot plots showing log 2 fold change in the level of TGs vs. the number of acyl chain unsaturations. The dot plot in the upper left quadrant is shown for FRDA subjects vs. healthy controls who were not dosed (see Example 1). The remaining dot plots are shown for samples from subjects dosed with 100 mg (Group 3), 50 mg (Group 2) and 25 mg (Group 1) of the exemplary FXN replacement therapeutic compound on day 15 after dosing vs. day −2 before dosing. The TGs graphed in the dot plots are 49 unique TGs selected based on results for Group 3, with p-value<0.03, FC>1.25 or FC<0.8 for day 15 vs. day-2 for this group.

For Group 3, it was observed that levels of TGs with >7 acyl chain unsaturations are increased following dosing with the exemplary FXN replacement therapeutic compound, while those with a total number of acyl chain unsaturations ≤7 are decreased following dosing with the exemplary FXN replacement therapeutic compound.

The results presented in FIGS. 22 and 23 indicate that there is acyl chain remodeling of TGs in samples from subjects treated with 100 mg of the exemplary FXN replacement therapeutic compound. This effect is not observed in samples from subjects treated with placebo, 25 mg and 50 mg of the exemplary replacement therapeutic compound.

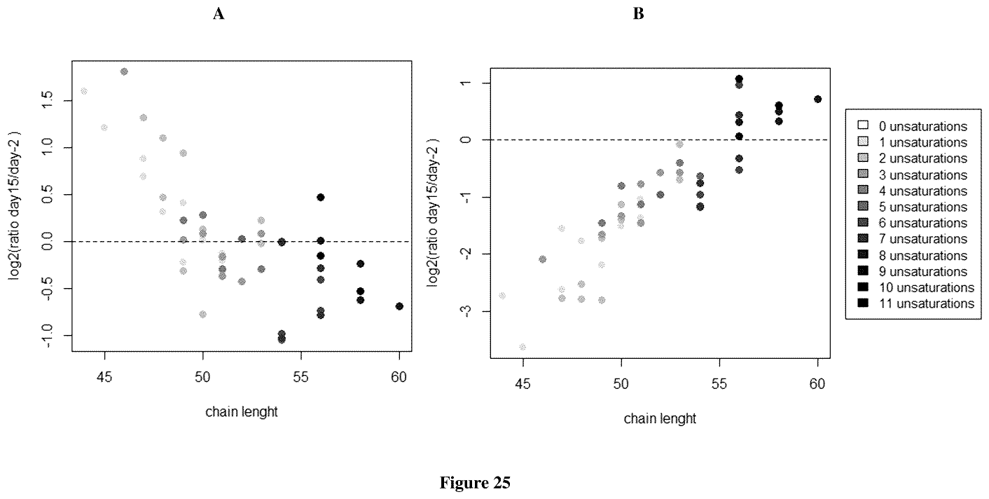

FIG. 25, panel A is a dot plot showing log 2 fold change in the levels of 49 unique TGs vs. the number of acyl chain carbons in sample from a representative patient treated with placebo. FIG. 25, panel B is a dot plot showing log 2 fold change in the levels of 49 unique TGs vs. the number of acyl chain carbons in sample from a representative patient treated with 100 mg of the exemplary FXN replacement therapeutic compound. The number of unsaturations in each TG is presented using grayscale, with white dots representing 0 unsaturations and darkers dots representing an increasing number of unsaturations. The results presented in FIG. 25 illustrate acyl chain remodeling of TGs in samples from subjects treated with 100 mg of the exemplary FXN replacement therapeutic compound.

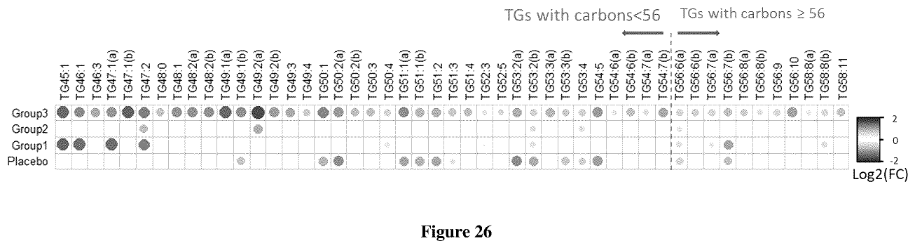

FIG. 26 is a graphic summary of the comparisons of day 15 vs. day −2 for subjects treated with placebo, 25 mg (Group 1), 50 mg (Group 2) and 100 mg (Group 3) of the exemplary FXN replacement therapeutic compound for 49 TGs. The 49 TGs have been selected as being most significantly modulated TGs for Group 3. Dot intensity represents fold changes in log 2 of day 15 vs. day −2 for each group with p-value <0.05. A red dot represents an increase with the treatment, while a blue dot represents a decrease with the treatment. The dot color intensity is proportional to the value of fold-change: the stronger the color intensity, the larger the fold change.

The results presented in FIG. 26 indicate that for Group 3, the levels of TGs with acyl chain length <56 is decreased, while the levels of TGs with acyl chain length >56 is increased as compared to the levels of TGs for the placebo-treated group.

Ether phospholipids, e.g., PCO- and PEO-, were found to be decreased in FRDA subjects prior to treatment, as compared to healthy controls, and increased following FXN replacement therapy. Table 14 below presents the results for the selected PCO- species.

TABLE 14
Levels of selected PCO— species in healthy FRDA subjects vs. healthy
control subjects and FRDA subjects before and after FXN replacement therapy.

| | | FRDA subjects at Day −2 vs. | | | |
|---|---|---|---|---|---|
| | | healthy control subjects* | | Group 3: Day 15 vs. Day −2 | |
| | Lipid | Log₂ fold change | | Log₂ fold change | |
| Feature | Annotation | (FRDA/control) | p-value | (Day 15/Day −2) | p-value |
| POS: 743.5837@24.22 | PC(O-16:0/18:2) | −0.44 | 1.44E−04 | 0.57 | 1.28E−04 |
| POS: 791.5831@23.05 | PC(O-16:0/22:6) | −0.60 | 1.87E−04 | 0.26 | 7.85E−03 |
| POS: 771.6142@28.12 | PC(O-18:0/18:2) | −0.54 | 7.67E−07 | 0.25 | 3.88E−02 |
| POS: 791.5828@22.01 | PC(O-18:1/20:5) | −0.63 | 1.28E−03 | 0.38 | 2.51E−02 |
| POS: 769.5992@24.74 | PCO-36:3 | −0.38 | 7.75E−04 | 0.37 | 1.59E−04 |
| NEG: 789.5875@22.26 | PCO-34:2 | −0.38 | 7.25E−04 | 0.39 | 5.28E−04 |
| POS: 827.6779@35.56 | PCO-40:2 | −0.44 | 1.72E−02 | 0.39 | 1.80E−02 |
| POS: 873.6603@30.96 | PCO-44:7 | −0.41 | 1.48E−02 | 0.43 | 3.88E−02 |

*Lipid features were selected based on the significance of the FRDA subjects vs. control subjects value: p
value <0.03; |fold change| >1.25.

The results presented in Table 14 demonstrate that levels of the PCO- species are decreased in FRDA subjects vs. healthy control subjects and that levels of PCO- species increase in FRDA subjects following administration of FXN replacement therapy.

### Pearson Correlations

Pearson correlations were calculated for each lipid feature with its corresponding p-value in order to correlate levels of lipids, such as TGs and CEs, with frataxin levels measured in the skin, platelets and buccal cells from each subject. A subjective threshold of p-value=0.2 was chosen.

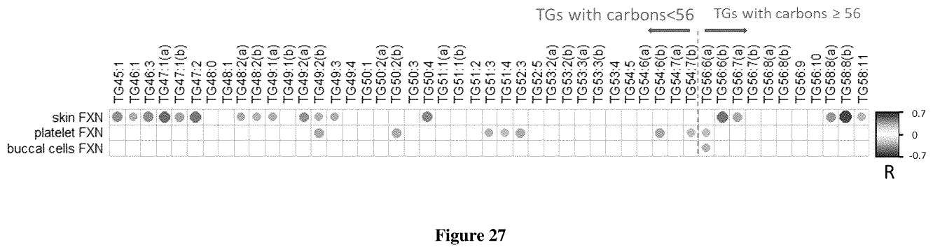

FIG. 27 is a graphic summary of the correlation between frataxin levels and the levels of selected 49 TGs that were most significantly modulated in Group 3. The intensity of each dot reflects the strength of correlations between levels of lipids and frataxin levels with p-value <0.2, with blue dot representing negative correlation and red dot representing positive correlation.

The results presented in FIG. 27 indicate that TGs with acyl chain length ≥56 carbons correlate positively with frataxin levels measured in skin, while TGs with acyl chain length <56 carbons correlate negatively with frataxin levels measured in skin. There is only a small number of TGs that correlate with frataxin levels in platelets or buccal cells.

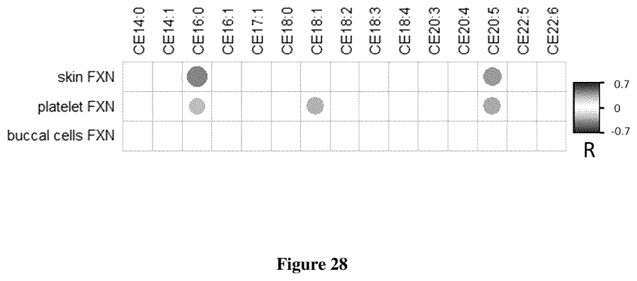

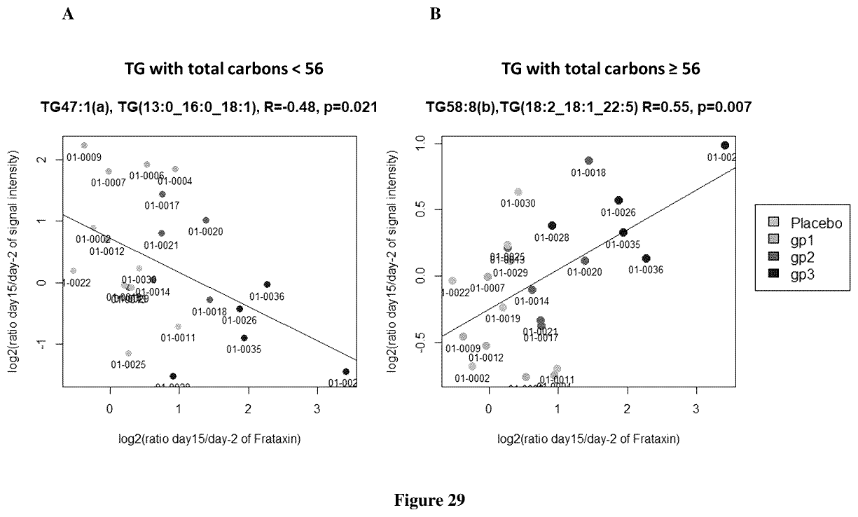

FIG. 28 is a graphic summary of the correlation between frataxin levels and the levels of cholesteryls esters (CE). The results presented in FIG. 28 indicate that only CE16:0 and CE20:5 are correlated in a positive manner with frataxin levels from skin, with p-value <0.2, and to a lesser extent with frataxin levels in platelets.

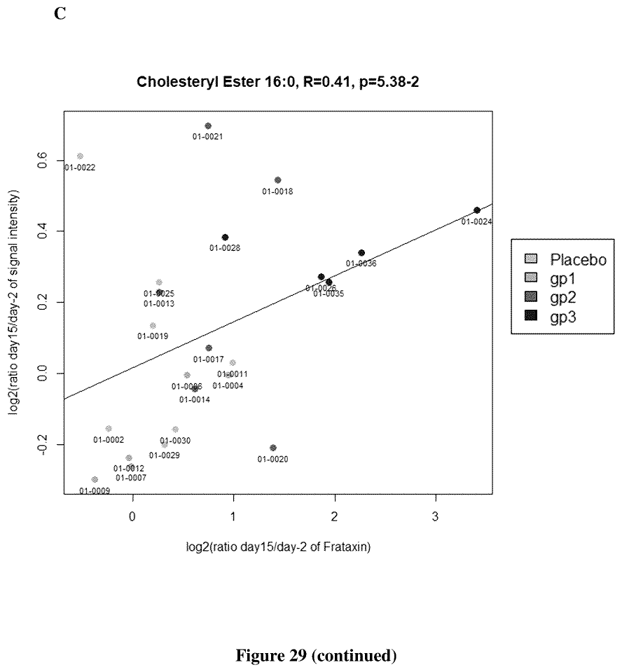

FIG. 29, panel A is a dot plot of ratio of lipid levels at day 15 vs. day −2 vs. ratio of frataxin levels in skin samples at day 15 vs. day −2 for representative TGs with acyl chain length <56 carbons. FIG. 29, panel B is a dot plot of ratio of lipid levels at day 15 vs. day −2 vs. ratio of frataxin levels in skin samples at day 15 vs. day −2 for representative TGs with acyl chain length ≥56 carbons. FIG. 29, panel C is a dot plot of ratio of lipid levels at day 15 vs. day −2 vs. ratio of frataxin levels in skin samples at day 15 vs. day −2 for CD16:0.

The results presented in FIG. 29, panel A illustrate that there is a negative correlation between frataxin levels in skin and levels of TGs with acyl chain length <56 carbons. The results presented in FIG. 29, panel B illustrate that there is a positive correlation between frataxin levels in skin and levels of TGs with acyl chain length >56 carbons. The results presented in FIG. 29, panel C illustrate that there is a positive correlation between frataxin levels in skin and levels of CE16:0.

A linear regression analysis using log 2 of levels of frataxin in skin was performed on all features, independent of their significance with respect to the impact of treatment with the exemplary FXN replacement therapeutic compound. The goal of this analysis was to determine if lipids other than TGs and CEs are correlated with frataxin levels in skin.

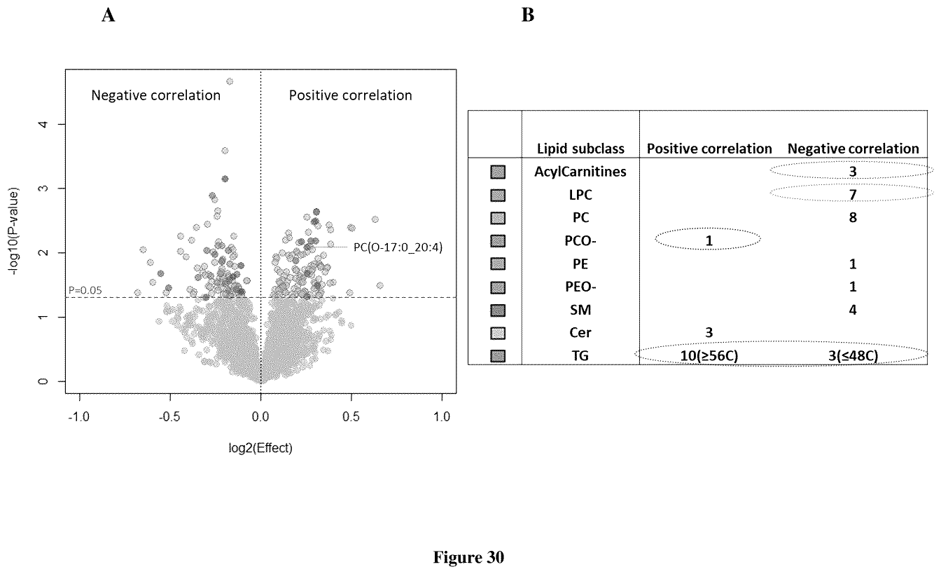

The results of the analysis are presented in the in FIG. 30. Specifically, FIG. 30, panel A is a volcano plot showing an effect (positive or negative) and the associated p-value for each feature. The threshold chosen was p-value=0.05, which is more stringent than for the Pearson correlation analysis, since the goal was to identify the most significant lipids that correlate with frataxin levels in skin other than TGs and CEs. FIG. 30, panel B is a summary table of the data presented in the volcano plot in FIG. 30, panel A.

The results presented in FIG. 30, panels A and B indicate that of all 3328 features, 218 correlate with frataxin levels in skin with a p-value<0.05. From these 218 features, 41 were annotated to a lipid ID: 119 features (14 annotated) were positively associated, and 99 features (27 annotated) were negatively associated with frataxin levels in skin. The results show that, besides TGs and CEs, the following lipid subclasses also correlate with frataxin levels in skin: 1) acylcarnitines (proxies of defects in mitochondrial fatty acid oxidation) and saturated LPC are negatively correlated with frataxin levels; and PC ethers (PC(O-17:0/20:4)) (reflecting peroxisomal metabolism) are positively correlated with frataxin levels.

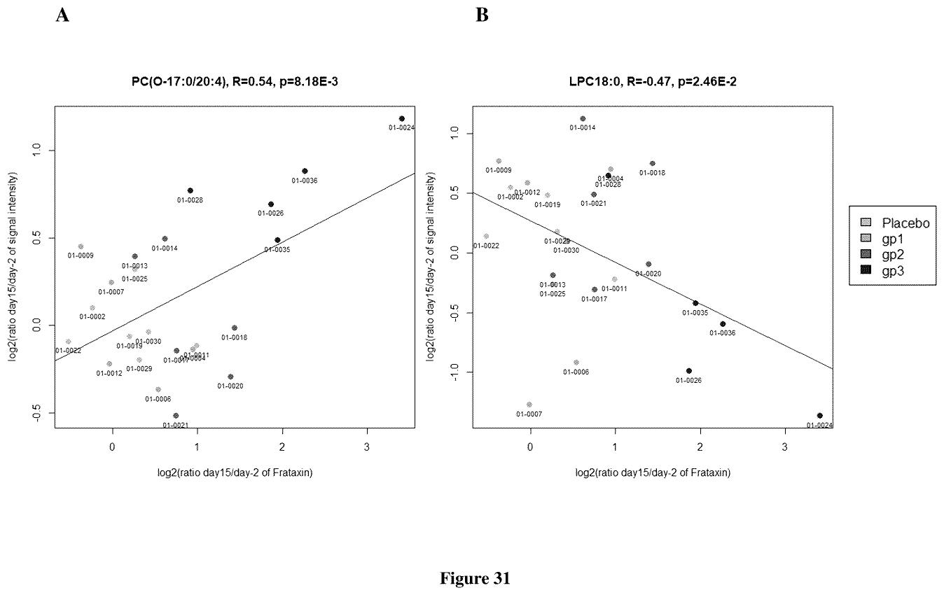

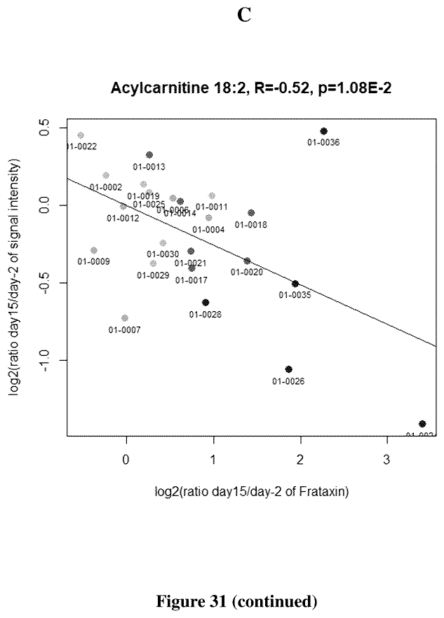

FIG. 31, panel A is a dot plot of ratio of lipid levels at day 15 vs. day −2 vs. ratio of frataxin levels in skin samples at day 15 vs. day −2 for a representative PC ether PC(O-17:0/20:4). FIG. 31, panel B is a dot plot of ratio of lipid levels at day 15 vs. day −2 vs. ratio of frataxin levels in skin samples at day 15 vs. day −2 for a representative LPC18:0. FIG. 31, panel C is a dot plot of ratio of lipid levels at day 15 vs. day −2 vs. ratio of frataxin levels in skin samples at day 15 vs. day −2 for a representative acylcarnitine 18:2.

The results presented in FIG. 31 indicate that levels of PC(O-17:0/20:4) are positively correlated with the frataxin levels in skin. This lipid contains an odd number of carbons in the acyl chain (C17:0), which may arise as a result of microbial metabolism. This lipid is also significantly modulated at day 15 vs. day −2 in Group 3, but is not significantly modulated in the placebo group.

The results presented in FIG. 31 also indicate that levels of LPC18:0 and acylcartinine 18:2 are negatively correlated with frataxin levels in skin.

### Network Analysis Using Fruchterman-Reingold Method

The network analysis was conducted using annotated lipids that were shown to be differentially expressed at day 15 vs. day −2 in subjects who received 100 mg of the exemplary FXN replacement therapeutic compound (Group 3). Positive correlation (R>0.6) between day vs. day −2 associated features was projected onto a 2-dimensional display using Fruchterman-Reingold layout algorithm.

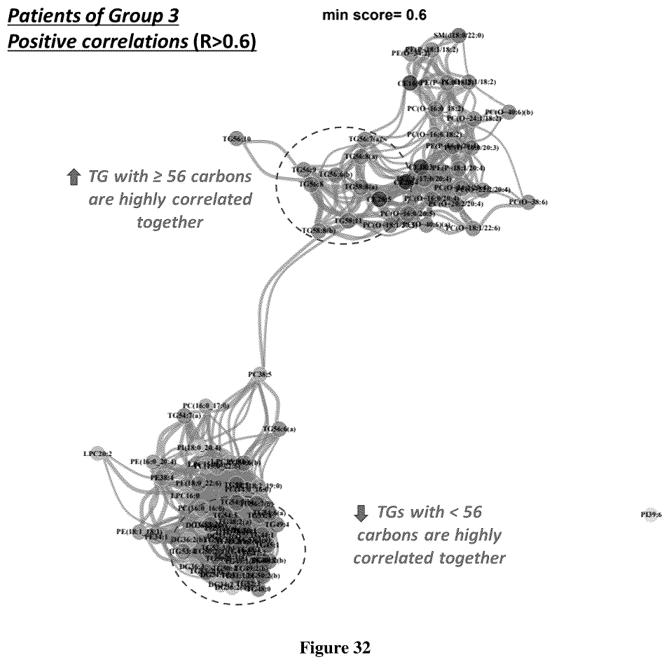

FIG. 32 is an illustration of a 2-dimensional display obtained using the Fruchterman-Reingold layout algorithm of positive correlations between annotated lipids differentially expressed at day 15 vs. day −2 in subjects treated with 100 mg of the exemplary FXN replacement therapeutic compound (Group 3). Edges represent connections or correlations (R>0.6) between the nodes (lipids). The width of the link is proportional to the correlation.

The results presented in FIG. 32 indicate that although there are more than 100 lipids that are differentially expressed between two conditions, there are only two main, interconnected, correlation clusters or networks (groups of strongly correlated lipids) explaining the difference between the two time points, likely reflecting a common mechanism. The first cluster includes TGs with acyl chain length ≥56 carbons that are highly correlated together and are increased at day 15 vs. day −2 following treatment with 100 mg of the exemplary FXN replacement therapeutic compound. The second cluster includes TGs with acyl chain length <56 carbons and are decreased at day 15 vs. day −2 following treatment with 100 mg of the exemplary FXN replacement therapeutic compound.

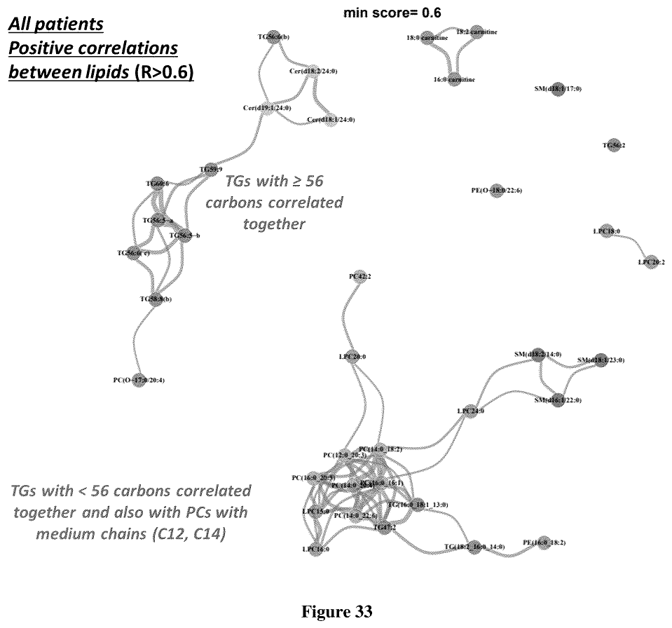

FIG. 33 is an illustration of a 2-dimensional display obtained using the Fruchterman-Reingold layout algorithm of positive correlations between annotated lipids associated with frataxin.

The results presented in FIG. 33 indicate that a limited number of correlation clusters of lipids are associated with frataxin, including one for TGs with acyl chain length ≥56 carbons. The correlation clusters also include ceramides (Cer) with very long acyl chains (≥22 carbons), a PC ether with C17:0 and a separate one for TGs with acyl chain length <56 that includes PC with medium chain fatty acyl side chain (12 and 14 carbons). There is also an additional correlation cluster that includes all acylcarnitines with long chains (16 and 18 carbons), which was not observed for the comparison between day 15 vs. day-2 for Group 3. FIG. 33 demonstrates that, although there are 37 unique lipids that are associated with frataxin levels, most of these lipids are highly correlated. Some lipids are common to lipids differentially expressed in subjects who received 100 mg of the exemplary FXN replacement therapeutic compound at day 15 vs. day-2 with a p-value <0.05.

### Conclusions

The results of Example 3 indicate that TGs with acyl chain length <56 carbons are decreased and TGs with acyl chain length ≥56 are increased at day 15 vs day −2 in subjects treated with 100 mg of the exemplary FXN replacement therapeutic compound (Group 3). Complete annotation using MS/MS of acyl chains in TGs with acyl chain length ≥56 carbons reveals that they all contain a C22:5 or C20:5. The pattern of TG remodeling observed for subjects in Group 3 is consistent with their correlation with frataxin levels in skin.

Cholesteryl esters (CEs) follow a pattern of changes similar to those of TGs with acyl chain length >56. Accordingly, CEs and TGs belong to the same correlation cluster (R>0.6). Among CEs, CE 16:0 and CE 20:5 are also correlated positively with frataxin levels in skin, but above the threshold p-value (p=0.054 and 0.099 respectively).

Ether phospholipids, e.g., PCO- and PEO-, were found to be decreased in FRDA subjects as compared to healthy controls, and increased following FXN replacement therapy.

Phosphatidylcholines (PC) with acyl chains containing 12 or 14 carbons (i.e., medium chain length fatty acids) were found to be correlated negatively with frataxin levels in skin and TGs with acyl chain length <56 carbons. Some of PCs show a significant decrease between day vs. day-2 in Group 3.

Phosphatidylcholine ethers were found to be increased at day 15 vs. day −2 for Group 3. This is especially evident for PC(O-17:0/20:4), which correlated positively with frataxin levels in skin and showed strong correlation (R>0.6) with TGs with acyl chain length ≥56 carbons, phosphatidylethanolamine ethers (PEO-), and CEs. Some PC ethers are also affected in the placebo group, but not PC(O-17:0/20:4).

Lysophosphatidylcholines (LPCs), mostly with a saturated fatty acyl chains, were found to be negatively associated with frataxin levels in skin.

Acylcarnitines (ACs), specifically long chain ACs, were found also to be negatively associated with frataxin levels in skin.

Altogether, the observed correlations (positive or negative) between the aforementioned changes in circulating lipid subclasses with frataxin levels in skin in subjects following treatment with the exemplary FXN replacement therapeutic compound support a role of frataxin in mediating the reported changes in the circulating levels of these lipid subclasses.

Given that the systemic lipid changes observed can be explained by a limited number of correlation clusters of lipids or group of lipids, it can be proposed that they could potentially be summarized by a composite global score that remains to be defined, which would likely reflect a limited number of different metabolic mechanisms modulated by frataxin. For example, TG acyl chain remodeling, specifically a shift from TGs with acyl chain length ≥56 carbons (highly unsaturated) to TGs with acyl chain length <56 carbons (mostly saturated), has been reported in humans and linked to insulin resistance (see Rhee et al., J. Clin. Invest. 2018, 121:1402). TGs with acyl chain length <56 carbons include acyl chains that are predominantly saturated fatty acids, such as lauric acid (C12:0), myristic acid (C14:0), palmitic acid (C16:0), stearic acid (C18:0) and oleic acid (C18:1). This reflects de novo lipogenesis, most likely predominantly in liver, but the involvement of other tissues is not excluded. Enhanced de novo lipogenesis in FRDA patients is also supported by the presence of PCs with saturated medium length chain fatty acids (12 and 14 carbons) in the same correlation clusters as TGs with acyl chain length <56 carbons.

Circulating levels of long chain ACs are recognized proxies of defects in mitochondrial fatty acid oxidation (Review: McCoin et al., Nat. Rev. Endocrinol. 2015, 11:617).

The aforementioned metabolic mechanisms (mitochondrial metabolic dysfunction, enhanced lipogenesis and insulin resistance) concur with the reported metabolic perturbations resulting from frataxin deficiency in in a mouse model Friedreich's ataxia (FRDA; Turchi et al., Cell Death and Disease 2020, 11:51). Furthermore, the metabolic mechanisms are also consistent with the reported metabolic reprogramming of mammalian cells following acute loss of iron-sulfur clusters, which are essential parts of several mitochondrial enzymes, including mitochondrial respiratory chain complexes and aconitase, whose mitochondrial synthesis is governed by frataxin (Crooks et al., J. Biol. Chem. 2018, 293(21):8297).

### Example 4. Lipidomic Analysis of Samples from FRDA Subjects Treated with FXN Replacement Therapeutic Compound and Healthy Volunteers

The goal of this experiment was: a) assess the difference in baseline levels of select lipid subclasses between untreated FRDA subjects and healthy volunteers; b) analyze a second aliquot of the samples used in the experiment described in Example 3 in order to determine reproducibility of results described in Example 3; and c) assess the impact of treatment by comparing lipid levels in FRDA subjects at baseline (Day −2) and at Day 15, and the impact of disease by comparing lipid levels at baseline (Day −2) in FRDA subjects and healthy volunteers.

### Methods

The validation of the metabolomics platform lipidomic workflow used in the experiment was previously reported for 48 replicates of a single human plasma sample (Forest et al., J. of Proteome Research 2018, 17(11):3657-3670). This is a comprehensive, label-free, untargeted, semi-quantitative, high-resolution liquid chromatography (LC) quadrupole time-of-flight-(QTOF) based workflow optimized for coverage of polar and nonpolar lipids, as well as resolution of their isomers. It enables measurement of reproducible >1500 high-quality mass spectrometry (MS) signals or features, defined by mass/charge (m/z) ratio, retention time (RT), and signal intensity from 100 μL of plasma or serum with a single instrument (0.33 μL of volume equivalent injected).

MS signal intensity spanned 4 orders of magnitude for 92% of these features and had a relative standard deviation (RSD)<30% inside their linear intensity range. Half of the features were reported to be redundant due to adducts, dimers, in-source fragmentation, contaminations, or positive and negative ion duplicates. To date, 509 structurally unique lipid molecular species covering 25 lipid subclasses have been annotated by tandem mass spectrometry (MS/MS) analyses across different studies in human plasma in the MHI in-house database. Median inter- and intra-assay coefficients of variations (or RSD) for the resulting annotated lipids meet the upper criteria tolerated by the Food and Drug Administration (i.e., >80% lipid with RSD <20%) thereby enabling robust semi-quantitation.

Cohorts and dosing regimen of the exemplary FXN replacement therapeutic compound for the subjects involved in the study are shown in Table 15 below.

TABLE 15
Cohorts and Dosing Regimen

| | Dose | Subjects | |
|---|---|---|---|
| Cohort | (mg) | (n) | Dosing Regimen |
| 1 | 25 | 6 | Once daily Days 1 through 4; |
| | Placebo | 2 | every third day until Day 13 |
| 2 | 50 | 7 | Once daily Days 1 through 7; |
| | Placebo | 2 | every other day until Day 13 |
| 3 | 100 | 7 | Once daily Days 1 through 13 |
| | Placebo | 3 | |
| NA | — | 31^{b} | Non-interventional study; samples |
| | | | collected from healthy |
| | | | volunteers at T₀ |
| NA | — | 31^{b} | Non-interventional study; samples |
| | | | collected from healthy volunteers |
| | | | at T₃ = 3-hour time point |

### Instrumentation

LC-MS/MS analyses were done using a high-resolution LC-QTOF 6550 Agilent operating in positive and negative mode. Lipids were eluted on a Zorbax Eclipse plus C18 column.

### Sample Preparation

Plasma samples collected from FRDA subjects described in Example 3 and healthy volunteers were randomized. Lipids were extracted and analyzed according to the workflow described above. Each sample extract was split into 4 aliquots, and stored at −80° C. Samples were analyzed at injection volumes of 1 μL in positive ionization mode and 2 μL in negative ionization mode, corresponding to 0.33 μL and 0.67 μL, respectively, of plasma equivalent. Samples were injected in the same order as batch extraction to allow a better correction.

### Quality Controls of Sample Analysis

Internal standards (IS) were spiked into human plasma to prepare IS quality control (QC) samples before extraction. These IS QC samples were extracted and monitored at the beginning, middle, and end of the LC-MS analysis. A pooled plasma QC sample was also extracted and monitored as plasma QC. Both IS QC and plasma QC samples were injected during LC-MS analysis in positive mode and in negative mode (Table 16 and Table 17). The plasma QC samples correspond to human plasma samples from control subjects (healthy volunteers from MHI MAPoM project) collected using the same procedure and stored in aliquots at −80° C. The plasma QC sample results are presented for the raw data, corresponding to the original intensities value for features present in 80% of the samples, and for the corrected data, corresponding to correction of intensity data by normalization including imputation of missing values.

TABLE 16
Internal Standard QC Variability in Study Samples

| | Ionization Mode | | | | | | | |
|---|---|---|---|---|---|---|---|---|
| | Positive | | | | Negative | | | |
| Internal Standard | m/z | RT (min) | RSD (%) | Mean | m/z | RT (min) | RSD (%) | Mean |
| PS (12:0/12:0) | 624.389 | 12.00 | 15.78 | 1758003 | 622.372 | 10.14 | 15.09 | 3380673 |
| PC (14:0/14:0) | 678.508 | 17.30 | 11.71 | 30615704 | 722.498 | 15.38 | 15.44 | 2685887 |
| PG (15:0/15:0) | 712.514 | 18.01 | 13.06 | 510287 | 693.471 | 16.07 | 16.45 | 838241 |
| PC (19:0/19:0) | 818.666 | 35.37 | 18.72 | 46339181 | 862.654 | 33.37 | 14.91 | 3908792 |

Abbreviations: min = minutes; m/z = mass/charge; PC = phosphatidylcholine; PG = phosphatidylglycerol; PS = phosphatidylserine; QC = quality control; RSD = relative standard deviation; RT = retention time.

TABLE 17
Plasma QC Samples Variability Results

Ionization Mode

| | | Positive | Negative |
|---|---|---|---|
| | | (n = 11) | (n = 8)^{a} |
| Median RSD | Raw Data | 14.77% | 18.26% |
| | Data Corrected | 13.63% | 11.17% |

| | | |
|---|---|---|
| Total number of features | 2427 | 520 |
| Total number of features | 2115 | 471 |
| with RSD <30% | | |

Abbreviations: QC = quality control; RSD = relative standard deviation.
^{a}One QC analyzed in negative mode was lost (the third QC). For an unknown reason, no signal was acquired.

IS corrected for signal intensities had a median RSD <20%. Plasma QC samples met the variability criteria for the corrected data (median RSD <15%) and signal intensity (represented by the number of features detected with an RSD <30% in human plasma in 80% of samples >1300 in positive ionization mode and >350 in negative ionization mode).

### Data Processing

The raw data from each ionization mode were analyzed per the published data processing workflow (Forest et al., J. of Proteome Research 2018, 17(11):3657-3670).

A brief summary of the data processing is as follows:
Filter of frequency: data should be present in at least 80% in one group. This criterion was used to select only features with the highest intensity.
Normalization using cyclic loess algorithm using all MS scan signals present after filtering: cyclic loess normalization, commonly used for microarray data analysis, was selected among others as part of the validation method because it generates good alignment of signals, while allowing some variability in distribution, and thus, was more flexible than other algorithms such as quantile normalization.
Imputation of missing values using k-nearest neighbor (KNN) algorithm: this step was crucial to ensure robustness of subsequent statistical analyses, particularly with small datasets. Values are often not missing at random, and missing data can occur for different reasons. For imputation of missing values, the KNN algorithm (k=5), which classifies data based on similar measurements, was selected as it was previously shown to work best with strongly correlated features, such as lipids.
Correction for batch of sample preparation (sample lipid extraction) using the COMBAT algorithm: this step minimized variations due to sample processing, principally at the lipid extraction step, that were attributed to the inherent handling of samples across several days by different persons.
Filter of variation: RSD <80% was applied to corrected intensity data.

Following data processing, corrected raw data from positive and negative ionization for the entire experiment were merged together, resulting in a datafile of 3646 lipid features (defined by MS signal intensity, m/z, and RT).

### Statistical Analysis

A total of 3646 lipid features (defined by MS signal intensity, m/z, and RT) remained after data processing, of which approximately 50% represented potential unique lipids. The remainder were dimers, fragments, adducts, contamination, etc. This corrected dataset was used for all statistical analyses described below, which were conducted on corrected MS signal intensity expressed in log 2.

### Comparison Between Groups

FRDA subjects vs. healthy volunteers: linear regression analysis was done on each lipid feature, adjusted for age and sex, for the entire dataset of 3646 lipid features for FRDA subjects who were administered an exemplary FXN replacement therapeutic compound and healthy volunteers at Baseline (Day −2). A Storey correction was applied to estimate the false discovery rate (FDR, Q-value). Significance was set at a p-value <0.03, corresponding to a Q-value <0.14, and absolute fold-change >1.25. Lipid features were not annotated/identified at this step. The 95% confidence interval (CI) was also calculated for each feature fold-change between FRDA subjects and healthy volunteers.

Day 15 vs Day −2 for each of the 3 cohorts of FRDA subjects (i.e., subjects who were administered 25 mg, 50 mg or 100 mg of the exemplary FXN replacement therapeutic compound): a comparison between the MS signal intensity (log 2) for Day 15 vs Day −2 for all 3646 lipid features was performed using a paired t-test on each feature for each cohort. A Storey correction was applied to estimate the FDR (Q-value) in Cohorts 3 (i.e., subjects who were dosed with 100 mg of the exemplary FXN replacement therapeutic compound). Significance was set at a p-value <0.03, corresponding to a Q-value <0.08, for comparison of Day 15 vs Day −2 for Cohort 3. A subjective p-value was set at 0.03 for comparison in Cohorts 1 and 2. The 95% CI was also calculated for each feature fold-change between Day 15 vs Day −2 in each dose cohort individually, and 90% CI on fold-change of the results of this Example 4 vs. the results of Example 3 Day 15/Day −2 ratio for each FRDA patient for the reproducibility analysis of signal intensities.

### Correlation Analysis

Reproducibility of results from the MAD study (MAD1 vs MAD2): Pearson correlation was done between the MS signal intensity (log 2) of lipid features aligned between MAD2 and previously obtained results from MAD1 study to assess reproducibility of results between the 2 studies.

Network analysis was performed using the Fruchterman-Reingold layout algorithm on selected lipids. Positive correlations between selected lipid features or annotated unique lipids were projected onto a 2-dimensional display. A starting point was given to the algorithm as the first 2 principal component analysis coordinates.

### Lipid Annotations

Lipid annotation was performed using a proprietory database composed of more than 509 lipids previously annotated by MS/MS in human plasma and an algorithm to align mass and RT. This database is principally composed of glycerophospholipids and sphingolipids with their acyl chains and TGs with the sum of the 3 acyl chains. For the remaining lipids that were not annotated using the proprietory database, but were significantly different for the comparisons in this report, MS/MS analysis was performed to identify the lipid class and acyl chains when possible. Because acyl side chain positions were not validated with synthetic standards, phospholipids and glycerolipids were annotated along with the identified structure for the acyl side chains using the underscore convention for sn1 and sn2 positions.

Abbreviations for all lipid subclasses mentioned in this report are defined in Table 18.

TABLE 18
Lipid Subclass Abbreviations

| Abbreviation | Lipid Subclass |
|---|---|
| AC | acylcarnitine |
| CE | cholesteryl ester |
| Cer | ceramide |
| DG | diglyceride |
| Epoxy-TG | epoxy triglyceride |
| FFA | free fatty acid |
| Glc-Cer | glucosyl ceramide |
| LPC | lysophosphatidylcholine |
| LPE | lysophosphatidylethanolamine |
| PA | phosphatidic acid |
| PC | phosphatidylcholine |
| PCO— | ether phosphatidylcholine |
| PC(OH) | oxidized phosphatidylcholine |
| PE | phosphatidylethanolamine |
| PEO— | ether phosphatidylethanolamine |
| PI | phosphatidylinositol |
| SE | steryl ester |
| SM | sphingomyelin |
| TG | triglyceride |

For each subject with FRDA who was administered an exemplary FXN replacement therapeutic compound, samples were collected at Baseline (Day −2) and at Day 15. For each healthy volunteer, samples were collected from each subject at 2 time points, T₀ and T₃. All samples were analyzed by LC-MS/MS. Samples were injected into the LC-MS in the numeric order of MHI identification (ID).

### Results

### Assess the Differences in Baseline Levels of Select Lipid Subclasses Between Untreated FRDA Subjects and Healthy Volunteers

Comparisons were made between samples collected at Baseline (Day −2) for FRDA subjects (n=27) and T₀ for healthy volunteers (n=29). Comparisons were made based on the final corrected datasets, which included 3646 lipid features. A threshold p-value of 0.03 was chosen based on the comparison of Cohort 3 (paired t test Day 15 vs Day −2, Objective 2), corresponding to a Q value of 0.14.

Comparison of Plasma Lipid Features in Untreated FRDA Subjects vs. Healthy Volunteers

Of the 3646 lipid features, 390 discriminated FRDA subjects from healthy volunteers (p value <0.03, Q value <0.14, absolute fold change >1.25). Of these 390 significant features, 189 were annotated to a lipid ID. In total, 308 features (143 annotated) were higher and 82 features (46 annotated) were lower in FRDA subjects vs. healthy volunteers. The lipid subclasses with the highest number of annotated features were TGs, PCO-, and phosphatidylcholines (PCs).

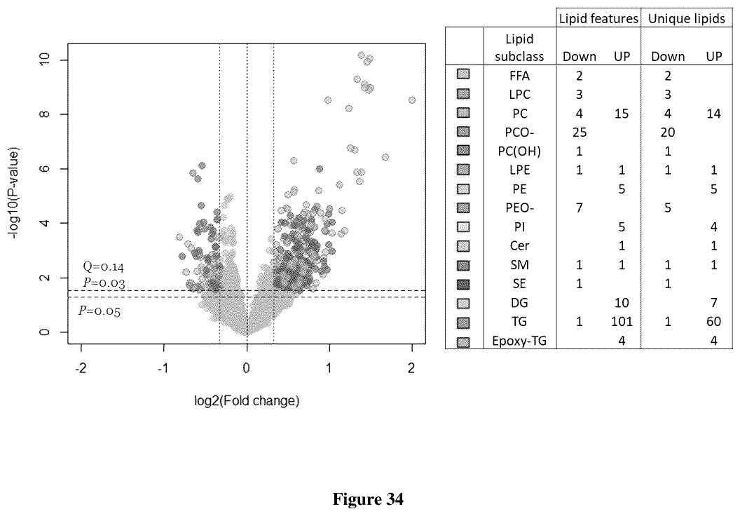

FIG. 34 shows a Volcano plot showing log 2 fold change for each lipid feature MS signal intensity for FRDA subjects (Day −2) vs. healthy volunteers (To) on the x-axis and the p-value (−log 10) on the y-axis. The horizontal dotted lines correspond to p-values of 0.05 and 0.03. The vertical dotted lines correspond to fold-changes of 0.8 (1/1.25; left) and 1.25 (right). The number of annotated lipids per subclass are shown in the table to the right of the volcano plot. The table displays the number of lipid features and unique lipids that were changed (up or down) for each lipid subclass. Lipid features showing higher values of fold-change in MS signal intensity for FRDA subjects vs. healthy volunteers are on the right of the graph, and those showing lower values are on the left of the graph.

### Triglycerides (TGs)

Overall, TGs had the greatest number of significant features. A total of 101 TG features were upregulated and 1 TG feature was downregulated between FRDA subjects and healthy volunteers (see FIG. 34).

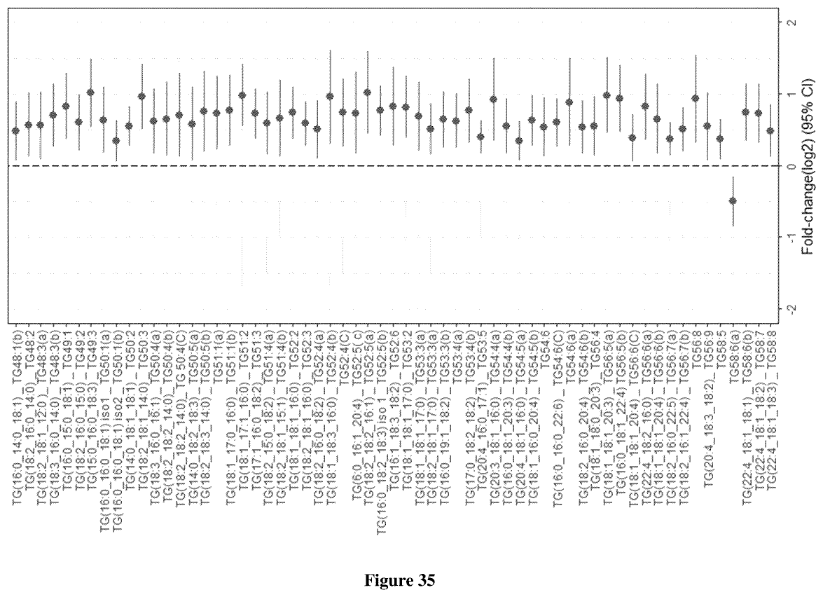

The number of unique TGs was obtained by removing duplicate signals from different adducts pertaining to corresponding significant TG features. This resulted in 61 unique TG species (p value <0.03, absolute fold change >1.25). FIG. 35 is a graph showing log 2 fold-change in various TG species that are significantly changed in untreated FRDA subjects vs. healthy volunteers.

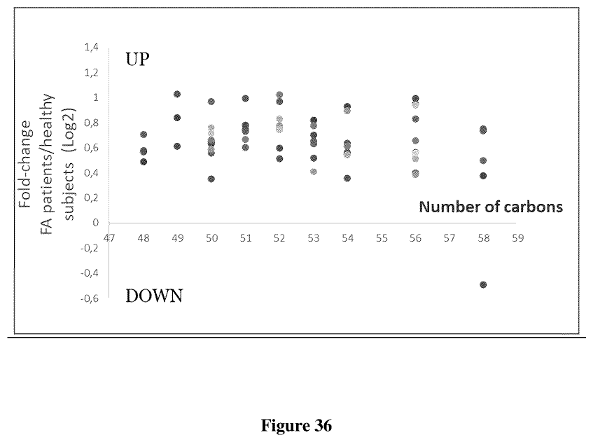

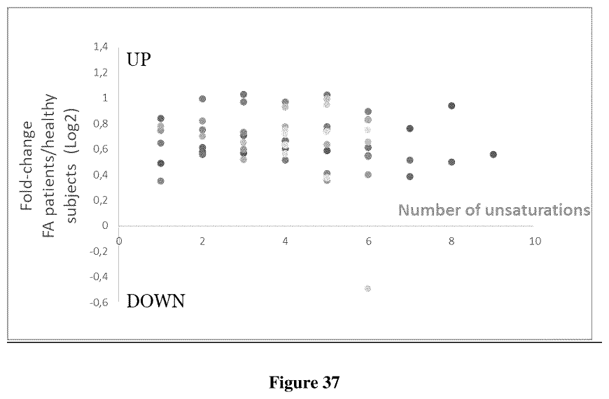

TG acyl chains were identified by MS/MS. Data are presented as the fold-change in MS signal intensity between FRDA subjects and healthy volunteers for the 61 significant unique TGs according to the structure of their acyl chain length, namely the number of carbons (FIG. 36) and unsaturations (FIG. 37). Specifically, FIG. 36 is a dot plot showing log 2 fold change in the levels of TGs vs. the number of acyl chain carbons for samples from untreated FRDA subjects vs. healthy volunteers. FIG. 37 is a dot plot showing log 2 fold change in the levels of TGs vs. the number of acyl chain unsaturations for samples from untreated FRDA subjects vs. healthy volunteers.

With the exception of TG58:6, the plasma levels of unique TGs, irrespective of the composition of their acyl chains, were higher in subjects with FRDA, as compared to healthy volunteers. The unique TGs that were upregulated in subjects with FRDA included TG48:1, TG48:2, TG48:3, TG49:1, TG49:2, TG49:3, TG50:1, TG50:2, TG50:3, TG50:4, TG50:5, TG51:1, TG51:2, TG51:3, TG51:4, TG52:2, TG52:3, TG52:4, TG52:5, TG52:6, TG53:2, TG53:3, TG53:4, TG53:5, TG54:4, TG54:5, TG54:6, TG56:4, TG56:5, TG56:6, TG56:7, TG56:8, TG56:9, TG58:5, TG58:7 and TG58:8. TG58:6 was the only TG species for which both the number of carbons (58) and unsaturations (6) in its acyl chain was higher in healthy volunteers than in subjects with FRDA.

### Ether Phosphatidylcholines (PCO-)

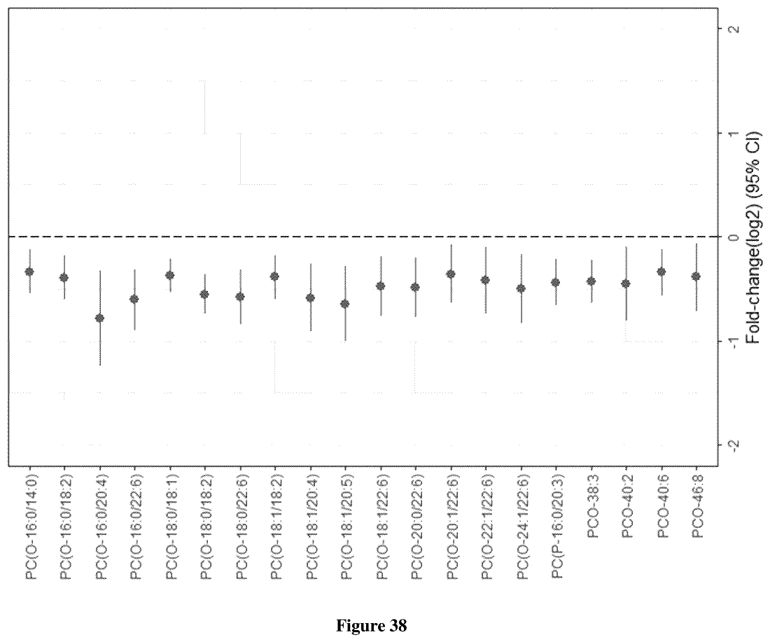

As shown in FIG. 34, a total of 25 PCO- features were downregulated between subjects with FRDA and healthy volunteers. There were 20 annotated unique PCO- lipid species that were significant for this comparison (p value <0.03, absolute fold change >1.25). FIG. 38 is a graph showing log 2 fold-change in various PCO- species that are significantly changed in untreated FRDA subjects vs. healthy volunteers.

Acyl chain compositions for these unique lipid species were identified by MS/MS. Plasma levels of all PCO- species, irrespective of the composition of their acyl chain, were lower in subjects with FRDA compared to healthy volunteers. The 20 annotated unique PCO- lipid species that were downregulated in untreated FRDA subjects included: PC(O-16:0/14:0), PC(O-16:0/18:2), PC(O-16:0/20:4), PC(O-16:0/22:6), PC(O-18:0/18:1), PC(O-18:0/18:2), PC(O-18:0/22:6), PC(O-18:1/18:2), PC(O-18:1/20:4), PC(O-18:1/20:5), PC(O-18:1/22:6), PC(O-(20:0/22:6), PC(O-20:1/22.6), PC(O-22:1/22:6), PC(O-(24:1/22:6), PC(O-16:0/20:3), PCO-38:3, PCO-40:2, PCO-40:6 and PCO-46:8.

### Phosphatidylcholines (PCs)

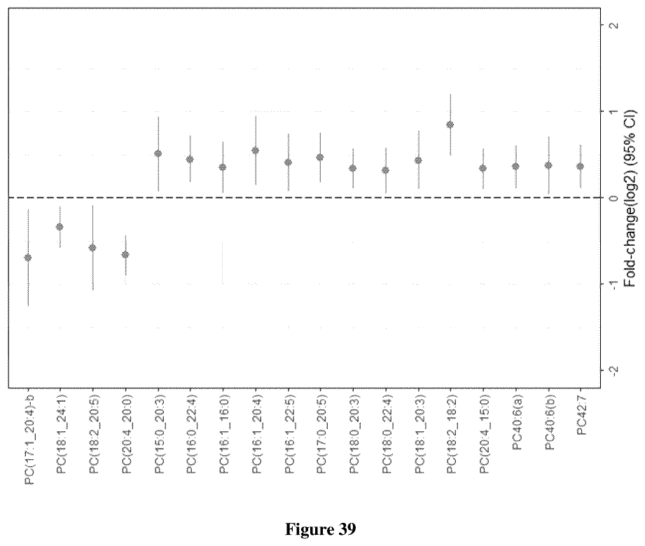

As shown in FIG. 34, a total of 15 PC features were upregulated and 4 PC features were downregulated between subjects with FRDA and healthy volunteers. There were 18 annotated unique PC lipid species that were significant for this comparison (p value <0.03, absolute fold change >1.25. FIG. 39 is a graph showing log 2 fold-change in various PC species that are significantly changed in untreated FRDA subjects vs. healthy volunteers.

Acyl chain compositions for these unique lipid species were identified by MS/MS. Plasma levels of most (14 out of 18) of the significant unique PC species were higher in subjects with FRDA compared to healthy volunteers. The 14 unique PC species that are upregulated in subjects with FRDA include PC(15:0/20:3), PC(16:0/22:4), PC(16:1/16:0), PC (16:1/20:4), PC(16:1/22:5), PC(17:0/20:5), PC(18:0/20:3), PC(18:0/22:4), PC(18:1/20:3), PC(18:2/18:2), PC(20:4/15:0), PC(40:6)(a), PC(40:6)(b) and PC(42:7). The 4 unique PC specs that are downregulated in subjects with FRDA include PC(17:1/20:4), PC(18:1/24:1), PC(18:2/20:5) and PC(20:4/20:0). The results indicate that changes in these lipid species may depend on their acyl chain composition.

### Network Analysis

Because high dimensional lipidomic data are expected to have a correlation structure, this was assessed for all 133 unique annotated lipids that were significant for the subjects with FRDA vs. healthy volunteer comparison (p-value <0.03; absolute fold-change >1.25, FIG. 34).

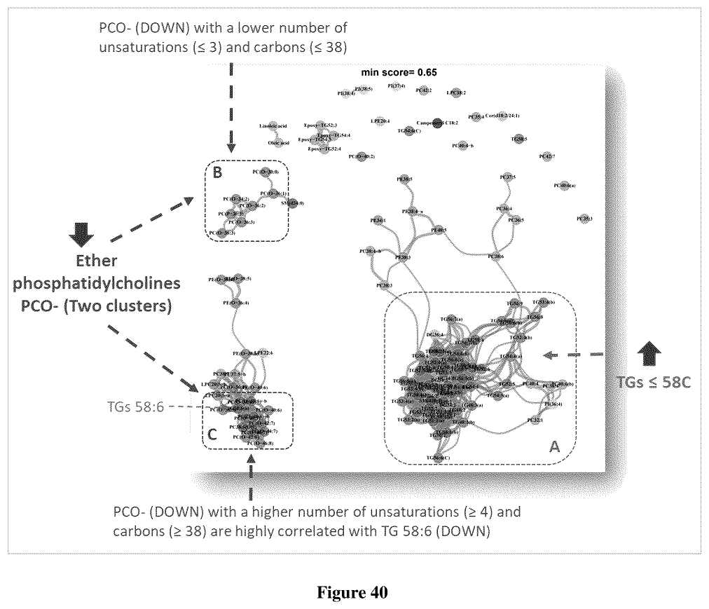

FIG. 40 is an illustration of a 2-dimensional display obtained using the Fruchterman-Reingold layout algorithm of positive correlations between annotated lipids differentially expressed in untreated FRDA subjects vs. healthy volunteers. FIG. 40 indicates that there are positive correlations (R>0.65) between lipids of the 2 datasets projected onto a 2 dimensional display using Fruchterman-Reingold layout algorithm.

Of the 133 annotated lipids significantly and differentially expressed in subjects with FRDA vs. healthy volunteers, all were highly correlated. There were 3 major independent correlation clusters or networks (groups of strongly correlated lipids) that demonstrated the difference between subjects with FRDA and healthy volunteers, likely reflecting a limited number of underlying mechanisms. One of these major correlation clusters (Cluster A) included all TGs that were higher in subjects with FRDA except TG58:6, which was lower in subjects with FRDA. The 2 other clusters (Cluster B and Cluster C) included PCO- species that differed according to their acyl chain composition: chain length ≤38C and number of unsaturations ≤3 (Cluster B), or chain length ≥38C and number of unsaturations ≥4 (Cluster C). Cluster C included TG58:6, which is lower in subjects with FRDA and contains both a high number of carbons and unsaturations. The chemical structure of the 3 acyl chains could not be identified by MS/MS.

Conclusions for the Analysis of Samples from Untreated FRDA Subjects Vs. Healthy Volunteers

The analysis of samples from untreated FRDA subjects vs. healthy volunteers reveals major perturbations in the plasma lipidome of untreated subjects with FRDA vs healthy volunteers (390/3646, or 11% of all lipid features in the dataset). The lipid subclasses with the highest number of annotated features were TGs, PCO-, and PCs.

TGs showed the highest number of lipid features modulated by FRDA, encompassing 102 (26%) of the 390 identified lipid features and corresponding to 61 unique lipids. Levels of most TG species (with the exception of TG58:6) were elevated in subjects with FRDA as compared to healthy volunteers, contained ≤58 carbons in their acyl chains, and were highly correlated in 1 cluster as revealed by network analysis.

PCO- represented 25 (6.4%) of all lipid features modulated by FRDA. Levels of all PCO- species were decreased in subjects with FRDA as compared to healthy volunteers and were associated with 2 correlation clusters, which differed according to the number of carbons and unsaturations. The cluster with PCO- species with a high number of carbons and unsaturations included TG58:6.

### Reproducibility of Results Between Example 3 and Example 4

PCs represented 19 (4.8%) of all lipid features modulated by FRDA. The majority (15/19) of PCs were higher in subjects with FRDA.

Observed changes in plasma lipids in subjects with FRDA are consistent with the proposed metabolic consequences of FXN deficiency in mitochondria (Crooks et al., J Biol Chem. 2018, 293(21):8297-8311). Specifically, the observed changed included increased de novo lipid synthesis as evidenced by the higher levels in TGs with <58 carbons and low unsaturations. These changes also suggest additional consequences on lipid metabolism in peroxisomes, as evidenced by the lower plasma levels of PCO-.

### Assessment of the Reproducibility of Results of Example 3

This example (Example 4) used the same samples from FRDA subjects dosed with an exemplary FXN replacement therapeutic compound for lipid analysis as the samples used in Example 3. One of the goals of this experiment was to determine reproducibility of the results obtained in Example 3.

### Methods and Results

Since this experiment used an untargeted approach, not all lipid features of the datasets were annotated. Features of interest were selected based on a significance threshold, and therefore datasets analyzed at different times may have different sets of annotated features (although subclasses may be the same). In order to compare signal intensities of the 2 untargeted lipidomic analyses, both datasets were aligned to ensure that the same lipid features were being compared.

The alignment of the 2 datasets from Example 3 and Example 4 was done on a subset of lipid features. The following 2 datasets were compared: (a) final corrected dataset with 3228 MS signals (lipid features) from Example 3; and (b) final corrected dataset with 3646 MS signals (lipid features) from this example (Example 4).

To align the Example 3 and Example 4 datasets, from the annotated features from Example 3, 593 features were selected from positive ionization since they covered all subclasses annotated in this dataset. Example 3 and Example 4 datasets were aligned to an in-house database using an in-house digital tool that enables RT correction between 2 LC-MS analyses. To include more lipids, data alignment was then completed manually using chromatograms. Overall, 192 annotated lipid features (188 unique lipids) were aligned in both datasets, which corresponded to 43% of unique lipid annotations for the Example 3 dataset and 42% for the Example 4 dataset.

The comparison of signal intensity values between the aligned Example 3 and Example 4 datasets was achieved using ratios of signal intensity values for Day 15 vs Day −2 in each subject. Comparison was done on signal intensity ratios based on the premise that if the analysis is reproducible, these ratios should be similar for Example 3 and Example 4. Signal intensity ratios were compared because absolute signal intensity values could be different between the 2 studies analyzed 7 months apart due to instrument sensitivity fluctuations. Each dataset was processed for correction and normalization independently.

The log 2 fold change between Example 3 and Example 4 datasets was calculated using the Day 15/Day −2 ratio. CI of 90% for each of the 192 annotated and aligned lipid features were also calculated. A given lipid was considered reproducible if the fold change and CI were within the threshold of fold change >0.8 and fold change <1.25. Overall, 169 lipid features (88%) were within the 90% CI threshold.

The 192 annotated and aligned lipid features were then restricted to include only lipid features that were significant (p<0.03) for Day 15 vs Day −2 in Cohort 3 (i.e., FRDA subjects who were administered 100 mg of the exemplary FXN replacement therapeutic compound). This resulted in 65 total lipid features and 56 lipid features (86%) within the 90% CI threshold.

Pearson correlations were calculated on normalized MS signal intensities (log 2) for each subject for the 192 annotated and aligned lipid features. In total, 146 lipid features (76%) were correlated between Example 3 and Example 4 (R>0.7), and 187 lipid features (97%) had a p value <0.01.

Linearity curves were plotted for selected lipid features and overall showed good reproducibility.

The comparison of Day 15 vs Day −2 for Cohort 3 was based on a discovery approach where a selected statistical significance threshold was applied independently on each dataset to identify lipid features that significantly discriminated the 2 time points.

From the 3228 lipid features in the Example 3 dataset, 452 features discriminated Day 15 vs Day 2 (p-value <0.03, Q-value <0.08, absolute fold change >1.25). Of these 452 features, 157 were annotated to a lipid ID. In total, 199 lipid features (44 annotated) were higher and 253 features (113 annotated) were lower at Day 15 vs Day −2.

From the 3646 lipid features in the Example 4 dataset, 227 features discriminated Day 15 vs Day 2 (p-value <0.03, Q-value <0.15, absolute fold change >1.25). Of these 227 features, 80 were annotated to a lipid ID. In total, 79 lipid features (17 annotated) were higher and 148 lipid features (63 annotated) were lower at Day 15 vs Day −2 (FIG. 13).

Overall, results demonstrated the same trends in both studies: TGs were predominantly downregulated and PCO- were predominantly upregulated. TGs with a high number of unsaturations were greater in Example 3 dataset (10 TGs) as compared to Example 4 dataset (3 TGs). However, although these lipids were below the selected thresholds chosen for the discovery approach (p value <0.03, absolute fold change >1.25), they followed the same trend in Example 3 and Example 4.

From the 192 lipid features (188 unique lipids) aligned between both datasets, those that were significant in Example 3 dataset (p-value <0.03, absolute fold-change >1.25) were selected for the comparison of Day 15 vs Day −2 in Cohort 3, resulting in 44 lipid features (43 unique lipids). Of the 43 unique lipids, 29 (67%) met the selected thresholds of reproducibility.

From the 192 lipid features (188 unique lipids) aligned between both datasets, those that were significant in Example 3 dataset (p-value <0.03, absolute fold change >1.25) were selected for the comparison of Day 15 vs Day −2 in Cohort 2, resulting in 13 lipid features (13 unique lipids). Of the 13 unique lipids, 6 (46%) met the selected thresholds of reproducibility.

From the 192 lipid features (188 unique lipids) aligned between both datasets, those that were significant in Example 3 dataset (p-value <0.03, absolute fold change >1.25) were selected for the comparison of Day 15 vs Day −2 in Cohort 1, resulting in 18 lipid features (18 unique lipids). Of the 18 unique lipids, 12 (67%), met the selected thresholds of reproducibility.

### Conclusions

Overall, there was good reproducibility between the Example 3 and Example 4 datasets based on comparative analyses using different statistical approaches, considering all lipid features aligned between the datasets and those that were significant after treatment.

Assessment of the Impact of on Lipid Profiles of FXN Deficiency and Treatment with an Exemplary FXN Replacement Therapeutic Compound

Comparisons were made using the dataset from this example only, since all samples from subjects with FRDA at baseline and after treatment with an exemplary FXN replacement therapeutic compound or placebo and healthy volunteers were injected simultaneously. This enabled comparison between data from the different groups.

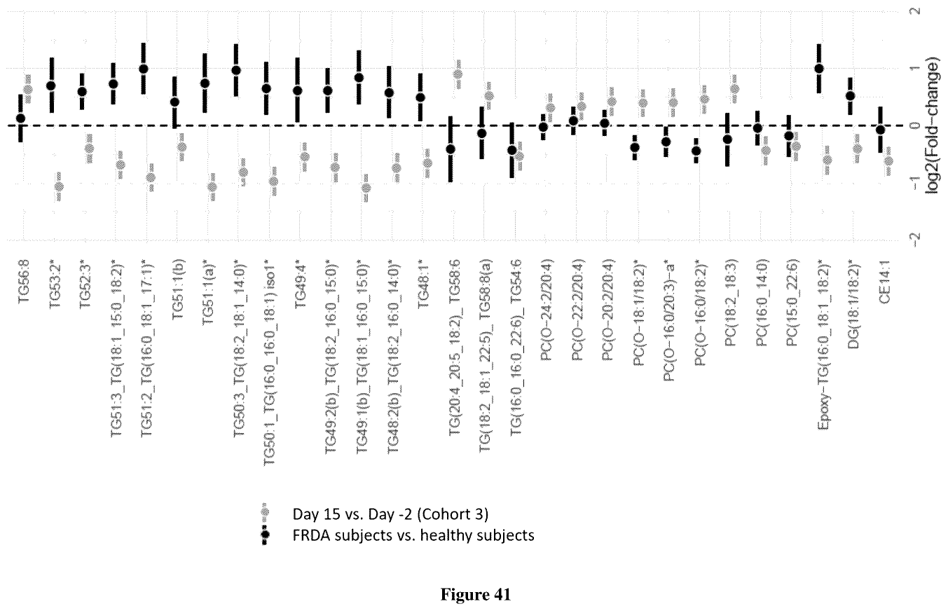

The 29 unique lipids identified as a result of comparison between Example 3 and Example 4 datasets that met the selected thresholds of reproducibility were compared as follows: (a) lipids were compared at Day 15 vs. Day −2 in Cohort 3 to determine impact of treatment; and (b) Day −2 in Cohort 3 vs. To in healthy volunteers to determine impact of disease. These results are presented in FIG. 41. Specifically, FIG. 41 is a forest plot representing log 2 fold change for the 29 unique lipids for FRDA subjects who were administered 100 mg of an exemplary FXN replacement therapeutic compound at Day 15 vs. Day −2 and for FRDA subjects vs. healthy volunteers.

In FIG. 41, fold-changes for a given lipid that fall on opposite sides of the 0 line indicate that the magnitude has shifted away from levels detected in untreated FRDA subjects. For many lipids that were elevated in FRDA subjects as compared to healthy volunteers, a reversal, or decrease was seen after treatment with an exemplary FXN replacement therapeutic compound. A similar phenomenon was seen for many lipids that were decreased in untreated FRDA subjects as compared to healthy volunteers. After treatment with an exemplary FXN replacement therapeutic compound, the levels shifted away from levels seen in untreated FRDA subjects and towards those of healthy volunteers.

Of the 29 unique lipids, 17 (59%) showed significant changes (p-value <0.05) in opposite directions for the 2 comparisons (indicated by * in FIG. 41). Twelve out of 17 of these lipids were TGs and showed changes that were opposite for the 2 comparisons (treatment vs. disease in FIG. 41). Three (50%) of the PCO- examined showed changes in opposite direction. These PCO- contained long chain fatty acids (16 and 18 carbons) compared to very long chain in sn1 position (20 to 24 carbons) for the 3 that did not change in opposite directions.

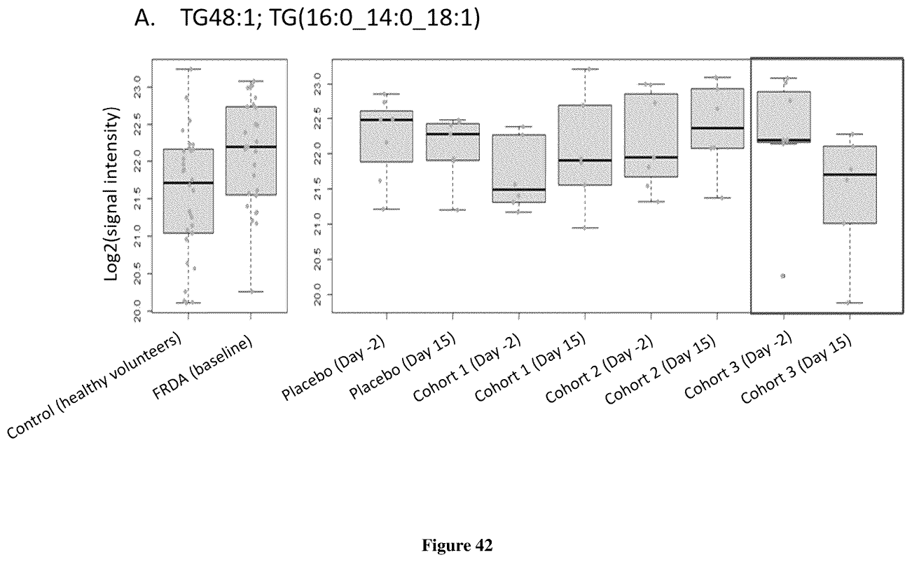

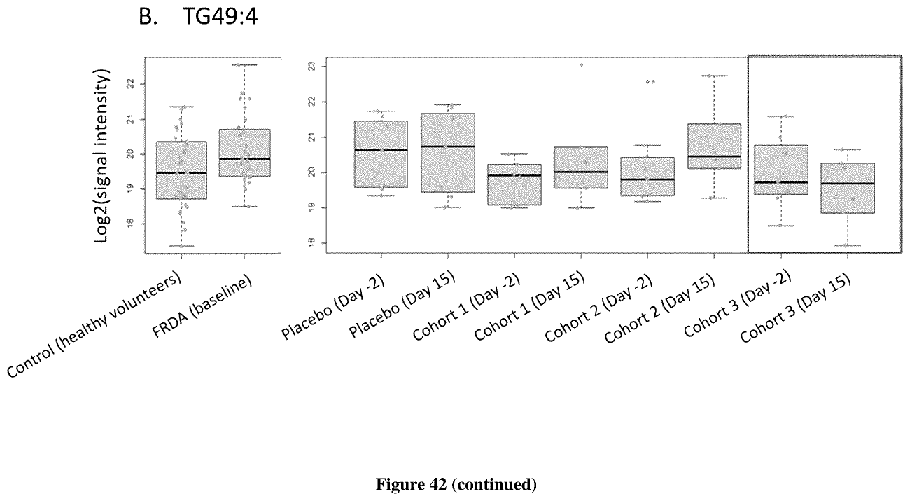

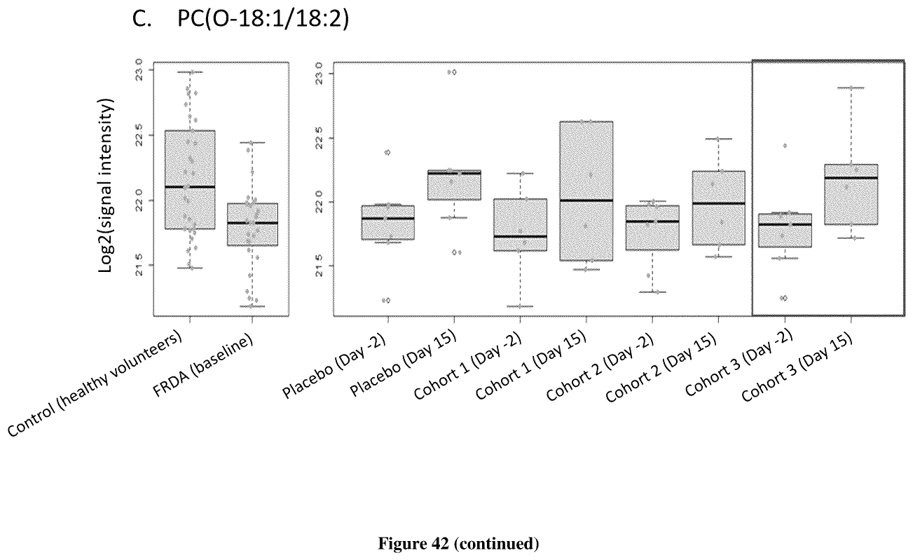

FIG. 42 shows representative boxplots of selected lipids showing the impact of treatment with a representative FXN replacement therapeutic compound on shifting levels towards those seen in healthy volunteers. Specifically, FIG. 42, panel A shows representative box plots for TG48:1, which shows changes in the opposite directions for the effect of disease (FRDA vs. healthy volunteers) and treatment (Day 15 vs Day −2) in FRDA subjects treated with 25 mg (Cohort 1), 50 mg (Cohort 2) and 100 mg (Cohort 3) of an exemplary FXN replacement therapeutic compound. FIG. 42, panel B shows representative box plots for TG49:4, which shows changes in the opposite directions for the effect of disease (FRDA vs. healthy volunteers) and treatment (Day 15 vs Day −2) in FRDA subjects treated with 25 mg (Cohort 1), 50 mg (Cohort 2) and 100 mg (Cohort 3) of an exemplary FXN replacement therapeutic compound. FIG. 42, panel C shows representative box plots for PC(O-18:1/18:2), which shows changes in the opposite directions for the effect of disease (FRDA vs. healthy volunteers) and treatment (Day 15 vs Day −2) in FRDA subjects treated with 25 mg (Cohort 1), 50 mg (Cohort 2) and 100 mg (Cohort 3) of an exemplary FXN replacement therapeutic compound.

### Conclusions

Among the 29 unique lipids that met selected thresholds of reproducibility and were significantly modulated in both Example 3 and Example 4, 17 (69%), predominantly TG and PCO- species, showed changes that were opposite for the impact of FRDA vs. treatment with an exemplary FXN replacement therapeutic compound, consistent with a return towards the profiles of healthy individuals.

1. (canceled)

2. A method for evaluating efficacy of a frataxin (FXN) replacement therapy, the method comprising:
(a) determining an FXN replacement lipid profile for one or more FXN-sensitive lipid markers (FSLMs) in a sample obtained from an FXN deficient subject following administration of an FXN replacement therapy;
(b) comparing the subject FXN replacement lipid profile determined in step (a) with a reference FXN lipid profile for the one or more FSLMs; and
(c) determining efficacy of the FXN replacement therapy based on the comparison in step (b);
wherein the one or more FSLMs are selected from a group consisting of one or more of:
triglycerides (TGs), wherein the three acyl groups in each triglyceride molecule contain less than 56 carbons and/or wherein the three acyl groups in each triglyceride molecule contain 7 or less unsaturations;
ether phospholipids;
phosphatidylcholines (PCs);
cholesteryl esters (CEs); and
diglycerides (DGs).

3. The method of claim 2, wherein the reference FXN lipid profile is a baseline FXN(−) lipid profile for the one or more FSLMs.

4. The method of claim 3, wherein the baseline FXN(−) lipid profile for the one or more FSLMs is determined in a sample obtained from an FXN deficient subject prior to administration of an FXN replacement therapy.

5. The method of claim 4, further comprising determining a baseline FXN(−) lipid profile for the one or more FXN-sensitive lipid markers (FSLMs) in a sample obtained from the FXN deficient subject prior to administration of the FXN replacement therapy.

6. The method of claim 2, wherein the one or more FSLMs are selected from a group consisting of one or more triglycerides (TGs), wherein the three acyl groups in each triglyceride molecule contain less than 56 carbons and/or wherein the three acyl groups in each triglyceride molecule contain 7 or less unsaturations.

7. The method of claim 6, wherein the one or more FSLMs are selected from the group consisting of TG45:1, TG46:1, TG46:3, TG47:1, TG47:2, TG48:0, TG48:1, TG48:2, TG48:3, TG49:1, TG49:2, TG49:3, TG49:4, TG50:1, TG50:2, TG50:3, TG50:4, TG50:5, TG51:1, TG51:2, TG51:3, TG51:4, TG52:2, TG52:3, TG52:4, TG52:5, TG52:6, TG53:2, TG53:3, TG53:4, TG53:5, TG54:4, TG54:5, TG54:6 and TG54:7.

8. (canceled)

9. The method of claim 2, wherein the one or more FSLMs are selected from one or more ether phospholipids.

10. The method of claim 9, wherein the one or more ether phospholipids comprise one or more ether diacylglycerophosphocholines (PCO-).

11. The method of claim 10, wherein the one or more PCO- are selected from the group consisting of PC(O-16:0/14:0), PC(O-16:0/18:2), PC(O-16:0/20:3), PC(O-16:0/20:4), PC(O-16:0/22:6), PC(O-17:0/20:4), PC(O-18:0/18:1), PC(O-18:0/22:6), PC(O-18:0/18:2), PC(O-18:1/18:2), PC(O-18:1/20:4), PC(O-18:1/20:5), PC(O-18:1/22:6), PC(O-(20:0/22:6), PC(O-20:1/22.6), PC(O-20:2/20:4), PC(O-22:2/20:4), PC(O-22:1/22:6), PC(O-22:2/20:4), PC(O-(24:1/22:6), PC(O-24:2/20:4), PCO-34:2, PCO-36:3, PCO-34:2, PCO-38:3, PCO-40:2, PCO-40:6, and PCO-44:7 and PCO-46.8.

12. (canceled)

13. The method of claim 9, wherein the one or more ether phospholipids comprise one or more phosphatidylethanolamine ethers (PEO-).

14. The method of claim 2, wherein the one or more FSLMs are selected from one or more phosphatidylcholines (PCs).

15. The method of claim 14, wherein the one or more PCs are selected from the group consisting of PC(15:0/20:3), PC(15:0/22:6), PC(16:0/14:0), PC(16:0/22:4), PC(16:1/16:0), PC(16:1/20:4), PC(16:1/22.5), PC(17:0/20:5), PC(18:0/20:3), PC(18:0/22:4), PC(18:1/20:3), PC(18:2/18:2), PC(20:4/15:0), PC40:6 and PC42:7.

16-18. (canceled)

19. The method of claim 2, wherein the one or more FSLMs are selected from one or more cholesteryl esters (CEs).

20. The method of claim 19, wherein the one or more CEs are selected from the group consisting of CE16:0, CE20:5 and CE14:1.

21. (canceled)

22. The method of claim 2, wherein the one or more FSLMs are selected from one or more diglycerides (DGs).

23. The method of claim 22, wherein the one or more DGs is DG18:1/18:2.

24. The method of claim 3, wherein the amount of at least one or more FSLMs is increased in the subject following treatment with FXN replacement therapy, wherein the one or more FSLMs are selected from the group consisting of PC(O-16:0/14:0), PC(O-16:0/18:2), PC(O-16:0/20:3), PC(O-16:0/20:4), PC(O-16:0/22:6), PC(O-17:0/20:4), PC(O-18:0/18:1), PC(O-18:0/22:6), PC(O-18:0/18:2), PC(O-18:1/18:2), PC(O-18:1/20:4), PC(O-18:1/20:5), PC(O-18:1/22:6), PC(O-(20:0/22:6), PC(O-20:1/22.6), PC(O-20:2/20:4), PC(O-22:2/20:4), PC(O-22:1/22:6), PC(O-22:2/20:4), PC(O-(24:1/22:6), PC(O-24:2/20:4), PCO-34:2, PCO-36:3, PCO-34:2, PCO-38:3, PCO-40:2, PCO-40:6, and PCO-44:7, PCO-46.8, PC(17:1/20:4), PC(18:2/18:3), PC(18:1/24:1), PC(18:2/20:5), PC(20:4/20:0), CE16:0 and CE20:5.

25. (canceled)

26. The method of claim 3, wherein the amount of at least one or more FSLMs is decreased in the subject following treatment with FXN replacement therapy, wherein the one or more FSLMs are selected from the group consisting of TG45:1, TG46:1, TG46:3, TG47:1, TG47:2, TG48:0, TG48:1, TG48:2, TG48:3, TG49:1, TG49:2, TG49:3, TG49:4, TG50:1, TG50:2, TG50:3, TG50:4, TG50:5, TG51:1, TG51:2, TG51:3, TG51:4, TG52:2, TG52:3, TG52:4, TG52:5, TG52:6, TG53:2, TG53:3, TG53:4, TG53:5, TG54:4, TG54:5, TG54:6, TG54:7, PC(15:0/20:3), PC(15:0/22:6), PC(16:0/14:0), PC(16:0/22:4), PC(16:1/16:0), PC(16:1/20:4), PC(16:1/22.5), PC(17:0/20:5), PC(18:0/20:3), PC(18:0/22:4), PC(18:1/20:3), PC(18:2/18:2), PC(20:4/15:0), PC40:6, PC42:7, DG18:1/18:2 and CE14:1.

27-29. (canceled)

30. The method of claim 3, wherein the FXN replacement therapy is determined to be effective when the amount of one or more FSLMs is increased in the FXN replacement lipid profile as compared to the baseline FXN(−) lipid profile,
wherein the one or more FSLMs are selected from the group consisting of PC(O-16:0/14:0), PC(O-16:0/18:2), PC(O-16:0/20:3), PC(O-16:0/20:4), PC(O-16:0/22:6), PC(O-17:0/20:4), PC(O-18:0/18:1), PC(O-18:0/22:6), PC(O-18:0/18:2), PC(O-18:1/18:2), PC(O-18:1/20:4), PC(O-18:1/20:5), PC(O-18:1/22:6), PC(O-(20:0/22:6), PC(O-20:1/22.6), PC(O-20:2/20:4), PC(O-22:2/20:4), PC(O-22:1/22:6), PC(O-22:2/20:4), PC(O-(24:1/22:6), PC(O-24:2/20:4), PCO-34:2, PCO-36:3, PCO-34:2, PCO-38:3, PCO-40:2, PCO-40:6, and PCO-44:7, PCO-46.8, PC(17:1/20:4), PC(18:2/18:3), PC(18:1/24:1), PC(18:2/20:5), PC(20:4/20:0), CE16:0 and CE20:5.

31. The method of claim 3, wherein the FXN replacement therapy is determined to be effective when the amount of one or more FSLMs is decreased in the FXN replacement lipid profile as compared to the baseline FXN(−) lipid profile,
wherein the one or more FSLMs are selected from the group consisting of TG45:1, TG46:1, TG46:3, TG47:1, TG47:2, TG48:0, TG48:1, TG48:2, TG48:3, TG49:1, TG49:2, TG49:3, TG49:4, TG50:1, TG50:2, TG50:3, TG50:4, TG50:5, TG51:1, TG51:2, TG51:3, TG51:4, TG52:2, TG52:3, TG52:4, TG52:5, TG52:6, TG53:2, TG53:3, TG53:4, TG53:5, TG54:4, TG54:5, TG54:6, TG54:7, PC(15:0/20:3), PC(15:0/22:6), PC(16:0/14:0), PC(16:0/22:4), PC(16:1/16:0), PC(16:1/20:4), PC(16:1/22.5), PC(17:0/20:5), PC(18:0/20:3), PC(18:0/22:4), PC(18:1/20:3), PC(18:2/18:2), PC(20:4/15:0), PC40:6, PC42:7, DG18:1/18:2 and CE14:1.

32-38. (canceled)

39. The method of claim 2, wherein the FXN lipid profile is determined by mass spectrometry.

40. (canceled)

41. The method of claim 2, wherein the subject has Friedreich's Ataxia (FRDA).

42. (canceled)

43. The method of claim 2, wherein the sample is selected from the group consisting of a buccal sample, a skin sample, a hair follicle or a blood-derived sample.

44. (canceled)

45. The method of claim 43, wherein the blood-derived sample is a plasma sample.

46. A method of monitoring treatment of a subject with a frataxin (FXN) replacement therapy, the method comprising:
(a) determining a first FXN replacement lipid profile for one or more FXN-sensitive lipid markers (FSLMs) in a first sample obtained from an FXN deficient subject at a first time point following administration of an FXN replacement therapy to the subject,
(b) determining a second FXN replacement lipid profile for the one or more FXN-sensitive lipid markers (FSLMs) in a second sample obtained from the subject at a second time point that is later than the first time point;
(c) comparing the second FXN replacement lipid profile with the first FXN replacement profile; thereby monitoring treatment of the subject with the FXN replacement therapy;
wherein the one or more FSLMs are selected from a group consisting of one or more of:
triglycerides (TGs), wherein the three acyl groups in each triglyceride molecule contain less than 56 carbons and/or wherein the three acyl groups in each triglyceride molecule contain 7 or less unsaturations;
ether phospholipids;
phosphatidylcholines (PCs)
cholesteryl esters (CEs); and
diglycerides (DGs).

47-61. (canceled)

62. A method for treating an FXN deficiency, the method comprising:
(a) determining an FXN lipid profile in a sample obtained from an FXN deficient subject for one or more FXN-sensitive lipid markers (FSLMs),
(b) comparing the FXN lipid profile of the sample with at least one other lipid profile selected from the group consisting of normal FXN lipid profile for the one or more FSLMs, baseline FXN(−) lipid profile for the one or more FSLMs, and FXN replacement lipid profile for the one or more FSLMs,
(c) classifying the FXN lipid profile determined in step (a) as corresponding to a normal FXN lipid profile, baseline FXN(−) lipid profile or an FXN replacement lipid profile, and
(d) initiating or modulating an FXN replacement therapy based on the classification of the FXN lipid profile of the sample.

63. The method of claim 62, wherein modulating an FXN replacement therapy comprises increasing the dosage, decreasing the dosage, increasing the administration frequency, or decreasing the administration frequency, of the FXN replacement therapy.

64. The method of claim 62, wherein the FXN deficient subject has Friedreich's Ataxia (FRDA).

65. A method of treating an FXN deficiency in a subject, comprising:
(a) determining an FXN lipid profile for one or more FXN-sensitive lipid markers (FSLMs) in a sample from an FXN deficient subject; and
(b) recommending to a healthcare provider to administer an FXN replacement therapy to the subject based on the subject FXN lipid profile determined in step (a).

66. A method of treating an FXN deficiency in a subject, comprising:
(a) obtaining an FXN lipid profile for one or more FSLMs in a sample obtained from an FXN deficient subject; and
(b) administering an FXN replacement therapy to the subject based on the subject FXN lipid profile.

67-68. (canceled)

69. A method of detecting one or more frataxin-sensitive lipid markers (FSLMs) in a sample from a frataxin (FXN) deficient subject, comprising
subjecting the sample, or a portion thereof, to analysis by mass spectrometry, wherein the one or more FSLMs are selected from a group consisting of:
triglycerides (TGs), wherein the three acyl groups in each triglyceride molecule contain less than 56 carbons and/or wherein the three acyl groups in each triglyceride molecule contain 7 or less unsaturations;
ether phospholipids;
phosphatidylcholines (PCs);
cholesteryl esters (CEs); and
diglycerides (DGs).

70. The method of claim 69, wherein the subject is being treated or is scheduled to be treated with an FXN replacement therapy.

71. The method of claim 69, further comprising obtaining the sample from the FXN deficient subject.

72. (canceled)

73. The method of claim 69, wherein the one or more FSLMs are selected from a group consisting of one or more triglycerides (TGs), wherein the three acyl groups in each triglyceride molecule contain less than 56 carbons and/or wherein the three acyl groups in each triglyceride molecule contain 7 or less unsaturations.

74. The method of claim 73, wherein the one or more FSLMs are selected from the group consisting of TG45:1, TG46:1, TG46:3, TG47:1, TG47:2, TG48:0, TG48:1, TG48:2, TG48:3, TG49:1, TG49:2, TG49:3, TG49:4, TG50:1, TG50:2, TG50:3, TG50:4, TG50:5, TG51:1, TG51:2, TG51:3, TG51:4, TG52:2, TG52:3, TG52:4, TG52:5, TG52:6, TG53:2, TG53:3, TG53:4, TG53:5, TG54:4, TG54:5, TG54:6 and TG54:7.

75. The method of claim 69, wherein the one or more FSLMs are selected from one or more ether phospholipids.

76. The method of claim 75, wherein the one or more ether phospholipids comprise one or more ether diacylglycerophosphocholines (PCO-).

77. The method of claim 76, wherein the one or more PCO- are selected from the group consisting of PC(O-16:0/14:0), PC(O-16:0/18:2), PC(O-16:0/20:3), PC(O-16:0/20:4), PC(O-16:0/22:6), PC(O-17:0/20:4), PC(O-18:0/18:1), PC(O-18:0/22:6), PC(O-18:0/18:2), PC(O-18:1/18:2), PC(O-18:1/20:4), PC(O-18:1/20:5), PC(O-18:1/22:6), PC(O-(20:0/22:6), PC(O-20:1/22.6), PC(O-20:2/20:4), PC(O-22:2/20:4), PC(O-22:1/22:6), PC(O-22:2/20:4), PC(O-(24:1/22:6), PC(O-24:2/20:4), PCO-34:2, PCO-36:3, PCO-34:2, PCO-38:3, PCO-40:2, PCO-40:6, and PCO-44:7 and PCO-46.8.

78. The method of claim 75, wherein the one or more ether phospholipids comprise one or more phosphatidylethanolamine ethers (PEO-).

79. The method of claim 69, wherein the one or more FSLMs are selected from one or more phosphatidylcholines (PCs).

80. The method of claim 79, wherein the one or more PCs are selected from the group consisting of PC(15:0/20:3), PC(15:0/22:6), PC(16:0/14:0), PC(16:0/22:4), PC(16:1/16:0), PC(16:1/20:4), PC(16:1/22.5), PC(17:0/20:5), PC(18:0/20:3), PC(18:0/22:4), PC(18:1/20:3), PC(18:2/18:2), PC(20:4/15:0), PC40:6, PC42:7, PC(17:1/20:4), PC(18:2/18:3), PC(18:1/24:1), PC(18:2/20:5) and PC(20:4/20:0).

81. The method of claim 69, wherein the one or more FSLMs are selected from one or more cholesteryl esters (CEs).

82. The method of claim 81, wherein the one or more CEs are selected from the group consisting of CE14:1, CE16:0 and CE20:5.

83. The method of claim 69, wherein the one or more FSLMs are selected from one or more diglycerides (DGs).

84. The method of claim 83, wherein the one or more DGs is DG18:1/18:2.

85. The method of claim 69, wherein the FXN lipid profile is determined by mass spectrometry.

86. The method of claim 69, wherein the subject has Friedreich's Ataxia (FRDA).

87. The method of claim 69, further comprising obtaining a sample from the FXN deficient subject, wherein the sample is selected from the group consisting of a buccal sample, a skin sample, a hair follicle or a blood-derived sample.

88-90. (canceled)

91. The method of claim 69, wherein the FXN replacement therapy comprises administration of an FXN fusion protein.

92. The method of claim 91, wherein the FXN fusion protein comprises or consists of the amino acid sequence set forth in SEQ ID NO: 12.

93. A kit for detecting one or more frataxin-sensitive lipid markers (FSLMs) in a sample obtained from a frataxin (FXN) deficient subject, comprising
one or more isotopically labeled lipids for use as internal standards in a mass spectrometry-based analysis for detecting one or more FSLMs in the sample, and
a set of instructions for detecting the level of the one or more FSLMs in the sample from the subject by mass spectrometry.

94. (canceled)
